(19) United States
(12) Patent Application Publication
(10) Pub. No.: US 2020/0291366 A1
(43) Pub. Date: Sep. 17, 2020

(54) PROSS OPTIMIZED ENZYMES

(71) Applicant: IGC BIO, INC., Cambridge, MA (US)

(72) Inventors: Lior ZIMMERMAN, Tel Aviv (IL); Dror BARAN, Te! Aviv (IL)

(73) Assignee: IGC BIO, INC., Cambridge, MA (US)

(21) Appl. No.: 16/646,298

(22) PCT Filed: Sep. 12, 2018
(86) PCT No.: PCT/US18/50641

§ 371 Date: Mar. 11, 2020

Related U.S. Application Data
(60) Provisional application No. 62/557,160, filed on Sep. 12, 2017.

(51) Int. Cl. C12N 9/10; C12N 9/20; C12N 9/88; C12N 9/04; C12N 9/16; C12N 9/02; C12N 9/06

(52) U.S. Cl. CPC C12N 9/1096; C12Y 206/01002; C12N 9/20; C12Y 301/01003; C12N 9/88; C12Y 405/01; C12Y 104/01001; C12Y 101/01001; C12N 9/16; C12Y 301/03002; C12N 9/001; C12Y 103/01; C12N 9/0016; C12N 9/0006

(57) ABSTRACT

The present invention provides enzymes that have been optimized by implementation of Protein Repair One Stop Shop (PROSS), an algorithm that generates protein design(s) for enhanced stability without changing either enzymatic properties or enzyme active site conformation of the respective enzyme. The protein design(s) generated by PROSS introduce mutations to the amino acid sequence of a wild-type protein, resulting in a mutated amino acid sequence that encodes a variant of the wild-type enzyme, i.e., “an enzyme variant”, which has an enhanced stability, core packing, surface polarity and backbone rigidity, a higher functional expression, and/or a combination thereof, compared to the stability core packing, surface polarity and backbone rigidity, functional expression and/or a combination thereof, of the wild-type enzyme.

### CROSS-REFERENCE TO RELATED APPLICATIONS

This application claims priority of U.S. Provisional Application No. 62/557,160, filed Sep. 12, 2017, which is hereby incorporated by reference.

### FIELD OF THE INVENTION

The present invention provides enzymes that have been optimized by implementation of Protein Repair One Stop Shop (PROSS), an algorithm that generates protein designs for enhanced stability without changing either enzymatic properties or enzyme active site conformation of the respective enzyme. The protein designs generated by PROSS introduce mutations to the amino acid sequence of a wild-type protein, resulting in a mutated amino acid sequence that encodes a variant of the wild-type enzyme, i.e., “an enzyme variant,” which has an enhanced stability, core packing, surface polarity and backbone rigidity, a higher functional expression, or a combination thereof, compared to the stability, core packing, surface polarity and backbone rigidity, functional expression or a combination thereof of the wild-type enzyme.

### BACKGROUND OF THE INVENTION

Enzymes have been used in various manufacturing process, although indirectly, since the dawn of man. Isolated enzymes were first used in the 1914, and their large scale microbial production started in the 1960. The industrial enzyme business is steadily growing: in 2004, market cap for industrial enzymes was $2.5 billion with a predicted annual growth rate of 5%-10%, according to BioSpectrum, Industrial enzyme: High on radar, Mar. 15, 2006. Indeed, I1 years later it was valued at $5 billion, as reported by Global Industrial Enzymes Market-Segmented by Type, Application, and Geography-Trends and Forecasts (2017-2022), Mordor Intelligence, 2017.

In 2017, major applications of industrial enzymes are in medicine, textile industry, food and beverages industry and in recent times as analytical aids. Detergents (17%), food and feed (17%), leather and paper (17%), textiles (8%) and pharmaceutical (41%) are the major industries that use industrially produced enzymes, as described by Chandel A K, et al., Industrial enzymes in bioindustrial sector development: An Indian perspective. J Comm Biotechnol 2007; 13:283-91.

As enzymes are used for more applications, there is a desire to use them in extreme environmental conditions, such as temperature, pH extremes and in the presence of salts, alkalis and surfactants. Major applications of enzymes are at high temperature (e.g., washing 60-70° C., starch gelatinization 100° C., textile desizing 80-90° C., etc.), under high salt concentrations (food industry), under alkaline conditions and in the presence of surfactants (e.g., in detergents and in several biotransformation reaction systems among others). Thus, there is a need to engineer enzymes to be extremely stable, far beyond their wild-type environment.

Approaches to stabilize enzymes and protein in general are usually based on phylogenetic reconstruction of ancestral sequences' (see, e.g., Lehmann M., et al., The consensus concept for thermostability engineering of proteins. Biochim. Biophys. Acta. 2000; 1543:408-415), and structure based rational or computational design (see, e.g., Borgo B., et al., Automated selection of stabilizing mutations in designed and natural proteins, Proc. Natl. Acad. Sci. USA. 2012; 109:1494-1499, and Jacak R., et al., Computational protein design with explicit consideration of surface hydrophobic patches, Proteins. 2012; 80:825-838), and had some success in engineering proteins with improved stability and higher functional expression per Jacak.

Recently, Goldenzweig et al. developed PROSS—an automated method for protein stabilization—and demonstrated its robustness on 5 proteins from a wide spectrum of organisms, as described by Goldenzweig A, et al., Automated Structure- and Sequence-Based Design of Proteins for High Bacterial Expression and Stability. Mol Cell. 2016; 63(2):337-46, and by Campeotto I, et al., One-step design of a stable variant of the malaria invasion protein RH5 for use as a vaccine immunogen. PNAS 2017 January 17. One target was human acetylcholinesterase (hAChE), an enzyme mediating synaptic transmission, which was designed to have 2000-fold higher expression rate in E. coli compared to the wild-type and exhibited 20° C. higher thermostability with no change in enzymatic properties or in active site conformation as was determined by X-Ray crystallography.

In light of its extreme robustness, the inventors applied PROSS to a number of industry relevant enzymes. Each PROSS iteration resulted in an amino acid sequence of an enzyme with an improved stability.

### SUMMARY OF THE INVENTION

Provided herein are pyruvate transaminase variants of a wild-type pyruvate transaminase from Vibrio fluvialis of SEQ ID NO: 39 comprising one or more mutations selected from A74E, Y76M, V103M, S108M, F130Y, Q196E, Q210L, A214P, G219A, L243Q, S304A, K305Q, T309E, A313K or A313R, E315G, E316T, A323Y, K335E, N342D, N405E; T406A, T408E, T408F or T408Q, and E436D.

In one embodiment, provided herein is a pyruvate transaminase variant of a wild-type pyruvate transaminase from Vibrio fluvialis of SEQ ID NO: 39 comprising mutations:
(a) S108M, F130Y, A214P, L243Q, S304A, E315G, A323Y, N342D, T408E, and E436D;
(b) S108M, F130Y, A214P, L243Q, S304A, E315G, A323Y, T408F, and E436D;
(c) Y76M, V103M, S108M, F130Y, A214P, G219A, L243Q, S304A, T309E, A313K, E315G, E316T, A323Y, N342D, N405E, T408Q, and E436D;
(d) Y76M, V103M, S108M, F130Y, A214P, L243Q, S304A, T309E, A313K, E315G, A323Y, N342D, T408Q, and E436D;
(e) Y76M, V103M, S108M, F130Y, A214P, G219A, L243Q, S304A, T309E, A313K, E315G, A323Y, N342D, N405E, T408Q, and E436D;
(f) A74E, Y76M, V103M, S108M, F130Y, Q196E, Q210L, A214P, G219A, L243Q, S304A, K305Q, T309E, A313R, E315G, E316T, A323Y, K335E, N342D, N405E, T406A, T408E, and E436D;
(g) Y76M, V103M, S108M, F130Y, A214P, G219A, L243Q, S304A, T309E, A313R, E315G, E316T, A323Y, N342D, T408Q, and E436D;
(h) A74E, Y76M, V103M, S108M, F130Y, Q196E, Q210L, A214P, G219A, L243Q, S304A, K305Q, T309E, A313K, E315G, E316T, A323Y, K335E, N342D, N405E, T406A, T408Q, and E436D; or
(i) Y76M, V103M, S108M, F130Y, A214P, G219A, L243Q, S304A, T309E, A313K, E315G, A323Y, N342D, T408Q, and E436D.

Also provided herein are triacylglycerol lipase variants of a wild-type triacylglycerol lipase from Burkholderia glumae of SEQ ID NO: 40 comprising one or more mutations selected from A63G, N64G, S74E, S78R, A106E, S132A, F161Y, T168Y, T176P, N183W, D196N, R204K, Q210G, T211A, Y214F, R216Q, A234P, S241N, Q242T, A297G, Q300P, F312Y, S319N, V334I, N338F, T348N or T348Q, L355N, and V358L.

In one embodiment, provided herein is a triacylglycerol lipase variant of a wild-type triacylglycerol lipase from Burkholderia glumae of SEQ ID NO: 40 comprising mutations:
(a) A63G, N64G, S74E, S78R, A106E, S132A, F161Y, T168Y, T176P, N183W, D196N, R204K, Q210G, T211A, Y214F, R216Q, A234P, S241N, Q242T, A297G, Q300P, F312Y, S319N, V334I, N338F, T348N, L355N, and V358L;
(b) A63G, N64G, S74E, S78Q, S132A, F161Y, T168Y, T176P, D196N, R204K, Q210G, T211A, Y214F, A234P, S241N, Q242T, A297G, Q300P, F312Y, S319N, V334I, T348N, L355N, and V358L;
(c) N64G, S132A, D196N, A234P, S241N, Q242T, A297G, Q300P, S319N, T348N, L355N, and V358L;
(d) A63G, N64G, S74E, S78Q, S132A, F161Y, T168Y, T176P, D196N, R204K, Q210G, T211A, Y214F, A234P, S241N, Q242T, A297G, Q300P, F312Y, S319N, V334I, T348Q, L355N, and V358L;
(e) S241N, A297G, Q300P, S319N, and V358L;
(f) N64G;
(g) S132A, D196N, A234P, S241N, Q242T, A297G, Q300P, F312Y, S319N, T348N, L355N, and V358L; or
(h) S241N, A297G, Q300P, S319N, T348N, and V358L.

Provided herein are halohydrin dehalogenase variants of a wild-type halohydrin dehalogenase from Rhizobium radiobacter of SEQ ID NO: 41 comprising one or more mutations selected from T3V, K10R, F12G, S17A, S22A, E33P, Q37D, K38P, L41R, K52H, E56A, I63V, A65R, T67I; S68K or S68E; S78A, D80A, A83P, Q87R; K91Q or K91E; A93D or A93S; V94P; G99A or G99K; Q105L, R107G, A110R, N113R or N113Q, S117P, F136L, W139L, K140P, E141G, S147A; C153I or C153N; T154A, N157K, S160A, V205Q, T206V, K215E and M245V.

In one embodiment, provided is a halohydrin dehalogenase variant of a wild-type halohydrin dehalogenase from Rhizobium radiobacter of SEQ ID NO: 41 comprising mutations:
(a) T3V, F12G, S17A, E33P, Q37D, K38P, L41R, K52H, E56A, T67I, S68K, S78A, D80A, A83P, Q87R, K91Q, V94P, G99A, R107G, A110R, N113R, S117P, K140P, E141G, C153I, T154A, N157K, S160A, V205Q, and K215E;
(b) F12G, E33P, Q37D, L41R, T67I, S68K, D80A, A83P, Q87R, V94P, G99A, N113R, S117P, K140P, C153I, T154A, and N157K;
(c) F12G, E33P, Q37D, L41R, T67I, S68K, A83P, Q87R, V94P, G99A, N113R, S117P, C153I, T154A, and N157K;
(d) T3V, K10R, F12G, S17A, S22A, E33P, Q37D, K38P, L41R, K52H, E56A, I63V, T67I, S68K, S78A, D80A, A83P, Q87R, K91E, A93D, V94P, G99K, Q105L, R107G, A110R, N113Q, S117P, F136L, W139L, E141G, S147A, C153N, T154A, N157K, S160A, V205Q, K215E, and M245V;
(e) F12G, S17A, E33P, Q37D, K38P, L41R, K52H, E56A, T67I, S68K, D80A, A83P, Q87R, V94P, G99A, R107G, A110R, N113R, S117P, C153N, T154A, N157K, V205Q, and K215E;
(f) T3V, K10R, F12G, S17A, S22A, E33P, Q37D, K38P, L41R, K52H, I63V, A65R, T67I, S68E, S78A, D80A, A83P, Q87R, K91E, A93D, V94P, G99A, Q105L, R107G, A110R, N113R, S117P, F136L, W139L, E141G, S147A, C153I, T154A, N157K, S160A, V205Q, and K215E;
(g) F12G, S17A, E33P, Q37D, K38P, L41R, K52H, E56A, T67I, S68K, D80A, A83P, Q87R, V94P, G99A, R107G, A110R, N113Q, S117P, K140P, C153I, T154A, N157K, V205Q, and K215E;
(h) T3V, K10R, F12G, S17A, S22A, E33P, Q37D, K38P, L41R, K52H, E56A, T67I, S68E, S78A, D80A, A83P, Q87R, K91E, A93S, V94P, G99A, R107G, A110R, N113R, S117P, W139L, E141G, S147A, C153I, T154A, N157K, S160A, V205Q, T206V, and K215E;
(i) F12G, E33P, L41R, T67I, S68K, A83P, Q87R, V94P, G99A, N113R, S117P, K140P, C153I, T154A, and N157K;
(j) T3V, K10R, F12G, S17A, S22A, E33P, Q37D, K38P, L41R, K52H, E56A, T67I, S68E, S78A, D80A, A83P, Q87R, K91E, A93S, V94P, G99A, R107G, A110R, N113R, S117P, W139L, K140P, E141G, S147A, C153I, T154A, N157K, S160A, V205Q, and K215E;
(k) T3V, F12G, S17A, E33P, Q37D, K38P, L41R, K52H, E56A, T67I, S68K, S78A, D80A, A83P, Q87R, K91Q, V94P, G99A, R107G, A110R, N113Q, S117P, E141G, C153N, T154A, N157K, S160A, V205Q, and K215E;
(l) F12G, E33P, Q37D, K38P, L41R, K52H, E56A, T67I, S68K, D80A, A83P, Q87R, V94P, G99A, R107G, A110R, N113R, S117P, K140P, C153I, T154A, and N157K; or
(m) F12G, E33P, L41R, T67I, S68K, D80A, A83P, Q87R, V94P, G99A, N113R, S117P, K140P, C153N, T154A, and N157K.

Also provided herein are aminotransferase variants of a wild-type aminotransferase from Chromobacterium violaceum of SEQ ID NO: 42 comprising one or more mutations selected from M36L, G39A, D70E, F71L, R76Y, K90Q, V97I, S101E or S101K, A109P, D112N, R113H, S123A, V124N, T145V, L146I, G152A, S162A, V202L; V203H or V203K; A217P, D315E, A337R, V236I, A239D, H276Y; A286I or A286V; K304D, A312E, H333L; A337K or A337R; Q346E, K358Q, E368P; V379L; V379I or V379M; Q380A, N387D, K390T, D396N, F397E, T402W, R410N, S424A, T452E; and A455E or A455K.

In one embodiment, provided herein is an aminotransferase variant of a wild-type aminotransferase from Chromobacterium violaceum of SEQ ID NO: 42 comprising mutations:
(a) G39A, F71L, S101E, A109P, D112N, R113H, S123A, V124N, L146I, V202L, V203H, A217P, V236I, A239D, A286I, K304D, A312E, H333L, A337K, E368P, V379L, Q380A, D396N, F397E, T402W, and A455E;
(b) D70E, D112N, R113H, S123A, V124N, A217P, A337R, E368P, Q380A, D396N, and F397E;
(c) G39A, D70E, F71L, S101E, A109P, D112N, R113H, S123A, V124N, L146I, A217P, V236I, A286I, H333L, A337K, E368P, V379L, Q380A, D396N, F397E, and A455E;
(d) G39A, D112N, R113H, S123A, V124N, A217P, A337R, E368P, V379L, Q380A, D396N, and F397E;
(e) D70E, D112N, R113H, S123A, V124N, A217P, A286V, A337R, E368P, V379L, Q380A, D396N, and F397E;
(f) G39A, D70E, F71L, S101K, A109P, D112N, R113H, S123A, V124N, L146I, V202L, V203H, A217P, V236I, A239D, A286I, K304D, A312E, H333L, A337R, E368P, V379L, Q380A, D396N, F397E, T402W, and A455E;
(g) G39A, D70E, F71L, K90Q, V97I, S101E, A109P, D112N, R113H, S123A, V124N, T145V, L146I, G152A, V202L, V203H, A217P, V236I, A239D, H276Y, A286I, K304D, A312E, H333L, A337K, Q346E, E368P, V379I, Q380A, N387D, D396N, F397E, T402W, R410N, T452E, and A455K;
(h) M36L, G39A, D70E, F71L, K90Q, V97I, S101K, A109P, D112N, R113H, S123A, V124N, T145V, L146I, G152A, V202L, V203K, A217P, V236I, A239D, H276Y, A286I, K304D, A312E, H333L, A337K, Q346E, K358Q, E368P, V379M, Q380A, N387D, K390T, D396N, F397E, T402W, R410N, S424A, T452E, and A455K;
(i) M36L, G39A, D70E, F71L, R76Y, K90Q, V97I, S101K, A109P, D112N, R113H, S123A, V124N, T145V, L146I, G152A, V202L, V203K, A217P, V236I, A239D, H276Y, A286I, K304D, A312E, H333L, A337R, Q346E, K358Q, E368P, Q380A, N387D, K390T, D396N, F397E, T402W, R410N, S424A, T452E, and A455E;
(j) G39A, D70E, S101E, A109P, D112N, R113H, S123A, V124N, L146I, A217P, A286I, H333L, A337K, E368P, V379M, Q380A, D396N, and F397E;
(k) G39A, D70E, F71L, S101E, A109P, D112N, R113H, S123A, V124N, L146I, A217P, V236I, A286I, D315E, H333L, A337R, E368P, Q380A, D396N, F397E, and A455E;
(l) G39A, D70E, F71L, K90Q, V97I, S101E, A109P, D112N, R113H, S123A, V124N, T145V, L146I, G152A, S162A, V202L, V203H, A217P, V236I, A239D, H276Y, A286I, K304D, A312E, H333L, A337R, Q346E, E368P, V379I, Q380A, N387D, D396N, F397E, T402W, R410N, T452E, and A455K;
(m) G39A, D70E, D112N, R113H, S123A, V124N, A217P, A286V, A337R, E368P, V379L, Q380A, D396N, and F397E; or
(n) G39A, S101E, A109P, D112N, R113H, S123A, V124N, L146I, A217P, A286I, H333L, A337R, E368P, V379L, Q380A, D396N, and F397E.

Also provided herein are putative secreted lipase variants of a wild-type putative secreted lipase from Clostridium botulinum of SEQ ID NO: 43 comprising one or more mutations selected from D69N, E91Q, I101T, K137D, A140R, S141N, H143Y, V168K, K183G, K196P, N200D, K246H, S253V, S265A, T277M, K289N, N308D, E339Y, T346K, A375P, S395T, Q398K, G437T, M469I, N477Q, and S479P.

In one embodiment, also provided is a putative secreted lipase variant of a wild-type putative secreted lipase from Clostridium botulinum of SEQ ID NO: 43 comprising mutations:
(a) E91Q, I101T, A140R, K183G, K246H, S265A, T277M, K289N, N308D, E339Y, A375P, S395T, G437T, and S479P;
(b) I101T, K183G, T277M, K289N, N308D, A375P, S395T, and S479P;
(c) D69N, E91Q, I101T, K137D, A140R, S141N, H143Y, V168K, K183G, K196P, N200D, K246H, S265A, T277M, K289N, N308D, E339Y, T346K, A375P, S395T, Q398K, G437T, M469I, N477Q, and S479P;
(d) I101T, K183G, T277M, K289N, A375P, S395T, and S479P;
(e) D69N, E91Q, I101T, K137D, A140R, S141N, H143Y, V168K, K183G, K196P, N200D, K246H, S265A, T277M, K289N, N308D, E339Y, T346K, A375P, S395T, Q398K, G437T, N477Q, and S479P;
(f) I101T, K183G, K246H, S253V, T277M, K289N, N308D, A375P, S395T, G437T, and S479P;
(g) I101T, K183G, K246H, T277M, K289N, N308D, A375P, S395T, G437T, and S479P; or
(h) K183G, T277M, A375P, S395T, and S479P.

Further provided herein are alcohol dehydrogenase variants of a wild-type alcohol dehydrogenase from Geobacillus stearothermophilus of SEQ ID NO: 44 comprising one or more mutations selected from K10G; K11E or K11Q; Q14K or Q14V; V15I, E19P, K20V or K20I, K22T, S24G, V66I, I67V, E68V, E69A, Y89W, Q102W, R108H, Q110K, A112T, Y120F, A127D, K133H, F147L, T153V, V159E or V159M; V179M, L196I, G197D; E199D, K205R, Q206E or Q206K; K216L, H217K, D218E, A220P, Q222K, W223F, I224M, E226K, T234A, A242P, E245Q, S246Q, K249R or K249N; I251L, A256T, C257V or C257L; E265G, I267M or I267F, I269V or I269L; V279I, V28I, T306P or T306V; V308I, Q311R; N315K or N315D; D318E, L324R, N329D or N329K, K335D, V336L, D337E, K335D, V336L, and D337E.

Also provided herein is an alcohol dehydrogenase variant of a wild-type alcohol dehydrogenase from Geobacillus stearothermophilus of SEQ ID NO: 44 comprising mutations:
(a) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, I269V, V279I, V308I, Q311R, N315K, D318E, K335D, V336L, and D337E;
(b) K10G, K11E, Q14K, V15I, E19P, K20V, K22T, S24G, V66I, I67V, Q102W, R108H, Q110K, A112T, A127D, K133H, V179M, L196I, G197D, Q206E, K216L, H217K, D218E, A220P, Q222K, W223F, I224M, T234A, A242P, E245Q, S246Q, K249R, A256T, C257V, I267M, V279I, V308I, Q311R, N315K, D318E, K335D, V336L, and D337E;
(c) K10G, K11Q, Q14V, V15I, E19P, K20I, K22T, S24G, V66I, I67V, E68V, E69A, Y89W, Q102W, R108H, A112T, Y120F, A127D, K133H, T153V, V159E, V179M, L196I, G197D, E199D, Q206K, K216L, H217K, D218E, A220P, Q222K, W223F, I224M, E226K, T234A, A242P, S246Q, K249R, I251L, A256T, C257L, E265G, I267M, I269V, V279I, T306P, V308I, Q311R, N315D, D318E, L324R, N329D, K335D, V336L, and D337E;
(d) K10G, K11Q, Q14K, V15I, E19P, K20I, K22T, 524G, V66I, I67V, Q102W, R108H, Q110K, A112T, Y120F, A127D, K133H, F147L, T153V, V159E, V179M, L196I, G197D, Q206E, K216L, H217K, D218E, A220P, Q222K, W223F, I224M, T234A, A242P, E245Q, S246Q, K249R, A256T, C257L, I267M, I269V, V279I, T306V, V308I, Q311R, N315D, D318E, N329D, K335D, V336L, and D337E;
(e) K10G, K11Q, Q14V, V15I, E19P, K20I, K22T, S24G, V28I, V66I, I67V, E68V, E69A, Y89W, Q102W, R108H, A112T, Y120F, A127D, K133H, T153V, V159E, V179M, L196I, G197D, E199D, Q206K, K216L, H217K, D218E, A220P, Q222K, W223F, I224M, E226K, T234A, A242P, S246Q, K249R, I251L, A256T, C257L, E265G, I267M, I269V, V279I, T306P, V308I, Q311R, N315D, D318E, L324R, N329D, K335D, V336L, and D337E;
(f) K10G, K11Q, Q14K, V15I, E19P, K20I, K22T, S24G, V66I, I67V, Q102W, R108H, Q110K, A112T, A127D, K133H, V159M, V179M, L196I, G197D, Q206E, K216L, H217K, D218E, A220P, Q222K, W223F, I224M, T234A, A242P, E245Q, S246Q, K249R, A256T, C257V, I267F, I269L, V279I, T306P, V308I, Q311R, D318E, N329K, K335D, V336L, and D337E;
(g) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, V279I, V308I, N315K, D318E, K335D, V336L, and D337E;
(h) K10G, K11E, Q14K, V15I, E19P, K22T, S24G, V66I, I67V, Q102W, R108H, Q110K, A127D, K133H, V159E, V179M, L196I, G197D, Q206E, K216L, H217K, D218E, A220P, Q222K, W223F, I224M, T234A, A242P, E245Q, K249N, A256T, I267M, I269V, V279I, V308I, N315K, D318E, K335D, V336L, and D337E;
(i) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, I269V, V279I, V308I, N315K, D318E, K335D, V336L, and D337E;
(j) K10G, K11Q, Q14V, V15I, E19P, K20I, K22T, S24G, V66I, I67V, E69A, Y89W, Q102W, R108H, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249N, I251L, A256T, C257L, I267M, I269L, V279I, T306P, V308I, Q311R, N315D, D318E, N329D, K335D, V336L, and D337E;
(k) K10G, K11Q, Q14V, V15I, E19P, K20I, K22T, S24G, V66I, I67V, E69A, Y89W, Q102W, R108H, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249R, I251L, A256T, C257L, I267M, I269V, V279I, T306P, V308I, Q311R, N315D, D318E, N329D, K335D, V336L, and D337E;
(l) K10G, K11Q, Q14K, V15I, E19P, K20I, K22T, S24G, V66I, I67V, Q102W, R108H, Q110K, A112T, A127D, K133H, F147L, V159M, V179M, L196I, G197D, K205R, Q206E, K216L, H217K, D218E, A220P, Q222K, W223F, I224M, T234A, A242P, E245Q, S246Q, K249R, A256T, C257V, I267M, I269V, V279I, T306V, V308I, Q311R, D318E, N329K, K335D, V336L, and D337E;
(m) K10G, K11Q, Q14K, V15I, E19P, K20I, K22T, S24G, V66I, I67V, Y89W, Q102W, R108H, Q110K, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249R, A256T, C257L, I267F, I269L, V279I, T306V, V308I, Q311R, N315D, D318E, N329D, K335D, V336L, and D337E; or
(n) K10G, K11E, Q14K, V15I, E19P, K22T, S24G, V66I, I67V, Q102W, R108H, Q110K, A127D, K133H, V159E, V179M, L196I, G197D, Q206E, K216L, H217K, D218E, A220P, Q222K, W223F, I224M, T234A, A242P, E245Q, K249R, A256T, I267F, I269V, V279I, V308I, N315K, D318E, K335D, V336L, and D337E.

Provided herein are histidine acid phosphatase variants of a wild-type histidine acid phosphatase from Yersinia mollaretii of SEQ ID NO: 45 comprising one or more mutations selected from S42A, Q46P or Q46F, D52N, D56H or D56Q, K57P, A65P, T77R or T77K, G81Q or G81R, F82Y, Y83F, N89Q, T102E, Q106W, I109S or I109T, L115A, A136P, V141P or V141M, E149K, Q154T or Q154R, S157P, T158K or T158E, H161R or H161K, R162A, E165M, Q167R, A170G, S173D, E174A, K181D, P182D or P182S, Q185L, G187Q or G187E, E188Q, I189V, T193C or T193K, A194N, 5200A or 5200K, 5207T, N221S, Q222K, Q223E, S230E, S236A, G240A, N247Y, S248A, H257G, A262E, V266R, S267E, L269M, Q275K or Q275Y, K289Q, V298L, K306D, G324A, G334A, Q342T, Q345G, G355A, N363D, D365K, M380L, S386A, K392T, S393N, H394N, I409T, T411D, Q416P, and A425K.

In one embodiment, provided herein is a histidine acid phosphatase variant of a wild-type histidine acid phosphatase from Yersinia mollaretii of SEQ ID NO: 45 comprising mutations:
(a) S42A, Q46P, D52N, D56H, K57P, A65P, T77R, G81Q, F82Y, Y83F, N89Q, Q106W, I109 S, L115A, A136P, V141P, E149K, Q154T, S157P, T158K, H161R, R162A, E165M, Q167R, A170G, S173D, E174A, K181D, P182D, Q185L, G187Q, E188Q, T193C, A194N, S200A, 5207T, N221S, Q222K, Q223E, S230E, S236A, G240A, N247Y, S248A, H257G, A262E, V266R, Q275K, K289Q, V298L, K306D, G324A, G334A, Q342T, Q345G, G355A, N363D, D365K, M380L, S386A, K392T, S393N, H394N, I409T, T411D, Q416P, and A425K;
(b) Q46F, D56H, K57P, A65P, T77K, G81Q, F82Y, Y83F, N89Q, T102E, Q106W, L115A, V141M, Q154T, S157P, T158E, H161R, R162A, E165M, A170G, E174A, Q185L, G187Q, T193C, A194N, S200K, S230E, S236A, G240A, N247Y, S248A, A262E, V266R, V298L, K306D, G324A, G334A, Q345G, N363D, S386A, I409T, and T411D;
(c) Q46F, K57P, A65P, T77K, G81R, N89Q, Q106W, L115A, Q154R, S157P, T158E, H161K, E165M, A170G, E174A, Q185L, G187Q, T193C, S236A, G240A, N247Y, S248A, L269M, V298L, K306D, G324A, G334A, Q345G, and T411D;
(d) Q46F, D56Q, K57P, A65P, T77K, G81R, F82Y, Y83F, N89Q, T102E, Q106W, I109T, L115A, V141P, Q154T, S157P, T158E, H161R, R162A, E165M, A170G, E174A, Q185L, G187Q, E188Q, T193C, A194N, S200K, Q222K, Q223E, S230E, S236A, G240A, N247Y, S248A, H257G, A262E, V266R, S267E, L269M, V298L, K306D, G324A, G334A, Q342T, Q345G, G355A, N363D, S386A, S393N, H394N, I409T, T411D, and A425K;
(e) S42A, Q46F, D52N, D56H, K57P, A65P, T77R, G81Q, F82Y, Y83F, N89Q, T102E, Q106W, I109T, L115A, A136P, V141P, E149K, Q154T, S157P, T158K, H161K, R162A, E165M, Q167R, A170G, S173D, E174A, K181D, P182S, Q185L, G187E, E188Q, I189V, T193K, A194N, S200A, S207T, N221S, Q222K, Q223E, S230E, S236A, G240A, N247Y, S248A, H257G, A262E, V266R, Q275K, K289Q, V298L, K306D, G324A, G334A, Q342T, Q345G, G355A, N363D, D365K, S386A, K392T, S393N, H394N, I409T, T411D, Q416P, and A425K;
(f) Q46F, K57P, A65P, T77K, G81R, F82Y, Y83F, N89Q, T102E, Q106W, L115A, Q154T, S157P, T158E, H161K, E165M, A170G, E174A, Q185L, G187Q, T193C, S200K, S236A, G240A, N247Y, S248A, V266R, L269M, V298L, K306D, G324A, G334A, Q345G and T411D;
(g) Q46F, A65P, T77K, G81Q, N89Q, T102E, Q106W, S157P, A170G, Q185L, G187Q, T193C, S236A, G240A, N247Y, S248A, L269M, V298L, K306D, G324A, G334A, Q345G and T411D;
(h) Q46F, K57P, A65P, T77K, G81Q, F82Y, Y83F, N89Q, T102E, Q106W, L115A, Q154T, S157P, T158E, H161K, E165M, A170G, E174A, Q185L, G187Q, T193C, S200K, S236A, G240A, N247Y, S248A, V266R, Q275Y, V298L, K306D, G324A, G334A, Q345G and T411D;
(i) Q46F, D56H, K57P, A65P, T77K, G81R, F82Y, Y83F, N89Q, T102E, Q106W, L115A, V141M, Q154T, S157P, T158E, H161R, R162A, E165M, A170G, E174A, Q185L, G187Q, I189V, T193C, A194N, S200K, S230E, S236A, G240A, N247Y, S248A, A262E, V266R, L269M, V298L, K306D, G324A, G334A, Q345G, N363D, S386A, I409T, and T411D;
(j) N89Q, T102E, Q106W, S157P, A170G, Q185L, G187Q, S236A, G240A, N247Y, S248A, V298L, G324A, G334A, Q345G, and T411D;
(k) N89Q, Q106W, S157P, A170G, Q185L, G187Q, S236A, G240A, N247Y, S248A, L269M, Q275Y, V298L, G324A, G334A, Q345G, and T411D;
(l) Q46F, D56H, K57P, A65P, T77K, G81Q, F82Y, Y83F, N89Q, T102E, Q106W, I109T, L115A, V141P, Q154T, S157P, T158E, H161R, R162A, E165M, A170G, E174A, Q185L, G187Q, E188Q, T193C, A194N, S200K, Q222K, Q223E, S230E, S236A, G240A, N247Y, S248A, H257G, A262E, V266R, S267E, L269M, Q275Y, V298L, K306D, G324A, G334A, Q342T, Q345G, G355A, N363D, S386A, S393N, H394N, I409T, T411D, and A425K; or
(m) Q46F, K57P, A65P, T77K, G81Q, N89Q, T102E, Q106W, L115A, Q154R, S157P, T158E, H161K, E165M, A170G, E174A, Q185L, G187Q, T193C, S236A, G240A, N247Y, S248A, V298L, K306D, G324A, G334A, Q345G, and T411D.

Provided herein are chanoclavine-I aldehyde reductase easA variants of a wild-type chanoclavine-I aldehyde reductase easA from Neosartorya fumigata of SEQ ID NO: 46 comprising one or more mutations selected from A8S, K12Q, H20N, I26V, T30M, G37D, Q38N, G50A, D66F, T68S, M72G, P84E, R86I, E91K, S94D, R95A, I126V, C151Y or C151I, A153E, N164E, T184I, K186D, Q192T, T210V, V217I, E240D, A250E, M252L, D256K, L268Q, E270N, E304R, K305W, Q309W, Y368W, L371R, and H372N or H372Q.

In one embodiment, provided herein is a chanoclavine-I aldehyde reductase easA variant of a wild-type chanoclavine-I aldehyde reductase easA from Neosartorya fumigata of SEQ ID NO: 46 comprising mutations:
(a) A8S, K12Q, H20N, I26V, T30M, G37D, Q38N, G50A, D66F, T68S, M72G, P84E, R86I, E91K, S94D, R95A, I126V, C151Y, A153E, N164E, T184I, K186D, Q192T, T210V, V217I, E240D, A250E, M252L, D256K, L268Q, E270N, E304R, K305W, Q309W, Y368W, L371R and H372N;
(b) A8S, H20N, I26V, T30M, G37D, Q38N, G50A, D66F, T68S, M72G, R86I, E91K, S94D, C151Y, A153E, N164E, T184I, K186D, Q192T, V217I, A250E, Q309W, Y368W, L371R and H372Q;
(c) A8S, I26V, T30M, G37D, Q38N, G50A, D66F, T68S, M72G, R86I, S94D, C151Y, A153E, N164E, T184I, K186D, Q192T, V217I, Q309W, Y368W, and L371R;
(d) A8S, K12Q, H20N, I26V, T30M, G37D, Q38N, G50A, D66F, T68S, M72G, P84E, R86I, E91K, S94D, R95A, C151Y, A153E, N164E, T184I, K186D, Q192T, T210V, V217I, A250E, Q309W, Y368W, L371R, and H372Q;
(e) A8S, K12Q, H20N, I26V, T30M, G37D, Q38N, G50A, D66F, T68S, M72G, P84E, R86I, E91K, S94D, R95A, C151Y, A153E, N164E, T184I, K186D, Q192T, T210V, V217I, A250E, M252L, Q309W, Y368W, L371R, and H372N;
(f) A8S, K12Q, H20N, I26V, T30M, G37D, Q38N, G50A, T68S, M72G, P84E, R86I, E91K, S94D, R95A, I126V, C151I, A153E, N164E, T184I, K186D, Q192T, T210V, V217I, E240D, A250E, M252L, D256K, L268Q, E270N, E304R, K305W, Q309W, Y368W, L371R and H372N;
(g) I26V, G50A, D66F, T68S, M72G, R86I, C151Y, K186D, Q192T, and Y368W;
(h) I26V, T30M, G50A, D66F, T68S, M72G, R86I, S94D, C151Y, A153E, K186D, Q192T and Y368W; or
(i) A8S, I26V, T30M, G37D, G50A, D66F, T68S, M72G, R86I, S94D, C151Y, A153E, N164E, T184I, K186D, Q192T, Y368W, and L371R.

Also provided herein are alanine dehydrogenase variants of a wild-type alanine dehydrogenase from Archaeoglobus fulgidus of SEQ ID NO: 47 comprising one or more mutations selected from E2K, Q8R, S13Q, N22E, L33E, G72Q, S89D, T104I, S106A, S125A, F128V, T135E, K235R, D277P, V300A, S310E, S315T, K316F and I317V.

In one embodiment, provided herein is an alanine dehydrogenase variant of a wild-type alanine dehydrogenase from Archaeoglobus fulgidus of SEQ ID NO: 47 comprising mutations:
(a) E2K, Q8R, N22E, L33E, S89D, T104I, S106A, S125A, F128V, K235R, D277P, S310E, S315T, and K316F;
(b) Q8R, S106A, K235R, D277P, S310E, S315T, and K316F;
(c) E2K, Q8R, N22E, S89D, S106A, S125A, K235R, D277P, S310E, S315T, and K316F;
(d) E2K, Q8R, N22E, S89D, S106A, S125A, F128V, D277P, S310E, S315T, and K316F;
(e) E2K, Q8R, S13Q, N22E, L33E, G72Q, S89D, T104I, S106A, S125A, F128V, T135E, K235R, D277P, V300A, S310E, S315T, K316F, and I317V;
(f) E2K, Q8R, N22E, L33E, S89D, T104I, S106A, S125A, F128V, D277P, V300A, S310E, S315T, and K316F;
(g) E2K, Q8R, N22E, L33E, S89D, T104I, S106A, S125A, F128V, T135E, K235R, D277P, V300A, S310E, S315T, and K316F;
(h) Q8R, S106A, K235R, D277P, S315T, and K316F;
(i) E2K, Q8R, S13Q, N22E, L33E, G72Q, S89D, T104I, S106A, S125A, F128V, K235R, D277P, V300A, S310E, S315T, K316F, and I317V;
(j) E2K, Q8R, N22E, S89D, S106A, S125A, F128V, K235R, D277P, S310E, S315T and K316F; or
(k) E2K, Q8R, N22E, L33E, S89D, T104I, S106A, S125A, F128V, D277P, S310E, S315T and K316F.

Provided herein are acid phosphatase variants of a wild-type acid phosphatase from Shimwellia blattae of SEQ ID NO: 48 comprising one or more mutations selected from T28A, E41Q, N44D; N62A, N62L or N62Y; L72A; L81Q, A83K or A83Q; E84A, S90P, G91D, G92D, A94P, N95K; G99P or G99E; S103M, E107P, K108E, A112W, L113I, H114Y or H114W; I121F, 5170A; I171F, I171M, I171L or I171A; T175F or T175V; V178I, Q185E or Q185D, N188D, L197Y, A215G, T225A, Q234A, Q235D, and Q237A.

In one embodiment, provided herein is an acid phosphatase variant of a wild-type acid phosphatase from Shimwellia blattae of SEQ ID NO: 48 comprising mutations:
(a) T28A, E41Q, N44D, N62A, L72A, A83K, E84A, S90P, G91D, G92D, A94P, N95K, G99P, S103M, E107P, A112W, L113I, H114Y, S170A, I171F, Q185E, N188D, L197Y, A215G, T225A, Q234A, and Q235D;
(b) T28A, E41Q, N44D, N62A, L72A, A83K, E84A, S90P, G91D, G92D, A94P, N95K, G99P, S103M, E107P, A112W, L113I, H114Y, S170A, T175F, Q185D, N188D, L197Y, A215G, T225A, Q234A, and Q235D;
(c) E41Q, N44D, N62A, L72A, A83K, G91D, G92D, A94P, G99P, E107P, H114W, I171M, T175F, Q185D, L197Y, and Q234A;
(d) N44D, N62A, L72A, A83K, G91D, G92D, G99E, E107P, H114Y, I171L, Q185E, and L197Y;
(e) E41Q, N44D, N62A, L72A, A83K, 590P, G91D, G92D, A94P, N95K, G99P, E107P, A112W, L113I, H114Y, S170A, I171M, T175F, Q185D, L197Y, A215G, T225A, and Q234A;
(f) E41Q, N44D, N62A, L72A, A83K, G91D, G92D, A94P, G99P, E107P, H114Y, I171M, T175F, Q185E, L197Y, and Q234A;
(g) N44D, N62L, L72A, A83Q, G91D, G92D, G99E, E107P, H114Y, T175F, Q185D and L197Y;
(h) N44D, N62Y, L72A, G91D, G92D, E107P, H114Y, T175F, and L197Y;
(i) T28A, E41Q, N44D, N62A, L72A, L81Q, A83K, E84A, S90P, G91D, G92D, N95K, G99P, S103M, E107P, K108E, A112W, L113I, H114Y, I121F, S170A, V178I, Q185E, N188D, L197Y, A215G, T225A, Q234A, Q235D, and Q237A;
(j) N44D, N62A, L72A, A83Q, G91D, G92D, G99E, E107P, H114Y, I171L, T175V, Q185E and L197Y; or
(k) T28A, E41Q, N44D, N62A, L72A, L81Q, A83K, E84A, S90P, G91D, G92D, A94P, N95K, G99P, S103M, E107P, K108E, A112W, L113I, H114Y, S170A, I171A, V178I, Q185E, N188D, L197Y, A215G, T225A, Q234A, Q235D, and Q237A.

Further provided herein are major phosphate-irrepressible acid phosphatase variants of a wild-type major phosphate-irrepressible acid phosphatase from Morganella morganii of SEQ ID NO: 49 comprising one or more mutations selected from E40D, K47A, E54T, Q59D, L61K, N62R, M72A, A89S, A90K, G91D or G91E; T95K, G99E, E107K, M137Q, E148T, N151T, K153D, Q155E, S170A, T175V, V182I, A185D or A185E; N186R, Q187A or Q187R; L197Y, D213E, A215G, T225A, D229N, Q233R, Q235D, K246L, S247L or S247R; Q248R, and K249Q.

In one embodiment, provided herein is a major phosphate-irrepressible acid phosphatase variant of a wild-type major phosphate-irrepressible acid phosphatase from Morganella morganii of SEQ ID NO: 49 comprising mutations:
(a) E40D, K47A, E54T, Q59D, L61K, N62R, M72A, A89S, A90K, G91D, T95K, G99E, E107K, M137Q, E148T, N151T, K153D, Q155E, S170A, T175V, V182I, A185D, N186R, Q187A, L197Y, D213E, A215G, T225A, D229N, Q233R, Q235D, S247L, Q248R, and K249Q;
(b) M72A, A89S, G91E, T95K, G99E, M137Q, E148T, N151T, K153D, S170A, T175V, V182I, A185E, Q187R, L197Y, A215G, T225A, Q235D, and K246L;
(c) M72A, A89S, G91D, T95K, G99E, M137Q, E148T, N151T, K153D, S170A, T175V, V182I, A185E, Q187R, L197Y, A215G, T225A, Q235D, and K246L;
(d) A89S, T175V, Q187R, L197Y, and A215G;
(e) E40D, K47A, E54T, Q59D, L61K, N62R, M72A, A89S, A90K, G91D, T95K, G99E, E107K, M137Q, E148T, N151T, K153D, Q155E, S170A, T175V, V182I, A185E, N186R, Q187A, L197Y, D213E, A215G, T225A, D229N, Q233R, Q235D, S247L, Q248R, and K249Q;
(f) K47A, Q59D, M72A, A89S, G91D, T95K, G99E, E107K, M137Q, E148T, N151T, K153D, Q155E, S170A, T175V, V182I, A185E, N186R, Q187A, L197Y, A215G, T225A, D229N, Q235D, K246L, and S247R;
(g) M72A, A89S, G91E, T95K, G99E, M137Q, E148T, N151T, K153D, S170A, T175V, V182I, A185E, N186R, Q187R, L197Y, A215G, T225A, Q235D, and K246L;
(h) A89S, G91E, G99E, K153D, T175V, Q187R, L197Y, A215G, Q235D, and K246L;
(i) K47A, Q59D, M72A, A89S, G91E, T95K, G99E, E107K, M137Q, E148T, N151T, K153D, Q155E, S170A, T175V, V182I, A185E, N186R, Q187A, L197Y, A215G, T225A, D229N, Q235D, K246L, and S247R; or
(j) A89S, G91E, G99E, K153D, T175V, Q187R, L197Y, and A215G.

Provided herein are acid phosphatase variants of a wild-type acid phosphatase from Prevotella intermedia of SEQ ID NO: 50 comprising one or more mutations selected from N33H, D41E, G42S or G42E; Q44A or Q44S; T45V or T45I, S46D, T53P, L63K or L63E; Y64N, E66M or E66R; A67N, M74A, D82K, V85R, V90L, N97Q, E101C, I105M, K109P, T111K, V119L, L120R or L120Q; G127N, A145M, Y147F, M150S, N153T, E155K, Q157E, Q158E, S161R, T162K, T171A, I173L, T177V, V180I, S182A, I186P, Q189A, N190D, E194K, Y197R, Q198E, M199Y, S221A, D231N, Q237D and Q249R.

In one embodiment, provided herein is an acid phosphatase variant of a wild-type acid phosphatase from Prevotella intermedia of SEQ ID NO: 50 comprising mutations:
(a) Q44A, T45V, S46D, T53P, Y64N, E66M, M74A, V85R, V90L, E101C, V119L, Y147F, M150S, N153T, T162K, V180I, S182A, I186P, E194K, M199Y, S221A, and Q237D;
(b) N33H, D41E, G42S, Q44A, T45V, S46D, T53P, L63K, Y64N, E66M, A67N, M74A, D82K, V85R, V90L, N97Q, E101C, I105M, K109P, T111K, V119L, L120R, G127N, A145M, Y147F, M150S, N153T, E155K, Q157E, Q158E, S161R, T162K, I173L, T177V, V180I, S182A, I186P, Q189A, N190D, E194K, Y197R, Q198E, M199Y, S221A, D231N, Q237D and Q249R;
(c) G42S, Q44A, T45V, S46D, T53P, L63K, Y64N, E66M, M74A, D82K, V85R, V90L, E101C, K109P, V119L, L120R, G127N, A145M, Y147F, M150S, N153T, E155K, Q157E, Q158E, S161R, T162K, T171A, I173L, T177V, S182A, I186P, N190D, E194K, Y197R, Q198E, M199Y, S221A, D231N, Q237D, and Q249R;
(d) Q44A, S46D, T53P, Y64N, E66M, V85R, V90L, V119L, Y147F, M150S, N153T, T177V, I186P, M199Y, and S221A;
(e) G42E, Q44A, T45V, S46D, T53P, Y64N, E66M, M74A, D82K, V85R, V90L, E101C, V119L, L120Q, A145M, Y147F, M150S, N153T, E155K, Q157E, S161R, T162K, T177V, V180I, S182A, I186P, M199Y, S221A, and Q237D;
(f) Q44A, S46D, T53P, Y64N, E66M, V85R, V90L, V119L, N153T, I186P, M199Y, and S221A;
(g) G42S, Q44A, T45V, S46D, T53P, L63K, Y64N, E66M, M74A, D82K, V85R, V90L, E101C, K109P, V119L, L120R, G127N, A145M, Y147F, M150S, N153T, E155K, Q157E, Q158E, S161R, T162K, I173L, T177V, V180I, S182A, I186P, N190D, E194K, Y197R, Q198E, M199Y, S221A, D231N, Q237D, and Q249R;
(h) Q44A, T45V, S46D, T53P, Y64N, E66M, M74A, V85R, V90L, E101C, V119L, A145M, Y147F, M150S, N153T, T162K, V180I, S182A, I186P, E194K, M199Y, S221A, and Q237D
(i) N33H, D41E, G42S, Q44A, T45V, S46D, T53P, L63E, Y64N, E66R, A67N, M74A, D82K, V85R, V90L, N97Q, E101C, I105M, K109P, T111K, V119L, L120R, G127N, A145M, Y147F, M150S, N153T, E155K, Q157E, Q158E, S161R, T162K, T171A, I173L, T177V, V180I, S182A, I186P, Q189A, N190D, E194K, Y197R, Q198E, M199Y, S221A, D231N, Q237D, and Q249R;
(j) G42E, Q44A, T45V, S46D, T53P, Y64N, E66M, M74A, D82K, V85R, V90L, E101C, V119L, L120Q, A145M, Y147F, M150S, N153T, E155K, Q157E, S161R, T162K, V180I, S182A, I186P, E194K, M199Y, S221A, and Q237D;
(k) Q44A, T45V, S46D, T53P, Y64N, E66M, V85R, V90L, V119L, Y147F, M150S, N153T, T177V, S182A, I186P, M199Y, S221A, and Q237D; or
(l) Q44S, T45I, S46D, T53P, Y64N, E66M, V85R, V90L, V119L, Y147F, M150S, N153T, T177V, I186P, M199Y, and S221A.

Provided herein are tryptophan dimethylallyltransferase variants of a wild-type tryptophan dimethylallyltransferase from Neosartorya fumigata of SEQ ID NO: 51 comprising one or more mutations selected from K2Q, A3T, A9N, R17K, A18Y, L28R, T43D or T43M, T47S, T48L, C50Q or C50E; T57F, C61H, C68P, A74R, N96H, S97N, K114N, S125A, H128R or H128Q, L130K, K134P, H146N, S152R, S155A, F157Y, H160K, G167D, A185T or A185V; T188V or T188A; T202S, V207I, G209E, V211I, R213K, V216Q, N226D, S237P, A254D, Q265R, M266N or M266Q, A285P, S292E, 5304P, V319Q, I320V, Q335P, N336G, V339Y, E341Q, S370T, T378E, T379A, A387Q, D390S, D405N, R406N, T407K or T407Q; and V453K.

In another embodiment, provided herein is a tryptophan dimethylallyltransferase variant of a wild-type tryptophan dimethylallyltransferase from Neosartorya fumigata of SEQ ID NO: 51 comprising mutations:
(a) L28R, C50Q, T57F, A74R, S125A, H128R, K134P, H146N, H160K, A185T, T188V, V211I, V216Q, S237P, A285P, S292E, V319Q, Q335P, E341Q, T378E, A387Q, R406N, and T407K;
(b) L28R, C50Q, T57F, S125A, H128R, K134P, H146N, S152R, H160K, A185V, T188V, V211I, V216Q, S237P, A285P, S292E, 5304P, V319Q, Q335P, A387Q, and T407K;
(c) A3T, L28R, C50Q, T57F, C68P, A74R, N96H, S97N, S125A, H128R, K134P, H146N, S152R, H160K, A185T, T188V, T202S, V211I, V216Q, S237P, A285P, S292E, V319Q, Q335P, V339Y, E341Q, T378E, A387Q, R406N, and T407Q;
(d) K2Q, A3T, L28R, T43D, T47S, T48L, C50E, T57F, C61H, C68P, A74R, N96H, S97N, K114N, S125A, H128R, L130K, K134P, H146N, S152R, S155A, H160K, A185V, T188V, T202S, V211I, V216Q, N226D, S237P, M266N, A285P, S292E, V319Q, I320V, Q335P, V339Y, E341Q, T378E, A387Q, R406N, and T407Q;
(e) A3T, L28R, C50E, T57F, C68P, A74R, S97N, S125A, H128R, K134P, H146N, S152R, H160K, A185V, T188V, T202S, V211I, V216Q, S237P, A285P, S292E, 5304P, V319Q, Q335P, V339Y, E341Q, T378E, A387Q, R406N, and T407K;
(f) K2Q, A3T, L28R, T43D, T47S, T48L, C50Q, T57F, C61H, C68P, A74R, S97N, K114N, S125A, H128Q, L130K, K134P, H146N, S152R, S155A, H160K, A185T, T188V, T202S, V211I, V216Q, N226D, S237P, M266N, A285P, S292E, 5304P, V319Q, I320V, Q335P, V339Y, E341Q, T378E, A387Q, R406N, and T407K;
(g) L28R, C50Q, T57F, A74R, S125A, H128R, K134P, H146N, H160K, A185V, T188V, V211I, V216Q, S237P, A285P, S292E, 5304P, V319Q, Q335P, T378E, A387Q, R406N, and T407K;
(h) K2Q, A3T, A9N, R17K, A18Y, L28R, T43M, T47S, T48L, C50Q, T57F, C61H, C68P, A74R, N96H, S97N, K114N, S125A, H128Q, L130K, K134P, H146N, S152R, S155A, F157Y, H160K, G167D, A185T, T188A, T202S, V207I, G209E, V211I, R213K, V216Q, N226D, S237P, A254D, Q265R, M266Q, A285P, S292E, 5304P, V319Q, I320V, Q335P, N336G, V339Y, E341Q, S370T, T378E, T379A, A387Q, D390S, D405N, R406N, T407Q and V453K;
(i) C50E, T57F, S125A, H128R, K134P, A185T, S237P, A285P, S292E, 5304P, Q335P and T407K;
(j) L28R, C 50E, T57F, S125A, H128R, K134P, H146N, H160K, A185V, T188V, V211I, V216Q, S237P, A285P, S292E, S304P, V319Q, Q335P, A387Q, and T407K;
(k) L28R, C50E, T57F, S125A, H128R, K134P, A185T, S237P, A285P, S292E, S304P, Q335P, A387Q, and T407K;
(l) K2Q, A3T, A9N, R17K, A18Y, L28R, T43D, T47S, T48L, C50E, T57F, C61H, C68P, A74R, N96H, S97N, K114N, S125A, H128R, L130K, K134P, H146N, S152R, S155A, F157Y, H160K, G167D, A185V, T188A, T202S, V207I, G209E, V211I, R213K, V216Q, N226D, S237P, A254D, Q265R, M266Q, A285P, S292E, S304P, V319Q, I320V, Q335P, N336G, V339Y, E341Q, S370T, T378E, A387Q, D390S, D405N, R406N, T407Q, and V453K; or
(m) C50E, T57F, S125A, H128R, K134P, A185T, S237P, A285P, S292E, Q335P, and T407K.

Further provided herein are alcohol dehydrogenase variants of a wild-type alcohol dehydrogenase from Geobacillus stearothermophilus of SEQ ID NO: 52 comprising one or more mutations selected from N7R or N7T; K10G, A12P, E19P, R20I, K22T, L23I, E24G, E25P or E25R, V28I, I51V, E69A, A71G, K72P, K75T, I77V, Y89W, E94H or E94R; L99R, Q102W or Q102R; L110Q, G112T, Y120F, P127H, A132V, V141L, V143A, T153V, V159E, A162T, I179M, L181V, S197D, S201L, D206K, A212T, I213V, G215A, K222Q, H225Q, D226K, V228I, S236V, K241P, Q249R, K252R, L257I or L257V; V259L, N264P, A265G, L267M, V279I, S280T, V281I, K282R, K290Q, M292L, A311R, E312R or E312K; E318D, N329D, and K335D.

In a further embodiment, provided herein is an alcohol dehydrogenase variant of a wild-type alcohol dehydrogenase from Geobacillus stearothermophilus of SEQ ID NO: 52 comprising mutations:
(a) K10G, A12P, E19P, R20I, L23I, E24G, E25P, I51V, A71G, K72P, K75T, I77V, E94H, Q102W, L110Q, G112T, Y120F, A132V, V143A, T153V, I179M, L181V, S197D, S201L, D206K, A212T, G215A, H225Q, D226K, V228I, S236V, K241P, Q249R, K252R, L257I, V259L, N264P, A265G, L267M, V279I, S280T, K282R, K290Q, A311R, E312R and K335D;
(b) A12P, E19P, R20I, L23I, E24G, E25R, I51V, A71G, K72P, K75T, E94R, Q102W, G112T, A132V, V143A, L181V, S197D, S201L, A212T, G215A, D226K, S236V, K241P, Q249R, K252R, V259L, N264P, A265G, S280T, K282R, K290Q, A311R, E312K, and K335D;
(c) N7R, K10G, A12P, E19P, R20I, L23I, E24G, E25P, I51V, A71G, K72P, K75T, I77V, E94H, Q102W, L110Q, G112T, Y120F, A132V, V143A, T153V, I179M, L181V, S197D, S201L, D206K, A212T, I213V, G215A, H225Q, D226K, V228I, S236V, K241P, Q249R, K252R, L257V, V259L, N264P, A265G, L267M, V279I, S280T, V281I, K282R, K290Q, A311R, E312R, N329D, and K335D;
(d) A12P, E19P, R20I, L23I, E24G, E25R, A71G, K72P, E94H, Q102W, G112T, A132V, V143A, V159E, L181V, S201L, A212T, G215A, S236V, K241P, Q249R, K252R, V259L, N264P, A265G, S280T, K282R, K290Q, E312R, and K335D;
(e) A12P, E19P, R20I, L23I, E24G, E25R, I51V, A71G, K72P, K75T, E94H, Q102W, G112T, A132V, V143A, V159E, L181V, S197D, S201L, A212T, G215A, D226K, S236V, K241P, Q249R, K252R, L257V, V259L, N264P, A265G, S280T, K282R, K290Q, A311R, E312R and K335D;
(f) A12P, E19P, R20I, L23I, E24G, E25P, I51V, A71G, K72P, K75T, E94H, Q102W, L110Q, G112T, A132V, V143A, L181V, S197D, S201L, D206K, A212T, G215A, D226K, V228I, S236V, K241P, Q249R, K252R, V259L, N264P, A265G, S280T, K282R, K290Q, A311R, E312R, and K335D;
(g) A12P, E19P, R20I, L23I, E24G, E25R, I51V, A71G, K72P, E94H, Q102W, G112T, A132V, V143A, L181V, S201L, A212T, G215A, S236V, K241P, Q249R, K252R, V259L, N264P, A265G, S280T, K282R, K290Q, E312R, and K335D;
(h) K10G, A12P, E19P, R20I, L23I, E24G, E25P, I51V, A71G, K72P, K75T, I77V, E94H, Q102W, L110Q, G112T, Y120F, A132V, V143A, T153V, I179M, L181V, S197D, S201L, D206K, A212T, G215A, H225Q, D226K, V228I, S236V, K241P, Q249R, K252R, V259L, N264P, A265G, L267M, V279I, S280T, K282R, K290Q, M292L, A311R, E312R and K335D;
(i) K10G, A12P, E19P, R20I, L23I, E24G, E25P, V28I, I51V, A71G, K72P, K75T, I77V, E94H, Q102R, L110Q, G112T, Y120F, A132V, V143A, T153V, V159E, I179M, L181V, S197D, S201L, D206K, A212T, I213V, G215A, H225Q, D226K, V228I, S236V, K241P, Q249R, K252R, L257I, V259L, N264P, A265G, L267M, V279I, S280T, V281I, K282R, K290Q, A311R, E312R, N329D, and K335D;
(j) A12P, E19P, R20I, L23I, E24G, E25R, I51V, A71G, K72P, E94H, Q102W, G112T, A132V, V143A, V159E, L181V, S201L, A212T, G215A, S236V, K241P, Q249R, K252R, V259L, N264P, A265G, S280T, K282R, K290Q, E312R, and K335D;
(k) N7T, K10G, A12P, E19P, R20I, K22T, L23I, E24G, E25P, V28I, I51V, E69A, A71G, K72P, K75T, I77V, Y89W, E94H, Q102R, L110Q, G112T, Y120F, P127H, A132V, V143A, T153V, V159E, I179M, L181V, S197D, S201L, D206K, A212T, I213V, G215A, K222Q, H225Q, D226K, V228I, S236V, K241P, Q249R, K252R, V259L, N264P, A265G, L267M, V279I, S280T, V281I, K282R, K290Q, A311R, E312R, E318D, N329D, and K335D;
(l) N7T, K10G, A12P, E19P, R20I, K22T, L23I, E24G, E25P, V28I, I51V, E69A, A71G, K72P, K75T, I77V, Y89W, E94H, L99R, Q102W, L110Q, G112T, Y120F, P127H, V141L, V143A, T153V, V159E, A162T, I179M, L181V, S197D, S201L, D206K, A212T, I213V, G215A, K222Q, H225Q, D226K, V228I, S236V, K241P, Q249R, K252R, V259L, N264P, A265G, L267M, V279I, S280T, K282R, K290Q, M292L, A311R, E312R, E318D, N329D and K335D; or
(m) A12P, E19P, R20I, L23I, E24G, E25P, V28I, I51V, A71G, K72P, K75T, E94R, Q102W, L110Q, G112T, A132V, V143A, L181V, S197D, S201L, D206K, A212T, G215A, H225R, D226K, V228I, S236V, K241P, Q249R, K252R, V259L, N264P, A265G, S280T, K282R, K290Q, A311R, E312K, and K335D.

Also provided herein are alcohol dehydrogenase variants of a wild-type alcohol dehydrogenase from Rhodococcus jostii of SEQ ID NO: 53 comprising one or more mutations selected from V36A, K44E or K44D, A46L, D70P, S73T, I77V, V83A or V83C, T91R, S97T, Q99M, S100Q, L105A, N108G, V110A, T111A, E113C or E113A; L114Q or L114M; N116D, I128V, A132C, A133M, T136A, Q139E, F140Y, S141A or S141T; T154D, W159K, V160A, S161A, T164G or T164S; S173A, A177T, G178A, E179G, K181Q, A182P, A187V or A1871; V199I, R200Q, A201G, V203R, N206G, G208R, V210I, I213V, S218E or S218D, L220F, F222W, F222M or F222Q; Q225K, S226F, D229T, F230H, Y232F, T233A, A235M, H239A, D241L, Y247W, V249Q, T254V, V255I, V261G, F270M, and E271R.

In one embodiment, provided herein is an alcohol dehydrogenase variant of a wild-type alcohol dehydrogenase from Rhodococcus jostii of SEQ ID NO: 53 comprising mutations:
(a) V36A, K44E, A46L, D70P, S73T, I77V, V83A, T91R, S97T, Q99M, L105A, N108G, V110A, T111A, E113C, L114Q, N116D, A132C, A133M, T136A, Q139E, F140Y, S141A, T154D, V160A, S161A, T164G, E179G, A182P, A187V, R200Q, A201G, N206G, G208R, V210I, A211V, F222W, S226F, F230H, T233A, H239A, D241L, Y247W, T254V, V255I, V261G, F270M, and E271R;
(b) K44D, D70P, S73T, V83A, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, Y247W, V261G, and E271R;
(c) K44E, D70P, S73T, I77V, V83A, L105A, V110A, T111A, E113C, L114M, N116D, A132C, A133M, T136A, F140Y, S141T, T154D, V160A, T164S, A182P, R200Q, N206G, G208R, A211V, F222M, S226F, F230H, T233A, H239A, D241L, Y247W, V261G, F270M and E271R;
(d) V36A, K44D, A46L, D70P, S73T, I77V, V83A, S97T, Q99M, L105A, V110A, T111A, E113C, L114Q, N116D, A132C, A133M, T136A, Q139E, F140Y, S141A, T154D, V160A, S161A, T164G, A182P, A187I, R200Q, A201G, N206G, G208R, A211V, F222Q, S226F, F230H, T233A, H239A, D241L, Y247W, T254V, V255I, V261G, F270M, and E271R;
(e) V36A, K44D, A46L, D70P, S73T, I77V, V83A, T91R, S97T, Q99M, S100Q, L105A, N108G, V110A, T111A, E113C, L114M, N116D, I128V, A132C, A133M, T136A, Q139E, F140Y, S141A, T154D, W159K, V160A, S161A, T164G, S173A, A177T, G178A, E179G, K181Q, A182P, A187V, V199I, R200Q, A201G, V203R, N206G, G208R, V210I, A211V, I213V, S218E, F222W, Q225K, S226F, D229T, F230H, Y232F, T233A, A235M, H239A, D241L, Y247W, V249Q, T254V, V255I, V261G, F270M, and E271R;
(f) V36A, K44D, A46L, D70P, S73T, I77V, V83A, T91R, S97T, Q99M, S100Q, L105A, N108G, V110A, T111A, E113C, L114M, N116D, I128V, A132C, A133M, T136A, Q139E, F140Y, S141A, T154D, W159K, V160A, S161A, T164G, S173A, A177T, G178A, E179G, K181Q, A182P, A187V, V199I, R200Q, A201G, V203R, N206G, G208R, V210I, A211V, I213V, S218D, L220F, F222Q, Q225K, S226F, D229T, F230H, Y232F, T233A, A235M, H239A, D241L, Y247W, V249Q, T254V, V255I, V261G, F270M, and E271R;
(g) V36A, K44E, D70P, S73T, I77V, V83A, S97T, Q99M, L105A, V110A, T111A, E113A, L114M, N116D, A132C, A133M, T136A, Q139E, F140Y, S141A, T154D, V160A, T164S, A182P, R200Q, A201G, N206G, G208R, A211V, F222W, S226F, F230H, T233A, H239A, D241L, Y247W, T254V, V255I, V261G, F270M, and E271R;
(h) D70P, S73T, V83A, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, V261G and, E271R;
(i) V36A, K44E, D70P, S73T, I77V, V83A, S97T, Q99M, L105A, V110A, T111A, E113C, L114Q, N116D, A132C, A133M, T136A, Q139E, F140Y, S141A, T154D, V160A, T164S, A182P, A187I, R200Q, A201G, N206G, G208R, A211V, F222M, S226F, F230H, T233A, H239A, D241L, Y247W, T254V, V255I, V261G, F270M, and E271R;
(j) V36A, K44D, A46L, D70P, S73T, I77V, V83A, T91R, S97T, Q99M, L105A, N108G, V110A, T111A, E113C, L114Q, N116D, A132C, A133M, T136A, Q139E, F140Y, S141A, T154D, V160A, S161A, T164G, E179G, A182P, R200Q, A201G, N206G, G208R, V210I, A211V, F222M, S226F, F230H, T233A, H239A, D241L, Y247W, T254V, V255I, V261G, F270M and E271R;
(k) K44D, D70P, S73T, V83C, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, Y247W, V261G, and E271R;
(l) K44E, D70P, S73T, I77V, V83A, L105A, V110A, T111A, E113A, L114Q, N116D, A132C, A133M, T136A, F140Y, S141T, T154D, V160A, T164S, A182P, R200Q, N206G, G208R, A211V, F222M, S226F, F230H, T233A, H239A, D241L, Y247W, V261G, F270M and E271R;
(m) D70P, S73T, V83A, L105A, V110A, T111A, E113C, L114Q, N116D, A133M, T136A, F140Y, S141T, T154D, V160A, T164G, A182P, R200Q, N206G, A211V, S226F, F230H, T233A, H239A, D241L, V261G, and E271R; or
(n) V36A, K44E, A46L, D70P, S73T, I77V, V83A, S97T, Q99M, L105A, V110A, T111A, E113C, L114Q, N116D, A132C, A133M, T136A, Q139E, F140Y, S141A, T154D, V160A, S161A, T164S, A182P, R200Q, A201G, N206G, G208R, A211V, F222M, S226F, F230H, T233A, H239A, D241L, Y247W, T254V, V255I, V261G, F270M, and E271R.

Provided herein are acid phosphatase variants of a wild-type acid phosphatase from Shigella flexneri of SEQ ID NO: 54 comprising one or more mutations selected from D40S, N41Q, A425, I55P, G56D, A59D, L61K, L72A or L72K; L81Q, N95K, K108E, S110A, T118R or T118K, N119R, N151T, D156K or D156Q; R160K or R160T; 5170A, T1751 or T175M; K192Q, Q235D, and N246L.

In one embodiment, provided herein is an acid phosphatase variant of a wild-type acid phosphatase from Shigella flexneri of SEQ ID NO: 54 comprising mutations: (a) N41Q, I55P, G56D, L72A, N95K, S110A, N119R, R160K, T175I, and Q235D;
(b) N41Q, I55P, G56D, A59D, L72A, N95K, S110A, N119R, N151T, D156K, R160K, T175M, K192Q, Q235D, and N246L;
(c) D40S, N41Q, A425, I55P, G56D, A59D, L61K, L72A, L81Q, N95K, K108E, S110A, T118K, N119R, N151T, D156K, R160K, S170A, T175I, K192Q, Q235D, and N246L;
(d) D40S, N41Q, A42S, I55P, G56D, A59D, L61K, L72K, L81Q, N95K, K108E, S110A, T118R, N119R, N151T, D156K, R160K, S170A, T175I, K192Q, Q235D, and N246L;
(e) N41Q, L72A, S110A, T175I, and Q235D;
(f) L72A, S110A, and T1751;
(g) L72K, S110A, and T1751;
(h) N41Q, I55P, G56D, A59D, L72A, N95K, S110A, N119R, N151T, D156K, R160T, T175M, K192Q, Q235D, and N246L; or
(i) N41Q, I55P, G56D, L72A, N95K, S110A, N119R, N151T, D156Q, R160K, T175I, Q235D and N246L.

Also provided herein are alcohol dehydrogenase variants of a wild-type alcohol dehydrogenase from Aromatoleum aromaticum of SEQ ID NO: 55 comprising one or more mutations selected from K2R, S8A, R14N, E16T, L45Y, G46L, S49T, A51G, V54T, A55E, N57G, I71V, D76T, A79K, A83R, H91D, L93I, E96P, A97P, I99M or I99A; A100E, V110I, C115A or C115R, L117Y, R120A or R120F, T123D, S128A, Q136E, T142C, I152V, Y1535, Y153A or Y153G; N154E, N154H or N154D; A159P, S162T, T185R, A188V, H198R, R200K, 5205D, C211Q, S212R or S212T or S212Q, Q213N or Q213D; Q215D, A217V or A217R, S218K, C227R, V266E, K274M, R275K, Q277A, Q289M, N291E, T300H, F303K or F303R, 5309V, T3115 or T311P, L312F, T314Q or T314E, L321H, Q322L, K323E or K323Q, A324S and S332C.

In another embodiment, provided herein is an alcohol dehydrogenase variant of a wild-type alcohol dehydrogenase from Aromatoleum aromaticum of SEQ ID NO: 55 comprising mutations:
(a) A51G, V54T, D76T, A79K, A83R, E96P, A97P, I99M, C115A, L117Y, S128A, N154E, S162T, T185R, H198R, 5205D, S212R, Q213N, Q215D, A217V, S218K, C227R, V266E, Q277A, N291E, T300H, F303K, 5309V, L312F, T314Q, L321H, Q322L, and S332C;
(b) S8A, L45Y, A51G, V54T, N57G, I71V, D76T, A79K, A83R, H91D, E96P, A97P, I99M, C115A, L117Y, R120A, S128A, T142C, I152V, Y1535, N154H, A159P, S162T, T185R, H198R, 5205D, C211Q, S212R, Q213N, Q215D, A217V, S218K, C227R, V266E, K274M, R275K, Q277A, N291E, T300H, F303K, 5309V, T3115, L312F, T314E, L321H, Q322L, K323E, and S332C;
(c) K2R, S8A, L45Y, A51G, V54T, N57G, I71V, D76T, A79K, A83R, H91D, E96P, A97P, I99A, C115A, L117Y, R120A, S128A, T142C, I152V, Y153A, N154E, A159P, S162T, T185R, A188V, H198R, 5205D, C211Q, S212R, Q213D, Q215D, A217R, S218K, C227R, V266E, K274M, R275K, Q277A, Q289M, N291E, T300H, F303K, S309V, T311S, L312F, T314E, L321H, Q322L, K323E, A324S, and S332C;
(d) A51G, V54T, D76T, A79K, A83R, E96P, A97P, I99M, C115A, L117Y, R120A, S128A, T142C, Y153S, N154D, A159P, S162T, T185R, H198R, S205D, S212R, Q213N, Q215D, A217V, S218K, C227R, V266E, Q277A, N291E, T300H, F303K, S309V, L312F, T314Q, L321H, Q322L, and S332C;
(e) K2R, S8A, R14N, E16T, L45Y, G46L, S49T, A51G, V54T, A55E, N57G, I71V, D76T, A79K, A83R, H91D, L93I, E96P, A97P, I99A, A100E, V110I, C115A, L117Y, R120A, T123D, S128A, Q136E, T142C, I152V, Y153G, N154E, A159P, S162T, T185R, A188V, H198R, R200K, S205D, C211Q, S212T, Q213N, Q215D, A217R, S218K, C227R, V266E, K274M, R275K, Q277A, Q289M, N291E, T300H, F303R, S309V, T311S, L312F, T314Q, L321H, Q322L, K323Q, A324S, and S332C;
(f) A51G, V54T, D76T, A79K, A83R, E96P, A97P, I99M, C115A, L117Y, R120F, S128A, N154E, S162T, T185R, H198R, S205D, Q213N, Q215D, A217V, S218K, C227R, V266E, Q277A, N291E, T300H, F303K, S309V, L312F, T314Q, L321H, Q322L, and S332C;
(g) S8A, L45Y, A51G, V54T, N57G, I71V, D76T, A79K, A83R, H91D, E96P, A97P, I99M, C115A, L117Y, R120A, S128A, T142C, I152V, Y153S, N154E, A159P, S162T, T185R, H198R, S205D, C211Q, S212R, Q213N, Q215D, A217R, S218K, C227R, V266E, K274M, R275K, Q277A, Q289M, N291E, T300H, F303K, S309V, T311S, L312F, T314E, L321H, K323E, and S332C;
(h) A51G, V54T, D76T, A83R, E96P, A97P, L117Y, S128A, N154E, S162T, T185R, H198R, S205D, Q213N, Q215D, A217V, S218K, C227R, V266E, Q277A, N291E, T300H, F303K, S309V, L312F, T314Q, L321H, and S332C;
(i) A51G, V54T, D76T, A79K, A83R, E96P, A97P, I99M, C115A, L117Y, R120A, S128A, T142C, Y153S, N154D, A159P, S162T, T185R, H198R, S205D, S212R, Q213N, Q215D, A217V, S218K, C227R, V266E, Q277A, N291E, T300H, F303K, S309V, T311P, L312F, T314Q, L321H, Q322L, and S332C;
(j) K2R, S8A, R14N, E16T, L45Y, G46L, S49T, A51G, V54T, A55E, N57G, I71V, D76T, A79K, A83R, H91D, L93I, E96P, A97P, I99A, A100E, V110I, C115A, L117Y, R120A, T123D, S128A, Q136E, T142C, I152V, Y153A, N154E, A159P, S162T, T185R, A188V, H198R, S205D, C211Q, S212Q, Q213N, Q215D, A217R, S218K, C227R, V266E, K274M, R275K, Q277A, Q289M, N291E, T300H, F303R, S309V, T311S, L312F, T314Q, L321H, Q322L, K323E, A324S, and S332C;
(k) A51G, V54T, D76T, A83R, E96P, A97P, C115R, L117Y, S128A, N154E, S162T, T185R, H198R, S205D, Q213N, Q215D, A217V, S218K, C227R, V266E, Q277A, N291E, T300H, F303K, S309V, L312F, T314E, L321H, and S332C;
(l) A51G, V54T, D76T, A83R, E96P, A97P, L117Y, N154E, S162T, T185R, H198R, S205D, Q213D, Q215D, S218K, C227R, V266E, Q277A, T300H, F303K, S309V, L312F, T314Q, L321H, and S332C; or
(m) A51G, V54T, D76T, A83R, E96P, A97P, C115R, L117Y, N154E, S162T, T185R, H198R, S205D, Q213D, Q215D, S218K, C227R, V266E, Q277A, T300H, F303K, S309V, L312F, T314Q, L321H, and S332C.

Also provided herein are arylesterase variants of a wild-type arylesterase from Pseudomonas pseudoalcaligenes of SEQ ID NO: 56 comprising one or more mutations selected from A34G, F35Y, K49Q, S52K, E54H, E57D, S78P, E83R, E87D, Q102L, V115I, S118A, L128V, V138Q or V138R; T142Q or T142E, T149K or T149E; E153K, S158P, G168A, V170N, G172E, M173W or M173L; A182S, E183A, E187P or E187A, I188Q, L189I, and T196Q.

In another embodiment, provided herein is an arylesterase variant of a wild-type arylesterase from Pseudomonas pseudoalcaligenes of SEQ ID NO: 56 comprising mutations:
(a) F35Y, E54H, S78P, E87D, Q102L, L128V, S158P, G168A, G172E, and M173W;
(b) A34G, F35Y, K49Q, S52K, E54H, E57D, S78P, E83R, E87D, Q102L, S118A, L128V, V138Q, T142Q, T149K, S158P, G168A, G172E, M173L, E187P, and I188Q;
(c) A34G, F35Y, S52K, E54H, E57D, S78P, E87D, Q102L, S118A, L128V, T142Q, S158P, G168A, G172E, M173L, E187P, and I188Q;
(d) A34G, F35Y, S52K, E54H, E57D, S78P, E87D, Q102L, S118A, L128V, V138Q, T142Q, T149K, S158P, G168A, G172E, M173L, E187P, I188Q, and L1891;
(e) A34G, F35Y, K49Q, S52K, E54H, E57D, S78P, E83R, E87D, Q102L, V115I, S118A, L128V, V138Q, T142Q, T149E, E153K, S158P, G168A, V170N, G172E, M173L, A182S, E183A, E187A, I188Q, and T196Q;
(f) A34G, F35Y, K49Q, S52K, E54H, E57D, S78P, E83R, E87D, Q102L, V115I, S118A, L128V, V138R, T142E, T149A, E153K, S158P, G168A, V170N, G172E, M173L, A182S, E183A, E187A, I188Q, and T196Q;
(g) A34G, F35Y, S52K, E54H, E57D, S78P, E87D, Q102L, S118A, L128V, V138Q, T142Q, T149K, S158P, G168A, G172E, M173L, E187P, and I188Q;
(h) F35Y, E54H, E57D, S78P, E87D, Q102L, L128V, T142Q, S158P, G168A, G172E, M173L, E187P, and L1891;
(i) F35Y, E54H, S78P, E87D, Q102L, L128V, S158P, G168A, G172E, M173W, and L1891;
(j) A34G, F35Y, K49Q, S52K, E54H, E57D, S78P, E83R, E87D, Q102L, S118A, L128V, V138R, T142E, T149K, S158P, G168A, G172E, M173L, E187P, and I188Q; or
(k) F35Y, E54H, S78P, E87D, Q102L, L128V, S158P, and G168A.

Also provided herein are putrescine aminotransferase variants of a wild-type putrescine aminotransferase from Escherichia coli of SEQ ID NO: 57 comprising one or more mutations selected from A12E, A15Q, H16R, R24D, H28E, A33Q, R36K, K43R, E44K, F50Y, A59K, F84Y, V102K, S105K, N109E, A112D, Q114L, Q119N, L121F, D123N, K131H, A135E, T137A, K140D, S144V, S153A, K162R, A175T, A1925, S194E, T195E, F196Y, N214D, M218L, T220K, N223E, L248I, T255R, F264Y or F264H; M268L, K282R, N290G, Q292V, A299G, L322F, L344I, T346A, N348H, Q353E, A357E, Q361E, M365Y or M365R, V381I, Q382R, M389L, N398E or N398D; G401A, S406A, R412G, T4185, A4225, T424V, I435D, C438I, E439D, L440K or L440Q; M452I, and S455Q.

In one embodiment, provided herein is a putrescine aminotransferase variant of a wild-type putrescine aminotransferase from Escherichia coli of SEQ ID NO: 57 comprising mutations:
(a) A12E, H16R, H28E, A33Q, K43R, V102K, S105K, N109E, A112D, Q119N, L121F, D123N, K131H, A135E, T137A, K140D, S144V, S153A, K162R, S194E, T195E, F196Y, N214D, M218L, T220K, N223E, L248I, T255R, F264Y, M268L, K282R, Q292V, A299G, L344I, N348H, Q353E, A357E, Q361E, M365Y, V381I, Q382R, M389L, N398E, S406A, T4185, T424V, I435D, C438I, E439D, L440K, and M452I;
(b) N109E, L121F, K140D, S144V, S153A, T195E, M218L, N223E, L248I, T255R, K282R, Q292V, A299G, N348H, M365Y, Q382R, M389L, S406K, T424V, I435D, and C438L;
(c) N109E, L121F, A135E, K140D, S144V, S153A, K162R, T195E, M218L, T220K, N223E, L248I, T255R, K282R, Q292V, A299G, N348H, M365Y, Q382R, M389L, S406A, T424V, I435D, and C438I;
(d) A12E, H28E, A33Q, S105K, N109E, L121F, K131H, A135E, T137A, K140D, S144V, S153A, K162R, S194E, T195E, N214D, M218L, T220K, N223E, L248I, T255R, F264H, K282R, Q292V, A299G, N348H, Q353E, Q361E, M365Y, V381I, Q382R, M389L, S406A, T424V, I435D, C438I, and L440Q;
(e) N109E, L121F, K140D, S144V, S153A, T195E, M218L, N223E, L248I, T255R, K282R, Q292V, A299G, N348H, M365Y, Q382R, M389L, S406K, T424V, I435D, and C438I;
(f) A12E, S105K, N109E, L121F, K131H, A135E, T137A, K140D, S144V, S153A, K162R, S194E, T195E, N214D, M218L, T220K, N223E, L248I, T255R, F264H, K282R, Q292V, A299G, N348H, M365Y, Q382R, M389L, S406A, T424V, I435D, C438I, and L440R;
(g) N109E, L121F, A135E, K140D, S144V, S153A, K162R, T195E, T220K, N223E, L248I, T255R, K282R, Q292V, A299G, N348H, M365Y, Q382R, M389L, S406A, T424V, I435D, and C438I;
(h) A12E, H28E, A33Q, S105K, N109E, L121F, K131H, A135E, T137A, K140D, S144V, S153A, K162R, S194E, T195E, N214D, M218L, T220K, N223E, L248I, T255R, F264Y, K282R, Q292V, A299G, N348H, Q353E, Q361E, M365Y, V381I, Q382R, M389L, S406A, T424V, I435D, C438I, and L440Q;
(i) A12E, S105K, N109E, L121F, K131H, A135E, T137A, K140D, S144V, S153A, K162R, S194E, T195E, N214D, M218L, T220K, N223E, L248I, T255R, K282R, Q292V, A299G, N348H, M365Y, Q382R, M389L, S406A, T424V, I435D, and C438I;
(j) L121F, S144V, S153A, T195E, N223E, L248I, T255R, K282R, Q292V, A299G, N348H, M365Y, M389L, S406K, T424V, I435D, and C438I;
(k) A12E, A15Q, H16R, R24D, H28E, A33Q, R36K, E44K, F50Y, A59K, V102K, S105K, N109E, A112D, Q114L, Q119N, L121F, D123N, K131H, A135E, T137A, K140D, S144V, S153A, K162R, A175T, A1925, S194E, T195E, F196Y, N214D, M218L, T220K, N223E, L248I, T255R, F264H, M268L, K282R, N290G, Q292V, A299G, L322F, L344I, T346A, N348K, Q353E, A357E, Q361E, M365Y, V381I, Q382R, M389L, N398E, G401A, S406A, R412G, T4185, A4225, T424V, I435D, C438I, E439D, L440K, M452I, and S455Q;
(l) A12E, A15Q, H16R, R24D, H28E, A33Q, R36K, K43R, F50Y, A59K, F84Y, V102K, S105K, N109E, A112D, Q114L, Q119N, L121F, D123N, K131H, A135E, K140D, S144V, S153A, K162R, A175T, A1925, S194E, T195E, F196Y, N214D, M218L, T220K, N223E, L248I, T255R, F264Y, M268L, K282R, N290G, Q292V, A299G, L322F, L344I, T346A, N348K, Q353E, A357E, Q361E, M365Y, V381I, Q382R, M389L, N398D, G401A, S406A, R412G, T4185, A4225, T424V, I435D, C438I, E439D, L440K, M452I, and S455Q;
(m) A12E, H16R, H28E, A33Q, K43R, V102K, S105K, N109E, A1 12D, Q119N, L121F, D123N, K131H, A135E, T137A, K140D, S144V, S153A, K162R, S194E, T195E, F196Y, N214D, M218L, T220K, N223E, L248I, T255R, F264Y, M268L, K282R, Q292V, A299G, L344I, N348H, Q353E, A357E, Q361E, M365R, V381I, Q382R, M389L, N398E, S406A, T418S, T424V, I435D, C438I, E439D, L440K, and M452I; or
(n) L121F, S144V, S153A, T195E, N223E, L248I, T255R, K282R, Q292V, A299G, N348H, M365Y, M389L, S406K, T424V, and I435D.

Provided herein are green to red photoconvertible GFP-like protein EosFP variants of a wild-type green to red photoconvertible GFP-like protein EosFP from Lobophyllia hemprichii of SEQ ID NO: 58 comprising one or more mutations selected from N11K, D26E, D28E, K32N, F34Y, M40I, L93M, T94I, I100V or I102T; T113C, N116H, K117E, R119K or R119E, A127P, L137I, K145M, K145S or K145Y; T154V, I157C or I157V, A160F, N166G, A167G, Y169L, F173C, F173V or F1731; E181K, L186M, F191L, V192I, and C195R.

In one embodiment, provided herein is a green to red photoconvertible GFP-like protein EosFP variant of a wild-type green to red photoconvertible GFP-like protein EosFP from Lobophyllia hemprichii of SEQ ID NO: 58 comprising mutations:
(a) N11K, K32N, F34Y, L93M, T94I, I100V, I102T, T113C, N116H, R119K, A127P, K145M, T154V, I157C, A160F, N166G, A167G, F173C, E181K, F191L, V192I, and C195R;
(b) N11K, K32N, L93M, T94I, I100V, I102T, T113C, R119E, A127P, K145S, A160F, N166G, A167G, F173V, E181K, F191L, V192I, and C195R;
(c) N11K, K32N, F34Y, L93M, I100V, I102T, T113C, N116H, K117E, R119E, A127P, K145M, T154V, I157C, A160F, N166G, A167G, F173C, E181K, F191L, V192I, and C195R;
(d) N11K, K32N, L93M, I100V, R119E, A127P, K145M, T154V, I157C, A160F, N166G, A167G, F173C, E181K, F191L, V192I, and C195R;
(e) L93M, R119E, A127P, K145M, N166G, A167G, F173C, F191L, and C195R;
(f) N11K, D26E, D28E, K32N, F34Y, M40I, L93M, I100C, I102T, T113C, N116H, K117E, R119E, A127P, L137I, K145M, T154V, I157C, A160F, N166G, A167G, F173I, E181K, L186M, F191L, V192I, and C195R;
(g) N11K, K32N, L93M, I100V, R119E, A127P, K145M, T154V, A160F, N166G, A167G, F173V, E181K, F191L, V192I, and C195R;
(h) N11K, K32N, L93M, I100V, T113C, R119E, A127P, K145M, T154V, I157C, A160F, N166G, A167G, Y169L, F173V, E181K, F191L, V192I, and C195R;
(i) N11K, K32N, L93M, I100V, T113C, R119E, A127P, K145M, T154V, A160F, N166G, A167G, F173C, E181K, F191L, V192I, and C195R;
(j) N11K, D26E, D28E, K32N, F34Y, M40I, L93M, T94I, I100V, I102T, T113C, N116H, R119E, A127P, L137I, K145Y, T154V, I157V, A160F, N166G, A167G, F173V, E181K, L186M, F191L, V192I, and C195R;
(k) N11K, K32N, L93M, I100V, R119E, A127P, K145S, N166G, A167G, F173C, F191L, V192I, and C195R; or
(l) N11K, K32N, L93M, I100V, I102T, T113C, R119E, A127P, K145M, I157C, A160F, N166G, A167G, F173C, E181K, F191L, V192I, and C195R.

Also provided herein are red fluorescent protein drFP583 variants of a wild-type red fluorescent protein drFP583 from Discosoma sp. of SEQ ID NO: 59 comprising one or more mutations selected from M1A, N6S, R17H, T21S, V22M, H41T, N42Q, V44A, A57S, K70R, V71A, Y72F, V73T, K83Y, L85Q, N98V, S111T, Q114E, D115G, C117T, F118L, K123W, F124L, I125R, V127T, S131P, D132N, A145P, R153E, E160D, H162K, K163M, K166R, H172R, L174R or L174T; V175A, E176D or E176Q; S179T, I180T, M182K, L189M, Y192A or Y192S; Y194F, S197R, D200E, N205D, I210V, T217S or T217A; G219A, H222S, L223T, and F224G.

In another embodiment, provided herein is a red fluorescent protein drFP583 variant of a wild-type red fluorescent protein drFP583 from Discosoma sp. of SEQ ID NO: 59 comprising mutations:
(a) M1A, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, I210V, T217S, H222S, L223T, and F224G;
(b) N6S, R17H, T21S, V22M, H41T, N42Q, V44A, A57S, K70R, V71A, Y72F, V73T, K83Y, L85Q, N98V, S111T, Q114E, D115G, F118L, K123W, F124L, I125R, V127T, S131P, D132N, A145P, R153E, E160D, H162K, K163M, K166R, L174T, V175A, E176Q, S179T, 1180T, M182K, L189M, Y192S, Y194F, S197R, D200E, N205D, I210V, T217A, G219A, H222S, L223T, and F224G;
(c) M1A, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T217S, H222S, L223T, and F224G;
(d) N6S, R17H, T21S, V22M, H41T, N42Q, V44A, A57S, K70R, V71A, Y72F, V73T, K83Y, L85Q, S111T, Q114E, D115G, C117T, F118L, F124L, I125R, V127T, S131P, A145P, R153E, E160D, H162K, K163M, K166R, H172R, L174T, V175A, E176D, S179T, I180T, M182K, L189M, Y192A, Y194F, S197R, N205D, I210V, T217S, G219A, H222S, L223T, and F224G;
(e) N6S, R17H, T21S, V22M, H41T, N42Q, V44A, A57S, K70R, V71A, Y72F, V73T, K83Y, L85Q, S111T, Q114E, D115G, F118L, F124L, I125R, V127T, S131P, D132N, A145P, R153E, E160D, H162K, K163M, K166R, H172R, L174T, V175A, E176D, S179T, I180T, M182K, L189M, Y192A, Y194F, S197R, N205D, I210V, T217A, H222S, L223T, and F224G;
(f) N6S, R17H, T21S, V22M, H41T, N42Q, V44A, A57S, K70R, V71A, Y72F, V73T, K83Y, L85Q, S111T, Q114E, D115G, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T217S, G219A, H222S, L223T and F224G; or
(g) N6S, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T217S, G219A, H222S, L223T, and F224G.

Further provided herein are green fluorescent protein variants of a wild-type green fluorescent protein from Aequorea victoria of SEQ ID NO: 60 comprising one or more mutations selected from S28K, 530R, F64L, S72A, N105C, N146I, H148G, M153T, K158N, V163A, N164E, I167M, N170P, S175G, N198Y, 5202H, Q204R or Q204H; A206V, and V224L.

In another embodiment, provided herein is a green fluorescent protein variant of a wild-type green fluorescent protein from Aequorea victoria of SEQ ID NO: 60 comprising mutations:
(a) S28K, 530R, F64L, S72A, N105C, N146I, H148G, M153T, K158N, V163A, N164E, I167M, N170P, S175G, N198Y, 5202H, Q204R, and A206V;
(b) S28K, 530R, F64L, S72A, N105C, N146I, H148G, M153T, K158N, V163A, N164E, I167M, N170P, S175G, N198Y, Q204H, and A206V;
(c) S28K, 530R, F64L, S72A, N105C, N146I, H148G, M153T, K158N, V163A, N164E, I167M, N170P, S175G, N198Y, Q204H, A206V, and V224L;
(d) 530R, F64L, S72A, N105C, N146I, H148G, M153T, K158N, V163A, N170P, S175G, S202H, A206V, and V224L;
(e) 530R, F64L, S72A, N146I, H148G, M153T, K158N, V163A, S175G, S202H, and V224L;
(f) 530R, F64L, S72A, N105C, N146I, H148G, M153T, K158N, V163A, N170P, S175G, N198Y, S202H, A206V, and V224L; or
(g) S28K, 530R, F64L, S72A, N105C, N146I, H148G, M153T, K158N, V163A, N170P, S175G, N198Y, S202H, A206V, and 0V224L.

Also provided herein are luciferin 4-monooxygenase variants of a wild-type luciferin 4-monooxygenase from Photinus pyralis of SEQ ID NO: 61 comprising one or more mutations selected from A4G, K8I, A12P, F14R, D19P, Q25L, A29L, V36H, T39R, I47T, E48G, V49Q, Y56L, E58Q, M59W, 560A, V61N or V61C; L63M or L63V; M67L, T74K, N75G, R77V, Q87E, M90V, A101T or A101V; A103H or A103T; I108N or I108T, N110T, R112D, L114I, N116H, S117Q, M118L, T124K, V128T, K130P or K1305; G132A, Q134P, Q147K, T156Q, Q159N, V168M, T169K, H171Y, G175N, V182K, K190Q, T191Q, T214N, A215I, A222C, R223S, D234G, G246A, T251C or T251H; T252V, Y255H, I257R or I257Y; C258Q, L264V, Y266R or Y266K; S276A, L277V or L277I; K281R, Q283T, S284V, T290S or T290P, F292M, S293V, N3085, H310T, G316A, H332N or H332G; I349V, A361S, F368G, V384P, M396N, S399K, N409K, L411T, 5420T, W426F, E430G, S440E, S456A, I457V, Q460S, N463D or N463K; F465A, L472Y, E488K, H489P, V499I, T5075, T508P, A509H or A509Y; V522I, G525T, G525N or G5255; L526P, L530I, and A532R or A532K.

In one embodiment, provided herein is a luciferin 4-monooxygenase variant of a wild-type luciferin 4-monooxygenase from Photinus pyralis of SEQ ID NO: 61 comprising mutations:
(a) A4G, K8I, A12P, D19P, Q25L, V36H, T39R, I47T, E48G, V49Q, Y56L, E58Q, M59W, 560A, V61N, L63M, M67L, T74K, N75G, R77V, M90V, A101T, A103H, I108N, N110T, R112D, L114I, N116H, S117Q, M118L, T124K, V128T, K130P, G132A, Q134P, V168M, T169K, H171Y, G175N, V182K, K190Q, T191Q, T214N, A222C, D234G, G246A, T251C, T252V, I257R, C258Q, L264V, Y266R, S276A, Q283T, T290S, S293V, G316A, H332N, A361S, V384P, M396N, S399K, N409K, L411T, 5420T, W426F, E430G, S440E, S456A, I457V, Q460S, N463D, F465A, L472Y, E488K, H489P, V499I, T5075, T508P, A509H, V522I, G525T, L526P, L530I, and A532R;
(b) A4G, K8I, D19P, T39R, I47T, E48G, Y56L, M59W, V61C, L63V, M67L, R77V, M90V, A101T, A103H, I108N, N110T, N116H, S117Q, M118L, T124K, K130P, V168M, H171Y, T191Q, T214N, T251H, I257R, L264V, Y266K, S276A, L277V, Q283T, T290P, S293V, G316A, V384P, S420T, W426F, E430G, S440E, F465A, L472Y, E488K, H489P, T507S, T508P, A509Y, V522I, G525N, and A532K;
(c) A4G, K8I, D19P, T39R, I47T, E48G, Y56L, V61C, L63V, M67L, R77V, M90V, A101T, A103H, I108N, N110T, N116H, S117Q, M118L, T124K, K130P, V168M, H171Y, T191Q, T214N, T251H, I257R, L264V, Y266K, S276A, L277V, Q283T, T290P, S293V, G316A, V384P, S420T, W426F, E430G, S440E, F465A, L472Y, E488K, H489P, T507S, T508P, A509Y, V522I, G525N, and A532K;
(d) A4G, K8I, D19P, I47T, E48G, Y56L, V61C, L63V, M67L, R77V, M90V, A101T, A103H, I108N, N116H, S117Q, M118L, K130P, V168M, H171Y, T191Q, T214N, T251H, I257R, L264V, Y266K, S276A, K281R, Q283T, T290P, S293V, V384P, S420T, W426F, E430G, S440E, F465A, L472Y, E488K, H489P, T508P, A509Y, V522I, G525N, and A532K;
(e) A4G, K8 I, D19P, I47T, E48G, Y56L, M59W, V61C, L63V, M67L, R77V, M90V, A101T, A103H, I108N, S117Q, M118L, K130P, V168M, H171Y, T251H, I257R, L264V, Y266K, S276A, L277V, K281R, Q283T, T290P, S293V, V384P, S420T, W426F, E430G, S440E, F465A, L472Y, E488K, H489P, T508P, A509H, V522I, and A532K;
(f) A4G, K8I, A12P, F14R, D19P, Q25L, V36H, T39R, I47T, E48G, V49Q, Y56L, E58Q, M59W, S60A, V61N, L63M, M67L, T74K, N75G, R77V, Q87E, M90V, A101T, A103H, I108N, N110T, R112D, L114I, N116H, S117Q, M118L, T124K, V128T, K130P, G132A, Q134P, Q147K, T156Q, Q159N, V168M, T169K, H171Y, G175N, V182K, K190Q, T191Q, T214N, A222C, D234G, G246A, T251C, T252V, I257R, C258Q, L264V, Y266R, S276A, L277I, Q283T, T290S, S293V, N308S, H310T, G316A, H332G, I349V, A361S, F368G, V384P, M396N, S399K, N409K, L411T, S420T, W426F, E430G, S440E, S456A, I457V, Q460S, N463K, F465A, L472Y, E488K, H489P, V499I, T507S, T508P, A509Y, V522I, G525T, L526P, L530I, and A532R;
(g) A4G, K8I, A12P, D19P, T39R, I47T, E48G, V49Q, Y56L, S60A, V61C, L63V, M67L, T74K, R77V, M90V, A101T, A103H, I108T, N110T, N116H, S117Q, M118L, T124K, V128T, K130P, G132A, Q134P, V168M, H171Y, G175N, T191Q, T214N, A215I, D234G, G246A, Y255H, I257R, C258Q, L264V, Y266K, S276A, L277V, Q283T, T290P, S293V, G316A, H332N, A361S, V384P, S399K, N409K, S420T, W426F, E430G, S440E, S456A, I457V, Q460S, N463D, F465A, L472Y, E488K, H489P, V499I, T507S, T508P, A509Y, V522I, G525S, L526P, L530I, and A532K;
(h) A4G, K8I, A12P, F14R, D19P, Q25L, A29L, V36H, T39R, I47T, E48G, V49Q, Y56L, E58Q, M59W, S60A, V61N, L63M, M67L, T74K, N75G, R77V, Q87E, M90V, A101V, A103H, I108N, N110T, R112D, L114I, N116H, S117Q, M118L, T124K, V128T, K130P, G132A, Q134P, Q147K, T156Q, Q159N, V168M, T169K, H171Y, G175N, V182K, K190Q, T191Q, T214N, A215I, A222C, D234G, G246A, T251C, T252V, I257Y, C258Q, L264V, Y266R, S276A, L277I, Q283T, T290S, F292M, S293V, N308S, H310T, G316A, H332G, I349V, A361S, F368G, V384P, M396N, S399K, N409K, L411T, S420T, W426F, E430G, S440E, S456A, I457V, Q460S, N463K, F465A, L472Y, E488K, H489P, V499I, T507S, T508P, A509Y, V522I, G525T, L526P, L530I, and A532R;
(i) A4G, K8I, D19P, I47T, E48G, Y56L, M59W, V61C, L63V, M67L, R77V, M90V, A101T, A103T, I108N, N116H, S117Q, M118L, K130S, V168M, H171Y, T191Q, T214N, T251H, I257R, L264V, Y266K, S276A, L277V, K281R, Q283T, S284V, T290P, S293V, V384P, S420T, W426F, E430G, S440E, F465A, L472Y, E488K, H489P, T508P, A509Y, V522I, G525N, and A532K;
(j) A4G, K8I, A12P, D19P, Q25L, V36H, T39R, I47T, E48G, V49Q, Y56L, E58Q, M59W, S60A, V61N, L63M, M67L, T74K, N75G, R77V, M90V, A101T, A103H, I108N, N110T, R112D, L114I, N116H, S117Q, M118L, T124K, V128T, K130P, G132A, Q134P, V168M, T169K, H171Y, G175N, V182K, K190Q, T191Q, T214N, A222C, D234G, G246A, T251C, T252V, I257R, C258Q, L264I, Y266R, S276A, L277V, Q283T, T290S, S293V, G316A, H332N, A361S, V384P, M396N, S399K, N409K, L411T, S420T, W426F, E430G, S440E, S456A, I457V, Q460S, N463D, F465A, L472Y, E488K, H489P, V499I, T507S, T508P, A509H, V522I, G525T, L526P, L530I, and A532R;
(k) A4G, K8I, D19P, I47T, E48G, Y56L, V61C, L63V, M67L, R77V, M90V, A101T, A103H, I108N, S117Q, M118L, K130P, V168M, H171Y, T251H, I257R, L264V, Y266K, S276A, L277V, K281R, Q283T, S284V, T290P, S293V, V384P, S420T, W426F, E430G, S440E, F465A, L472Y, E488K, H489P, T508P, A509Y, V522I, and A532K;
(l) A4G, K8I, A12P, D19P, T39R, I47T, E48G, Y56L, M59W, V61C, L63V, M67L, R77V, M90V, A101V, A103T, I108T, N110T, N116H, S117Q, M118L, T124K, K130S, Q134P, V168M, H171Y, G175N, T191Q, T214N, R223S, T251H, I257Y, L264V, Y266K, S276A, Q283T, T290P, S293V, G316A, A361S, V384P, S399K, N409K, S420T, W426F, E430G, S440E, F465A, L472Y, E488K, H489P, V499I, T507S, T508P, A509H, V522I, G525N and A532K;
(m) A4G, K8I, A12P, D19P, T39R, I47T, E48G, V49Q, Y56L, M59W, 560A, V61C, L63V, M67L, T74K, R77V, M90V, A101V, A103H, I108T, N110T, N116H, S117Q, M118L, T124K, V128T, K130P, G132A, V168M, H171Y, G175N, T191Q, T214N, D234G, G246A, T251H, I257R, C258Q, L264V, Y266K, S276A, L277V, Q283T, T290P, S293V, G316A, H332N, A361S, V384P, S399K, N409K, S420T, W426F, E430G, S440E, S456A, I457V, Q460S, N463D, F465A, L472Y, E488K, H489P, V499I, T507S, T508P, A509Y, V522I, G525N, L526P, L530I, and A532K; or
(n) A4G, K8I, A12P, D19P, T39R, I47T, E48G, Y56L, V61C, L63V, M67L, R77V, M90V, A101V, A103T, I108T, N110T, N116H, S117Q, M118L, T124K, K130P, Q134P, V168M, H171Y, G175N, T191Q, T214N, I257R, L264V, Y266K, S276A, K281R, Q283T, T290P, S293V, G316A, A361S, V384P, S399K, N409K, S420T, W426F, E430G, S440E, F465A, L472Y, E488K, H489P, V499I, T507S, T508P, A509H, V522I, G525N, and A532K.

Provided herein are mOrange variants of a wild-type mOrange from Discosoma sp. of SEQ ID NO: 62 comprising one or more mutations selected from N8D, N9P, R22H, S26C, F46T, A49M, K50R, F88L, D120G, E122C, S136P, D137N, A150P, S152T or S152C, A161T, K167T, T179R, S180C, A197N, I199F, G201D or G201N, K203R; D205Y, D205E or D205V; N210D, Q218E, and G224A.

In one embodiment, provided herein is a mOrange variant of a wild-type mOrange from Discosoma sp. of SEQ ID NO: 62 comprising mutations:
(a) N8D, N9P, R22H, F46T, A49M, F88L, D120G, E122C, S136P, A150P, S152T, A161T, K167T, T179R, S180C, A197N, I199F, G201D, K203R, D205Y, N210D, and G224A;
(b) N8D, N9P, R22H, S26C, F46T, A49M, K50R, F88L, D120G, E122C, S136P, D137N, A150P, S152T, A161T, K167T, R169A, T179R, S180C, A197N, I199F, G201D, K203R, D205Y, N210D, Q218E, and G224A;
(c) N8D, R22H, F46T, F88L, E122C, A150P, S152T, A161T, T179R, S180C, I199F, G201D, K203R, D205Y, and G224A;
(d) N8D, N9P, R22H, F46T, A49M, F88L, D120G, E122C, S136P, A150P, S152T, A161T, K167T, T179R, S180C, A197N, I199F, G201D, K203R, D205E, N210D, and G224A;
(e) N8D, R22H, F46T, A49M, F88L, D120G, E122C, A150P, S152T, A161T, T179R, S180C, I199F, G201D, K203R, D205Y, N210D, and G224A;
(f) N8D, R22H, F46T, F88L, A150P, S152C, A161T, T179R, S180C, I199F, G201N, K203R, D205Y, and G224A; or
(g) N8D, R22H, F46T, F88L, A150P, S152C, A161T, T179R, S180C, I199F, G201N, D205V and G224A.

Also provided herein are sandercyanin fluorescent protein variants of a wild-type sandercyanin fluorescent Protein from Sander vitreus of SEQ ID NO: 63 comprising one or more mutations selected from M1L, K4R, A12P, E15P, D16N, A19P, A205, A42I, V25T, D28E; K35T, K35P or K35Q; A42I, I114T, N115D, A118D, S119F, A121V, A122E, V127L or V127M; G143E, T144I, M145L, G151P, T154E or T154K; L156I, A162N, and A163C.

In another embodiment, provided herein is a sandercyanin fluorescent protein variant of a wild-type sandercyanin fluorescent Protein from Sander vitreus of SEQ ID NO: 63 comprising mutations:
(a) A12P, D16N, A205, V25T, D28E, S119F, A122E, V127L, G143E, T144I, M145L, G151P, T154E, and A163C;
(b) A12P, D16N, A205, V25T, D28E, S119F, A122E, V127M, G143E, T144I, M145L, G151P, T154E, and A163C;
(c) M1L, A12P, E15P, D16N, A19P, A205, V25T, D28E, K35T, A42I, I114T, N115D, A118D, S119F, A121V, A122E, V127M, G143E, T144I, M145L, G151P, T154K, L156I, A162N and A163C;
(d) M1L, A12P, E15P, D16N, A19P, A205, V25T, D28E, K35T, A42I, I114T, N115D, A118D, S119F, A121V, A122E, V127L, G143E, T144I, M145L, G151P, T154E, L156I, A162N and A163C;
(e) K4R, A12P, D16N, A205, V25T, D28E, K35T, A42I, N115D, A118D, S119F, A122E, V127M, G143E, T144I, M145L, G151P, T154K, and A163C;
(f) A12P, D28E, K35P, S119F, A122E, V127M, G143E, M145L, G151P, T154E, and A163C; (g) K4R, A12P, D16N, A205, V25T, D28E, K35Q, A42I, N115D, A118D, S119F, A121V, A122E, V127L, G143E, T144I, M145L, G151P, T154K, and A163C;
(h) A12P, D16N, A205, V25T, D28E, K35T, N115D, S119F, A122E, V127M, G143E, T144I, M145L, G151P, T154K, and A163C;
(i) A12P, A20S, D28E, S119F, A122E, V127M, G143E, T144I, M145L, G151P, T154E and A163C;
(j) A12P, D28E, S119F, A122E, V127M, G143E, M145L, G151P, and T154E; or
(k) A12P, A20S, D28E, K35P, S119F, A122E, V127M, G143E, T144I, M145L, G151P, T154E and A163C.

Also provided herein are GFP-like fluorescent chromoprotein FP538 variants of a wild-type GFP-like fluorescent chromoprotein FP538 from Zoanthus sp. of SEQ ID NO: 64 comprising one or more mutations selected from K9T, T30E, I34R or I34E; K39E, I49V, E50K, G94E, S96T, I106T, C107A, N108R, N108S or N108T; I125E or I125K; N127H, M129T, D134N, K139Q, M141R or M141K; T143V, T1431 or T143L; N144G, A147P, M153Y, K157N, K162R, S166T, L170K, V184T, S189K, S192K, E196G, Q201E, L205E, T220Y, T220Q, T220E or T220H, I224V, F226H, and A229P.

In a further embodiment, provided herein is a GFP-like fluorescent chromoprotein FP538 variant of a wild-type GFP-like fluorescent chromoprotein FP538 from Zoanthus sp. of SEQ ID NO: 64 comprising mutations:
(a) K9T, T30E, I34R, K39E, I49V, E50K, G94E, S96T, I106T, C107A, N108R, I125E, N127H, M129T, D134N, K139Q, M141R, T143V, N144G, A147P, M153Y, K157N, K162R, S166T, L170K, V184T, S189K, S192K, E196G, Q201E, L205E, T220Y, I224V, F226H, and A229P;
(b) K9T, T30E, I34R, K39E, I49V, E50K, G94E, S96T, I106T, C107A, N108R, I125K, N127H, M129T, D134N, K139Q, M141R, T143V, N144G, A147P, M153Y, K157N, S166T, L170K, V184T, S189K, S192K, E196G, Q201E, L205E, T220Y, I224V, F226H, and A229P;
(c) G94E, K139Q, T143V, A147P, M153Y, K162R, and A229P;
(d) G94E, K139Q, T143I, A147P, M153Y, and A229P;
(e) K9T, T30E, I34R, K39E, G94E, S96T, I106T, C107A, N108S, I125K, N127H, M129T, K139Q, M141K, T143V, N144G, A147P, M153Y, K157N, K162R, S166T, L170K, V184T, E196G, T220Q, I224V, F226H, and A229P;
(f) K9T, T30E, I34R, K39E, G94E, S96T, I106T, C107A, N108S, I125K, N127H, M129T, K139Q, M141K, T143L, N144G, A147P, M153Y, K157N, S166T, L170K, V184T, E196G, T220H, I224V, F226H, and A229P;
(g) T30E, I34E, K39E, G94E, N108S, N127H, M129T, K139Q, M141R, T143V, A147P, M153Y, K162R, E196G, T220E, F226H, and A229P;
(h) I34E, G94E, N127H, K139Q, T143V, A147P, M153Y, K162R, T220E, and A229P;
(i) K9T, T30E, I34E, K39E, G94E, S96T, I106T, N108S, I125K, N127H, M129T, K139Q, M141R, T143V, A147P, M153Y, K162R, V184T, E196G, T220E, F226H, and A229P; or
(j) K9T, T30E, I34E, K39E, G94E, S96T, I106T, N108T, I125K, N127H, M129T, K139Q, M141K, T143V, A147P, M153Y, V184T, E196G, T220Q, F226H, and A229P.

Provided herein are mNeonGreen variants of a wild-type mNeonGreen from Branchiostoma lanceolatum of SEQ ID NO: 65 comprising one or more mutations selected from V52H, D53E, N65Y, E69M or E69Q, A110Q, S1311 or S131C, T139R, S143D, K152H, A158P, T164Q, S166K, A169G, W172P, S175E, K177M, T178L, T188D or T188E, K190T, T194K, G196K or G196E, N197D, 5203C, T204Q, T208H, N218D, and Y226F.

In a further embodiment, provided herein is a mNeonGreen variant of a wild-type mNeonGreen from Branchiostoma lanceolatum of SEQ ID NO: 65 comprising mutations:
(a) V52H, D53E, E69M, T139R, K152H, S166K, W172P, K177M, T188D, G196K, and 5203C;
(b) V52H, D53E, E69Q, S131 I, T139R, S143D, K152H, T164Q, S166K, A169G, W172P, K177M, T188D, K190T, T194K, G196E, N197D, 5203C, T208H, N218D, and Y226F;
(c) S166K, K177M, G196E, and 5203C;
(d) V52H, D53E, N65Y, E69Q, A110Q, S131I, T139R, S143D, K152H, A158P, T164Q, S166K, A169G, W172P, S175E, K177M, T178L, T188D, K190T, T194K, G196E, N197D, 5203C, T204Q, T208H, N218D, and Y226F;
(e) E69M, T139R, S166K, K177M, T188D, G196E, and 5203C;
(f) V52H, D53E, E69M, T139R, K152H, S166K, W172P, K177M, G196K, and 5203C;
(g) V52H, D53E, N65Y, E69Q, A110Q, S131C, T139R, S143D, K152H, A158P, T164Q, S166K, A169G, W172P, K177M, T188E, K190T, T194K, G196E, N197D, 5203C, T204Q, T208H, N218D, and Y226F;
(h) V52H, D53E, E69Q, S131 I, T139R, S143D, K152H, T164Q, S166K, A169G, W172P, S175E, K177M, T188D, K190T, T194K, G196E, N197D, 5203C, T208H, N218D, and Y226F;
(i) V52H, D53E, E69Q, T139R, K152H, S166K, W172P, K177M, K190T, G196E, N197D, 5203C, and Y226F;
(j) V52H, D53E, E69Q, S131 I, T139R, S143D, K152H, S166K, A169G, W172P, K177M, T188E, K190T, G196E, N197D, 5203C, and Y226F; or
(k) E69M, T139R, S166K, K177M, G196E, and 5203C.

Also provided herein are green fluorescent protein blFP-Y3 variants of a wild-type green fluorescent protein blFP-Y3 from Branchiostoma lanceolatum of SEQ ID NO: 66 comprising one or more mutations selected from M1A, A5T, V18H, D19E, E35Q, S49N, A76K, K79C, S97I, S100A, N101H, Y102F, T105R, S109N, K112I, I118H, S132K, W138P, T141E, S153G, T154E, T160K, G162K, T170E, N174T, N184D, and K213W.

In a further embodiment, provided herein is a green fluorescent protein blFP-Y3 variant of a wild-type green fluorescent protein blFP-Y3 from Branchiostoma lanceolatum of SEQ ID NO: 66 comprising mutations:
(a) E35Q, S97I, Y102F, I118H, W138P, T141E, T160K, G162K, N174T, and K213W;
(b) D19E, E35Q, K79C, S97I, N101H, Y102F, I118H, W138P, T141E, S153G, T160K, G162K, T170E, N174T, and K213W;
(c) E35Q, S97I, Y102F, I118H, W138P, T154E, T160K, G162K, T170E, N174T, and K213; (d) V18H, D19E, E35Q, S49N, A76K, K79C, S97I, N101H, Y102F, I118H, S132K, W138P, T141E, S153G, T160K, G162K, T170E, N174T, and K213W;
(e) D19E, E35Q, K79C, S97I, N101H, Y102F, I118H, W138P, T141E, S153G, T154E, T160K, G162K, T170E, N174T, and K213W;
(f) A5T, V18H, D19E, E35Q, S49N, A76K, K79C, S97I, N101H, Y102F, S109N, I118H, S132K, W138P, T141E, S153G, T160K, G162K, T170E, N174T, N184D, and K213W;
(g) E35Q, S97I, Y102F, I118H, W138P, T160K, G162K, N174T, and K213W; or
(h) M1A, A5T, V18H, D19E, E35Q, S49N, A76K, K79C, S97I, S100A, N101H, Y102F, T105R, S109N, K112I, I118H, S132K, W138P, T141E, S153G, T160K, G162K, T170E, N184D and K213W.

Provided herein are far-red fluorescent protein eqFP650 variants of a wild-type far-red fluorescent protein eqFP650 from Entacmaea quadricolor of SEQ ID NO: 67 comprising one or more mutations selected from G13N, T19E, 520G, K25N, A33M, K34R, A51M, I85V, T86M, Y88F, L94C, T100I, N104D, L107F, K112E, N114R, S120P, A134P, A142R or A142C; S144G, R147E, H149R, Q151N or Q151D; V157K, Y161H, H163I, S165H, F184V, F186Y, R189H or R189Y; K190R, E196H, K199D, T201N, and M208V.

In another embodiment, provided herein is a far-red fluorescent protein eqFP650 variant of a wild-type far-red fluorescent protein eqFP650 from Entacmaea quadricolor of SEQ ID NO: 67 comprising mutations:
(a) G13N, T19E, S20G, A33M, K34R, A51M, I85V, T86M, T100I, N104D, L107F, K112E, N114R, A134P, A142R, S144G, R147E, H149R, Q151N, V157K, Y161H, H163I, S165H, F184V, F186Y, R189H, K190R, E196H, K199D, and M208V;
(b) G13N, T19E, S20G, K25N, A33M, K34R, A51M, I85V, T86M, Y88F, L94C, T100I, N104D, L107F, N114R, S120P, A134P, A142R, S144G, R147E, H149R, Q151N, V157K, Y161H, H163I, S165H, F184V, F186Y, R189H, K190R, E196H, K199D, T201N, and M208V;
(c) G13N, T19E, S20G, A33M, K34R, A51M, I85V, T86M, L94C, T100I, N104D, L107F, N114R, A134P, A142R, S144G, R147E, H149R, Q151N, V157K, Y161H, H163I, S165H, F184V, F186Y, R189H, K190R, E196H, K199D, and M208V;
(d) G13N, S20G, A33M, K34R, I85V, T86M, N104D, L107F, N114R, A134P, A142C, S144G, R147E, H149R, Q151D, Y161H, H163I, S165H, F184V, F186Y, K190R, E196H, K199D and M208V;
(e) G13N, S20G, A33M, K34R, I85V, T86M, N104D, L107F, N114R, A134P, A142C, S144G, R147E, H149R, Q151D, V157K, Y161H, H163I, S165H, F184V, F186Y, K190R, E196H, K199D, and M208V;
(f) G13N, S20G, A33M, K34R, I85V, T86M, T100I, N104D, L107F, K112E, N114R, A134P, A142R, S144G, R147E, H149R, Q151N, V157K, Y161H, H163I, S165H, F184V, F186Y, R189Y, K190R, E196H, K199D, and M208V;
(g) G13N, S20G, A33M, K34R, I85V, T86M, N104D, L107F, N114R, A134P, A142C, S144G, R147E, H149R, Q151D, V157K, Y161H, H163I, S165H, F184V, F186Y, R189Y, K190R, E196H, K199D, and M208V;
(h) G13N, S20G, A33M, K34R, I85V, T86M, T100I, N104D, L107F, K112E, N114R, A134P, A142C, S144G, R147E, H149R, Q151D, V157K, Y161H, H163I, S165H, F184V, F186Y, R189Y, K190R, E196H, K199D, and M208V; or
(i) G13N, T19E, S20G, K25N, A33M, K34R, A51M, I85V, T86M, Y88F, L94C, T100I, N104D, L107F, K112E, N114R, S120P, A134P, A142C, S144G, R147E, H149R, Q151N, V157K, Y161H, H163I, S165H, F184V, F186Y, R189H, K190R, E196H, K199D, T201N and M208V.

Also provided herein are fluorescent protein cyOFP variants of a wild-type fluorescent protein cyOFP from Escherichia coli of SEQ ID NO: 68 comprising one or more mutations selected from L8V, E11P, H14K, S15T, L19M, H32G, K37N, N45M, R46K, V50T, A58S, A63M, M65L, K71E, L106C, T1121 or T112V; L114I, A132P, L151M; A154R, A154K or A154N; K164M, V169K, N177H, K179R, T189K, N211D, V220W, V220H or V220Q, and D227H.

In one embodiment, provided herein is a fluorescent protein cyOFP variant of a wild-type fluorescent protein cyOFP from Escherichia coli of SEQ ID NO: 68 comprising mutations:
(a) L8V, E11P, H14K, L19M, H32G, N45M, A63M, M65L, K71E, A132P, A154R, K164M, V169K, N177H, and D227H;
(b) E11P, H14K, L19M, H32G, K37N, N45M, R46K, A63M, K71E, A132P, A154R, K164M, V169K, N177H, T189K, N211D, V220W, and D227H;
(c) E11P, H14K, L19M, H32G, N45M, A63M, M65L, K71E, A154R, K164M, V169K and N177H;
(d) L8V, E11P, H14K, L19M, H32G, K37N, N45M, R46K, A63M, K71E, T112I, A132P, A154K, K164M, V169K, N177H, T189K, N211D, and D227H;
(e) L8V, E11P, H14K, L19M, H32G, N45M, A63M, K71E, T112I, A154R, K164M, V169K and N177H;
(f) E11P, H14K, L19M, H32G, K37N, N45M, R46K, V50T, A58S, A63M, K71E, L106C, T112V, L114I, A132P, L151M, A154R, K164M, V169K, N177H, T189K, N211D, V220H and D227H;
(g) E11P, H14K, L19M, H32G, K37N, N45M, R46K, V50T, A58S, A63M, M65L, K71E, L106C, T112I, A132P, L151M, A154N, K164M, V169K, N177H, T189K, N211D, V220Q and D227H;
(h) E11P, H14K, L19M, N45M, K71E, A154R, K164M, V169K, N177H, and K179R;
(i) E11P, H14K, S15T, L19M, H32G, K37N, N45M, R46K, A63M, K71E, T112I, A132P, A154K, K164M, V169K, N177H, T189K, N211D, and D227H;
(j) H14K, L19M, N45M, K71E, A154R, K164M, and V169K;
(k) E11P, H14K, L19M, H32G, N45M, A63M, K71E, A132P, A154R, K164M, V169K, N177H and D227H;
(l) E11P, H14K, L19M, N45M, K71E, A154R, K164M, V169K, and N177H; or
(m) E11P, H14K, L19M, H32G, K37N, N45M, R46K, A63M, K71E, T112I, A132P, A154R, K164M, V169K, N177H, T189K, N211D, and D227H.

Each of the enzyme variants provided herein retain at least 95% activity, at least 90% activity, at least 85% activity, at least 80% activity, or at least 75% activity, after 4 days at 20° C. and at a pH in the range of 3.5-8, such as a pH in the range of 5-7.

Other features and advantages of this invention will become apparent from the following detailed description, and examples. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

In the following detailed description, numerous specific details are set forth in order to provide a thorough understanding of the invention. However, it will be understood by those skilled in the art that this invention may be practiced without these specific details. In other instances, well-known methods, procedures, and components have not been described in detail so as not to obscure this invention.

### Pyruvate Transaminase Variants

Pyruvate transaminase is an enzyme in the class-III pyridoxal-phosphate-dependent aminotransferase (“ATIII”) family having transaminase activity and pyridoxal phosphate binding activity. Pyridoxal phosphate is the active form of vitamin B6, and is a coenzyme in transamination reactions, as well as in numerous other enzymatic reactions. Pyruvate transaminase has been purified from Vibrio fluvialis JS17 and has been characterized as having activity toward chiral amines and a lack of activity toward beta-alanine, an optimal pH of 9.2 and an optimal temperature of 37° C. The aminotransferases of the ATIII family also are important in plant growth and development, and plant responses under abiotic stresses, i.e., environmental conditions that reduce growth, including but not limited to water deficit or excess, extreme temperatures, soil salinity and/or acidy, and mineral deficiency or toxicity.

Provided herein are pyruvate transaminase variants of a wild-type pyruvate transaminase from Vibrio fluvialis of SEQ ID NO: 39 comprising one or more mutations selected from A74E, Y76M, V103M, S108M, F130Y, Q196E, Q210L, A214P, G219A, L243Q, S304A, K305Q, T309E, A313K or A313R, E315G, E316T, A323Y, K335E, N342D, N405E; T406A, T408E, T408F or T408Q, and E436D.

In one embodiment, provided herein is a pyruvate transaminase variant of a wild-type pyruvate transaminase from Vibrio fluvialis of SEQ ID NO: 39 comprising mutations:
(a) S108M, F130Y, A214P, L243Q, S304A, E315G, A323Y, N342D, T408E, and E436D;
(b) S108M, F130Y, A214P, L243Q, S304A, E315G, A323Y, T408F, and E436D;
(c) Y76M, V103M, S108M, F130Y, A214P, G219A, L243Q, S304A, T309E, A313K, E315G, E316T, A323Y, N342D, N405E, T408Q, and E436D;
(d) Y76M, V103M, S108M, F130Y, A214P, L243Q, S304A, T309E, A313K, E315G, A323Y, N342D, T408Q, and E436D;
(e) Y76M, V103M, S108M, F130Y, A214P, G219A, L243Q, S304A, T309E, A313K, E315G, A323Y, N342D, N405E, T408Q, and E436D;
(f) A74E, Y76M, V103M, S108M, F130Y, Q196E, Q210L, A214P, G219A, L243Q, S304A, K305Q, T309E, A313R, E315G, E316T, A323Y, K335E, N342D, N405E, T406A, T408E and E436D;
(g) Y76M, V103M, S108M, F130Y, A214P, G219A, L243Q, S304A, T309E, A313R, E315G, E316T, A323Y, N342D, T408Q, and E436D;
(h) A74E, Y76M, V103M, S108M, F130Y, Q196E, Q210L, A214P, G219A, L243Q, S304A, K305Q, T309E, A313K, E315G, E316T, A323Y, K335E, N342D, N405E, T406A, T408Q and E436D; or
(i) Y76M, V103M, S108M, F130Y, A214P, G219A, L243Q, S304A, T309E, A313K, E315G, A323Y, N342D, T408Q, and E436D.

In one embodiment, provided herein are nucleic acids encoding an above-described pyruvate transaminase variant of SEQ ID NO: 39. In another embodiment, provided herein are expression vectors comprising the nucleic acid encoding the pyruvate transaminase variant of SEQ ID NO: 39. In another embodiment, provided herein are host cells transformed with the expression vector comprising the nucleic acid encoding the pyruvate transaminase variant of SEQ ID NO: 39. In a further embodiment, provided herein are methods for producing a pyruvate transaminase variant, said method comprising expressing the pyruvate transaminase variant in the transformed host cell, wherein the host cell is transformed with the expression vector comprising the nucleic acid encoding the pyruvate transaminase variant of SEQ ID NO: 39.

### Triacylglycerol Lipase Variants

Triacylglycerol lipase is an enzyme that hydrolyzes triacylglycerol to diacylglycerol and a carboxylate. Pancreatic lipase, also called pancreatic triacylglycerol lipase is an enzyme secreted into the duodenum by the pancreas that breaks down dietary fat (triglyceride) to monoglycerides and free fatty acids. Normal levels of pancreatic lipase are low, but during pancreatitis, an inflammation of the pancreas, or in pancreatic adenocarcinoma, the pancreas secretes three or more times as much pancreatic lipase. In certain diseases, such as celiac disease, cystic fibrosis, Crohn disease, not enough lipase is produced; patents may be prescribed a pancreatic enzyme therapy of protease, amylase and lipase, or enteric-coated lipase supplements.

Also provided herein are triacylglycerol lipase variants of a wild-type triacylglycerol lipase from Burkholderia glumae of SEQ ID NO: 40 comprising one or more mutations selected from A63G, N64G, S74E, S78R, A106E, S132A, F161Y, T168Y, T176P, N183W, D196N, R204K, Q210G, T211A, Y214F, R216Q, A234P, S241N, Q242T, A297G, Q300P, F312Y, S319N, V334I, N338F, T348N or T348Q, L355N, and V358L.

In one embodiment, provided herein is a triacylglycerol lipase variant of a wild-type triacylglycerol lipase from Burkholderia glumae of SEQ ID NO: 40 comprising mutations:
(a) A63G, N64G, S74E, S78R, A106E, S132A, F161Y, T168Y, T176P, N183W, D196N, R204K, Q210G, T211A, Y214F, R216Q, A234P, S241N, Q242T, A297G, Q300P, F312Y, S319N, V334I, N338F, T348N, L355N, and V358L;
(b) A63G, N64G, S74E, S78Q, S132A, F161Y, T168Y, T176P, D196N, R204K, Q210G, T211A, Y214F, A234P, S241N, Q242T, A297G, Q300P, F312Y, S319N, V334I, T348N, L355N and V358L;
(c) N64G, S132A, D196N, A234P, S241N, Q242T, A297G, Q300P, S319N, T348N, L355N and V358L;
(d) A63G, N64G, S74E, S78Q, S132A, F161Y, T168Y, T176P, D196N, R204K, Q210G, T211A, Y214F, A234P, S241N, Q242T, A297G, Q300P, F312Y, S319N, V334I, T348Q, L355N and V358L;
(e) S241N, A297G, Q300P, S319N, and V358L;
(f) N64G, S132A, D196N, A234P, S241N, Q242T, A297G, Q300P, F312Y, S319N, T348N, L355N, and V358L; or
(g) S241N, A297G, Q300P, S319N, T348N, and V358L.

In one embodiment, provided herein are nucleic acids encoding an above-described triacylglycerol lipase variant of SEQ ID NO: 40. In another embodiment, provided herein are expression vectors comprising the nucleic acid encoding the triacylglycerol lipase variant of SEQ ID NO: 40. In another embodiment, provided herein are host cells transformed with the expression vector comprising the nucleic acid encoding the triacylglycerol lipase variant of SEQ ID NO: 40. In a further embodiment, provided herein are methods for producing a triacylglycerol lipase variant, said method comprising expressing the triacylglycerol lipase variant in the transformed host cell, wherein the host cell is transformed with the expression vector comprising the nucleic acid encoding the triacylglycerol lipase variant of SEQ ID NO: 40.

### Halohydrin Dehalogenase Variants

Halohydrin dehalogenase, also called haloalcohol dehalogenase or halohydrin hydrogen-halide lyase, is an enzyme in certain bacteria that cleaves the carbon-halogen bond in organic compounds to yield epoxides from a vicinal hydroxyl group, which is a reversible reaction. There are three different isoform classes of halohydrin dehalogenase, the A, B and C groups. Halogenated aliphatics are environmental pollutants, thus, the ability of halohydrin dehalogenase to dehalogenase these compounds render the bacteria encoding this enzyme, as well as the enzyme itself, as bioremediation tools to degrade halogenated compounds in polluted soil, and contaminated groundwater and wastewater. Industrial application of halohydrin dehalogenases for their reversible dehalogenation; in the reverse reaction negatively charged nucleophiles, such as azide, cyanide, or nitrite, are accepted, besides halides, to open the epoxide ring, thereby, novel C—N, C—C, or C—O bonds are formed by halohydrin dehalogenases, making halohydrin dehalogenase an attractive biocatalyst for the production of various β-substituted alcohols through epoxide ring opening. Isoform HheC from grain-negative bacterium Agrobacterium radiobacter strain AD1 has been studied the most because its encoded halohydrin dehalogenase is enantio-selective.

Provided herein are halohydrin dehalogenase variants of a wild-type halohydrin dehalogenase from Rhizobium radiobacter of SEQ ID NO: 41 comprising one or more mutations selected from T3V, K10R, F12G, S17A, S22A, E33P, Q37D, K38P, L41R, K52H, E56A, I63V, A65R, T67I; S68K or S68E; S78A, D80A, A83P, Q87R; K91Q or K91E; A93D or A93S; V94P; G99A or G99K; Q105L, R107G, A110R, N113R or N113Q, S117P, F136L, W139L, K140P, E141G, S147A; C153I or C153N; T154A, N157K, S160A, V205Q, T206V, K215E and M245V.

In one embodiment, provided is a halohydrin dehalogenase variant of a wild-type halohydrin dehalogenase from Rhizobium radiobacter of SEQ ID NO: 41 comprising mutations:
(a) T3V, F12G, S17A, E33P, Q37D, K38P, L41R, K52H, E56A, T67I, S68K, S78A, D80A, A83P, Q87R, K91Q, V94P, G99A, R107G, A110R, N113R, S117P, K140P, E141G, C153I, T154A, N157K, S160A, V205Q, and K215E;
(b) F12G, E33P, Q37D, L41R, T67I, S68K, D80A, A83P, Q87R, V94P, G99A, N113R, S117P, K140P, C153I, T154A, and N157K;
(c) F12G, E33P, Q37D, L41R, T67I, S68K, A83P, Q87R, V94P, G99A, N113R, S117P, C153I, T154A, and N157K;
(d) T3V, K10R, F12G, S17A, S22A, E33P, Q37D, K38P, L41R, K52H, E56A, I63V, T67I, S68K, S78A, D80A, A83P, Q87R, K91E, A93D, V94P, G99K, Q105L, R107G, A110R, N113Q, S117P, F136L, W139L, E141G, S147A, C153N, T154A, N157K, S160A, V205Q, K215E and M245V;
(e) F12G, S17A, E33P, Q37D, K38P, L41R, K52H, E56A, T67I, S68K, D80A, A83P, Q87R, V94P, G99A, R107G, A110R, N113R, S117P, C153N, T154A, N157K, V205Q, and K215E;
(f) T3V, K10R, F12G, S17A, S22A, E33P, Q37D, K38P, L41R, K52H, I63V, A65R, T67I, S68E, S78A, D80A, A83P, Q87R, K91E, A93D, V94P, G99A, Q105L, R107G, A110R, N113R, S117P, F136L, W139L, E141G, S147A, C153I, T154A, N157K, S160A, V205Q, and K215E;
(g) F12G, S17A, E33P, Q37D, K38P, L41R, K52H, E56A, T67I, S68K, D80A, A83P, Q87R, V94P, G99A, R107G, A110R, N113Q, S117P, K140P, C153I, T154A, N157K, V205Q and K215E;
(h) T3V, K10R, F12G, S17A, S22A, E33P, Q37D, K38P, L41R, K52H, E56A, T67I, S68E, S78A, D80A, A83P, Q87R, K91E, A93S, V94P, G99A, R107G, A110R, N113R, S117P, W139L, E141G, S147A, C153I, T154A, N157K, S160A, V205Q, T206V, and K215E;
(i) F12G, E33P, L41R, T67I, S68K, A83P, Q87R, V94P, G99A, N113R, S117P, K140P, C153I, T154A, and N157K;
(j) T3V, K10R, F12G, S17A, S22A, E33P, Q37D, K38P, L41R, K52H, E56A, T67I, S68E, S78A, D80A, A83P, Q87R, K91E, A93S, V94P, G99A, R107G, A110R, N113R, S117P, W139L, K140P, E141G, S147A, C153I, T154A, N157K, S160A, V205Q, and K215E;
(k) T3V, F12G, S17A, E33P, Q37D, K38P, L41R, K52H, E56A, T67I, S68K, S78A, D80A, A83P, Q87R, K91Q, V94P, G99A, R107G, A110R, N113Q, S117P, E141G, C153N, T154A, N157K, S160A, V205Q, and K215E;
(l) F12G, E33P, Q37D, K38P, L41R, K52H, E56A, T67I, S68K, D80A, A83P, Q87R, V94P, G99A, R107G, A110R, N113R, S117P, K140P, C153I, T154A, and N157K; or
(m) F12G, E33P, L41R, T67I, S68K, D80A, A83P, Q87R, V94P, G99A, N113R, S117P, K140P, C153N, T154A, and N157K.

In one embodiment, provided herein are nucleic acids encoding an above-described halohydrin dehalogenase variant of SEQ ID NO: 41. In another embodiment, provided herein are expression vectors comprising the nucleic acid encoding the halohydrin dehalogenase variant of SEQ ID NO: 41. In another embodiment, provided herein are host cells transformed with the expression vector comprising the nucleic acid encoding the halohydrin dehalogenase variant of SEQ ID NO: 41. In a further embodiment, provided herein are methods for producing a halohydrin dehalogenase variant, said method comprising expressing the halohydrin dehalogenase variant in the transformed host cell, wherein the host cell is transformed with the expression vector comprising the nucleic acid encoding the halohydrin dehalogenase variant of SEQ ID NO: 41.

### Aminotransferase Variants

Aminotransferases, also called transaminases, are enzymes that catalyze the amino-transfer reaction between an amino acid and a 2-keto acid, i.e., an alpha-keto acid, in which the amino group of the amino acid and keto group of a keto acid are exchanged: the amino acid becomes a keto acid and the keto acid becomes an amino acid; this reaction is reversible. Transaminases are used to produce amino acids and chiral amines. Alanine transaminase, previously called serum glutamate-pyruvate transaminase, is found in plasma and body tissues, primarily in the liver. Alanine transaminase catalyzes the transfer of an amino group from L-alanine to α-ketoglutarate, reversibly producing pyruvate and L-glutamate.

Also provided herein are aminotransferase variants of a wild-type aminotransferase from Chromobacterium violaceum of SEQ ID NO: 42 comprising one or more mutations selected from M36L, G39A, D70E, F71L, R76Y, K90Q, V97I, S101E or S101K, A109P, D112N, R113H, S123A, V124N, T145V, L146I, G152A, S162A, V202L; V203H or V203K; A217P, D315E, A337R, V236I, A239D, H276Y; A286I or A286V; K304D, A312E, H333L; A337K or A337R; Q346E, K358Q, E368P, V379L, V379I or V379M; Q380A, N387D, K390T, D396N, F397E, T402W, R410N, S424A, T452E, and A455E or A455K.

In one embodiment, provided herein is an aminotransferase variant of a wild-type aminotransferase from Chromobacterium violaceum of SEQ ID NO: 42 comprising mutations:
(a) G39A, F71L, S101E, A109P, D112N, R113H, S123A, V124N, L146I, V202L, V203H, A217P, V236I, A239D, A286I, K304D, A312E, H333L, A337K, E368P, V379L, Q380A, D396N, F397E, T402W, and A455E;
(b) D70E, D112N, R113H, S123A, V124N, A217P, A337R, E368P, Q380A, D396N, and F397E;
(c) G39A, D70E, F71L, S101E, A109P, D112N, R113H, S123A, V124N, L146I, A217P, V236I, A286I, H333L, A337K, E368P, V379L, Q380A, D396N, F397E, and A455E;
(d) G39A, D112N, R113H, S123A, V124N, A217P, A337R, E368P, V379L, Q380A, D396N and F397E;
(e) D70E, D112N, R113H, S123A, V124N, A217P, A286V, A337R, E368P, V379L, Q380A, D396N, and F397E;
(f) G39A, D70E, F71L, S101K, A109P, D112N, R113H, S123A, V124N, L146I, V202L, V203H, A217P, V236I, A239D, A286I, K304D, A312E, H333L, A337R, E368P, V379L, Q380A, D396N, F397E, T402W, and A455E;
(g) G39A, D70E, F71L, K90Q, V97I, S101E, A109P, D112N, R113H, S123A, V124N, T145V, L146I, G152A, V202L, V203H, A217P, V236I, A239D, H276Y, A286I, K304D, A312E, H333L, A337K, Q346E, E368P, V379I, Q380A, N387D, D396N, F397E, T402W, R410N, T452E, and A455K;
(h) M36L, G39A, D70E, F71L, K90Q, V97I, S101K, A109P, D112N, R113H, S123A, V124N, T145V, L146I, G152A, V202L, V203K, A217P, V236I, A239D, H276Y, A286I, K304D, A312E, H333L, A337K, Q346E, K358Q, E368P, V379M, Q380A, N387D, K390T, D396N, F397E, T402W, R410N, S424A, T452E, and A455K;
(i) M36L, G39A, D70E, F71L, R76Y, K90Q, V97I, S101K, A109P, D112N, R113H, S123A, V124N, T145V, L146I, G152A, V202L, V203K, A217P, V236I, A239D, H276Y, A286I, K304D, A312E, H333L, A337R, Q346E, K358Q, E368P, Q380A, N387D, K390T, D396N, F397E, T402W, R410N, S424A, T452E, and A455E;
(j) G39A, D70E, S101E, A109P, D112N, R113H, S123A, V124N, L146I, A217P, A286I, H333L, A337K, E368P, V379M, Q380A, D396N, and F397E;
(k) G39A, D70E, F71L, S101E, A109P, D112N, R113H, S123A, V124N, L146I, A217P, V236I, A286I, D315E, H333L, A337R, E368P, Q380A, D396N, F397E, and A455E;
(l) G39A, D70E, F71L, K90Q, V97I, S101E, A109P, D112N, R113H, S123A, V124N, T145V, L146I, G152A, S162A, V202L, V203H, A217P, V236I, A239D, H276Y, A286I, K304D, A312E, H333L, A337R, Q346E, E368P, V379I, Q380A, N387D, D396N, F397E, T402W, R410N, T452E, and A455K;
(m) G39A, D70E, D112N, R113H, S123A, V124N, A217P, A286V, A337R, E368P, V379L, Q380A, D396N, and F397E; or
(n) G39A, S101E, A109P, D112N, R113H, S123A, V124N, L146I, A217P, A286I, H333L, A337R, E368P, V379L, Q380A, D396N, and F397E.

In one embodiment, provided herein are nucleic acids encoding an above-described aminotransferase variant of SEQ ID NO: 42. In another embodiment, provided herein are expression vectors comprising the nucleic acid encoding the aminotransferase variant of SEQ ID NO: 42. In another embodiment, provided herein are host cells transformed with the expression vector comprising the nucleic acid encoding the aminotransferase variant of SEQ ID NO: 42. In a further embodiment, provided herein are methods for producing a aminotransferase variant, said method comprising expressing the aminotransferase variant in the transformed host cell, wherein the host cell is transformed with the expression vector comprising the nucleic acid encoding the aminotransferase variant of SEQ ID NO: 42.

### Putative Secreted Lipase Variants

Lipases are enzymes that catalyze the hydrolysis of fats, oils and triglycerides. In humans, the pancreas secretes pancreatic lipase, the main enzyme to break down dietary fats and triglycerides in digested oils to monoglycerides and two fatty acids. Humans also produce a lingual lipase, a bile salt-dependent lipase, hepatic lipase, lipoprotein lipase, as well as gastric lipase in the infant. Bacteria and fungi secrete lipase to absorb nutrients from external medium. Pathogenic organisms express and secrete lipases to promote host invasion. Lipases also catalyse transesterification, aminolysis and acidolysis reactions. Bacterial and fungal lipases are used as industrial catalysts the food, detergent and pharmaceutical industries.

Clostridium botulinum is the bacterium that produces botulinum toxin. C. botulinum has two genes encoding two putative secreted lipases, putative secreted lipase CB00863 and CB02061. Identification of lipase-positive C. botulinum is made its growth at a pH of between 4.8 and 7.0 and an inability to use lactose as a primary source of carbon. Lipase activity by this Gram-positive anaerobe produces a thin pearly layer around colonies grown on egg yolk-containing medium, which property is utilized as a diagnostic test for C. botulinum.

Also provided herein are putative secreted lipase variants of a wild-type putative secreted lipase from Clostridium botulinum of SEQ ID NO: 43 comprising one or more mutations selected from D69N, E91Q, I101T, K137D, A140R, S141N, H143Y, V168K, K183G, K196P, N200D, K246H, S253V, S265A, T277M, K289N, N308D, E339Y, T346K, A375P, S395T, Q398K, G437T, M469I, N477Q, and S479P.

In one embodiment, also provided is a putative secreted lipase variant of a wild-type putative secreted lipase from Clostridium botulinum of SEQ ID NO: 43 comprising mutations:
(a) E91Q, I101T, A140R, K183G, K246H, S265A, T277M, K289N, N308D, E339Y, A375P, S395T, G437T, and S479P;
(b) I101T, K183G, T277M, K289N, N308D, A375P, S395T, and S479P;
(c) D69N, E91Q, I101T, K137D, A140R, S141N, H143Y, V168K, K183G, K196P, N200D, K246H, S265A, T277M, K289N, N308D, E339Y, T346K, A375P, S395T, Q398K, G437T, M469I, N477Q, and S479P;
(d) I101T, K183G, T277M, K289N, A375P, S395T, and S479P;
(e) D69N, E91Q, I101T, K137D, A140R, S141N, H143Y, V168K, K183G, K196P, N200D, K246H, S265A, T277M, K289N, N308D, E339Y, T346K, A375P, S395T, Q398K, G437T, N477Q, and S479P;
(f) I101T, K183G, K246H, S253V, T277M, K289N, N308D, A375P, S395T, G437T, and S479P;
(g) I101T, K183G, K246H, T277M, K289N, N308D, A375P, S395T, G437T, and S479P; or (h) K183G, T277M, A375P, S395T, and S479P.

In one embodiment, provided herein are nucleic acids encoding an above-described putative secreted lipase variant of SEQ ID NO: 43. In another embodiment, provided herein are expression vectors comprising the nucleic acid encoding the putative secreted lipase variant of SEQ ID NO: 43. In another embodiment, provided herein are host cells transformed with the expression vector comprising the nucleic acid encoding the putative secreted lipase variant of SEQ ID NO: 43. In a further embodiment, provided herein are methods for producing a putative secreted lipase variant, said method comprising expressing the putative secreted lipase variant in the transformed host cell, wherein the host cell is transformed with the expression vector comprising the nucleic acid encoding the putative secreted lipase variant of SEQ ID NO: 43.

### Alcohol Dehydrogenase Variants

Alcohol dehydrogenases (ADH) are a group of enzymes that catalyze both the oxidation of primary and secondary alcohol to aldehydes and ketones, respectively, and the reverse reaction. In humans, there are five classes of alcohol dehydrogenases, of which the primary ADH is a class 1 form, in the liver catalyzes the/oxidation reaction of ethanol to acetaldehyde using the coenzyme nicotinamide adenine dinucleotide (NAD+), which is reduced to NADH. In yeast and bacteria, alcohol dehydrogenase ferments glucose to ethanol and carbon dioxide. Geobacillus stearothermophilus, an obligate anaerobe, encodes an alcohol dehydrogenase that is thermophilic and NAD+-dependent and that catalyzes the oxidation of an alcohol, e.g., ethanol and methanol, with NAD+ to an aldehyde or ketone and NADH. The alcohol dehydrogenase from G. stearothermophilus is utilized in the biocatalytic synthesis of w-oxo lauric acid methyl ester, which is a key intermediate for bio-based polyamide 12 production, from the corresponding long-chain fatty alcohol by oxidation.

Further provided herein are alcohol dehydrogenase variants of a wild-type alcohol dehydrogenase from Geobacillus stearothermophilus of SEQ ID NO: 44 comprising one or more mutations selected from K10G; K11E or K11Q; Q14K or Q14V; V15I, E19P, K20V or K20I, K22T, S24G, V66I, I67V, E68V, E69A, Y89W, Q102W, R108H, Q110K, A112T, Y120F, A127D, K133H, F147L, T153V, V159E or V159M; V179M, L196I, G197D; E199D, K205R, Q206E or Q206K; K216L, H217K, D218E, A220P, Q222K, W223F, I224M, E226K, T234A, A242P, E245Q, S246Q, K249R or K249N; I251L, A256T, C257V or C257L; E265G, I267M or I267F, I269V or I269L; V279I, V28I, T306P or T306V; V308I, Q311R; N315K or N315D; D318E, L324R, N329D or N329K, K335D, V336L, D337E, K335D, V336L, and D337E.

Also provided herein is an alcohol dehydrogenase variant of a wild-type alcohol dehydrogenase from Geobacillus stearothermophilus of SEQ ID NO: 44 comprising mutations:
(a) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, I269V, V279I, V308I, Q311R, N315K, D318E, K335D, V336L, and D337E;
(b) K10G, K11E, Q14K, V15I, E19P, K20V, K22T, S24G, V66I, I67V, Q102W, R108H, Q110K, A112T, A127D, K133H, V179M, L196I, G197D, Q206E, K216L, H217K, D218E, A220P, Q222K, W223F, I224M, T234A, A242P, E245Q, S246Q, K249R, A256T, C257V, I267M, V279I, V308I, Q311R, N315K, D318E, K335D, V336L, and D337E;
(c) K10G, K11Q, Q14V, V15I, E19P, K20I, K22T, S24G, V66I, I67V, E68V, E69A, Y89W, Q102W, R108H, A112T, Y120F, A127D, K133H, T153V, V159E, V179M, L196I, G197D, E199D, Q206K, K216L, H217K, D218E, A220P, Q222K, W223F, I224M, E226K, T234A, A242P, S246Q, K249R, I251L, A256T, C257L, E265G, I267M, I269V, V279I, T306P, V3081, Q311R, N315D, D318E, L324R, N329D, K335D, V336L, and D337E;
(d) K10G, K11Q, Q14K, V15I, E19P, K20I, K22T, S24G, V66I, I67V, Q102W, R108H, Q110K, A112T, Y120F, A127D, K133H, F147L, T153V, V159E, V179M, L196I, G197D, Q206E, K216L, H217K, D218E, A220P, Q222K, W223F, I224M, T234A, A242P, E245Q, S246Q, K249R, A256T, C257L, I267M, I269V, V279I, T306V, V308I, Q311R, N315D, D318E, N329D, K335D, V336L, and D337E;
(e) K10G, K11Q, Q14V, V15I, E19P, K20I, K22T, S24G, V28I, V66I, I67V, E68V, E69A, Y89W, Q102W, R108H, A112T, Y120F, A127D, K133H, T153V, V159E, V179M, L196I, G197D, E199D, Q206K, K216L, H217K, D218E, A220P, Q222K, W223F, I224M, E226K, T234A, A242P, S246Q, K249R, I251L, A256T, C257L, E265G, I267M, I269V, V279I, T306P, V308I, Q311R, N315D, D318E, L324R, N329D, K335D, V336L, and D337E;
(f) K10G, K11Q, Q14K, V15I, E19P, K20I, K22T, S24G, V66I, I67V, Q102W, R108H, Q110K, A112T, A127D, K133H, V159M, V179M, L196I, G197D, Q206E, K216L, H217K, D218E, A220P, Q222K, W223F, I224M, T234A, A242P, E245Q, S246Q, K249R, A256T, C257V, I267F, I269L, V279I, T306P, V308I, Q311R, D318E, N329K, K335D, V336L, and D337E;
(g) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, V279I, V308I, N315K, D318E, K335D, V336L, and D337E;
(h) K10G, K11E, Q14K, V15I, E19P, K22T, S24G, V66I, I67V, Q102W, R108H, Q110K, A127D, K133H, V159E, V179M, L196I, G197D, Q206E, K216L, H217K, D218E, A220P, Q222K, W223F, I224M, T234A, A242P, E245Q, K249N, A256T, I267M, I269V, V279I, V308I, N315K, D318E, K335D, V336L, and D337E;
(i) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, I269V, V279I, V308I, N315K, D318E, K335D, V336L, and D337E;
(j) K10G, K11Q, Q14V, V15I, E19P, K20I, K22T, S24G, V66I, I67V, E69A, Y89W, Q102W, R108H, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249N, I251L, A256T, C257L, I267M, I269L, V279I, T306P, V308I, Q311R, N315D, D318E, N329D, K335D, V336L, and D337E;
(k) K10G, K11Q, Q14V, V15I, E19P, K20I, K22T, S24G, V66I, I67V, E69A, Y89W, Q102W, R108H, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249R, I251L, A256T, C257L, I267M, I269V, V279I, T306P, V308I, Q311R, N315D, D318E, N329D, K335D, V336L, and D337E;
(l) K10G, K11Q, Q14K, V15I, E19P, K20I, K22T, S24G, V66I, I67V, Q102W, R108H, Q110K, A112T, A127D, K133H, F147L, V159M, V179M, L196I, G197D, K205R, Q206E, K216L, H217K, D218E, A220P, Q222K, W223F, I224M, T234A, A242P, E245Q, S246Q, K249R, A256T, C257V, I267M, I269V, V279I, T306V, V308I, Q311R, D318E, N329K, K335D, V336L, and D337E;
(m) K10G, K11Q, Q14K, V15I, E19P, K20I, K22T, S24G, V66I, I67V, Y89W, Q102W, R108H, Q110K, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249R, A256T, C257L, I267F, I269L, V279I, T306V, V308I, Q311R, N315D, D318E, N329D, K335D, V336L, and D337E; or
(n) K10G, K11E, Q14K, V15I, E19P, K22T, S24G, V66I, I67V, Q102W, R108H, Q110K, A127D, K133H, V159E, V179M, L196I, G197D, Q206E, K216L, H217K, D218E, A220P, Q222K, W223F, I224M, T234A, A242P, E245Q, K249R, A256T, I267F, I269V, V279I, V308I, N315K, D318E, K335D, V336L, and D337E.

Further provided herein are alcohol dehydrogenase variants of a wild-type alcohol dehydrogenase from Geobacillus stearothermophilus of SEQ ID NO: 52 comprising one or more mutations selected from N7R or N7T; K10G, A12P, E19P, R20I, K22T, L23I, E24G, E25P or E25R, V28I, I51V, E69A, A71G, K72P, K75T, I77V, Y89W, E94H or E94R; L99R, Q102W or Q102R; L110Q, G112T, Y120F, P127H, A132V, V141L, V143A, T153V, V159E, A162T, I179M, L181V, S197D, S201L, D206K, A212T, I213V, G215A, K222Q, H225Q, D226K, V228I, S236V, K241P, Q249R, K252R, L257I or L257V; V259L, N264P, A265G, L267M, V279I, S280T, V281I, K282R, K290Q, M292L, A311R, E312R or E312K; E318D, N329D, and K335D.

In another embodiment, provided herein is an alcohol dehydrogenase variant of a wild-type alcohol dehydrogenase from Geobacillus stearothermophilus of SEQ ID NO: 52 comprising mutations:
(a) K10G, A12P, E19P, R20I, L23I, E24G, E25P, I51V, A71G, K72P, K75T, I77V, E94H, Q102W, L110Q, G112T, Y120F, A132V, V143A, T153V, I179M, L181V, S197D, S201L, D206K, A212T, G215A, H225Q, D226K, V228I, S236V, K241P, Q249R, K252R, L257I, V259L, N264P, A265G, L267M, V279I, S280T, K282R, K290Q, A311R, E312R and K335D;
(b) A12P, E19P, R20I, L23I, E24G, E25R, I51V, A71G, K72P, K75T, E94R, Q102W, G112T, A132V, V143A, L181V, S197D, S201L, A212T, G215A, D226K, S236V, K241P, Q249R, K252R, V259L, N264P, A265G, S280T, K282R, K290Q, A311R, E312K, and K335D;
(c) N7R, K10G, A12P, E19P, R20I, L23I, E24G, E25P, I51V, A71G, K72P, K75T, I77V, E94H, Q102W, L110Q, G112T, Y120F, A132V, V143A, T153V, I179M, L181V, S197D, S201L, D206K, A212T, I213V, G215A, H225Q, D226K, V228I, S236V, K241P, Q249R, K252R, L257V, V259L, N264P, A265G, L267M, V279I, S280T, V281I, K282R, K290Q, A311R, E312R, N329D, and K335D;
(d) A12P, E19P, R20I, L23I, E24G, E25R, A71G, K72P, E94H, Q102W, G112T, A132V, V143A, V159E, L181V, S201L, A21 and 2T, G215A, S236V, K241P, Q249R, K252R, V259L, N264P, A265G, S280T, K282R, K290Q, E312R, and K335D;
(e) A12P, E19P, R20I, L23I, E24G, E25R, I51V, A71G, K72P, K75T, E94H, Q102W, G112T, A132V, V143A, V159E, L181V, S197D, S201L, A212T, G215A, D226K, S236V, K241P, Q249R, K252R, L257V, V259L, N264P, A265G, S280T, K282R, K290Q, A311R, E312R and K335D;
(f) A12P, E19P, R20I, L23I, E24G, E25P, I51V, A71G, K72P, K75T, E94H, Q102W, L110Q, G112T, A132V, V143A, L181V, S197D, S201L, D206K, A212T, G215A, D226K, V228I, S236V, K241P, Q249R, K252R, V259L, N264P, A265G, S280T, K282R, K290Q, A311R, E312R, and K335D;
(g) A12P, E19P, R20I, L23I, E24G, E25R, I51V, A71G, K72P, E94H, Q102W, G112T, A132V, V143A, L181V, S201L, A212T, G215A, S236V, K241P, Q249R, K252R, V259L, N264P, A265G, S280T, K282R, K290Q, E312R, and K335D;
(h) K10G, A12P, E19P, R20I, L23I, E24G, E25P, I51V, A71G, K72P, K75T, I77V, E94H, Q102W, L110Q, G112T, Y120F, A132V, V143A, T153V, I179M, L181V, S197D, S201L, D206K, A212T, G215A, H225Q, D226K, V228I, S236V, K241P, Q249R, K252R, V259L, N264P, A265G, L267M, V279I, S280T, K282R, K290Q, M292L, A311R, E312R and K335D;
(i) K10G, A12P, E19P, R20I, L23I, E24G, E25P, V28I, I51V, A71G, K72P, K75T, I77V, E94H, Q102R, L110Q, G112T, Y120F, A132V, V143A, T153V, V159E, I179M, L181V, S197D, S201L, D206K, A212T, I213V, G215A, H225Q, D226K, V228I, S236V, K241P, Q249R, K252R, L257I, V259L, N264P, A265G, L267M, V279I, S280T, V281I, K282R, K290Q, A311R, E312R, N329D, and K335D;
(j) A12P, E19P, R20I, L23I, E24G, E25R, I51V, A71G, K72P, E94H, Q102W, G112T, A132V, V143A, V159E, L181V, S201L, A212T, G215A, S236V, K241P, Q249R, K252R, V259L, N264P, A265G, S280T, K282R, K290Q, E312R, and K335D;
(k) N7T, K10G, A12P, E19P, R20I, K22T, L23I, E24G, E25P, V28I, I51V, E69A, A71G, K72P, K75T, I77V, Y89W, E94H, Q102R, L110Q, G112T, Y120F, P127H, A132V, V143A, T153V, V159E, I179M, L181V, S197D, S201L, D206K, A212T, I213V, G215A, K222Q, H225Q, D226K, V228I, S236V, K241P, Q249R, K252R, V259L, N264P, A265G, L267M, V279I, S280T, V281I, K282R, K290Q, A311R, E312R, E318D, N329D, and K335D;
(l) N7T, K10G, A12P, E19P, R20I, K22T, L23I, E24G, E25P, V28I, I51V, E69A, A71G, K72P, K75T, I77V, Y89W, E94H, L99R, Q102W, L110Q, G112T, Y120F, P127H, V141L, V143A, T153V, V159E, A162T, I179M, L181V, S197D, S201L, D206K, A212T, I213V, G215A, K222Q, H225Q, D226K, V228I, S236V, K241P, Q249R, K252R, V259L, N264P, A265G, L267M, V279I, S280T, K282R, K290Q, M292L, A311R, E312R, E318D, N329D and K335D; or
(m) A12P, E19P, R20I, L23I, E24G, E25P, V28I, I51V, A71G, K72P, K75T, E94R, Q102W, L110Q, G112T, A132V, V143A, L181V, S197D, S201L, D206K, A212T, G215A, H225R, D226K, V228I, S236V, K241P, Q249R, K252R, V259L, N264P, A265G, S280T, K282R, K290Q, A311R, E312K, and K335D.

Also provided herein are alcohol dehydrogenase variants of a wild-type alcohol dehydrogenase from Rhodococcus jostii of SEQ ID NO: 53 comprising one or more mutations selected from V36A, K44E or K44D, A46L, D70P, S73T, I77V, V83A or V83C, T91R, S97T, Q99M, S100Q, L105A, N108G, V110A, T111A, E113C or E113A; L114Q or L114M; N116D, I128V, A132C, A133M, T136A, Q139E, F140Y, S141A or S141T; T154D, W159K, V160A, S161A, T164G or T164S; S173A, A177T, G178A, E179G, K181Q, A182P, A187V or A1871; V199I, R200Q, A201G, V203R, N206G, G208R, V210I, I213V, S218E or S218D, L220F, F222W, F222M or F222Q; Q225K, S226F, D229T, F230H, Y232F, T233A, A235M, H239A, D241L, Y247W, V249Q, T254V, V255I, V261G, F270M, and E271R.

In one embodiment, provided herein is an alcohol dehydrogenase variant of a wild-type alcohol dehydrogenase from Rhodococcus jostii of SEQ ID NO: 53 comprising mutations: (a) V36A, K44E, A46L, D70P, S73T, I77V, V83A, T91R, S97T, Q99M, L105A, N108G, V110A, T111A, E113C, L114Q, N116D, A132C, A133M, T136A, Q139E, F140Y, S141A, T154D, V160A, S161A, T164G, E179G, A182P, A187V, R200Q, A201G, N206G, G208R, V210I, A211V, F222W, S226F, F230H, T233A, H239A, D241L, Y247W, T254V, V255I, V261G, F270M, and E271R;
(b) K44D, D70P, S73T, V83A, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, Y247W, V261G, and E271R;
(c) K44E, D70P, S73T, I77V, V83A, L105A, V110A, T111A, E113C, L114M, N116D, A132C, A133M, T136A, F140Y, S141T, T154D, V160A, T164S, A182P, R200Q, N206G, G208R, A211V, F222M, S226F, F230H, T233A, H239A, D241L, Y247W, V261G, F270M and E271R;
(d) V36A, K44D, A46L, D70P, S73T, I77V, V83A, S97T, Q99M, L105A, V110A, T111A, E113C, L114Q, N116D, A132C, A133M, T136A, Q139E, F140Y, S141A, T154D, V160A, S161A, T164G, A182P, A187I, R200Q, A201G, N206G, G208R, A211V, F222Q, S226F, F230H, T233A, H239A, D241L, Y247W, T254V, V255I, V261G, F270M, and E271R;
(e) V36A, K44D, A46L, D70P, S73T, I77V, V83A, T91R, S97T, Q99M, S100Q, L105A, N108G, V110A, T111A, E113C, L114M, N116D, I128V, A132C, A133M, T136A, Q139E, F140Y, S141A, T154D, W159K, V160A, S161A, T164G, S173A, A177T, G178A, E179G, K181Q, A182P, A187V, V199I, R200Q, A201G, V203R, N206G, G208R, V210I, A211V, I213V, S218E, F222W, Q225K, S226F, D229T, F230H, Y232F, T233A, A235M, H239A, D241L, Y247W, V249Q, T254V, V255I, V261G, F270M, and E271R;
(f) V36A, K44D, A46L, D70P, S73T, I77V, V83A, T91R, S97T, Q99M, S100Q, L105A, N108G, V110A, T111A, E113C, L114M, N116D, I128V, A132C, A133M, T136A, Q139E, F140Y, S141A, T154D, W159K, V160A, S161A, T164G, S173A, A177T, G178A, E179G, K181Q, A182P, A187V, V199I, R200Q, A201G, V203R, N206G, G208R, V210I, A211V, I213V, S218D, L220F, F222Q, Q225K, S226F, D229T, F230H, Y232F, T233A, A235M, H239A, D241L, Y247W, V249Q, T254V, V255I, V261G, F270M, and E271R;
(g) V36A, K44E, D70P, S73T, I77V, V83A, S97T, Q99M, L105A, V110A, T111A, E113A, L114M, N116D, A132C, A133M, T136A, Q139E, F140Y, S141A, T154D, V160A, T164S, A182P, R200Q, A201G, N206G, G208R, A211V, F222W, S226F, F230H, T233A, H239A, D241L, Y247W, T254V, V255I, V261G, F270M, and E271R;
(h) D70P, S73T, V83A, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, V261G, and E271R;
(i) V36A, K44E, D70P, S73T, I77V, V83A, S97T, Q99M, L105A, V110A, T111A, E113C, L114Q, N116D, A132C, A133M, T136A, Q139E, F140Y, S141A, T154D, V160A, T164S, A182P, A187I, R200Q, A201G, N206G, G208R, A211V, F222M, S226F, F230H, T233A, H239A, D241L, Y247W, T254V, V255I, V261G, F270M, and E271R;
(j) V36A, K44D, A46L, D70P, S73T, I77V, V83A, T91R, S97T, Q99M, L105A, N108G, V110A, T111A, E113C, L114Q, N116D, A132C, A133M, T136A, Q139E, F140Y, S141A, T154D, V160A, S161A, T164G, E179G, A182P, R200Q, A201G, N206G, G208R, V210I, A211V, F222M, S226F, F230H, T233A, H239A, D241L, Y247W, T254V, V255I, V261G, F270M, and E271R;
(k) K44D, D70P, S73T, V83C, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, Y247W, V261G, and E271R;
(l) K44E, D70P, S73T, I77V, V83A, L105A, V110A, T111A, E113A, L114Q, N116D, A132C, A133M, T136A, F140Y, S141T, T154D, V160A, T164S, A182P, R200Q, N206G, G208R, A211V, F222M, S226F, F230H, T233A, H239A, D241L, Y247W, V261G, F270M and E271R;
(m) D70P, S73T, V83A, L105A, V110A, T111A, E113C, L114Q, N116D, A133M, T136A, F140Y, S141T, T154D, V160A, T164G, A182P, R200Q, N206G, A211V, S226F, F230H, T233A, H239A, D241L, V261G, and E271R; or
(n) V36A, K44E, A46L, D70P, S73T, I77V, V83A, S97T, Q99M, L105A, V110A, T111A, E113C, L114Q, N116D, A132C, A133M, T136A, Q139E, F140Y, S141A, T154D, V160A, S161A, T164S, A182P, R200Q, A201G, N206G, G208R, A211V, F222M, S226F, F230H, T233A, H239A, D241L, Y247W, T254V, V255I, V261G, F270M, and E271R.

Also provided herein are alcohol dehydrogenase variants of a wild-type alcohol dehydrogenase from Aromatoleum aromaticum of SEQ ID NO: 55 comprising one or more mutations selected from K2R, S8A, R14N, E16T, L45Y, G46L, S49T, A51G, V54T, A55E, N57G, I71V, D76T, A79K, A83R, H91D, L93I, E96P, A97P, I99M or I99A; A100E, V110I, C115A or C115R, L117Y, R120A or R120F, T123D, S128A, Q136E, T142C, I152V, Y1535, Y153A or Y153G; N154E, N154H or N154D; A159P, S162T, T185R, A188V, H198R, R200K, 5205D, C211Q, S212R or S212T or S212Q, Q213N or Q213D; Q215D, A217V or A217R, S218K, C227R, V266E, K274M, R275K, Q277A, Q289M, N291E, T300H, F303K or F303R, 5309V, T3115 or T311P, L312F, T314Q or T314E, L321H, Q322L, K323E or K323Q, A324S and S332C.

In another embodiment, provided herein is an alcohol dehydrogenase variant of a wild-type alcohol dehydrogenase from Aromatoleum aromaticum of SEQ ID NO: 55 comprising mutations:
(a) A51G, V54T, D76T, A79K, A83R, E96P, A97P, I99M, C115A, L117Y, S128A, N154E, S162T, T185R, H198R, 5205D, S212R, Q213N, Q215D, A217V, S218K, C227R, V266E, Q277A, N291E, T300H, F303K, 5309V, L312F, T314Q, L321H, Q322L, and S332C;
(b) S8A, L45Y, A51G, V54T, N57G, I71V, D76T, A79K, A83R, H91D, E96P, A97P, I99M, C115A, L117Y, R120A, S128A, T142C, I152V, Y1535, N154H, A159P, S162T, T185R, H198R, 5205D, C211Q, S212R, Q213N, Q215D, A217V, S218K, C227R, V266E, K274M, R275K, Q277A, N291E, T300H, F303K, 5309V, T3115, L312F, T314E, L321H, Q322L, K323E, and S332C;
(c) K2R, S8A, L45Y, A51G, V54T, N57G, I71V, D76T, A79K, A83R, H91D, E96P, A97P, I99A, C115A, L117Y, R120A, S128A, T142C, I152V, Y153A, N154E, A159P, S162T, T185R, A188V, H198R, 5205D, C211Q, S212R, Q213D, Q215D, A217R, S218K, C227R, V266E, K274M, R275K, Q277A, Q289M, N291E, T300H, F303K, 5309V, T3115, L312F, T314E, L321H, Q322L, K323E, A324S, and S332C;
(d) A51G, V54T, D76T, A79K, A83R, E96P, A97P, I99M, C115A, L117Y, R120A, S128A, T142C, Y1535, N154D, A159P, S162T, T185R, H198R, 5205D, S212R, Q213N, Q215D, A217V, S218K, C227R, V266E, Q277A, N291E, T300H, F303K, S309V, L312F, T314Q, L321H, Q322L, and S332C;
(e) K2R, S8A, R14N, E16T, L45Y, G46L, S49T, A51G, V54T, A55E, N57G, I71V, D76T, A79K, A83R, H91D, L93I, E96P, A97P, I99A, A100E, V110I, C115A, L117Y, R120A, T123D, S128A, Q136E, T142C, I152V, Y153G, N154E, A159P, S162T, T185R, A188V, H198R, R200K, S205D, C211Q, S212T, Q213N, Q215D, A217R, S218K, C227R, V266E, K274M, R275K, Q277A, Q289M, N291E, T300H, F303R, S309V, T311S, L312F, T314Q, L321H, Q322L, K323Q, A324S, and S332C;
(f) A51G, V54T, D76T, A79K, A83R, E96P, A97P, I99M, C115A, L117Y, R120F, S128A, N154E, S162T, T185R, H198R, S205D, Q213N, Q215D, A217V, S218K, C227R, V266E, Q277A, N291E, T300H, F303K, S309V, L312F, T314Q, L321H, Q322L, and S332C;
(g) S8A, L45Y, A51G, V54T, N57G, I71V, D76T, A79K, A83R, H91D, E96P, A97P, I99M, C115A, L117Y, R120A, S128A, T142C, I152V, Y153S, N154E, A159P, S162T, T185R, H198R, S205D, C211Q, S212R, Q213N, Q215D, A217R, S218K, C227R, V266E, K274M, R275K, Q277A, Q289M, N291E, T300H, F303K, S309V, T311S, L312F, T314E, L321H, K323E, and S332C;
(h) A51G, V54T, D76T, A83R, E96P, A97P, L117Y, S128A, N154E, S162T, T185R, H198R, S205D, Q213N, Q215D, A217V, S218K, C227R, V266E, Q277A, N291E, T300H, F303K, S309V, L312F, T314Q, L321H, and S332C;
(i) A51G, V54T, D76T, A79K, A83R, E96P, A97P, I99M, C115A, L117Y, R120A, S128A, T142C, Y153S, N154D, A159P, S162T, T185R, H198R, S205D, S212R, Q213N, Q215D, A217V, S218K, C227R, V266E, Q277A, N291E, T300H, F303K, S309V, T311P, L312F, T314Q, L321H, Q322L, and S332C;
(j) K2R, S8A, R14N, E16T, L45Y, G46L, S49T, A51G, V54T, A55E, N57G, I71V, D76T, A79K, A83R, H91D, L93I, E96P, A97P, I99A, A100E, V110I, C115A, L117Y, R120A, T123D, S128A, Q136E, T142C, I152V, Y153A, N154E, A159P, S162T, T185R, A188V, H198R, S205D, C211Q, S212Q, Q213N, Q215D, A217R, S218K, C227R, V266E, K274M, R275K, Q277A, Q289M, N291E, T300H, F303R, S309V, T311S, L312F, T314Q, L321H, Q322L, K323E, A324S, and S332C;
(k) A51G, V54T, D76T, A83R, E96P, A97P, C115R, L117Y, S128A, N154E, S162T, T185R, H198R, S205D, Q213N, Q215D, A217V, S218K, C227R, V266E, Q277A, N291E, T300H, F303K, S309V, L312F, T314E, L321H, and S332C;
(l) A51G, V54T, D76T, A83R, E96P, A97P, L117Y, N154E, S162T, T185R, H198R, S205D, Q213D, Q215D, S218K, C227R, V266E, Q277A, T300H, F303K, S309V, L312F, T314Q, L321H, and S332C; or
(m) A51G, V54T, D76T, A83R, E96P, A97P, C115R, L117Y, N154E, S162T, T185R, H198R, S205D, Q213D, Q215D, S218K, C227R, V266E, Q277A, T300H, F303K, S309V, L312F, T314Q, L321H, and S332C.

In one embodiment, provided herein are nucleic acids encoding an above-described alcohol dehydrogenase variant of any one of SEQ ID NO: 44, SEQ ID NO: 52, SEQ ID NO: 53 or SEQ ID NO: 55. In another embodiment, provided herein are expression vectors comprising the nucleic acid encoding the alcohol dehydrogenase variant of any one of SEQ ID NO: 44, SEQ ID NO: 52, SEQ ID NO: 53 or SEQ ID NO: 55. In another embodiment, provided herein are host cells transformed with the expression vector comprising the nucleic acid encoding the alcohol dehydrogenase variant of any one of SEQ ID NO: 44, SEQ ID NO: 52, SEQ ID NO: 53 or SEQ ID NO: 55. In a further embodiment, provided herein are methods for producing an alcohol dehydrogenase variant, said method comprising expressing the alcohol dehydrogenase variant in the transformed host cell, wherein the host cell is transformed with the expression vector comprising the nucleic acid encoding the alcohol dehydrogenase variant of any one of SEQ ID NO: 44, SEQ ID NO: 52, SEQ ID NO: 53 or SEQ ID NO: 55.

### Histidine Acid Phosphatase (Phytase) Variants

Phytase is a class of phosphatase enzyme that hydrolyzes phytic acid to release a useable form of organic phosphorus. Phytic acid a storage form of phosphorus found in plants, seeds, suhc as oil seeds, cereals and grains, which is not bioavailable to nonruminants, since these animals lack the phytase enzyme. Phytases are found in plants, fungi, bacteria and ruminant animal, such as cattle and sheep. Nonruminants, such as humans, dogs, and birds, do not produce phytase. Animal feed may be supplemented with phytase to release phytate-bound nutrients, such as calcium, phosphorus, other minerals, such as zinc, iron and magnesium, carbohydrates, and proteins. Phytase may be added to grains, legumes, seeds and corn to release the above-enumerated minerals, thereby producing functional food. A phytase encoded by Gram-negative bacteria species Yersinia mollaretii has been characterized as having a specific activity of 1,073 U/mg which is about 10 times higher than widely used fungal phytases.

The phytase (histidine acid phosphatase) variants provided herein may be used as described above, e.g., to supplement feed and hydrolyze indigestible phytate, i.e., myo-inositol 1,2,3,4,5,6-hexakis dihydrogen phosphate, to increase absorption in nonruminants of inorganic phosphates, minerals, as well as trace elements, from plant-based food, seeds, and grains. The provided phytase variants release from two to ten times more inorganic phosphorus, are thermostable at high temperature, e.g., 55° C. to 90° C., are active at body temperature of humans and animals (30° C.−40° C.) and at a pH range from 1.5 to 6.0, thereby withstanding the acidic environment of the stomach without a loss of specific activity.

Provided herein are histidine acid phosphatase variants of a wild-type histidine acid phosphatase from Yersinia mollaretii of SEQ ID NO: 45 comprising one or more mutations selected from S42A, Q46P or Q46F, D52N, D56H or D56Q, K57P, A65P, T77R or T77K, G81Q or G81R, F82Y, Y83F, N89Q, T102E, Q106W, I109S or I109T, L115A, A136P, V141P or V141M, E149K, Q154T or Q154R, S157P, T158K or T158E, H161R or H161K, R162A, E165M, Q167R, A170G, S173D, E174A, K181D, P182D or P182S, Q185L, G187Q or G187E, E188Q, I189V, T193C or T193K, A194N, 5200A or 5200K, 5207T, N221S, Q222K, Q223E, S230E, S236A, G240A, N247Y, S248A, H257G, A262E, V266R, S267E, L269M, Q275K or Q275Y, K289Q, V298L, K306D, G324A, G334A, Q342T, Q345G, G355A, N363D, D365K, M380L, S386A, K392T, S393N, H394N, I409T, T411D, Q416P, and A425K.

In one embodiment, provided herein is a histidine acid phosphatase variant of a wild-type histidine acid phosphatase from Yersinia mollaretii of SEQ ID NO: 45 comprising mutations:
(a) S42A, Q46P, D52N, D56H, K57P, A65P, T77R, G81Q, F82Y, Y83F, N89Q, Q106W, I109S, L115A, A136P, V141P, E149K, Q154T, S157P, T158K, H161R, R162A, E165M, Q167R, A170G, S173D, E174A, K181D, P182D, Q185L, G187Q, E188Q, T193C, A194N, S200A, 5207T, N221S, Q222K, Q223E, S230E, S236A, G240A, N247Y, S248A, H257G, A262E, V266R, Q275K, K289Q, V298L, K306D, G324A, G334A, Q342T, Q345G, G355A, N363D, D365K, M380L, S386A, K392T, S393N, H394N, I409T, T411D, Q416P, and A425K;
(b) Q46F, D56H, K57P, A65P, T77K, G81Q, F82Y, Y83F, N89Q, T102E, Q106W, L115A, V141M, Q154T, S157P, T158E, H161R, R162A, E165M, A170G, E174A, Q185L, G187Q, T193C, A194N, S200K, S230E, S236A, G240A, N247Y, S248A, A262E, V266R, V298L, K306D, G324A, G334A, Q345G, N363D, S386A, I409T, and T411D;
(c) Q46F, K57P, A65P, T77K, G81R, N89Q, Q106W, L115A, Q154R, S157P, T158E, H161K, E165M, A170G, E174A, Q185L, G187Q, T193C, S236A, G240A, N247Y, S248A, L269M, V298L, K306D, G324A, G334A, Q345G, and T411D;
(d) Q46F, D56Q, K57P, A65P, T77K, G81R, F82Y, Y83F, N89Q, T102E, Q106W, I109T, L115A, V141P, Q154T, S157P, T158E, H161R, R162A, E165M, A170G, E174A, Q185L, G187Q, E188Q, T193C, A194N, S200K, Q222K, Q223E, S230E, S236A, G240A, N247Y, S248A, H257G, A262E, V266R, S267E, L269M, V298L, K306D, G324A, G334A, Q342T, Q345G, G355A, N363D, S386A, S393N, H394N, I409T, T411D, and A425K;
(e) S42A, Q46F, D52N, D56H, K57P, A65P, T77R, G81Q, F82Y, Y83F, N89Q, T102E, Q106W, I109T, L115A, A136P, V141P, E149K, Q154T, S157P, T158K, H161K, R162A, E165M, Q167R, A170G, S173D, E174A, K181D, P182S, Q185L, G187E, E188Q, I189V, T193K, A194N, S200A, S207T, N221S, Q222K, Q223E, S230E, S236A, G240A, N247Y, S248A, H257G, A262E, V266R, Q275K, K289Q, V298L, K306D, G324A, G334A, Q342T, Q345G, G355A, N363D, D365K, S386A, K392T, S393N, H394N, I409T, T411D, Q416P, and A425K;
(f) Q46F, K57P, A65P, T77K, G81R, F82Y, Y83F, N89Q, T102E, Q106W, L115A, Q154T, S157P, T158E, H161K, E165M, A170G, E174A, Q185L, G187Q, T193C, S200K, S236A, G240A, N247Y, S248A, V266R, L269M, V298L, K306D, G324A, G334A, Q345G, and T411D;
(g) Q46F, A65P, T77K, G81Q, N89Q, T102E, Q106W, S157P, A170G, Q185L, G187Q, T193C, S236A, G240A, N247Y, S248A, L269M, V298L, K306D, G324A, G334A, Q345G, and T411D;
(h) Q46F, K57P, A65P, T77K, G81Q, F82Y, Y83F, N89Q, T102E, Q106W, L115A, Q154T, S157P, T158E, H161K, E165M, A170G, E174A, Q185L, G187Q, T193C, S200K, S236A, G240A, N247Y, S248A, V266R, Q275Y, V298L, K306D, G324A, G334A, Q345G and T411D;
(i) Q46F, D56H, K57P, A65P, T77K, G81R, F82Y, Y83F, N89Q, T102E, Q106W, L115A, V141M, Q154T, S157P, T158E, H161R, R162A, E165M, A170G, E174A, Q185L, G187Q, I189V, T193C, A194N, S200K, S230E, S236A, G240A, N247Y, S248A, A262E, V266R, L269M, V298L, K306D, G324A, G334A, Q345G, N363D, S386A, I409T, and T411D;
(j) N89Q, T102E, Q106W, S157P, A170G, Q185L, G187Q, S236A, G240A, N247Y, S248A, V298L, G324A, G334A, Q345G, and T411D;
(k) N89Q, Q106W, S157P, A170G, Q185L, G187Q, S236A, G240A, N247Y, S248A, L269M, Q275Y, V298L, G324A, G334A, Q345G, and T411D;
(l) Q46F, D56H, K57P, A65P, T77K, G81Q, F82Y, Y83F, N89Q, T102E, Q106W, I109T, L115A, V141P, Q154T, S157P, T158E, H161R, R162A, E165M, A170G, E174A, Q185L, G187Q, E188Q, T193C, A194N, S200K, Q222K, Q223E, S230E, S236A, G240A, N247Y, S248A, H257G, A262E, V266R, S267E, L269M, Q275Y, V298L, K306D, G324A, G334A, Q342T, Q345G, G355A, N363D, S386A, S393N, H394N, I409T, T411D, and A425K; or
(m) Q46F, K57P, A65P, T77K, G81Q, N89Q, T102E, Q106W, L115A, Q154R, S157P, T158E, H161K, E165M, A170G, E174A, Q185L, G187Q, T193C, S236A, G240A, N247Y, S248A, V298L, K306D, G324A, G334A, Q345G, and T411D.

In one embodiment, provided herein are nucleic acids encoding an above-described histidine acid phosphatase variant of SEQ ID NO: 45. In another embodiment, provided herein are expression vectors comprising the nucleic acid encoding the histidine acid phosphatase variant of SEQ ID NO: 45. In another embodiment, provided herein are host cells transformed with the expression vector comprising the nucleic acid encoding the histidine acid phosphatase variant of SEQ ID NO: 45. In a further embodiment, provided herein are methods for producing a histidine acid phosphatase variant, said method comprising expressing the histidine acid phosphatase variant in the transformed host cell, wherein the host cell is transformed with the expression vector comprising the nucleic acid encoding the histidine acid phosphatase variant of SEQ ID NO: 45.

### Chanoclavine-I Aldehyde Reductase easA Variants

Chanoclavine-I aldehyde reductase easA is an enzyme that is an aldehyde reductase, this enzyme is involved in the biosynthesis of fumiclavanine C, which is an ergot alkaloid (a mycotoxin) produced by some fungi, including Neosartorya Claviceps, Aspergillus and Penicillium genera. EasA catalyzes the reduction of chanoclavine-I aldehyde to dihydrochanoclavine-I aldehyde that spontaneously dehydrates to form 6,8-dimethyl-6,7-didehydroergoline, which is which is converted to festuclavine by festuclavine dehydrogenase (EasG). Festuclavine is hydrolyzed by easM, leading to the formation of fumigaclavine B, which, in turn, is acetylated by easN to fumigaclavine A. Next, easL catalyzes the conversion of fumigaclavine A into fumigaclavine C by attaching a dimethylallyl moiety to C-2 of the indole nucleus. Ergot alkaloids are used in therapeutic drugs, such as migraine reduction and blood pressure regulation. The herein provided chanoclavine-I aldehyde reductase easA variants maybe used to produce improved ergot alkaloids in vitro via biotechnological processes.

Provided herein are chanoclavine-I aldehyde reductase easA variants of a wild-type chanoclavine-I aldehyde reductase easA from Neosartorya fumigata of SEQ ID NO: 46 comprising one or more mutations selected from A8S, K12Q, H20N, I26V, T30M, G37D, Q38N, G50A, D66F, T68S, M72G, P84E, R86I, E91K, S94D, R95A, I126V, C151Y or C151I, A153E, N164E, T184I, K186D, Q192T, T210V, V217I, E240D, A250E, M252L, D256K, L268Q, E270N, E304R, K305W, Q309W, Y368W, L371R, and H372N or H372Q.

In one embodiment, provided herein is a chanoclavine-I aldehyde reductase easA variant of a wild-type chanoclavine-I aldehyde reductase easA from Neosartorya fumigata of SEQ ID NO: 46 comprising mutations:
(a) A8S, K12Q, H20N, I26V, T30M, G37D, Q38N, G50A, D66F, T68S, M72G, P84E, R86I, E91K, S94D, R95A, I126V, C151Y, A153E, N164E, T184I, K186D, Q192T, T210V, V217I, E240D, A250E, M252L, D256K, L268Q, E270N, E304R, K305W, Q309W, Y368W, L371R and H372N;
(b) A8S, H20N, I26V, T30M, G37D, Q38N, G50A, D66F, T68S, M72G, R86I, E91K, S94D, C151Y, A153E, N164E, T184I, K186D, Q192T, V217I, A250E, Q309W, Y368W, L371R and H372Q;
(c) A8S, I26V, T30M, G37D, Q38N, G50A, D66F, T68S, M72G, R86I, S94D, C151Y, A153E, N164E, T184I, K186D, Q192T, V217I, Q309W, Y368W, and L371R;
(d) A8S, K12Q, H20N, I26V, T30M, G37D, Q38N, G50A, D66F, T68S, M72G, P84E, R86I, E91K, S94D, R95A, C151Y, A153E, N164E, T184I, K186D, Q192T, T210V, V217I, A250E, Q309W, Y368W, L371R, and H372Q;
(e) A8S, K12Q, H20N, I26V, T30M, G37D, Q38N, G50A, D66F, T68S, M72G, P84E, R86I, E91K, S94D, R95A, C151Y, A153E, N164E, T184I, K186D, Q192T, T210V, V217I, A250E, M252L, Q309W, Y368W, L371R, and H372N;
(f) A8S, K12Q, H20N, I26V, T30M, G37D, Q38N, G50A, T68S, M72G, P84E, R86I, E91K, S94D, R95A, I126V, C151I, A153E, N164E, T184I, K186D, Q192T, T210V, V217I, E240D, A250E, M252L, D256K, L268Q, E270N, E304R, K305W, Q309W, Y368W, L371R and H372N;
(g) I26V, G50A, D66F, T68S, M72G, R86I, C151Y, K186D, Q192T, and Y368W;
(h) I26V, T30M, G50A, D66F, T68S, M72G, R86I, S94D, C151Y, A153E, K186D, Q192T and

Y368W; or
(i) A8S, I26V, T30M, G37D, G50A, D66F, T68S, M72G, R86I, S94D, C151Y, A153E, N164E, T184I, K186D, Q192T, Y368W, and L371R.

In one embodiment, provided herein are nucleic acids encoding an above-described chanoclavine-I aldehyde reductase easA variant of SEQ ID NO: 46. In another embodiment, provided herein are expression vectors comprising the nucleic acid encoding the chanoclavine-I aldehyde reductase easA variant of SEQ ID NO: 46. In another embodiment, provided herein are host cells transformed with the expression vector comprising the nucleic acid encoding the chanoclavine-I aldehyde reductase easA variant of SEQ ID NO: 46. In a further embodiment, provided herein are methods for producing a chanoclavine-I aldehyde reductase easA variant, said method comprising expressing the chanoclavine-I aldehyde reductase easA variant in the transformed host cell, wherein the host cell is transformed with the expression vector comprising the nucleic acid encoding the chanoclavine-I aldehyde reductase easA variant of SEQ ID NO: 46.

### Alanine Dehydrogenase Variants

Alanine dehydrogenase is an enzyme that catalyzes the NAD+-dependent oxidative deamination of L-alanine to pyruvate and ammonium, and the reverse reaction, i.e., the reductive amination of pyruvate to L-alanine. Alanine dehydrogenase is required for growth when alanine is the sole carbon or nitrogen source. The amino acid L-alanine is used in various industries, including food, pharmaceutical, veterinary and production of engineered thermoplastics. The alanine dehydrogenase variants provided herein are hyperthermostable, maintaining structural stability and function at high temperatures of equal to or greater than 60° C., and high acidity add/or high radiation levels, enabling faster and improved production of L-alanine in bioreactors at high temperatures from 33° C. to 42° C.

Also provided herein are alanine dehydrogenase variants of a wild-type alanine dehydrogenase from Archaeoglobus fulgidus of SEQ ID NO: 47 comprising one or more mutations selected from E2K, Q8R, S13Q, N22E, L33E, G72Q, S89D, T104I, S106A, S125A, F128V, T135E, K235R, D277P, V300A, S310E, S315T, K316F and I317V.

In one embodiment, provided herein is an alanine dehydrogenase variant of a wild-type alanine dehydrogenase from Archaeoglobus fulgidus of SEQ ID NO: 47 comprising mutations:
(a) E2K, Q8R, N22E, L33E, S89D, T104I, S106A, S125A, F128V, K235R, D277P, S310E, S315T, and K316F;
(b) Q8R, S106A, K235R, D277P, S310E, S315T, and K316F;
(c) E2K, Q8R, N22E, S89D, S106A, S125A, K235R, D277P, S310E, S315T, and K316F;
(d) E2K, Q8R, N22E, S89D, S106A, S125A, F128V, D277P, S310E, S315T, and K316F;
(e) E2K, Q8R, S13Q, N22E, L33E, G72Q, S89D, T104I, S106A, S125A, F128V, T135E, K235R, D277P, V300A, S310E, S315T, K316F, and I317V;
(f) E2K, Q8R, N22E, L33E, S89D, T104I, S106A, S125A, F128V, D277P, V300A, S310E, S315T, and K316F;
(g) E2K, Q8R, N22E, L33E, S89D, T104I, S106A, S125A, F128V, T135E, K235R, D277P, V300A, S310E, S315T, and K316F;
(h) Q8R, S106A, K235R, D277P, S315T, and K316F;
(i) E2K, Q8R, S13Q, N22E, L33E, G72Q, S89D, T104I, S106A, S125A, F128V, K235R, D277P, V300A, S310E, S315T, K316F, and I317V;
(j) E2K, Q8R, N22E, S89D, S106A, S125A, F128V, K235R, D277P, S310E, S315T and K316F; or
(k) E2K, Q8R, N22E, L33E, S89D, T104I, S106A, S125A, F128V, D277P, S310E, S315T and K316F.

In one embodiment, provided herein are nucleic acids encoding an above-described alanine dehydrogenase variant of SEQ ID NO: 47. In another embodiment, provided herein are expression vectors comprising the nucleic acid encoding the alanine dehydrogenase variant of SEQ ID NO: 47. In another embodiment, provided herein are host cells transformed with the expression vector comprising the nucleic acid encoding the alanine dehydrogenase variant of SEQ ID NO: 47. In a further embodiment, provided herein are methods for producing a alanine dehydrogenase variant, said method comprising expressing the alanine dehydrogenase variant in the transformed host cell, wherein the host cell is transformed with the expression vector comprising the nucleic acid encoding the alanine dehydrogenase variant of SEQ ID NO: 47.

### Acid Phosphatase Variants

Acid phosphatase is an enzyme that cleaves phosphoryl groups from an orthophosphoric monoester during digestion. This phosphatase has an optimum activity in the pH range of 4-6. Soil microorganism use acid phosphatase to obtain phosphate nutrients; measuring the rate of activity of acid phosphatases determines the need for phosphates in soil. The hereoin provided acid phosphatase variants have improved stability in an acidic pH.

Provided herein are acid phosphatase variants of a wild-type acid phosphatase from Shimwellia blattae of SEQ ID NO: 48 comprising one or more mutations selected from T28A, E41Q, N44D; N62A, N62L or N62Y; L72A; L81Q, A83K or A83Q; E84A, S90P, G91D, G92D, A94P, N95K; G99P or G99E; S103M, E107P, K108E, A112W, L113I, H114Y or H114W; I121F, 5170A; I171F, I171M, I171L or I171A; T175F or T175V; V178I, Q185E or Q185D, N188D, L197Y, A215G, T225A, Q234A, Q235D, and Q237A.

In one embodiment, provided herein is an acid phosphatase variant of a wild-type acid phosphatase from Shimwellia blattae of SEQ ID NO: 48 comprising mutations:
(a) T28A, E41Q, N44D, N62A, L72A, A83K, E84A, S90P, G91D, G92D, A94P, N95K, G99P, S103M, E107P, A112W, L113I, H114Y, S170A, I171F, Q185E, N188D, L197Y, A215G, T225A, Q234A, and Q235D;
(b) T28A, E41Q, N44D, N62A, L72A, A83K, E84A, S90P, G91D, G92D, A94P, N95K, G99P, S103M, E107P, A112W, L113I, H114Y, S170A, T175F, Q185D, N188D, L197Y, A215G, T225A, Q234A, and Q235D;
(c) E41Q, N44D, N62A, L72A, A83K, G91D, G92D, A94P, G99P, E107P, H114W, I171M, T175F, Q185D, L197Y, and Q234A;
(d) N44D, N62A, L72A, A83K, G91D, G92D, G99E, E107P, H114Y, I171L, Q185E, and L197Y;
(e) E41Q, N44D, N62A, L72A, A83K, 590P, G91D, G92D, A94P, N95K, G99P, E107P, A112W, L113I, H114Y, S170A, I171M, T175F, Q185D, L197Y, A215G, T225A, and Q234A;
(f) E41Q, N44D, N62A, L72A, A83K, G91D, G92D, A94P, G99P, E107P, H114Y, I171M, T175F, Q185E, L197Y, and Q234A;
(g) N44D, N62L, L72A, A83Q, G91D, G92D, G99E, E107P, H114Y, T175F, Q185D and L197Y;
(h) N44D, N62Y, L72A, G91D, G92D, E107P, H114Y, T175F, and L197Y;
(i) T28A, E41Q, N44D, N62A, L72A, L81Q, A83K, E84A, S90P, G91D, G92D, N95K, G99P, S103M, E107P, K108E, A112W, L113I, H114Y, I121F, S170A, V178I, Q185E, N188D, L197Y, A215G, T225A, Q234A, Q235D, and Q237A;
(j) N44D, N62A, L72A, A83Q, G91D, G92D, G99E, E107P, H114Y, I171L, T175V, Q185E and L197Y; or
(k) T28A, E41Q, N44D, N62A, L72A, L81Q, A83K, E84A, S90P, G91D, G92D, A94P, N95K, G99P, S103M, E107P, K108E, A112W, L113I, H114Y, S170A, I171A, V178I, Q185E, N188D, L197Y, A215G, T225A, Q234A, Q235D, and Q237A.

Provided herein are acid phosphatase variants of a wild-type acid phosphatase from Prevotella intermedia of SEQ ID NO: 50 comprising one or more mutations selected from N33H, D41E, G42S or G42E; Q44A or Q44S; T45V or T45I, S46D, T53P, L63K or L63E; Y64N, E66M or E66R; A67N, M74A, D82K, V85R, V90L, N97Q, E101C, I105M, K109P, T111K, V119L, L120R or L120Q; G127N, A145M, Y147F, M150S, N153T, E155K, Q157E, Q158E, S161R, T162K, T171A, I173L, T177V, V180I, S182A, I186P, Q189A, N190D, E194K, Y197R, Q198E, M199Y, S221A, D231N, Q237D and Q249R.

In one embodiment, provided herein is an acid phosphatase variant of a wild-type acid phosphatase from Prevotella intermedia of SEQ ID NO: 50 comprising mutations:
(a) Q44A, T45V, S46D, T53P, Y64N, E66M, M74A, V85R, V90L, E101C, V119L, Y147F, M150S, N153T, T162K, V180I, S182A, I186P, E194K, M199Y, S221A, and Q237D;
(b) N33H, D41E, G42S, Q44A, T45V, S46D, T53P, L63K, Y64N, E66M, A67N, M74A, D82K, V85R, V90L, N97Q, E101C, I105M, K109P, T111K, V119L, L120R, G127N, A145M, Y147F, M150S, N153T, E155K, Q157E, Q158E, S161R, T162K, I173L, T177V, V180I, S182A, I186P, Q189A, N190D, E194K, Y197R, Q198E, M199Y, S221A, D231N, Q237D and Q249R;
(c) G42S, Q44A, T45V, S46D, T53P, L63K, Y64N, E66M, M74A, D82K, V85R, V90L, E101C, K109P, V119L, L120R, G127N, A145M, Y147F, M150S, N153T, E155K, Q157E, Q158E, S161R, T162K, T171A, I173L, T177V, S182A, I186P, N190D, E194K, Y197R, Q198E, M199Y, S221A, D231N, Q237D, and Q249R;
(d) Q44A, S46D, T53P, Y64N, E66M, V85R, V90L, V119L, Y147F, M150S, N153T, T177V, I186P, M199Y, and S221A;
(e) G42E, Q44A, T45V, S46D, T53P, Y64N, E66M, M74A, D82K, V85R, V90L, E101C, V119L, L120Q, A145M, Y147F, M150S, N153T, E155K, Q157E, S161R, T162K, T177V, V180I, S182A, I186P, M199Y, S221A, and Q237D;
(f) Q44A, S46D, T53P, Y64N, E66M, V85R, V90L, V119L, N153T, I186P, M199Y, and S221A;
(g) G42S, Q44A, T45V, S46D, T53P, L63K, Y64N, E66M, M74A, D82K, V85R, V90L, E101C, K109P, V119L, L120R, G127N, A145M, Y147F, M150S, N153T, E155K, Q157E, Q158E, S161R, T162K, I173L, T177V, V180I, S182A, I186P, N190D, E194K, Y197R, Q198E, M199Y, S221A, D231N, Q237D, and Q249R;
(h) Q44A, T45V, S46D, T53P, Y64N, E66M, M74A, V85R, V90L, E101C, V119L, A145M, Y147F, M150S, N153T, T162K, V180I, S182A, I186P, E194K, M199Y, S221A, and Q237D;
(i) N33H, D41E, G42S, Q44A, T45V, S46D, T53P, L63E, Y64N, E66R, A67N, M74A, D82K, V85R, V90L, N97Q, E101C, I105M, K109P, T111K, V119L, L120R, G127N, A145M, Y147F, M150S, N153T, E155K, Q157E, Q158E, S161R, T162K, T171A, I173L, T177V, V180I, S182A, I186P, Q189A, N190D, E194K, Y197R, Q198E, M199Y, S221A, D231N, Q237D, and Q249R;
(j) G42E, Q44A, T45V, S46D, T53P, Y64N, E66M, M74A, D82K, V85R, V90L, E101C, V119L, L120Q, A145M, Y147F, M150S, N153T, E155K, Q157E, S161R, T162K, V180I, S182A, I186P, E194K, M199Y, S221A, and Q237D;
(k) Q44A, T45V, S46D, T53P, Y64N, E66M, V85R, V90L, V119L, Y147F, M150S, N153T, T177V, S182A, I186P, M199Y, S221A, and Q237D; or
(l) Q44S, T45I, S46D, T53P, Y64N, E66M, V85R, V90L, V119L, Y147F, M150S, N153T, T177V, I186P, M199Y, and S221A.

Provided herein are acid phosphatase variants of a wild-type acid phosphatase from Shigella flexneri of SEQ ID NO: 54 comprising one or more mutations selected from D40S, N41Q, A425, I55P, G56D, A59D, L61K, L72A or L72K; L81Q, N95K, K108E, S110A, T118R or T118K, N119R, N151T, D156K or D156Q; R160K or R160T; S170A, T175I or T175M; K192Q, Q235D, and N246L.

In one embodiment, provided herein is an acid phosphatase variant of a wild-type acid phosphatase from Shigella flexneri of SEQ ID NO: 54 comprising mutations:
(a) N41Q, I55P, G56D, L72A, N95K, S110A, N119R, R160K, T175I, and Q235D;
(b) N41Q, I55P, G56D, A59D, L72A, N95K, S110A, N119R, N151T, D156K, R160K, T175M, K192Q, Q235D, and N246L;
(c) D40S, N41Q, A42S, I55P, G56D, A59D, L61K, L72A, L81Q, N95K, K108E, S110A, T118K, N119R, N151T, D156K, R160K, S170A, T175I, K192Q, Q235D, and N246L;
(d) D40S, N41Q, A42S, I55P, G56D, A59D, L61K, L72K, L81Q, N95K, K108E, S110A, T118R, N119R, N151T, D156K, R160K, S170A, T175I, K192Q, Q235D, and N246L;
(e) N41Q, L72A, S110A, T175I, and Q235D;
(f) L72A, S110A, and T1751;
(g) L72K, S110A, and T1751;
(h) N41Q, I55P, G56D, A59D, L72A, N95K, S110A, N119R, N151T, D156K, R160T, T175M, K192Q, Q235D, and N246L; or
(i) N41Q, I55P, G56D, L72A, N95K, S110A, N119R, N151T, D156Q, R160K, T175I, Q235D and N246L.

In one embodiment, provided herein are nucleic acids encoding an above-described acid phosphatase variant of any one of SEQ ID NO: 48, SEQ ID NO: 50, or SEQ ID NO: 54. In another embodiment, provided herein are expression vectors comprising the nucleic acid encoding the acid phosphatase variant of any one of SEQ ID NO: 48, SEQ ID NO: 50, or SEQ ID NO: 54. In another embodiment, provided herein are host cells transformed with the expression vector comprising the nucleic acid encoding the acid phosphatase variant of any one of SEQ ID NO: 48, SEQ ID NO: 50, or SEQ ID NO: 54. In a further embodiment, provided herein are methods for producing an acid phosphatase variant, said method comprising expressing the acid phosphatase variant in the transformed host cell, wherein the host cell is transformed with the expression vector comprising the nucleic acid encoding the acid phosphatase variant of any one of SEQ ID NO: 48, SEQ ID NO: 50, or SEQ ID NO: 54.

### Major Phosphate-Irrepressible Acid Phosphatase Variants

Morganella morganii is one of the few enterobacterial species producing high-level phosphate-irrepressible acid phosphatase (“PhoC”) activity. PhoC activity has been shown to prevent induction of alkaline phosphatase when a PhoC-hydrolysable organic phosphate ester, such as glycerol 2-phosphate, was the sole phosphate source.

Further provided herein are major phosphate-irrepressible acid phosphatase variants of a wild-type major phosphate-irrepressible acid phosphatase from Morganella morganii of SEQ ID NO: 49 comprising one or more mutations selected from E40D, K47A, E54T, Q59D, L61K, N62R, M72A, A89S, A90K, G91D or G91E; T95K, G99E, E107K, M137Q, E148T, N151T, K153D, Q155E, S170A, T175V, V182I, A185D or A185E; N186R, Q187A or Q187R; L197Y, D213E, A215G, T225A, D229N, Q233R, Q235D, K246L, S247L or S247R; Q248R, and K249Q.

In one embodiment, provided herein is a major phosphate-irrepressible acid phosphatase variant of a wild-type major phosphate-irrepressible acid phosphatase from Morganella morganii of SEQ ID NO: 49 comprising mutations:
(a) E40D, K47A, E54T, Q59D, L61K, N62R, M72A, A89S, A90K, G91D, T95K, G99E, E107K, M137Q, E148T, N151T, K153D, Q155E, S170A, T175V, V182I, A185D, N186R, Q187A, L197Y, D213E, A215G, T225A, D229N, Q233R, Q235D, S247L, Q248R, and K249Q;
(b) M72A, A89S, G91E, T95K, G99E, M137Q, E148T, N151T, K153D, S170A, T175V, V182I, A185E, Q187R, L197Y, A215G, T225A, Q235D, and K246L;
(c) M72A, A89S, G91D, T95K, G99E, M137Q, E148T, N151T, K153D, S170A, T175V, V182I, A185E, Q187R, L197Y, A215G, T225A, Q235D, and K246L;
(d) A89S, T175V, Q187R, L197Y, and A215G;
(e) E40D, K47A, E54T, Q59D, L61K, N62R, M72A, A89S, A90K, G91D, T95K, G99E, E107K, M137Q, E148T, N151T, K153D, Q155E, S170A, T175V, V182I, A185E, N186R, Q187A, L197Y, D213E, A215G, T225A, D229N, Q233R, Q235D, S247L, Q248R, and K249Q;
(f) K47A, Q59D, M72A, A89S, G91D, T95K, G99E, E107K, M137Q, E148T, N151T, K153D, Q155E, S170A, T175V, V182I, A185E, N186R, Q187A, L197Y, A215G, T225A, D229N, Q235D, K246L, and S247R;
(g) M72A, A89S, G91E, T95K, G99E, M137Q, E148T, N151T, K153D, S170A, T175V, V182I, A185E, N186R, Q187R, L197Y, A215G, T225A, Q235D, and K246L;
(h) A89S, G91E, G99E, K153D, T175V, Q187R, L197Y, A215G, Q235D, and K246L;
(i) K47A, Q59D, M72A, A89S, G91E, T95K, G99E, E107K, M137Q, E148T, N151T, K153D, Q155E, S170A, T175V, V182I, A185E, N186R, Q187A, L197Y, A215G, T225A, D229N, Q235D, K246L, and S247R; or
(j) A89S, G91E, G99E, K153D, T175V, Q187R, L197Y, and A215G.

In one embodiment, provided herein are nucleic acids encoding an above-described major phosphate-irrepressible acid phosphatase variant of SEQ ID NO: 49. In another embodiment, provided herein are expression vectors comprising the nucleic acid encoding the major phosphate-irrepressible acid phosphatase variant of SEQ ID NO: 49. In another embodiment, provided herein are host cells transformed with the expression vector comprising the nucleic acid encoding the major phosphate-irrepressible acid phosphatase variant of SEQ ID NO: 49. In a further embodiment, provided herein are methods for producing a major phosphate-irrepressible acid phosphatase variant, said method comprising expressing the major phosphate-irrepressible acid phosphatase variant in the transformed host cell, wherein the host cell is transformed with the expression vector comprising the nucleic acid encoding the major phosphate-irrepressible acid phosphatase variant of SEQ ID NO: 49.

Tryptophan Dimethylallyltransferase Variants

Tryptophan dimethylallyltransferase catalyzes the reaction of dimethylallyl diphosphate and L-tryptophan, to produce diphosphate and 4-(3-methylbut-2-enyl)-L-tryptophan. This enzyme is a member of the family of transferases, specifically those that transfers aryl or alkyl groups other than methyl groups. This enzyme catalyzes the first reaction in a biosynthetic pathway of the ergot alkyloids in Claviceps sp.

Provided herein are tryptophan dimethylallyltransferase variants of a wild-type tryptophan dimethylallyltransferase from Neosartorya fumigata of SEQ ID NO: 51 comprising one or more mutations selected from K2Q, A3T, A9N, R17K, A18Y, L28R, T43D or T43M, T47S, T48L, C50Q or C50E; T57F, C61H, C68P, A74R, N96H, S97N, K114N, S125A, H128R or H128Q, L130K, K134P, H146N, S152R, S155A, F157Y, H160K, G167D, A185T or A185V; T188V or T188A; T202S, V207I, G209E, V211I, R213K, V216Q, N226D, S237P, A254D, Q265R, M266N or M266Q, A285P, S292E, 5304P, V319Q, I320V, Q335P, N336G, V339Y, E341Q, S370T, T378E, T379A, A387Q, D390S, D405N, R406N, T407K or T407Q; and V453K.

In another embodiment, provided herein is a tryptophan dimethylallyltransferase variant of a wild-type tryptophan dimethylallyltransferase from Neosartorya fumigata of SEQ ID NO: 51 comprising mutations:
(a) L28R, C50Q, T57F, A74R, S125A, H128R, K134P, H146N, H160K, A185T, T188V, V211I, V216Q, S237P, A285P, S292E, V319Q, Q335P, E341Q, T378E, A387Q, R406N, and T407K;
(b) L28R, C50Q, T57F, S125A, H128R, K134P, H146N, S152R, H160K, A185V, T188V, V211I, V216Q, S237P, A285P, S292E, 5304P, V319Q, Q335P, A387Q, and T407K;
(c) A3T, L28R, C50Q, T57F, C68P, A74R, N96H, S97N, S125A, H128R, K134P, H146N, S152R, H160K, A185T, T188V, T202S, V211I, V216Q, S237P, A285P, S292E, V319Q, Q335P, V339Y, E341Q, T378E, A387Q, R406N, and T407Q;
(d) K2Q, A3T, L28R, T43D, T47S, T48L, C50E, T57F, C61H, C68P, A74R, N96H, S97N, K114N, S125A, H128R, L130K, K134P, H146N, S152R, S155A, H160K, A185V, T188V, T202S, V211I, V216Q, N226D, S237P, M266N, A285P, S292E, V319Q, I320V, Q335P, V339Y, E341Q, T378E, A387Q, R406N, and T407Q;
(e) A3T, L28R, C50E, T57F, C68P, A74R, S97N, S125A, H128R, K134P, H146N, S152R, H160K, A185V, T188V, T202S, V211I, V216Q, S237P, A285P, S292E, S304P, V319Q, Q335P, V339Y, E341Q, T378E, A387Q, R406N, and T407K;
(f) K2Q, A3T, L28R, T43D, T47S, T48L, C50Q, T57F, C61H, C68P, A74R, S97N, K114N, S125A, H128Q, L130K, K134P, H146N, S152R, S155A, H160K, A185T, T188V, T202S, V211I, V216Q, N226D, S237P, M266N, A285P, S292E, S304P, V319Q, I320V, Q335P, V339Y, E341Q, T378E, A387Q, R406N, and T407K;
(g) L28R, C50Q, T57F, A74R, S125A, H128R, K134P, H146N, H160K, A185V, T188V, V211I, V216Q, S237P, A285P, S292E, S304P, V319Q, Q335P, T378E, A387Q, R406N, and T407K;
(h) K2Q, A3T, A9N, R17K, A18Y, L28R, T43M, T47S, T48L, C50Q, T57F, C61H, C68P, A74R, N96H, S97N, K114N, S125A, H128Q, L130K, K134P, H146N, S152R, S155A, F157Y, H160K, G167D, A185T, T188A, T202S, V207I, G209E, V211I, R213K, V216Q, N226D, S237P, A254D, Q265R, M266Q, A285P, S292E, S304P, V319Q, I320V, Q335P, N336G, V339Y, E341Q, S370T, T378E, T379A, A387Q, D390S, D405N, R406N, T407Q, and V453K;
(i) C50E, T57F, S125A, H128R, K134P, A185T, S237P, A285P, S292E, S304P, Q335P and T407K;
(j) L28R, C 50E, T57F, S125A, H128R, K134P, H146N, H160K, A185V, T188V, V211I, V216Q, S237P, A285P, S292E, S304P, V319Q, Q335P, A387Q, and T407K;
(k) L28R, C50E, T57F, S125A, H128R, K134P, A185T, S237P, A285P, S292E, S304P, Q335P, A387Q, and T407K;
(l) K2Q, A3T, A9N, R17K, A18Y, L28R, T43D, T47S, T48L, C50E, T57F, C61H, C68P, A74R, N96H, S97N, K114N, S125A, H128R, L130K, K134P, H146N, S152R, S155A, F157Y, H160K, G167D, A185V, T188A, T202S, V207I, G209E, V211I, R213K, V216Q, N226D, S237P, A254D, Q265R, M266Q, A285P, S292E, S304P, V319Q, I320V, Q335P, N336G, V339Y, E341Q, S370T, T378E, A387Q, D390S, D405N, R406N, T407Q, and V453K; or
(m) C50E, T57F, S125A, H128R, K134P, A185T, S237P, A285P, S292E, Q335P, and T407K.

In one embodiment, provided herein are nucleic acids encoding an above-described tryptophan dimethylallyltransferase variant of SEQ ID NO: 51. In another embodiment, provided herein are expression vectors comprising the nucleic acid encoding the tryptophan dimethylallyltransferase variant of SEQ ID NO: 51. In another embodiment, provided herein are host cells transformed with the expression vector comprising the nucleic acid encoding the tryptophan dimethylallyltransferase variant of SEQ ID NO: 51. In a further embodiment, provided herein are methods for producing a tryptophan dimethylallyltransferase variant, said method comprising expressing the tryptophan dimethylallyltransferase variant in the transformed host cell, wherein the host cell is transformed with the expression vector comprising the nucleic acid encoding the tryptophan dimethylallyltransferase variant of SEQ ID NO: 51.

### Arylesterase Variants

Arylesterase is an enzyme of the hydrolase family that catalyzes the reaction of phenyl acetate and H₂O to phenol and acetate. Arylesterase takes part in bisphenol A degradation. The serum enzymes paraoxonases/arylesterases, found in the liver and blood, hydrolyze a broad spectrum of organophosphate substrates, including paraoxon and a number of aromatic carboxylic acid esters, e.g., phenyl acetate, and thus confer resistance to organophosphate toxicity. Human arylesterase (PON1) is associated with HDL and may protect against LDL oxidation, making it a useful therapeutic.

Also provided herein are arylesterase variants of a wild-type arylesterase from Pseudomonas pseudoalcaligenes of SEQ ID NO: 56 comprising one or more mutations selected from A34G, F35Y, K49Q, S52K, E54H, E57D, S78P, E83R, E87D, Q102L, V115I, S118A, L128V, V138Q or V138R; T142Q or T142E, T149K or T149E; E153K, S158P, G168A, V170N, G172E, M173W or M173L; A182S, E183A, E187P or E187A, I188Q, L189I, and T196Q.

In another embodiment, provided herein is an arylesterase variant of a wild-type arylesterase from Pseudomonas pseudoalcaligenes of SEQ ID NO: 56 comprising mutations:
(a) F35Y, E54H, S78P, E87D, Q102L, L128V, S158P, G168A, G172E, and M173W;
(b) A34G, F35Y, K49Q, S52K, E54H, E57D, S78P, E83R, E87D, Q102L, S118A, L128V, V138Q, T142Q, T149K, S158P, G168A, G172E, M173L, E187P, and I188Q;
(c) A34G, F35Y, S52K, E54H, E57D, S78P, E87D, Q102L, S118A, L128V, T142Q, S158P, G168A, G172E, M173L, E187P, and I188Q;
(d) A34G, F35Y, S52K, E54H, E57D, S78P, E87D, Q102L, S118A, L128V, V138Q, T142Q, T149K, S158P, G168A, G172E, M173L, E187P, I188Q, and L1891;
(e) A34G, F35Y, K49Q, S52K, E54H, E57D, S78P, E83R, E87D, Q102L, V115I, S118A, L128V, V138Q, T142Q, T149E, E153K, S158P, G168A, V170N, G172E, M173L, A182S, E183A, E187A, I188Q, and T196Q;
(f) A34G, F35Y, K49Q, S52K, E54H, E57D, S78P, E83R, E87D, Q102L, V115I, S118A, L128V, V138R, T142E, T149A, E153K, S158P, G168A, V170N, G172E, M173L, A182S, E183A, E187A, I188Q, and T196Q;
(g) A34G, F35Y, S52K, E54H, E57D, S78P, E87D, Q102L, S118A, L128V, V138Q, T142Q, T149K, S158P, G168A, G172E, M173L, E187P, and I188Q;
(h) F35Y, E54H, E57D, S78P, E87D, Q102L, L128V, T142Q, S158P, G168A, G172E, M173L, E187P, and L1891;
(i) F35Y, E54H, S78P, E87D, Q102L, L128V, S158P, G168A, G172E, M173W, and L1891;
(j) A34G, F35Y, K49Q, S52K, E54H, E57D, S78P, E83R, E87D, Q102L, S118A, L128V, V138R, T142E, T149K, S158P, G168A, G172E, M173L, E187P, and I188Q; or
(k) F35Y, E54H, S78P, E87D, Q102L, L128V, S158P, and G168A.

In one embodiment, provided herein are nucleic acids encoding an above-described arylesterase variant of SEQ ID NO: 56. In another embodiment, provided herein are expression vectors comprising the nucleic acid encoding the arylesterase variant of SEQ ID NO: 56. In another embodiment, provided herein are host cells transformed with the expression vector comprising the nucleic acid encoding the arylesterase variant of SEQ ID NO: 56. In a further embodiment, provided herein are methods for producing an arylesterase variant, said method comprising expressing the arylesterase variant in the transformed host cell, wherein the host cell is transformed with the expression vector comprising the nucleic acid encoding the arylesterase variant of SEQ ID NO: 56.

### Putrescine Aminotransferase Variants

Putrescine aminotransferase is an enzyme that reversibly catalyzes the aminotransferase reaction between putrescine and 2-oxoglutarate to produce 1-pyrroline, L-glutamate and water. This enzyme has an optimum pH of 9.0 and is active at an alkaline pH, and is highly active at 20° C.-80° C., with an optimum temperature of 60° C. Putrescine aminotransferase has been found to catalyze the transamination of 12-aminododecanoic acid (Nylon 12) rendering it a valuable industrial biocatalyst in the production of polyamide.

Also provided herein are putrescine aminotransferase variants of a wild-type putrescine aminotransferase from Escherichia coli of SEQ ID NO: 57 comprising one or more mutations selected from A12E, A15Q, H16R, R24D, H28E, A33Q, R36K, K43R, E44K, F50Y, A59K, F84Y, V102K, S105K, N109E, A112D, Q114L, Q119N, L121F, D123N, K131H, A135E, T137A, K140D, S144V, S153A, K162R, A175T, A1925, S194E, T195E, F196Y, N214D, M218L, T220K, N223E, L248I, T255R, F264Y or F264H; M268L, K282R, N290G, Q292V, A299G, L322F, L344I, T346A, N348H, Q353E, A357E, Q361E, M365Y or M365R, V381I, Q382R, M389L, N398E or N398D; G401A, S406A, R412G, T4185, A4225, T424V, I435D, C438I, E439D, L440K or L440Q; M452I, and S455Q.

In one embodiment, provided herein is a putrescine aminotransferase variant of a wild-type putrescine aminotransferase from Escherichia coli of SEQ ID NO: 57 comprising mutations:
(a) A12E, H16R, H28E, A33Q, K43R, V102K, S105K, N109E, A112D, Q119N, L121F, D123N, K131H, A135E, T137A, K140D, S144V, S153A, K162R, S194E, T195E, F196Y, N214D, M218L, T220K, N223E, L248I, T255R, F264Y, M268L, K282R, Q292V, A299G, L344I, N348H, Q353E, A357E, Q361E, M365Y, V381I, Q382R, M389L, N398E, S406A, T4185, T424V, I435D, C438I, E439D, L440K, and M452I;
(b) N109E, L121F, K140D, S144V, S153A, T195E, M218L, N223E, L248I, T255R, K282R, Q292V, A299G, N348H, M365Y, Q382R, M389L, S406K, T424V, I435D, and C438L;
(c) N109E, L121F, A135E, K140D, S144V, S153A, K162R, T195E, M218L, T220K, N223E, L248I, T255R, K282R, Q292V, A299G, N348H, M365Y, Q382R, M389L, S406A, T424V, I435D, and C438I;
(d) A12E, H28E, A33Q, S105K, N109E, L121F, K131H, A135E, T137A, K140D, S144V, S153A, K162R, S194E, T195E, N214D, M218L, T220K, N223E, L248I, T255R, F264H, K282R, Q292V, A299G, N348H, Q353E, Q361E, M365Y, V381I, Q382R, M389L, S406A, T424V, I435D, C438I, and L440Q;
(e) N109E, L121F, K140D, S144V, S153A, T195E, M218L, N223E, L248I, T255R, K282R, Q292V, A299G, N348H, M365Y, Q382R, M389L, S406K, T424V, I435D, and C438I;
(f) A12E, S105K, N109E, L121F, K131H, A135E, T137A, K140D, S144V, S153A, K162R, S194E, T195E, N214D, M218L, T220K, N223E, L248I, T255R, F264H, K282R, Q292V, A299G, N348H, M365Y, Q382R, M389L, S406A, T424V, I435D, C438I, and L440R;
(g) N109E, L121F, A135E, K140D, S144V, S153A, K162R, T195E, T220K, N223E, L248I, T255R, K282R, Q292V, A299G, N348H, M365Y, Q382R, M389L, S406A, T424V, I435D, and C438I;
(h) A12E, H28E, A33Q, S105K, N109E, L121F, K131H, A135E, T137A, K140D, S144V, S153A, K162R, S194E, T195E, N214D, M218L, T220K, N223E, L248I, T255R, F264Y, K282R, Q292V, A299G, N348H, Q353E, Q361E, M365Y, V381I, Q382R, M389L, S406A, T424V, I435D, C438I, and L440Q;
(i) A12E, S105K, N109E, L121F, K131H, A135E, T137A, K140D, S144V, S153A, K162R, S194E, T195E, N214D, M218L, T220K, N223E, L248I, T255R, K282R, Q292V, A299G, N348H, M365Y, Q382R, M389L, S406A, T424V, I435D, and C438I;
(j) L121F, S144V, S153A, T195E, N223E, L248I, T255R, K282R, Q292V, A299G, N348H, M365Y, M389L, S406K, T424V, I435D, and C438I;
(k) A12E, A15Q, H16R, R24D, H28E, A33Q, R36K, E44K, F50Y, A59K, V102K, S105K, N109E, A112D, Q114L, Q119N, L121F, D123N, K131H, A135E, T137A, K140D, S144V, S153A, K162R, A175T, A1925, S194E, T195E, F196Y, N214D, M218L, T220K, N223E, L248I, T255R, F264H, M268L, K282R, N290G, Q292V, A299G, L322F, L344I, T346A, N348K, Q353E, A357E, Q361E, M365Y, V381I, Q382R, M389L, N398E, G401A, S406A, R412G, T4185, A4225, T424V, I435D, C438I, E439D, L440K, M452I, and S455Q;
(l) A12E, A15Q, H16R, R24D, H28E, A33Q, R36K, K43R, F50Y, A59K, F84Y, V102K, S105K, N109E, A112D, Q114L, Q119N, L121F, D123N, K131H, A135E, K140D, S144V, S153A, K162R, A175T, A1925, S194E, T195E, F196Y, N214D, M218L, T220K, N223E, L248I, T255R, F264Y, M268L, K282R, N290G, Q292V, A299G, L322F, L344I, T346A, N348K, Q353E, A357E, Q361E, M365Y, V381I, Q382R, M389L, N398D, G401A, S406A, R412G, T4185, A4225, T424V, I435D, C438I, E439D, L440K, M452I, and S455Q;
(m) A12E, H16R, H28E, A33Q, K43R, V102K, S105K, N109E, A112D, Q119N, L121F, D123N, K131H, A135E, T137A, K140D, S144V, S153A, K162R, S194E, T195E, F196Y, N214D, M218L, T220K, N223E, L248I, T255R, F264Y, M268L, K282R, Q292V, A299G, L344I, N348H, Q353E, A357E, Q361E, M365R, V381I, Q382R, M389L, N398E, S406A, T418S, T424V, I435D, C438I, E439D, L440K, and M452I; or
(n) L121F, S144V, S153A, T195E, N223E, L248I, T255R, K282R, Q292V, A299G, N348H, M365Y, M389L, S406K, T424V, and I435D.

In one embodiment, provided herein are nucleic acids encoding an above-described putrescine aminotransferase variant of SEQ ID NO: 57. In another embodiment, provided herein are expression vectors comprising the nucleic acid encoding the putrescine aminotransferase variant of SEQ ID NO: 57. In another embodiment, provided herein are host cells transformed with the expression vector comprising the nucleic acid encoding the putrescine aminotransferase variant of SEQ ID NO: 57. In a further embodiment, provided herein are methods for producing a putrescine aminotransferase variant, said method comprising expressing the putrescine aminotransferase variant in the transformed host cell, wherein the host cell is transformed with the expression vector comprising the nucleic acid encoding the putrescine aminotransferase variant of SEQ ID NO: 57.

### Green to Red Photoconvertible GFP-like Protein EosFP Variants

Green to Red Photoconvertible GFP-like Protein EosFP (“EosFP”) was isolated from the stony coral Lobophyllia hemprichii. This protein matures in a green fluorescent state with an emission maximum at 516 nm. Upon irradiation with violet-blue light the chromophore undergoes an irreversible photoconversion to a red state emitting at 581 nm. The wavelengths required for photoconversion and detection of the green and red fluorescent states can be easily separated, rendering EosFP an excellent choice for regional optical marking. EosFP is used as a marker for tracking cells, including cell fate mapping and tracking of metastases, compartments and proteins in live cells, based on the principle that the marker is regionally photoconverted from green to red. Subsequently, the red fluorescent fraction can be tracked independently. EosFP is introduced into cells by transfection of expression vectors or microinjection of in vitro transcribed mRNA or purified recombinant protein. Subcellular components also can be tracked with EosFP. To track a protein of interest, EosFP is fused thereto; after photoconversion, the movement of the marked fraction of the protein is followed by red fluorescence. Likewise, EosFP is fused to a protein of interest as a nanoscopy marker to determine its subcellular localization with using photoactivated localization microscopy (PALM).

Provided herein are green to red photoconvertible GFP-like protein EosFP variants of a wild-type green to red photoconvertible GFP-like protein EosFP from Lobophyllia hemprichii of SEQ ID NO: 58 comprising one or more mutations selected from N11K, D26E, D28E, K32N, F34Y, M40I, L93M, T94I, I100V or I102T; T113C, N116H, K117E, R119K or R119E, A127P, L137I, K145M, K145S or K145Y; T154V, I157C or I157V, A160F, N166G, A167G, Y169L, F173C, F173V or F1731; E181K, L186M, F191L, V192I, and C195R.

In one embodiment, provided herein is a green to red photoconvertible GFP-like protein EosFP variant of a wild-type green to red photoconvertible GFP-like protein EosFP from Lobophyllia hemprichii of SEQ ID NO: 58 comprising mutations:
(a) N11K, K32N, F34Y, L93M, T94I, I100V, I102T, T113C, N116H, R119K, A127P, K145M, T154V, I157C, A160F, N166G, A167G, F173C, E181K, F191L, V192I, and C195R;
(b) N11K, K32N, L93M, T94I, I100V, I102T, T113C, R119E, A127P, K145S, A160F, N166G, A167G, F173V, E181K, F191L, V192I, and C195R;
(c) N11K, K32N, F34Y, L93M, I100V, I102T, T113C, N116H, K117E, R119E, A127P, K145M, T154V, I157C, A160F, N166G, A167G, F173C, E181K, F191L, V192I, and C195R;
(d) N11K, K32N, L93M, I100V, R119E, A127P, K145M, T154V, I157C, A160F, N166G, A167G, F173C, E181K, F191L, V192I, and C195R;
(e) L93M, R119E, A127P, K145M, N166G, A167G, F173C, F191L, and C195R;
(f) N11K, D26E, D28E, K32N, F34Y, M40I, L93M, I100C, I102T, T113C, N116H, K117E, R119E, A127P, L137I, K145M, T154V, I157C, A160F, N166G, A167G, F173I, E181K, L186M, F191L, V192I, and C195R;
(g) N11K, K32N, L93M, I100V, R119E, A127P, K145M, T154V, A160F, N166G, A167G, F173V, E181K, F191L, V192I, and C195R;
(h) N11K, K32N, L93M, I100V, T113C, R119E, A127P, K145M, T154V, I157C, A160F, N166G, A167G, Y169L, F173V, E181K, F191L, V192I, and C195R;
(i) N11K, K32N, L93M, I100V, T113C, R119E, A127P, K145M, T154V, A160F, N166G, A167G, F173C, E181K, F191L, V192I, and C195R;
(j) N11K, D26E, D28E, K32N, F34Y, M40I, L93M, T94I, I100V, I102T, T113C, N116H, R119E, A127P, L137I, K145Y, T154V, I157V, A160F, N166G, A167G, F173V, E181K, L186M, F191L, V192I, and C195R;
(k) N11K, K32N, L93M, I100V, R119E, A127P, K145S, N166G, A167G, F173C, F191L, V192I, and C195R; or
(l) N11K, K32N, L93M, I100V, I102T, T113C, R119E, A127P, K145M, I157C, A160F, N166G, A167G, F173C, E181K, F191L, V192I, and C195R.

In one embodiment, provided herein are nucleic acids encoding an above-described green to red photoconvertible GFP-like protein EosFP variant of SEQ ID NO: 58. In another embodiment, provided herein are expression vectors comprising the nucleic acid encoding the green to red photoconvertible GFP-like protein EosFP variant of SEQ ID NO: 58. In another embodiment, provided herein are host cells transformed with the expression vector comprising the nucleic acid encoding the green to red photoconvertible GFP-like protein EosFP variant of SEQ ID NO: 58. In a further embodiment, provided herein are methods for producing a green to red photoconvertible GFP-like protein EosFP variant, said method comprising expressing the green to red photoconvertible GFP-like protein EosFP variant in the transformed host cell, wherein the host cell is transformed with the expression vector comprising the nucleic acid encoding the green to red photoconvertible GFP-like protein EosFP variant of SEQ ID NO: 58.

### Red Fluorescent Protein drFP583 Variants

Red Fluorescent Protein drFP583 is found in sea anemone of the Discosoma sp. This protein is theorized to have a role in photoprotection of the coral's resident symbiont microalgae's photosystems from photoinhibition caused by high light levels found near the surface of coral reefs. In deeper water, the fluorescence may be to convert blue light into longer wavelengths more suitable for use in photosynthesis by the microalgal symbionts. Fluorescent proteins such as red fluorescent protein drFP583 are a useful and ubiquitous tool for making chimeric proteins, where they function as a fluorescent protein tag. These proteins have been expressed in most known cell types and are used as a noninvasive fluorescent marker in living cells and organisms. They enable a wide range of applications where they have functioned as a cell lineage tracer, reporter of gene expression, or as a measure of protein-protein interactions.

Also provided herein are red fluorescent protein drFP583 variants of a wild-type red fluorescent protein drFP583 from Discosoma sp. of SEQ ID NO: 59 comprising one or more mutations selected from M1A, N6S, R17H, T21S, V22M, H41T, N42Q, V44A, A57S, K70R, V71A, Y72F, V73T, K83Y, L85Q, N98V, S111T, Q114E, D115G, C117T, F118L, K123W, F124L, I125R, V127T, S131P, D132N, A145P, R153E, E160D, H162K, K163M, K166R, H172R, L174R or L174T; V175A, E176D or E176Q; S179T, I180T, M182K, L189M, Y192A or Y192S; Y194F, S197R, D200E, N205D, I210V, T217S or T217A; G219A, H222S, L223T, and F224G.

In another embodiment, provided herein is a red fluorescent protein drFP583 variant of a wild-type red fluorescent protein drFP583 from Discosoma sp. of SEQ ID NO: 59 comprising mutations:
(a) M1A, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, I210V, T217S, H222S, L223T, and F224G;
(b) N6S, R17H, T21S, V22M, H41T, N42Q, V44A, A57S, K70R, V71A, Y72F, V73T, K83Y, L85Q, N98V, S111T, Q114E, D115G, F118L, K123W, F124L, I125R, V127T, S131P, D132N, A145P, R153E, E160D, H162K, K163M, K166R, L174T, V175A, E176Q, S179T, I180T, M182K, L189M, Y192S, Y194F, S197R, D200E, N205D, I210V, T217A, G219A, H222S, L223T, and F224G;
(c) M1A, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T217S, H222S, L223T, and F224G;
(d) N6S, R17H, T21S, V22M, H41T, N42Q, V44A, A57S, K70R, V71A, Y72F, V73T, K83Y, L85Q, S111T, Q114E, D115G, C117T, F118L, F124L, I125R, V127T, S131P, A145P, R153E, E160D, H162K, K163M, K166R, H172R, L174T, V175A, E176D, S179T, I180T, M182K, L189M, Y192A, Y194F, S197R, N205D, I210V, T217S, G219A, H222S, L223T, and F224G;
(e) N6S, R17H, T21S, V22M, H41T, N42Q, V44A, A57S, K70R, V71A, Y72F, V73T, K83Y, L85Q, S111T, Q114E, D115G, F118L, F124L, I125R, V127T, S131P, D132N, A145P, R153E, E160D, H162K, K163M, K166R, H172R, L174T, V175A, E176D, S179T, I180T, M182K, L189M, Y192A, Y194F, S197R, N205D, I210V, T217A, H222S, L223T, and F224G;
(f) N6S, R17H, T21S, V22M, H41T, N42Q, V44A, A57S, K70R, V71A, Y72F, V73T, K83Y, L85Q, S111T, Q114E, D115G, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T217S, G219A, H222S, L223T and F224G; or
(g) N6S, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T2175, G219A, H222S, L223T, and F224G.

In one embodiment, provided herein are nucleic acids encoding an above-described red fluorescent protein drFP583 variant of SEQ ID NO: 59. In another embodiment, provided herein are expression vectors comprising the nucleic acid encoding the red fluorescent protein drFP583 variant of SEQ ID NO: 59. In another embodiment, provided herein are host cells transformed with the expression vector comprising the nucleic acid encoding the red fluorescent protein drFP583 variant of SEQ ID NO: 59. In a further embodiment, provided herein are methods for producing a red fluorescent protein drFP583 variant, said method comprising expressing the red fluorescent protein drFP583 variant in the transformed host cell, wherein the host cell is transformed with the expression vector comprising the nucleic acid encoding the red fluorescent protein drFP583 variant of SEQ ID NO: 59.

### Green Fluorescent Protein Variants

The green fluorescent protein (GFP) is a protein composed of 238 amino acid residues (26.9 kDa) that exhibits bright green fluorescence when exposed to light in the blue to ultraviolet range. Traditionally, GFP refers to the protein first isolated from the jellyfish Aequorea victoria., which has a major excitation peak at a wavelength of 395 nm and a minor one at 475 nm; its emission peak is at 509 nm, which is in the lower green portion of the visible spectrum. The GFP gene is frequently used as a reporter of expression and also has been used in modified forms to make bio sensors. A modified GFP was made by a single point mutation 565T), which improved its spectral characteristics, resulting in increased fluorescence. Since then, many mutations, including color mutants, have been made, including blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet). GFP is used as a reported gene, in fluorescence microscopy for cell biology, such as tracking cancer cells and metastases, and in tracking biological processes, including spread of virus infections.

Further provided herein are green fluorescent protein variants of a wild-type green fluorescent protein from Aequorea victoria of SEQ ID NO: 60 comprising one or more mutations selected from S28K, 530R, F64L, S72A, N105C, N146I, H148G, M153T, K158N, V163A, N164E, I167M, N170P, S175G, N198Y, 5202H, Q204R or Q204H; A206V, and V224L.

In another embodiment, provided herein is a green fluorescent protein variant of a wild-type green fluorescent protein from Aequorea victoria of SEQ ID NO: 60 comprising mutations:
(a) S28K, 530R, F64L, S72A, N105C, N146I, H148G, M153T, K158N, V163A, N164E, I167M, N170P, S175G, N198Y, 5202H, Q204R, and A206V;
(b) S28K, 530R, F64L, S72A, N105C, N146I, H148G, M153T, K158N, V163A, N164E, I167M, N170P, S175G, N198Y, Q204H, and A206V;
(c) S28K, 530R, F64L, S72A, N105C, N146I, H148G, M153T, K158N, V163A, N164E, I167M, N170P, S175G, N198Y, Q204H, A206V, and V224L;
(d) 530R, F64L, S72A, N105C, N146I, H148G, M153T, K158N, V163A, N170P, S175G, 5202H, A206V, and V224L;
(e) 530R, F64L, S72A, N146I, H148G, M153T, K158N, V163A, S175G, 5202H, and V224L;
(f) 530R, F64L, S72A, N105C, N146I, H148G, M153T, K158N, V163A, N170P, S175G, N198Y, 5202H, A206V, and V224L; or
(g) S28K, 530R, F64L, S72A, N105C, N146I, H148G, M153T, K158N, V163A, N170P, S175G, N198Y, 5202H, A206V, and V224L.

In one embodiment, provided herein are nucleic acids encoding an above-described green fluorescent protein variant of SEQ ID NO: 60. In another embodiment, provided herein are expression vectors comprising the nucleic acid encoding the green fluorescent protein variant of SEQ ID NO: 60. In another embodiment, provided herein are host cells transformed with the expression vector comprising the nucleic acid encoding the green fluorescent protein variant of SEQ ID NO: 60. In a further embodiment, provided herein are methods for producing a green fluorescent protein variant, said method comprising expressing the green fluorescent protein variant in the transformed host cell, wherein the host cell is transformed with the expression vector comprising the nucleic acid encoding the green fluorescent protein variant of SEQ ID NO: 60.

### Luciferin 4-monooxygenase Variants

Firefly luciferin 4-monooxygenase, also known as luciferase, is found in Photinus pyralis, the Common eastern firefly. Luciferase is a general term for the class of oxidative enzymes used in bioluminescence and is distinct from a photoprotein. Luciferase catalyzes a bioluminescent reaction which involves the substrate luciferin as well as Mg2+ and ATP, produces green light with a wavelength of 562 nm. Luciferase from firefly is broadly used as a reporter for studying gene regulation and function, and for pharmaceutical screening.

Also provided herein are luciferin 4-monooxygenase variants of a wild-type luciferin 4-monooxygenase from Photinus pyralis of SEQ ID NO: 61 comprising one or more mutations selected from A4G, K8I, A12P, F14R, D19P, Q25L, A29L, V36H, T39R, I47T, E48G, V49Q, Y56L, E58Q, M59W, 560A, V61N or V61C; L63M or L63V; M67L, T74K, N75G, R77V, Q87E, M90V, A101T or A101V; A103H or A103T; I108N or I108T, N110T, R112D, L1141, N116H, S117Q, M118L, T124K, V128T, K130P or K1305; G132A, Q134P, Q147K, T156Q, Q159N, V168M, T169K, H171Y, G175N, V182K, K190Q, T191Q, T214N, A215I, A222C, R223S, D234G, G246A, T251C or T251H; T252V, Y255H, I257R or I257Y; C258Q, L264V, Y266R or Y266K; S276A, L277V or L277I; K281R, Q283T, S284V, T290S or T290P, F292M, S293V, N3085, H310T, G316A, H332N or H332G; I349V, A361S, F368G, V384P, M396N, S399K, N409K, L411T, 5420T, W426F, E430G, S440E, S456A, I457V, Q460S, N463D or N463K; F465A, L472Y, E488K, H489P, V499I, T5075, T508P, A509H or A509Y; V522I, G525T, G525N or G5255; L526P, L530I, and A532R or A532K.

In one embodiment, provided herein is a luciferin 4-monooxygenase variant of a wild-type luciferin 4-monooxygenase from Photinus pyralis of SEQ ID NO: 61 comprising mutations:
(a) A4G, K8I, A12P, D19P, Q25L, V36H, T39R, I47T, E48G, V49Q, Y56L, E58Q, M59W, 560A, V61N, L63M, M67L, T74K, N75G, R77V, M90V, A101T, A103H, I108N, N110T, R112D, L114I, N116H, S117Q, M118L, T124K, V128T, K130P, G132A, Q134P, V168M, T169K, H171Y, G175N, V182K, K190Q, T191Q, T214N, A222C, D234G, G246A, T251C, T252V, I257R, C258Q, L264V, Y266R, S276A, Q283T, T290S, S293V, G316A, H332N, A361S, V384P, M396N, S399K, N409K, L411T, 5420T, W426F, E430G, S440E, S456A, I457V, Q460S, N463D, F465A, L472Y, E488K, H489P, V499I, T5075, T508P, A509H, V522I, G525T, L526P, L530I, and A532R;
(b) A4G, K8I, D19P, T39R, I47T, E48G, Y56L, M59W, V61C, L63V, M67L, R77V, M90V, A101T, A103H, I108N, N110T, N116H, S117Q, M118L, T124K, K130P, V168M, H171Y, T191Q, T214N, T251H, I257R, L264V, Y266K, S276A, L277V, Q283T, T290P, S293V, G316A, V384P, 5420T, W426F, E430G, S440E, F465A, L472Y, E488K, H489P, T507S, T508P, A509Y, V522I, G525N, and A532K;
(c) A4G, K8I, D19P, T39R, I47T, E48G, Y56L, V61C, L63V, M67L, R77V, M90V, A101T, A103H, I108N, N110T, N116H, S117Q, M118L, T124K, K130P, V168M, H171Y, T191Q, T214N, T251H, I257R, L264V, Y266K, S276A, L277V, Q283T, T290P, S293V, G316A, V384P, S420T, W426F, E430G, S440E, F465A, L472Y, E488K, H489P, T507S, T508P, A509Y, V522I, G525N, and A532K;
(d) A4G, K8I, D19P, I47T, E48G, Y56L, V61C, L63V, M67L, R77V, M90V, A101T, A103H, I108N, N116H, S117Q, M118L, K130P, V168M, H171Y, T191Q, T214N, T251H, I257R, L264V, Y266K, S276A, K281R, Q283T, T290P, S293V, V384P, S420T, W426F, E430G, S440E, F465A, L472Y, E488K, H489P, T508P, A509Y, V522I, G525N, and A532K;
(e) A4G, K8 I, D19P, I47T, E48G, Y56L, M59W, V61C, L63V, M67L, R77V, M90V, A101T, A103H, I108N, S117Q, M118L, K130P, V168M, H171Y, T251H, I257R, L264V, Y266K, S276A, L277V, K281R, Q283T, T290P, S293V, V384P, S420T, W426F, E430G, S440E, F465A, L472Y, E488K, H489P, T508P, A509H, V522I, and A532K;
(f) A4G, K8I, A12P, F14R, D19P, Q25L, V36H, T39R, I47T, E48G, V49Q, Y56L, E58Q, M59W, S60A, V61N, L63M, M67L, T74K, N75G, R77V, Q87E, M90V, A101T, A103H, I108N, N110T, R112D, L114I, N116H, S117Q, M118L, T124K, V128T, K130P, G132A, Q134P, Q147K, T156Q, Q159N, V168M, T169K, H171Y, G175N, V182K, K190Q, T191Q, T214N, A222C, D234G, G246A, T251C, T252V, I257R, C258Q, L264V, Y266R, S276A, L277I, Q283T, T290S, S293V, N308S, H310T, G316A, H332G, I349V, A361S, F368G, V384P, M396N, S399K, N409K, L411T, S420T, W426F, E430G, S440E, S456A, I457V, Q460S, N463K, F465A, L472Y, E488K, H489P, V499I, T507S, T508P, A509Y, V522I, G525T, L526P, L530I, and A532R;
(g) A4G, K8I, A12P, D19P, T39R, I47T, E48G, V49Q, Y56L, S60A, V61C, L63V, M67L, T74K, R77V, M90V, A101T, A103H, I108T, N110T, N116H, S117Q, M118L, T124K, V128T, K130P, G132A, Q134P, V168M, H171Y, G175N, T191Q, T214N, A215I, D234G, G246A, Y255H, I257R, C258Q, L264V, Y266K, S276A, L277V, Q283T, T290P, S293V, G316A, H332N, A361S, V384P, S399K, N409K, S420T, W426F, E430G, S440E, S456A, I457V, Q460S, N463D, F465A, L472Y, E488K, H489P, V499I, T507S, T508P, A509Y, V522I, G525S, L526P, L530I, and A532K;
(h) A4G, K8I, A12P, F14R, D19P, Q25L, A29L, V36H, T39R, I47T, E48G, V49Q, Y56L, E58Q, M59W, S60A, V61N, L63M, M67L, T74K, N75G, R77V, Q87E, M90V, A101V, A103H, I108N, N110T, R112D, L114I, N116H, S117Q, M118L, T124K, V128T, K130P, G132A, Q134P, Q147K, T156Q, Q159N, V168M, T169K, H171Y, G175N, V182K, K190Q, T191Q, T214N, A215I, A222C, D234G, G246A, T251C, T252V, I257Y, C258Q, L264V, Y266R, S276A, L277I, Q283T, T290S, F292M, S293V, N308S, H310T, G316A, H332G, I349V, A361S, F368G, V384P, M396N, S399K, N409K, L411T, S420T, W426F, E430G, S440E, S456A, I457V, Q460S, N463K, F465A, L472Y, E488K, H489P, V499I, T507S, T508P, A509Y, V522I, G525T, L526P, L530I, and A532R;
(i) A4G, K8I, D19P, I47T, E48G, Y56L, M59W, V61C, L63V, M67L, R77V, M90V, A101T, A103T, I108N, N116H, S117Q, M118L, K130S, V168M, H171Y, T191Q, T214N, T251H, I257R, L264V, Y266K, S276A, L277V, K281R, Q283T, S284V, T290P, S293V, V384P, S420T, W426F, E430G, S440E, F465A, L472Y, E488K, H489P, T508P, A509Y, V522I, G525N, and A532K;
(j) A4G, K8I, A12P, D19P, Q25L, V36H, T39R, I47T, E48G, V49Q, Y56L, E58Q, M59W, S60A, V61N, L63M, M67L, T74K, N75G, R77V, M90V, A101T, A103H, I108N, N110T, R112D, L114I, N116H, S117Q, M118L, T124K, V128T, K130P, G132A, Q134P, V168M, T169K, H171Y, G175N, V182K, K190Q, T191Q, T214N, A222C, D234G, G246A, T251C, T252V, I257R, C258Q, L264I, Y266R, S276A, L277V, Q283T, T290S, S293V, G316A, H332N, A361S, V384P, M396N, S399K, N409K, L411T, S420T, W426F, E430G, S440E, S456A, I457V, Q460S, N463D, F465A, L472Y, E488K, H489P, V499I, T507S, T508P, A509H, V522I, G525T, L526P, L530I, and A532R;
(k) A4G, K8I, D19P, I47T, E48G, Y56L, V61C, L63V, M67L, R77V, M90V, A101T, A103H, I108N, S117Q, M118L, K130P, V168M, H171Y, T251H, I257R, L264V, Y266K, S276A, L277V, K281R, Q283T, S284V, T290P, S293V, V384P, S420T, W426F, E430G, S440E, F465A, L472Y, E488K, H489P, T508P, A509Y, V522I, and A532K;
(l) A4G, K8I, A12P, D19P, T39R, I47T, E48G, Y56L, M59W, V61C, L63V, M67L, R77V, M90V, A101V, A103T, I108T, N110T, N116H, S117Q, M118L, T124K, K130S, Q134P, V168M, H171Y, G175N, T191Q, T214N, R223S, T251H, I257Y, L264V, Y266K, S276A, Q283T, T290P, S293V, G316A, A361S, V384P, S399K, N409K, S420T, W426F, E430G, S440E, F465A, L472Y, E488K, H489P, V499I, T507S, T508P, A509H, V522I, G525N and A532K;
(m) A4G, K8I, A12P, D19P, T39R, I47T, E48G, V49Q, Y56L, M59W, S60A, V61C, L63V, M67L, T74K, R77V, M90V, A101V, A103H, I108T, N110T, N116H, S117Q, M118L, T124K, V128T, K130P, G132A, V168M, H171Y, G175N, T191Q, T214N, D234G, G246A, T251H, I257R, C258Q, L264V, Y266K, S276A, L277V, Q283T, T290P, S293V, G316A, H332N, A361S, V384P, S399K, N409K, S420T, W426F, E430G, S440E, S456A, I457V, Q460S, N463D, F465A, L472Y, E488K, H489P, V499I, T507S, T508P, A509Y, V522I, G525N, L526P, L530I, and A532K; or
(n) A4G, K8I, A12P, D19P, T39R, I47T, E48G, Y56L, V61C, L63V, M67L, R77V, M90V, A101V, A103T, I108T, N110T, N116H, S117Q, M118L, T124K, K130P, Q134P, V168M, H171Y, G175N, T191Q, T214N, I257R, L264V, Y266K, S276A, K281R, Q283T, T290P, S293V, G316A, A361S, V384P, S399K, N409K, S420T, W426F, E430G, S440E, F465A, L472Y, E488K, H489P, V499I, T507S, T508P, A509H, V522I, G525N, and A532K.

In one embodiment, provided herein are nucleic acids encoding an above-described luciferin 4-monooxygenase variant of SEQ ID NO: 61. In another embodiment, provided herein are expression vectors comprising the nucleic acid encoding the luciferin 4-monooxygenase variant of SEQ ID NO: 61. In another embodiment, provided herein are host cells transformed with the expression vector comprising the nucleic acid encoding the luciferin 4-monooxygenase variant of SEQ ID NO: 61. In a further embodiment, provided herein are methods for producing a luciferin 4-monooxygenase variant, said method comprising expressing the luciferin 4-monooxygenase variant in the transformed host cell, wherein the host cell is transformed with the expression vector comprising the nucleic acid encoding the luciferin 4-monooxygenase variant of SEQ ID NO: 61.

### mOrange Variants

In a mOrange and mOrange2, called “living colors” are extremely bright orange fluorescent protein monomers which can be used as tags or reporters. Both mOrange fluorescent proteins are mutants derived from mRFP1, a monomeric mutant of DsRed, which was developed in Dr. Roger Tsien's lab by directed mutagenesis. mOrange excitation and emission maxima are 548 and 562 nm, respectively. mOrange2 excitation and emission maxima are 549 and 565 nm, respectively. The mOrange variant fluorescent proteins provided herein are variants of mOrange, which is designated as the “wild-type mOrange” from Discosoma sp.

Provided herein are mOrange variants of a wild-type mOrange from Discosoma sp. of SEQ ID NO: 62 comprising one or more mutations selected from N8D, N9P, R22H, S26C, F46T, A49M, K50R, F88L, D120G, E122C, S136P, D137N, A150P, S152T or S152C, A161T, K167T, T179R, S180C, A197N, I199F, G201D or G201N, K203R, D205Y, D205E or D205V; N210D, Q218E, and G224A.

In one embodiment, provided herein is a mOrange variant of a wild-type mOrange from Discosoma sp. of SEQ ID NO: 62 comprising mutations:
(a) N8D, N9P, R22H, F46T, A49M, F88L, D120G, E122C, S136P, A150P, S152T, A161T, K167T, T179R, S180C, A197N, I199F, G201D, K203R, D205Y, N210D, and G224A;
(b) N8D, N9P, R22H, S26C, F46T, A49M, K50R, F88L, D120G, E122C, S136P, D137N, A150P, S152T, A161T, K167T, R169A, T179R, S180C, A197N, I199F, G201D, K203R, D205Y, N210D, Q218E, and G224A;
(c) N8D, R22H, F46T, F88L, E122C, A150P, S152T, A161T, T179R, S180C, I199F, G201D, K203R, D205Y, and G224A;
(d) N8D, N9P, R22H, F46T, A49M, F88L, D120G, E122C, S136P, A150P, S152T, A161T, K167T, T179R, S180C, A197N, I199F, G201D, K203R, D205E, N210D, and G224A;
(e) N8D, R22H, F46T, A49M, F88L, D120G, E122C, A150P, S152T, A161T, T179R, S180C, I199F, G201D, K203R, D205Y, N210D, and G224A;
(f) N8D, R22H, F46T, F88L, A150P, S152C, A161T, T179R, S180C, I199F, G201N, K203R, D205Y, and G224A; or
(g) N8D, R22H, F46T, F88L, A150P, S152C, A161T, T179R, S180C, I199F, G201N, D205V and G224A.

In one embodiment, provided herein are nucleic acids encoding an above-described mOrange variant of SEQ ID NO: 62. In another embodiment, provided herein are expression vectors comprising the nucleic acid encoding the mOrange variant of SEQ ID NO: 62. In another embodiment, provided herein are host cells transformed with the expression vector comprising the nucleic acid encoding the mOrange variant of SEQ ID NO: 62. In a further embodiment, provided herein are methods for producing a mOrange variant, said method comprising expressing the mOrange variant in the transformed host cell, wherein the host cell is transformed with the expression vector comprising the nucleic acid encoding the mOrange variant of SEQ ID NO: 62.

### Sandercyanin Fluorescent Protein Variants

Sandercyanin fluorescent protein is a blue protein found in the mucus coating blue forms of walleye fish, Sander vitreus. Solutions of Sandercyanin fluorescent protein are deep blue in color and show absorbance maxima at 383 and 633 nm, respectively. The naturally occurring Sandercyanin fluorescent protein is a homotetramer of four identical subunits that are associated but not covalently bound. Monomeric variants of Sandercyanin fluorescent protein are one-quarter the size of the tetrameric protein form, i.e., about 18.6 kDa, and have a large stokes shift (375 nm/675 nm), like the tetrametric form, and fluoresce in the far-red or near infrared region, which is useful for a variety of applications, including studying protein-protein interactions, spatial and temporal gene expression, assessing cell biology distribution and mobility, studying protein activity and protein interactions in vivo, as well as cancer research, immunology, and stem cell research and sub-cellular localization. The far-red fluorescence of monomeric variants of Sandercyanin fluorescent protein also are useful for in vivo deep-tissue imaging. The Sandercyanin fluorescent protein variants provided herein are improved in their brightness of fluorescence and stability compared to the wild-type Sandercyanin fluorescent protein and to previously described monomeric variants of Sandercyanin fluorescent protein, and thus are better fluorescent probes than the naturally occurring blue protein.

Also provided herein are sandercyanin fluorescent protein variants of a wild-type sandercyanin fluorescent Protein from Sander vitreus of SEQ ID NO: 63 comprising one or more mutations selected from M1L, K4R, A12P, E15P, D16N, A19P, A205, A42I, V25T, D28E; K35T, K35P or K35Q; A42I, I114T, N115D, A118D, S119F, A121V, A122E, V127L or V127M; G143E, T144I, M145L, G151P, T154E or T154K; L156I, A162N, and A163C.

In another embodiment, provided herein is a sandercyanin fluorescent protein variant of a wild-type sandercyanin fluorescent Protein from Sander vitreus of SEQ ID NO: 63 comprising mutations:
(a) A12P, D16N, A205, V25T, D28E, S119F, A122E, V127L, G143E, T144I, M145L, G151P, T154E, and A163C;
(b) A12P, D16N, A205, V25T, D28E, S119F, A122E, V127M, G143E, T144I, M145L, G151P, T154E, and A163C;
(c) M1L, A12P, E15P, D16N, A19P, A205, V25T, D28E, K35T, A42I, I114T, N115D, A118D, S119F, A121V, A122E, V127M, G143E, T144I, M145L, G151P, T154K, L156I, A162N and A163C;
(d) M1L, A12P, E15P, D16N, A19P, A20S, V25T, D28E, K35T, A42I, I114T, N115D, A118D, S119F, A121V, A122E, V127L, G143E, T144I, M145L, G151P, T154E, L156I, A162N and A163C;
(e) K4R, A12P, D16N, A20S, V25T, D28E, K35T, A42I, N115D, A118D, S119F, A122E, V127M, G143E, T144I, M145L, G151P, T154K, and A163C;
(f) A12P, D28E, K35P, S119F, A122E, V127M, G143E, M145L, G151P, T154E, and A163C;
(g) K4R, A12P, D16N, A20S, V25T, D28E, K35Q, A42I, N115D, A118D, S119F, A121V, A122E, V127L, G143E, T144I, M145L, G151P, T154K, and A163C;
(h) A12P, D16N, A20S, V25T, D28E, K35T, N115D, S119F, A122E, V127M, G143E, T1441, M145L, G151P, T154K, and A163C;
(i) A12P, A20S, D28E, S119F, A122E, V127M, G143E, T144I, M145L, G151P, T154E and A163C;
(j) A12P, D28E, S119F, A122E, V127M, G143E, M145L, G151P, and T154E; or
(k) A12P, A20S, D28E, K35P, S119F, A122E, V127M, G143E, T144I, M145L, G151P, T154E and A163C.

In one embodiment, provided herein are nucleic acids encoding an above-described sandercyanin fluorescent protein variant of SEQ ID NO: 63. In another embodiment, provided herein are expression vectors comprising the nucleic acid encoding the sandercyanin fluorescent protein variant of SEQ ID NO: 63. In another embodiment, provided herein are host cells transformed with the expression vector comprising the nucleic acid encoding the sandercyanin fluorescent protein variant of SEQ ID NO: 63. In a further embodiment, provided herein are methods for producing a sandercyanin fluorescent protein variant, said method comprising expressing the sandercyanin fluorescent protein variant in the transformed host cell, wherein the host cell is transformed with the expression vector comprising the nucleic acid encoding the sandercyanin fluorescent protein variant of SEQ ID NO: 63.

### GFP-like Fluorescent Chromoprotein FP538 Variants

GFP-like fluorescent chromoprotein FP538 is a yellow pigment protein found in Zoanthus sp., in present in soft coral, and neon green sea mats and polyps. GFP-like fluorescent chromoprotein FP538 mutants previously described are the Glu-66 mutant, having a fluorescence excitation of is at 493 nm and 550 nm with intense green emission at 405 nm and a weak red emission at 576 nm, and a Asp-66 mutant, having a fluorescence emission of the is at 524 nm and 552 nm and with a broad red emission shoulder extending from 650 nm. GFP-like fluorescent chromoprotein FP538 are used in a variety of application, including as a fluorescent protein tag in chimeric proteins, a fluorescent marker in living cells and organisms, tracer of cell lineage, a reporter of gene expression, and a measure of protein-protein interactions. The herein provided GFP-like fluorescent chromoprotein FP538 variants have improved fluorescence and stability compared to the above-described mutants.

Also provided herein are GFP-like fluorescent chromoprotein FP538 variants of a wild-type GFP-like fluorescent chromoprotein FP538 from Zoanthus sp. of SEQ ID NO: 64 comprising one or more mutations selected from K9T, T30E, I34R or 134E; K39E, I49V, E50K, G94E, S96T, I106T, C107A, N108R, N108S or N108T; I125E or I125K; N127H, M129T, D134N, K139Q, M141R or M141K; T143V, T1431 or T143L; N144G, A147P, M153Y, K157N, K162R, S166T, L170K, V184T, S189K, S192K, E196G, Q201E, L205E, T220Y, T220Q, T220E or T220H, I224V, F226H, and A229P.

In a further embodiment, provided herein is a GFP-like fluorescent chromoprotein FP538 variant of a wild-type GFP-like fluorescent chromoprotein FP538 from Zoanthus sp. of SEQ ID NO: 64 comprising mutations:
(a) K9T, T30E, I34R, K39E, I49V, E50K, G94E, S96T, I106T, C107A, N108R, I125E, N127H, M129T, D134N, K139Q, M141R, T143V, N144G, A147P, M153Y, K157N, K162R, S166T, L170K, V184T, S189K, S192K, E196G, Q201E, L205E, T220Y, I224V, F226H, and A229P;
(b) K9T, T30E, I34R, K39E, I49V, E50K, G94E, S96T, I106T, C107A, N108R, I125K, N127H, M129T, D134N, K139Q, M141R, T143V, N144G, A147P, M153Y, K157N, S166T, L170K, V184T, S189K, S192K, E196G, Q201E, L205E, T220Y, I224V, F226H, and A229P; (c) G94E, K139Q, T143V, A147P, M153Y, K162R, and A229P;
(d) G94E, K139Q, T143I, A147P, M153Y, and A229P;
(e) K9T, T30E, I34R, K39E, G94E, S96T, I106T, C107A, N108S, I125K, N127H, M129T, K139Q, M141K, T143V, N144G, A147P, M153Y, K157N, K162R, S166T, L170K, V184T, E196G, T220Q, I224V, F226H, and A229P;
(f) K9T, T30E, I34R, K39E, G94E, S96T, I106T, C107A, N108S, I125K, N127H, M129T, K139Q, M141K, T143L, N144G, A147P, M153Y, K157N, S166T, L170K, V184T, E196G, T220H, I224V, F226H, and A229P;
(g) T30E, I34E, K39E, G94E, N108S, N127H, M129T, K139Q, M141R, T143V, A147P, M153Y, K162R, E196G, T220E, F226H, and A229P;
(h) I34E, G94E, N127H, K139Q, T143V, A147P, M153Y, K162R, T220E, and A229P;
(i) K9T, T30E, I34E, K39E, G94E, S96T, I106T, N108S, I125K, N127H, M129T, K139Q, M141R, T143V, A147P, M153Y, K162R, V184T, E196G, T220E, F226H, and A229P; or
(j) K9T, T30E, I34E, K39E, G94E, S96T, I106T, N108T, I125K, N127H, M129T, K139Q, M141K, T143V, A147P, M153Y, V184T, E196G, T220Q, F226H, and A229P.

In one embodiment, provided herein are nucleic acids encoding an above-described GFP-like fluorescent chromoprotein FP538 variant of SEQ ID NO: 64. In another embodiment, provided herein are expression vectors comprising the nucleic acid encoding the GFP-like fluorescent chromoprotein FP538 variant of SEQ ID NO: 64. In another embodiment, provided herein are host cells transformed with the expression vector comprising the nucleic acid encoding the GFP-like fluorescent chromoprotein FP538 variant of SEQ ID NO: 64. In a further embodiment, provided herein are methods for producing a GFP-like fluorescent chromoprotein FP538 variant, said method comprising expressing the GFP-like fluorescent chromoprotein FP538 variant in the transformed host cell, wherein the host cell is transformed with the expression vector comprising the nucleic acid encoding the GFP-like fluorescent chromoprotein FP538 variant of SEQ ID NO: 64.

### mNeonGreen Variants

mNeonGreen is a monomeric yellow-green fluorescent protein derived from a tetrameric fluorescent protein from the cephalochordate Branchiostoma lanceolatum and is described as the “brightest monomeric green or yellow fluorescent protein.” The previously developed mNeonGreen monomeric protein is called here the wild-type mNeonGreen. The herein provided mNeonGreen variants are improved compared to the wild-type protein in their photostability and fluorescence, and may be used as fusion tags for general and single-molecule super-resolution imaging, and as fluorescent probes.

Provided herein are mNeonGreen variants of a wild-type mNeonGreen from Branchiostoma lanceolatum of SEQ ID NO: 65 comprising one or more mutations selected from V52H, D53E, N65Y, E69M or E69Q, A110Q, S1311 or S131C, T139R, S143D, K152H, A158P, T164Q, S166K, A169G, W172P, S175E, K177M, T178L, T188D or T188E, K190T, T194K, G196K or G196E, N197D, 5203C, T204Q, T208H, N218D, and Y226F.

In a further embodiment, provided herein is a mNeonGreen variant of a wild-type mNeonGreen from Branchiostoma lanceolatum of SEQ ID NO: 65 comprising mutations:
(a) V52H, D53E, E69M, T139R, K152H, S166K, W172P, K177M, T188D, G196K, and 5203C;
(b) V52H, D53E, E69Q, S131 I, T139R, S143D, K152H, T164Q, S166K, A169G, W172P, K177M, T188D, K190T, T194K, G196E, N197D, 5203C, T208H, N218D, and Y226F;
(c) S166K, K177M, G196E, and 5203C;
(d) V52H, D53E, N65Y, E69Q, A110Q, S131 I, T139R, S143D, K152H, A158P, T164Q, S166K, A169G, W172P, S175E, K177M, T178L, T188D, K190T, T194K, G196E, N197D, 5203C, T204Q, T208H, N218D, and Y226F;
(e) E69M, T139R, S166K, K177M, T188D, G196E, and 5203C;
(f) V52H, D53E, E69M, T139R, K152H, S166K, W172P, K177M, G196K, and 5203C;
(g) V52H, D53E, N65Y, E69Q, A110Q, S131C, T139R, S143D, K152H, A158P, T164Q, S166K, A169G, W172P, K177M, T188E, K190T, T194K, G196E, N197D, 5203C, T204Q, T208H, N218D, and Y226F;
(h) V52H, D53E, E69Q, S131I, T139R, S143D, K152H, T164Q, S166K, A169G, W172P, S175E, K177M, T188D, K190T, T194K, G196E, N197D, 5203C, T208H, N218D, and Y226F;
(i) V52H, D53E, E69Q, T139R, K152H, S166K, W172P, K177M, K190T, G196E, N197D, 5203C, and Y226F;
(j) V52H, D53E, E69Q, S131 I, T139R, S143D, K152H, S166K, A169G, W172P, K177M, T188E, K190T, G196E, N197D, 5203C, and Y226F; or
(k) E69M, T139R, S166K, K177M, G196E, and 5203C.

In one embodiment, provided herein are nucleic acids encoding an above-described mNeonGreen variant of SEQ ID NO: 65. In another embodiment, provided herein are expression vectors comprising the nucleic acid encoding the mNeonGreen variant of SEQ ID NO: 65. In another embodiment, provided herein are host cells transformed with the expression vector comprising the nucleic acid encoding the mNeonGreen variant of SEQ ID NO: 65. In a further embodiment, provided herein are methods for producing a mNeonGreen variant, said method comprising expressing the mNeonGreen variant in the transformed host cell, wherein the host cell is transformed with the expression vector comprising the nucleic acid encoding the mNeonGreen variant of SEQ ID NO: 65.

### Green Fluorescent Protein bIFP-Y3 Variants

Green fluorescent protein blFP-Y3 is derived from the cephalochordate Branchiostoma lanceolatum (Common lancelet). This fluorescent protein also is called the brightest monomeric green or yellow fluorescent protein to date and is an excellent fusion tag for traditional imaging as well as stochastic single-molecule super resolution imaging. The presently provided Green Fluorescent Protein blFP-Y3 variants have an improved fluorescence brightness compared to wild-type Green fluorescent protein blFP-Y3.

Also provided herein are green fluorescent protein blFP-Y3 variants of a wild-type green fluorescent protein blFP-Y3 from Branchiostoma lanceolatum of SEQ ID NO: 66 comprising one or more mutations selected from M1A, AST, V18H, D19E, E35Q, S49N, A76K, K79C, S97I, S100A, N101H, Y102F, T105R, S109N, K112I, I118H, S132K, W138P, T141E, S153G, T154E, T160K, G162K, T170E, N174T, N184D, and K213W.

In a further embodiment, provided herein is a green fluorescent protein blFP-Y3 variant of a wild-type green fluorescent protein blFP-Y3 from Branchiostoma lanceolatum of SEQ ID NO: 66 comprising mutations:
(a) E35Q, S97I, Y102F, I118H, W138P, T141E, T160K, G162K, N174T, and K213W;
(b) D19E, E35Q, K79C, S97I, N101H, Y102F, I118H, W138P, T141E, S153G, T160K, G162K, T170E, N174T, and K213W;
(c) E35Q, S97I, Y102F, I118H, W138P, T154E, T160K, G162K, T170E, N174T, and K213; (d) V18H, D19E, E35Q, S49N, A76K, K79C, S97I, N101H, Y102F, I118H, S132K, W138P, T141E, S153G, T160K, G162K, T170E, N174T, and K213W;
(e) D19E, E35Q, K79C, S97I, N101H, Y102F, I118H, W138P, T141E, S153G, T154E, T160K, G162K, T170E, N174T, and K213W;
(f) AST, V18H, D19E, E35Q, S49N, A76K, K79C, S97I, N101H, Y102F, S109N, I118H, S132K, W138P, T141E, S153G, T160K, G162K, T170E, N174T, N184D, and K213W;
(g) E35Q, S97I, Y102F, I118H, W138P, T160K, G162K, N174T, and K213W; or
(h) M1A, A5T, V18H, D19E, E35Q, S49N, A76K, K79C, S97I, S100A, N101H, Y102F, T105R, S109N, K112I, I118H, S132K, W138P, T141E, S153G, T160K, G162K, T170E, N184D and K213W.

In one embodiment, provided herein are nucleic acids encoding an above-described green fluorescent protein blFP-Y3 variant of SEQ ID NO: 66. In another embodiment, provided herein are expression vectors comprising the nucleic acid encoding the green fluorescent protein blFP-Y3 variant of SEQ ID NO: 66. In another embodiment, provided herein are host cells transformed with the expression vector comprising the nucleic acid encoding the green fluorescent protein blFP-Y3 variant of SEQ ID NO: 66. In a further embodiment, provided herein are methods for producing a green fluorescent protein blFP-Y3 variant, said method comprising expressing the green fluorescent protein blFP-Y3 variant in the transformed host cell, wherein the host cell is transformed with the expression vector comprising the nucleic acid encoding the green fluorescent protein blFP-Y3 variant of SEQ ID NO: 66.

### Far-red Fluorescent Protein eqFP650 Variants

Far-red Fluorescent Protein eqFP650 is found in Entacmaea quadricolor, a bubble-tip anemone. The far-red fluorescent protein eqFP650 variants provided herein are enhanced in brightness over the wild-type far-red fluorescent protein eqFP650 and may be used for whole-body fluorescence imaging in animal models for among others, the study of tumorigenesis, embryogenesis, and inflammation.

Provided herein are far-red fluorescent protein eqFP650 variants of a wild-type far-red fluorescent protein eqFP650 from Entacmaea quadricolor of SEQ ID NO: 67 comprising one or more mutations selected from G13N, T19E, 520G, K25N, A33M, K34R, A51M, I85V, T86M, Y88F, L94C, T100I, N104D, L107F, K112E, N114R, S120P, A134P, A142R or A142C; S144G, R147E, H149R, Q151N or Q151D; V157K, Y161H, H163I, S165H, F184V, F186Y, R189H or R189Y; K190R, E196H, K199D, T201N, and M208V.

In another embodiment, provided herein is a far-red fluorescent protein eqFP650 variant of a wild-type far-red fluorescent protein eqFP650 from Entacmaea quadricolor of SEQ ID NO: 67 comprising mutations:
(a) G13N, T19E, S20G, A33M, K34R, A51M, I85V, T86M, T100I, N104D, L107F, K112E, N114R, A134P, A142R, S144G, R147E, H149R, Q151N, V157K, Y161H, H163I, S165H, F184V, F186Y, R189H, K190R, E196H, K199D, and M208V;
(b) G13N, T19E, S20G, K25N, A33M, K34R, A51M, I85V, T86M, Y88F, L94C, T100I, N104D, L107F, N114R, S120P, A134P, A142R, S144G, R147E, H149R, Q151N, V157K, Y161H, H163I, S165H, F184V, F186Y, R189H, K190R, E196H, K199D, T201N, and M208V;
(c) G13N, T19E, S20G, A33M, K34R, A51M, I85V, T86M, L94C, T100I, N104D, L107F, N114R, A134P, A142R, S144G, R147E, H149R, Q151N, V157K, Y161H, H163I, S165H, F184V, F186Y, R189H, K190R, E196H, K199D, and M208V;
(d) G13N, S20G, A33M, K34R, I85V, T86M, N104D, L107F, N114R, A134P, A142C, S144G, R147E, H149R, Q151D, Y161H, H163I, S165H, F184V, F186Y, K190R, E196H, K199D, and M208V;
(e) G13N, S20G, A33M, K34R, I85V, T86M, N104D, L107F, N114R, A134P, A142C, S144G, R147E, H149R, Q151D, V157K, Y161H, H163I, S165H, F184V, F186Y, K190R, E196H, K199D, and M208V;
(f) G13N, S20G, A33M, K34R, I85V, T86M, T100I, N104D, L107F, K112E, N114R, A134P, A142R, S144G, R147E, H149R, Q151N, V157K, Y161H, H163I, S165H, F184V, F186Y, R189Y, K190R, E196H, K199D, and M208V;
(g) G13N, S20G, A33M, K34R, I85V, T86M, N104D, L107F, N114R, A134P, A142C, S144G, R147E, H149R, Q151D, V157K, Y161H, H163I, S165H, F184V, F186Y, R189Y, K190R, E196H, K199D, and M208V;
(h) G13N, S20G, A33M, K34R, I85V, T86M, T100I, N104D, L107F, K112E, N114R, A134P, A142C, S144G, R147E, H149R, Q151D, V157K, Y161H, H163I, S165H, F184V, F186Y, R189Y, K190R, E196H, K199D, and M208V; or
(i) G13N, T19E, S20G, K25N, A33M, K34R, A51M, I85V, T86M, Y88F, L94C, T100I, N104D, L107F, K112E, N114R, S120P, A134P, A142C, S144G, R147E, H149R, Q151N, V157K, Y161H, H163I, S165H, F184V, F186Y, R189H, K190R, E196H, K199D, T201N and M208V.

In one embodiment, provided herein are nucleic acids encoding an above-described far-red fluorescent protein eqFP650 variant of SEQ ID NO: 67. In another embodiment, provided herein are expression vectors comprising the nucleic acid encoding the far-red fluorescent protein eqFP650 variant of SEQ ID NO: 67. In another embodiment, provided herein are host cells transformed with the expression vector comprising the nucleic acid encoding the far-red fluorescent protein eqFP650 variant of SEQ ID NO: 67. In a further embodiment, provided herein are methods for producing a far-red fluorescent protein eqFP650 variant, said method comprising expressing the far-red fluorescent protein eqFP650 variant in the transformed host cell, wherein the host cell is transformed with the expression vector comprising the nucleic acid encoding the far-red fluorescent protein eqFP650 variant of SEQ ID NO: 67.

### Fluorescent Protein cyOFP Variants

Fluorescent protein cyOFP is a tetrameric cyan-excitable orange-red fluorescent protein has been genetically engineered and expressed as a 242 amino acid fluorescent protein in Escherichia coli; the previously described fluorescent protein cyOFP is herein called the wild-type Fluorescent Protein cyOFP. The herein provided fluorescent protein cyOFP variants have enhanced properties compared to the wild-type fluorescent protein cyOFP.

Also provided herein are fluorescent protein cyOFP variants of a wild-type fluorescent protein cyOFP from Escherichia coli of SEQ ID NO: 68 comprising one or more mutations selected from L8V, E11P, H14K, S15T, L19M, H32G, K37N, N45M, R46K, V50T, A58S, A63M, M65L, K71E, L106C, T112I or T112V; L114I, A132P, L151M; A154R, A154K or A154N; K164M, V169K, N177H, K179R, T189K, N211D, V220W, V220H or V220Q and D227H.

In one embodiment, provided herein is a fluorescent protein cyOFP variant of a wild-type fluorescent protein cyOFP from Escherichia coli of SEQ ID NO: 68 comprising mutations:
(a) L8V, E11P, H14K, L19M, H32G, N45M, A63M, M65L, K71E, A132P, A154R, K164M, V169K, N177H, and D227H;
(b) E11P, H14K, L19M, H32G, K37N, N45M, R46K, A63M, K71E, A132P, A154R, K164M, V169K, N177H, T189K, N211D, V220W, and D227H;
(c) E11P, H14K, L19M, H32G, N45M, A63M, M65L, K71E, A154R, K164M, V169K and N177H;
(d) L8V, E11P, H14K, L19M, H32G, K37N, N45M, R46K, A63M, K71E, T112I, A132P, A154K, K164M, V169K, N177H, T189K, N211D, and D227H;
(e) L8V, E11P, H14K, L19M, H32G, N45M, A63M, K71E, T112I, A154R, K164M, V169K and N177H;
(f) E11P, H14K, L19M, H32G, K37N, N45M, R46K, V50T, A58S, A63M, K71E, L106C, T112V, L114I, A132P, L151M, A154R, K164M, V169K, N177H, T189K, N211D, V220H and D227H;
(g) E11P, H14K, L19M, H32G, K37N, N45M, R46K, V50T, A58S, A63M, M65L, K71E, L106C, T112I, A132P, L151M, A154N, K164M, V169K, N177H, T189K, N211D, V220Q and D227H;
(h) E11P, H14K, L19M, N45M, K71E, A154R, K164M, V169K, N177H, and K179R;
(i) E11P, H14K, S15T, L19M, H32G, K37N, N45M, R46K, A63M, K71E, T112I, A132P, A154K, K164M, V169K, N177H, T189K, N211D, and D227H;
(j) H14K, L19M, N45M, K71E, A154R, K164M, and V169K;
(k) E11P, H14K, L19M, H32G, N45M, A63M, K71E, A132P, A154R, K164M, V169K, N177H and D227H;
(l) E11P, H14K, L19M, N45M, K71E, A154R, K164M, V169K, and N177H; or
(m) E11P, H14K, L19M, H32G, K37N, N45M, R46K, A63M, K71E, T112I, A132P, A154R, K164M, V169K, N177H, T189K, N211D, and D227H.

In one embodiment, provided herein are nucleic acids encoding an above-described fluorescent protein cyOFP variant of SEQ ID NO: 68. In another embodiment, provided herein are expression vectors comprising the nucleic acid encoding the fluorescent protein cyOFP variant of SEQ ID NO: 68. In another embodiment, provided herein are host cells transformed with the expression vector comprising the nucleic acid encoding the fluorescent protein cyOFP variant of SEQ ID NO: 68. In a further embodiment, provided herein are methods for producing a fluorescent protein cyOFP variant, said method comprising expressing the fluorescent protein cyOFP variant in the transformed host cell, wherein the host cell is transformed with the expression vector comprising the nucleic acid encoding the fluorescent protein cyOFP variant of SEQ ID NO: 68.

### EXAMPLES

The list of enzymes used in the PROSS algorithm, the enzyme Uniprot Accession number, host organism, and wild-type enzyme sequence length, respectively, are given in Table 1.

TABLE 1
List of enzymes, and corresponding host organism

| Uniprot Accession | Protein names | Organism | Length |
|---|---|---|---|
| F2XBU9 | Pyruvate transaminase | Vibrio fluvialis | 453 |
| (SEQ ID NO: 39) | | | |
| Q05489 | Triacylglycerol lipase (EC 3.1.1.3) | Burkholderia glumae | 358 |
| (SEQ ID NO: 40) | (Extracellular lipase) (Triacylglycerol | (Pseudomonas glumae) | |
| | ester hydrolase) | | |
| Q93D82 | Halohydrin dehalogenase | Rhizobium radiobacter | 254 |
| (SEQ ID NO: 41) | | (Agrobacterium tumefaciens) | |
| | | (Agrobacterium radiobacter) | |
| Q7NWG4 | Probable aminotransferase | Chromobacterium violaceum | 459 |
| (SEQ ID NO: 42) | (EC 2.6.1.62) | (strain ATCC 12472/DSM | |
| | | 30191/JCM 1249/NBRC | |
| | | 12614/NCIMB 9131/NCTC | |
| | | 9757) | |
| A5I055 | Putative secreted lipase | Clostridium botulinum | 480 |
| (SEQ ID NO: 43) | | (strain Hall/ATCC 3502/ | |
| | | NCTC 13319/Type A) | |
| P12311 | Alcohol dehydrogenase (EC 1.1.1.1) | Geobacillus stearothermophilus | 337 |
| (SEQ ID NO: 44) | (ADH-T) | (Bacillus stearothermophilus) | |
| C4S833 | Histidine acid phosphatase (Phytase) | Yersinia mollaretii ATCC | 434 |
| (SEQ ID NO: 45) | | 43969 | |
| Q4WZ70 | Chanoclavine-I aldehyde reductase easA | Neosartorya fumigata (strain | 376 |
| (SEQ ID NO: 46) | (EC 1.3.1.100) (Ergot alkaloid synthesis | ATCC MYA-4609/Af293/ | |
| | protein A) (Old yellow enzyme 3 | CBS 101355/FGSC A1100) | |
| | homolog) | (Aspergillus fumigatus) | |
| O28608 | Alanine dehydrogenase (AlaDH) | Archaeoglobus fulgidus (strain | 322 |
| (SEQ ID NO: 47) | (EC 1.4.1.1) | ATCC 49558/VC-16/DSM | |
| | | 4304/JCM 9628/NBRC | |
| | | 100126) | |
| Q9S1A6 | Acid phosphatase (EC 3.1.3.2) | Shimwellia blattae (Escherichia | 249 |
| (SEQ ID NO: 48) | | blattae) | |
| P28581 | Major phosphate-irrepressible acid | Morganella morganii (Proteus | 249 |
| (SEQ ID NO: 49) | phosphatase (HPAP) (EC 3.1.3.2) | morganii) | |
| Q87188 | Acid phosphatase (EC 3.1.3.2) | Prevotella intermedia | 263 |
| (SEQ ID NO: 50) | | | |
| Q50EL0 | Tryptophan dimethylallyltransferase | Neosartorya fumigata (strain | 459 |
| (SEQ ID NO: 51) | (EC 2.5.1.34) (4-dimethylallyltryptophan | ATCC MYA-4609/Af293/CBS | |
| | synthase) (All-trans-hexaprenyl- | 101355/FGSC A1100) | |
| | diphosphate synthase) (L-tryptophan | (Aspergillus fumigatus) | |
| | dimethylallyl transferase) (DMATS) | | |
| P42327 | Alcohol dehydrogenase (ADH) | Geobacillus stearothermophilus | 339 |
| (SEQ ID NO: 52) | (EC 1.1.1.1) | (Bacillus stearothermophilus) | |
| Q0RV69 | Alcohol dehydrogenase (EC 1.1.1.1) | Rhodococcus jostii (strain | 369 |
| (SEQ ID NO: 53) | | RHA1) | |
| Q7BEK9 | Acid phosphatase (EC 3.1.3.2) | Shigella flexneri 5a | 249 |
| (SEQ ID NO: 54) | | | |
| Q5P8T0 | Alcohol dehydrogenase | Aromatoleum aromaticum | 335 |
| (SEQ ID NO: 55) | | (strain EbN1) (Azoarcus sp. | |
| | | (strain EbN1)) | |
| W6R2Y2 | Arylesterase (EC 3.1.2.-) | Pseudomonas | 201 |
| (SEQ ID NO: 56) | | pseudoalcaligenes (strain | |
| | | CECT 5344) | |
| P42588 | Putrescine aminotransferase (EC | Escherichia coli (strain K12) | 459 |
| (SEQ ID NO: 57) | 2.6.1.82) (Putrescine-2-oxoglutaric acid | | |
| | transaminase) (PAT) (PATase) | | |
| Q5S6Z9 | Green to red photoconvertible GFP-like | Lobophyllia hemprichii (Lobed | 226 |
| (SEQ ID NO: 58) | protein EosFP | brain coral) | |
| Q9U6Y8 | Red fluorescent protein drFP583 (DsRed) | Discosoma sp. (Sea anemone) | 225 |
| (SEQ ID NO: 59) | | | |
| P42212 | Green fluorescent protein | Aequorea victoria (Jellyfish) | 238 |
| (SEQ ID NO: 60) | | | |
| P08659 | Luciferin 4-monooxygenase (Luciferase) | Photinus pyralis (Common | 550 |
| (SEQ ID NO: 61) | (EC 1.13.12.7) | eastern firefly) (Lampyris | |
| | | pyralis) | |
| D0VWW2 | mOrange | Discosoma sp. (Sea anemone) | 236 |
| (SEQ ID NO: 62) | | | |
| A0A1D5B367 | Sandercyanin Fluorescent Protein | Sander vitreus (Walleye) | 183 |
| (SEQ ID NO: 63) | | (Perea vitrea) | |
| Q9U6Y4 | GFP-like fluorescent chromoprotein | Zoanthus sp. (Green polyp) | 231 |
| (SEQ ID NO: 64) | FP538 (zFP538) [Cleaved into: GFP-like | | |
| | fluorescent chromoprotein FP538 chain | | |
| | 1; GFP-like fluorescent chromoprotein | | |
| | FP538 chain 2] | | |
| A0A1S4NYF2 | mNeonGreen | Branchiostoma lanceolatum | 260 |
| (SEQ ID NO: 65) | | (Common lancelet) | |
| | | (Amphioxus lanceolatum) | |
| B1PNC0 | Green fluorescent protein blFP-Y3 | Branchiostoma lanceolatum | 219 |
| (SEQ ID NO: 66) | | (Common lancelet) | |
| | | (Amphioxus lanceolatum) | |
| J9PBR7 | Far-red fluorescent protein eqFP650 | Entacmaea quadricolor | 223 |
| (SEQ ID NO: 67) | | (Bubble-tip anemone) | |
| | | (Parasicyonis actinostoloides) | |
| A0A182DW87 | Fluorescent protein cyOFP | Escherichia coli | 244 |
| (SEQ ID NO: 68) | | | |

Table 2 enumerates the wildtype amino acid sequence for each of the enzymes listed in Table 1; each enzyme is identified by its respective Uniprot Accession number.

TABLE 2
Wild-type sequences of the above enzymes, according to the Uniprot IDs

| Uniprot | |
|---|---|
| Accession | Sequence |
| F2XBU9 | MNKPQSWEARAETYSLYGFTDMPSLHQRGTVVVTHGEGPYIVDVNGRRYLDANSGLW |
| | NMVAGFDHKGLIDAAKAQYERFPGYHAFFGRMSDQTVMLSEKLVEVSPFDSGRVFYTN |
| | SGSEANDTMVKMLWFLHAAEGKPQKRKILTRWNAYHGVTAVSASMTGKPYNSVFGLPL |
| | PGFVHLTCPHYWRYGEEGETEEQFVARLARELEETIQREGADTIAGFFAEPVMGAGGVI |
| | PPAKGYFQAILPILRKYDIPVISDEVICGFGRTGNTWGCVTYDFTPDAIISSKNLTAGFFPM |
| | GAVILGPELSKRLETAIEAIEEFPHGFTASGHPVGCAIALKAIDVVMNEGLAENVRRLAPR |
| | FEERLKHIAERPNIGEYRGIGFMWALEAVKDKASKTPFDGNLSVSERIANTCTDLGLICRP |
| | LGQSVVLCPPFILTEAQMDEMFDKLEKALDKVFAEVA (SEQ ID NO: 39) |
| Q05489 | MVRSMRSRVAARAVAWALAVMPLAGAAGLTMAASPAAVAADTYAATRYPVILVHGLAG |
| | TDKFANVVDYWYGIQSDLQSHGAKVYVANLSGFQSDDGPNGRGEQLLAYVKQVLAAT |
| | GATKVNLIGHSQGGLTSRYVAAVAPQLVASVTTIGTPHRGSEFADFVQDVLKTDPTGLS |
| | STVIAAFVNVFGTLVSSSHNTDQDALAALRTLTTAQTATYNRNFPSAGLGAPGSCQTGA |
| | ATETVGGSQHLLYSWGGTAIQPTSTVLGVTGATDTSTGTLDVANVTDPSTLALLATGAV |
| | MINRASGQNDGLVSRCSSLFGQVISTSYHWNHLDEINQLLGVRGANAEDPVAVIRTHVN |
| | RLKLQGV (SEQ ID NO: 40) |
| Q93D82 | MSTAIVTNVKHFGGMGSALRLSEAGHTVACHDESFKQKDELEAFAETYPQLKPMSEQE |
| | PAELIEAVTSAYGQVDVLVSNDIFAPEFQPIDKYAVEDYRGAVEALQIRPFALVNAVASQ |
| | MKKRKSGHIIFITSATPFGPWKELSTYTSARAGACTLANALSKELGEYNIPVFAIGPNYLH |
| | SEDSPYFYPTEPWKTNPEHVAHVKKVTALQRLGTQKELGELVAFLASGSCDYLTGQVF |
| | WLAGGFPMIERWPGMPE (SEQ ID NO: 41) |
| Q7NWG4 | MQKQRTTSQWRELDAAHHLHPFTDTASLNQAGARVMTRGEGVYLWDSEGNKIIDGMA |
| | GLWCVNVGYGRKDFAEAARRQMEELPFYNTFFKTTHPAVVELSSLLAEVTPAGFDRVF |
| | YTNSGSESVDTMIRMVRRYWDVQGKPEKKTLIGRWNGYHGSTIGGASLGGMKYMHEQ |
| | GDLPIPGMAHIEQPWWYKHGKDMTPDEFGVVAARWLEEKILEIGADKVAAFVGEPIQGA |
| | GGVIVPPATYWPEIERICRKYDVLLVADEVICGFGRTGEWFGHQHFGFQPDLFTAAKGL |
| | SSGYLPIGAVFVGKRVAEGLIAGGDFNHGFTYSGHPVCAAVAHANVAALRDEGIVQRVK |
| | DDIGPYMQKRWRETFSRFEHVDDVRGVGMVQAFTLVKNKAKRELFPDFGEIGTLCRDIF |
| | FRNNLIMRACGDHIVSAPPLVMTRAEVDEMLAVAERCLEEFEQTLKARGLA (SEQ ID |
| | NO: 42) |
| A5I055 | MKKNLNKIATIILLVFSMTLTNFSMIVRAAEPKAQGTQKVESSTTKKEVKDAEETIKIPTLE |
| | DIDNLIDSAEEVKSEEDINKMPPLKFPVEFPEVNTRSIIGGNNYPIVLVHGFMGFGRDELL |
| | GYKYWGGVVDLQEKLNASGHETYTATVGPVSSNWDRACELYAYIVGGTVDYGEAHAK |
| | KFKHNRYGRTYPGIYKNISNENKIHLIGHSMGGQTIRTLTQLLSEGSEEEINCGQENISPL |
| | FEGGKHWIHSVSTISTPNDGTTLSDLMPAKDLISYTFGVLGTITGKNKLFSSIYDLKLDQW |
| | GLKKQNGESQRDYIERVLDSNIWNSTKDIATYDLSTEGAQELNTWVKAQPDVYYFSWTT |
| | QATKESILTGHSVAQIGPMNPIFYPTANLMGRYSRNQKDLPIIDKKWFPNDGVVNCISQD |
| | GPKLGSNDVIEQYNGGVKIGQWNAMPRIINTDHMDIVGTFGNVKDWYMDYASFLSNLS |
| | R (SEQ ID NO: 43) |
| P12311 | MKAAVVEQFKKPLQVKEVEKPKISYGEVLVRIKACGVCHTDLHAAHGDWPVKPKLPLIP |
| | GHEGVGVIEEVGPGVTHLKVGDRVGIPWLYSACGHCDYCLSGQETLCERQQNAGYSV |
| | DGGYAEYCRAAADYVVKIPDNLSFEEAAPIFCAGVTTYKALKVTGAKPGEWVAIYGIGGL |
| | GHVAVQYAKAMGLNVVAVDLGDEKLELAKQLGADLVVNPKHDDAAQWIKEKVGGVHAT |
| | VVTAVSKAAFESAYKSIRRGGACVLVGLPPEEIPIPIFDTVLNGVKIIGSIVGTRKDLQEAL |
| | QFAAEGKVKTIVEVQPLENINDVFDRMLKGQINGRVVLKVD (SEQ ID NO: 44) |
| C4S833 | MRLTALGLMLGSFAISAAPVAAPVTGYTLERVVILSRHGVRSPTKQTELMNDVTPDKWP |
| | QWPVPAGYLTPRGAQLVTLMGGFYGDYFRNQGLLPAGCPADGTLYAQADIDQRTRLT |
| | GQAFLDGIAPGCGLKVHYQADLKKVDPLFHPVEAGVCQLDSTQTHRAIEAQLGAPLSEL |
| | SQRYAKPFAQMGEILNFTASPYCKSLQQQGKSCDFATFAANEVKVNQQGTKVSLSGPL |
| | ALSSTLGEIFLLQNSQGMPDVAWHRLSGAENWVSLLSLHNAQFDLMAKTPYIARHKGTP |
| | LLQQIVTALVLQRKGQGQTLPLSEQTKLLFLGGHDTNIANIGGMLGANWQLPQQPDNTP |
| | PGGGLVFELWQNPDNHQQYVAVKMFYQTMDQLRNSEKLDLKSHPAGIVPIEIEGCENIG |
| | TDKLCQLDTFQKRVAQVIEPACHI (SEQ ID NO: 45) |
| Q4WZ70 | MREEPSSAQLFKPLKVGRCHLQHRMIMAPTTRFRADGQGVPLPFVQEYYGQRASVPG |
| | TLLITEATDITPKAMGYKHVPGIWSEPQREAWREIVSRVHSKKCFIFCQLWATGRAADPD |
| | VLADMKDLISSSAVPVEEKGPLPRALTEDEIQQCIADFAQAARNAINAGFDGVEIHGANG |
| | YLIDQFTQKSCNHRQDRWGGSIENRARFAVEVTRAVIEAVGADRVGVKLSPYSQYLGM |
| | GTMDELVPQFEYLIAQMRRLDVAYLHLANSRWLDEEKPHPDPNHEVFVRVWGQSSPIL |
| | LAGGYDAASAEKVTEQMAAATYTNVAIAFGRYFISTPDLPFRVMAGIQLQKYDRASFYST |
| | LSREGYLDYPFSAEYMALHNFPV (SEQ ID NO: 46) |
| O28608 | METLILTQEEVESLISMDEAMNAVEEAFRLYALGKAQMPPKVYLEFEKGDLRAMPAHLM |
| | GYAGLKWVNSHPGNPDKGLPTVMALMILNSPETGFPLAVMDATYTTSLRTGAAGGIAAK |
| | YLARKNSSVFGFIGCGTQAYFQLEALRRVFDIGEVKAYDVREKAAKKFVSYCEDRGISAS |
| | VQPAEEASRCDVLVTTTPSRKPVVKAEWVEEGTHINAIGADGPGKQELDVEILKKAKIVV |
| | DDLEQAKHGGEINVAVSKGVIGVEDVHATIGEVIAGLKDGRESDEEITIFDSTGLAIQDVA |
| | VAKVVYENALSKNVGSKIKFFRI (SEQ ID NO: 47) |
| Q9S1A6 | MKKRVLAVCFAALFSSQALALVATGNDTTTKPDLYYLKNSEAINSLALLPPPPAVGSIAFL |
| | NDQAMYEQGRLLRNTERGKLAAEDANLSSGGVANAFSGAFGSPITEKDAPALHKLLTN |
| | MIEDAGDLATRSAKDHYMRIRPFAFYGVSTCNTTEQDKLSKNGSYPSGHTSIGWATALV |
| | LAEINPQRQNEILKRGYELGQSRVICGYHWQSDVDAARVVGSAVVATLHTNPAFQQQL |
| | QKAKAEFAQHQKK (SEQ ID NO: 48) |
| P28581 | MKKNIIAGCLFSLFSLSALAAIPAGNDATTKPDLYYLKNEQAIDSLKLLPPPPEVGSIQFLN |
| | DQAMYEKGRMLRNTERGKQAQADADLAAGGVATAFSGAFGYPITEKDSPELYKLLTNM |
| | IEDAGDLATRSAKEHYMRIRPFAFYGTETCNTKDQKKLSTNGSYPSGHTSIGWATALVLA |
| | EVNPANQDAILERGYQLGQSRVICGYHWQSDVDAARIVGSAAVATLHSDPAFQAQLAK |
| | AKQEFAQKSQK (SEQ ID NO: 49) |
| O87188 | MTKKTLLVGLLAMFSVTTFAQNAAKKIKDARTNPDLYYLQDGQQTSSLELLPTPPQPGSI |
| | QFLYDEAQYQWGKMQRNTPRGDQAVADARVGGDGVPNAFSEAFGIKISKETTPEIYKL |
| | VLNMREDAGDLATRSAKDHYMRVRPFAFYNEMTCNPEQQQELSTNGSYPSGHTAIGW |
| | ATALVLSEINIDRQNEILERGYQMGQSRVICGYHWQSDVDAARVVSAAVVARLHADPAF |
| | QAQLEKAKKEFAKLQKEGKVAKSTFKAAN (SEQ ID NO: 50) |
| Q50EL0 | MKAANASSAEAYRVLSRAFRFDNEDQKLWWHSTAPMFAKMLETANYTTPCQYQYLITY |
| | KECVIPSLGCYPTNSAPRWLSILTRYGTPFELSLNCSNSIVRYTFEPINQHTGTDKDPFNT |
| | HAIWESLQHLLPLEKSIDLEWFRHFKHDLTLNSEESAFLAHNDRLVGGTIRTQNKLALDL |
| | KDGRFALKTYIYPALKAVVTGKTIHELVFGSVRRLAVREPRILPPLNMLEEYIRSRGSKST |
| | ASPRLVSCDLTSPAKSRIKIYLLEQMVSLEAMEDLWTLGGRRRDASTLEGLSLVRELWD |
| | LIQLSPGLKSYPAPYLPLGVIPDERLPLMANFTLHQNDPVPEPQVYFTTFGMNDMAVAD |
| | ALTTFFERRGWSEMARTYETTLKSYYPHADHDKLNYLHAYISFSYRDRTPYLSVYLQSF |
| | ETGDWAVANLSESKVKCQDAACQPTSLPPDLSKTGVYYSGLH (SEQ ID NO: 51) |
| P42327 | MKAAVVNEFKKALEIKEVERPKLEEGEVLVKIEACGVCHTDLHAAHGDWPIKPKLPLIPG |
| | HEGVGIVVEVAKGVKSIKVGDRVGIPWLYSACGECEYCLTGQETLCPHQLNGGYSVDG |
| | GYAEYCKAPADYVAKIPDNLDPVEVAPILCAGVTTYKALKVSGARPGEWVAIYGIGGLGH |
| | IALQYAKAMGLNVVAVDISDEKSKLAKDLGADIAINGLKEDPVKAIHDQVGGVHAAISVAV |
| | NKKAFEQAYQSVKRGGTLVVVGLPNADLPIPIFDTVLNGVSVKGSIVGTRKDMQEALDFA |
| | ARGKVRPIVETAELEEINEVFERMEKGKINGRIVLKLKED (SEQ ID NO: 52) |
| Q0RV69 | MKTKAAALTGVHRDWEVLEFDLDDPGPGEVLVQMKVAGLCHSDKHAKFGGMTYPLVG |
| | GHEGAGVVEAVGDGVSRVSIGDHVAVSWIPSCGTCRWCASGQSNLCDLGANMVTGEL |
| | ANGGFRFHRDGTDIGAMAATGTFSQFSVVDQRSVVKVDPTIPFEWVSLVTCGVATGWG |
| | SVVNAGEVKAGDTVAVYGCGGIGSNAVRAAVGANAGLVAAIDPVESKLEFAKQSGADF |
| | VYTSAEEAHADLWERTYGVGADVTVVTVGVVTSEVVRAAFELTRKGGTVVLTGVSDDV |
| | MENTIELPGGILTLFQKRIVGSLYGHCNPLVDIPRLLRMAQAGKLVLDDLVTRRYALEDIN |
| | QGFDDMLAGHNIRGLIVHDH (SEQ ID NO: 53) |
| Q7BEK9 | MKRQLFTLSIVGVFSLNTFASFPPGNDVTTKPDLYYLTNDNAIDSLALLPPPPQIGSIAFLN |
| | DQAMYEKGRLLRNTERGKLAAEDANLSSGGVANVFSAAFGSPITAKDSPELHKLLTNMI |
| | EDAGDLATRSAKEYYMRIRPFAFYGVSTCNTKEQDTLSRNGSYPSGHTSIGWATALVLS |
| | EINPARQDTILKRGYELGDSRVICGYHWQSDVDAARIVGSAIVATLHSNPVFQAQLQKAK |
| | DEFANNQKK (SEQ ID NO: 54) |
| Q5P8T0 | MKAITLRSPGGLDRLELVELPDPGQPGPGEIRVRIHASSLNFHDLGVVSGAMPVADNRIP |
| | MSDGAGVVEAIGEGVDEFAVGDAVVSTFFPHWLDGEAPIADFSTTPGDGVDGYACELV |
| | VRPATAFTHSPRGYSHAQAATLTTAGLTAWRALIYNGGLKAGDSVLVLGTGGVSIFALQ |
| | FAKAMGATVIATSSSDEKLEHARALGASHTINYCSQAQWASRVLDFTGGCGVDHVIEVG |
| | GPGTLAQSIQACRIGGHIALIGVLTGGAGVVPTAALMKRQQRLQGLIVGNRRQQNELVR |
| | AIEATGIFPVIDRSFTLETIADAFRLQKAGGHFGKISLEF (SEQ ID NO: 55) |
| W6R2Y2 | MRAWWLSGALALMFWAQGAVAGTLLVVGDSISAAFGLDSRQGWVALLEKRLSEEGFE |
| | HSVVNASISGDTSAGGAARLSALLAEHKPELVIIELGGNDGLRGQPPAQLQQNLASMVE |
| | QSQQAGAKVLLLGMKLPPNYGVRYTTAFAQVFTDLAEQKQVSLVPFFLEGVGGVPGMM |
| | QADGIHPAEAAQEILLDNVWPTLKPML (SEQ ID NO: 56) |
| P42588 | MNRLPSSASALACSAHALNLIEKRTLDHEEMKALNREVIEYFKEHVNPGFLEYRKSVTAG |
| | GDYGAVEWQAGSLNTLVDTQGQEFIDCLGGFGIFNVGHRNPVVVSAVQNQLAKQPLHS |
| | QELLDPLRAMLAKTLAALTPGKLKYSFFCNSGTESVEAALKLAKAYQSPRGKFTFIATSG |
| | AFHGKSLGALSATAKSTFRKPFMPLLPGFRHVPFGNIEAMRTALNECKKTGDDVAAVILE |
| | PIQGEGGVILPPPGYLTAVRKLCDEFGALMILDEVQTGMGRTGKMFACEHENVQPDILC |
| | LAKALGGGVMPIGATIATEEVFSVLFDNPFLHTTTFGGNPLACAAALATINVLLEQNLPAQ |
| | AEQKGDMLLDGFRQLAREYPDLVQEARGKGMLMAIEFVDNEIGYNFASEMFRQRVLVA |
| | GTLNNAKTIRIEPPLTLTIEQCELVIKAARKALAAMRVSVEEA (SEQ ID NO: 57) |
| Q5S6Z9 | MSAIKPDMKINLRMEGNVNGHHFVIDGDGTGKPFEGKQSMDLEVKEGGPLPFAFDILTT |
| | AFHYGNRVFAEYPDHIQDYFKQSFPKGYSWERSLTFEDGGICIARNDITMEGDTFYNKV |
| | RFHGVNFPANGPVMQKKTLKWEPSTEKMYVRDGVLTGDITMALLLEGNAHYRCDFRTT |
| | YKAKEKGVKLPGYHFVDHCIEILSHDKDYNKVKLYEHAVAHSGLPDNARR (SEQ ID NO: 58) |
| Q9U6Y8 | MRSSKNVIKEFMRFKVRMEGTVNGHEFEIEGEGEGRPYEGHNTVKLKVTKGGPLPFAW |
| | DILSPQFQYGSKVYVKHPADIPDYKKLSFPEGFKWERVMNFEDGGVVTVTQDSSLQDG |
| | CFIYKVKFIGVNFPSDGPVMQKKTMGWEASTERLYPRDGVLKGEIHKALKLKDGGHYLV |
| | EFKSIYMAKKPVQLPGYYYVDSKLDITSHNEDYTIVEQYERTEGRHHLFL (SEQ ID NO: 59) |
| P42212 | MSKGEELFTGVVPILVELDGDVNGHKFSVSGEGEGDATYGKLTLKFICTTGKLPVPWPT |
| | LVTTFSYGVQCFSRYPDHMKQHDFFKSAMPEGYVQERTIFFKDDGNYKTRAEVKFEGD |
| | TLVNRIELKGIDFKEDGNILGHKLEYNYNSHNVYIMADKQKNGIKVNFKIRHNIEDGSVQL |
| | ADHYQQNTPIGDGPVLLPDNHYLSTQSALSKDPNEKRDHMVLLEFVTAAGITHGMDELY |
| | K (SEQ ID NO: 60) |
| P08659 | MEDAKNIKKGPAPFYPLEDGTAGEQLHKAMKRYALVPGTIAFTDAHIEVNITYAEYFEMS |
| | VRLAEAMKRYGLNTNHRIVVCSENSLQFFMPVLGALFIGVAVAPANDIYNERELLNSMNI |
| | SQPTVVFVSKKGLQKILNVQKKLPIIQKIIIMDSKTDYQGFQSMYTFVTSHLPPGFNEYDF |
| | VPESFDRDKTIALIMNSSGSTGLPKGVALPHRTACVRFSHARDPIFGNQIIPDTAILSVVPF |
| | HHGFGMFTTLGYLICGFRVVLMYRFEEELFLRSLQDYKIQSALLVPTLFSFFAKSTLIDKY |
| | DLSNLHEIASGGAPLSKEVGEAVAKRFHLPGIRQGYGLTETTSAILITPEGDDKPGAVGK |
| | VVPFFEAKVVDLDTGKTLGVNQRGELCVRGPMIMSGYVNNPEATNALIDKDGWLHSGDI |
| | AYWDEDEHFFIVDRLKSLIKYKGYQVAPAELESILLQHPNIFDAGVAGLPDDDAGELPAA |
| | VVVLEHGKTMTEKEIVDYVASQVTTAKKLRGGVVFVDEVPKGLTGKLDARKIREILIKAKK |
| | GGKSKL (SEQ ID NO: 61) |
| D0VWW2 | MVSKGEENNMAIIKEFMRFKVRMEGSVNGHEFEIEGEGEGRPYEGFQTAKLKVTKGGP |
| | LPFAWDILSPQFGYGSKAYVKHPADIPDYFKLSFPEGFKWERVMNFEDGGVVTVTQDS |
| | SLQDGEFIYKVKLRGTNFPSDGPVMQKKTMGWEASSERMYPEDGALKGEIKMRLKLKD |
| | GGHYTSEVKTTYKAKKPVQLPGAYIVGIKLDITSHNEDYTIVEQYERAEGRHSTGGMDEL |
| | YK (SEQ ID NO: 62) |
| A0A1D5B367 | MFIKPGRCPKPAVQEDFDAARYLGVWYDIQRLPNKFQKGECATATYSLSPGVGFSVFN |
| | RERLANGTIKSVIGSAIAEDPCEPAKLQFFHENAAPVPYWVLSTDYDNYALVYSCINLGA |
| | SHAAYASIVSRQPTLPEETIKKLQGTMSSFGVGVDTLLTTNQDAAYCSAMNQKLAAALE |
| | HHHHHH (SEQ ID NO: 63) |
| Q9U6Y4 | MAHSKHGLKEEMTMKYHMEGCVNGHKFVITGEGIGYPFKGKQTINLCVIEGGPLPFSED |
| | ILSAGFKYGDRIFTEYPQDIVDYFKNSCPAGYTWGRSFLFEDGAVCICNVDITVSVKENCI |
| | YHKSIFNGMNFPADGPVMKKMTTNWEASCEKIMPVPKQGILKGDVSMYLLLKDGGRYR |
| | CQFDTVYKAKSVPSKMPEWHFIQHKLLREDRSDAKNQKWQLTEHAIAFPSALA (SEQ ID |
| | NO: 64) |
| A0A1S4NYF2 | MASMTGGQQMGRDLYDDDDKDPLEMVSKGEEDNMASLPATHELHIFGSINGVDFDMV |
| | GQGTGNPNDGYEELNLKSTKGDLQFSPWILVPHIGYGFHQYLPYPDGMSPFQAAMVDG |
| | SGYQVHRTMQFEDGASLTVNYRYTYEGSHIKGEAQVKGTGFPADGPVMTNSLTAADW |
| | CRSKKTYPNDKTIISTFKWSYTTGNGKRYRSTARTTYTFAKPMAANYLKNQPMYVFRKT |
| | ELKHSKTELNFKEWQKAFTDVMGMDELYK (SEQ ID NO: 65) |
| B1PNC0 | MSLPATHELHIFGSFNGVDFDMVGRGTGNPNDGYEELNLKSTKGALQFSPWILVPQIGY |
| | GFHQYLPFPDGMSPFQAAMKDGSGYQVHRTMQFEDGASLTSNYRYTYEGSHIKGEFQ |
| | VIGTGFPADGPVMTNSLTAADWCVTKMLYPNDKTIISTFDWTYTTGSGKRYQSTVRTNY |
| | TFAKPMAANILKNQPMFVFRKTELKHSKTELNFKEWQKAFTDVM (SEQ ID NO: 66) |
| J9PBR7 | MHMKLYMEGTVNGHHFKCTSEGEGKPYEGTQTAKIKVVEGGPLPFAFDILATSFMYGS |
| | KTFINHTQGIPDFFKQSFPEGFTWERITTYEDGGVLTATQDTSLQNGCLIYNVKINGVNFP |
| | SNGPVMQKKTLGWEASTEMLYPADSGLRGHSQMALKLVGGGYLHCSLKTTYRSKKPA |
| | KNLKMPGFYFVDRKLERIKEADKETYVEQHEMAVARYCDLPSKLGHS (SEQ ID NO: 67) |
| A0A182DW87 | MVSKGEELIKENMHSKLYLEGSVNGHQFKCTHEGEGKPYEGTQTNRIKVVEGGPLPFA |
| | FDILATMFMYGSKTFIKYPKGLPDYFKQSFPEGFTWERVMVFEDGGVLTATQDTSLQDG |
| | CLIYNVKLRGVNFPANGPVMQKKTLGWEPSTETLYPADGGLEGRCDKALKLVGGGHLH |
| | CNFKTTYRSKKPATNLKMPGVHYVDRRLERIKEADNETYVEQHEVAVARYCDLPSKLGH |
| | KLNGMDELYK (SEQ ID NO: 68) |

Table 3 lists the stabilizing mutations introduced by PROSS to the respective wild-type amino acid sequence enumerated in Table 2 for each enzyme that is listed in Table 1; each enzyme is identified by its respective Uniprot Accession number and the number of enzyme variants for each respective wild-type enzyme is indicated as “variant number”.

TABLE 3
Enzyme variants and the mutations introduced to the respective
wild-type enzyme sequence.

| | Variant | |
|---|---|---|
| id | number | Mutations |
| Q5S6Z9 | 1 | N11K, K32N, F34Y, L93M, T94I, I100V, |
| | | I102T, T113C, N116H, R119K, A127P, |
| | | K145M, T154V, I157C, A160F, N166G, |
| | | A167G, F173C, E181K, F191L, V192I, |
| | | C195R |
| Q5S6Z9 | 2 | N11K, K32N, L93M, T94I, I100V, I102T, |
| | | T113C, R119E, A127P, K145S, A160F, |
| | | N166G, A167G, F173V, E181K, F191L, |
| | | V192I, C195R |
| Q5S6Z9 | 3 | N11K, K32N, F34Y, L93M, I100V, I102T, |
| | | T113C, N116H, K117E, R119E, A127P, |
| | | K145M, T154V, I157C, A160F, N166G, |
| | | A167G, F173C, E181K, F191L, V192I, |
| | | C195R |
| Q5S6Z9 | 4 | N11K, K32N, L93M, I100V, R119E, A127P, |
| | | K145M, T154V, I157C, A160F, N166G, |
| | | A167G, F173C, E181K, F191L, V192I, |
| | | C195R |
| Q5S6Z9 | 5 | L93M, R119E, A127P, K145M, N166G, |
| | | A167G, F173C, F191L, C195R |
| Q5S6Z9 | 6 | N11K, D26E, D28E, K32N, F34Y, M40I, |
| | | L93M, I100C, I102T, T113C, N116H, |
| | | K117E, R119E, A127P, L137I, K145M, |
| | | T154V, I157C, A160F, N166G, A167G, |
| | | F173I, E181K, L186M, F191L, V192I, |
| | | C195R |
| Q5S6Z9 | 7 | N11K, K32N, L93M, I100V, R119E, A127P, |
| | | K145M, T154V, A160F, N166G, A167G, |
| | | F173V, E181K, F191L, V192I, C195R |
| Q5S6Z9 | 8 | N11K, K32N, L93M, I100V, T113C, R119E, |
| | | A127P, K145M, T154V, I157C, A160F, |
| | | N166G, A167G, Y169L, F173V, E181K, |
| | | F191L, V192I, C195R |
| Q5S6Z9 | 9 | N11K, K32N, L93M, I100V, T113C, R119E, |
| | | A127P, K145M, T154V, A160F, N166G, |
| | | A167G, F173C, E181K, F191L, V192I, |
| | | C195R |
| Q5S6Z9 | 10 | N11K, D26E, D28E, K32N, F34Y, M40I, |
| | | L93M, T94I, I100V, I102T, T113C, N116H, |
| | | R119E, A127P, L137I, K145Y, T154V, |
| | | I157V, A160F, N166G, A167G, F173V, |
| | | E181K, L186M, F191L, V192I, C195R |
| Q5S6Z9 | 11 | N11K, K32N, L93M, I100V, R119E, A127P, |
| | | K145S, N166G, A167G, F173C, F191L, |
| | | V192I, C195R |
| Q5S6Z9 | 12 | N11K, K32N, L93M, I100V, I102T, T113C, |
| | | R119E, A127P, K145M, I157C, A160F, |
| | | N166G, A167G, F173C, E181K, F191L, |
| | | V192I, C195R |
| Q9U6Y8 | 1 | M1A, R17H, T21S, V22M, H41T, N42Q, |
| | | V44A, A57S, K70R, V71A, Y72F, V73T, |
| | | K83Y, L85Q, S111T, Q114E, C117T, |
| | | F118L, F124L, I125R, V127T, S131P, |
| | | A145P, R153E, E160D, H162K, K163M, |
| | | K166R, H172R, L174R, V175A, E176D, |
| | | S179T, I180T, M182K, L189M, Y192A, |
| | | Y194F, S197R, I210V, T217S, H222S, |
| | | L223T, F224G |
| Q9U6Y8 | 2 | N6S, R17H, T21S, V22M, H41T, N42Q, |
| | | V44A, A57S, K70R, V71A, Y72F, V73T, |
| | | K83Y, L85Q, N98V, S111T, Q114E, |
| | | D115G, F118L, K123W, F124L, I125R, |
| | | V127T, S131P, D132N, A145P, R153E, |
| | | E160D, H162K, K163M, K166R, L174T, |
| | | V175A, E176Q, S179T, I180T, M182K, |
| | | L189M, Y192S, Y194F, S197R, D200E, |
| | | N205D, I210V, T217A, G219A, H222S, |
| | | L223T, F224G |
| Q9U6Y8 | 3 | M1A, R17H, T21S, V22M, H41T, N42Q, |
| | | V44A, A57S, K70R, V71A, Y72F, V73T, |
| | | K83Y, L85Q, S111T, Q114E, C117T, |
| | | F118L, F124L, I125R, V127T, S131P, |
| | | A145P, R153E, E160D, H162K, K163M, |
| | | K166R, H172R, L174R, V175A, E176D, |
| | | S179T, I180T, M182K, L189M, Y192A, |
| | | Y194F, S197R, N205D, I210V, T217S, |
| | | H222S, L223T, F224G |
| Q9U6Y8 | 4 | N6S, R17H, T21S, V22M, H41T, N42Q, |
| | | V44A, A57S, K70R, V71A, Y72F, V73T, |
| | | K83Y, L85Q, S111T, Q114E, D115G, |
| | | C117T, F118L, F124L, I125R, V127T, |
| | | S131P, A145P, R153E, E160D, H162K, |
| | | K163M, K166R, H172R, L174T, V175A, |
| | | E176D, S179T, I180T, M182K, L189M, |
| | | Y192A, Y194F, S197R, N205D, I210V, |
| | | T217S, G219A, H222S, L223T, F224G |
| Q9U6Y8 | 5 | N6S, R17H, T21S, V22M, H41T, N42Q, |
| | | V44A, A57S, K70R, V71A, Y72F, V73T, |
| | | K83Y, L85Q, S111T, Q114E, D115G, |
| | | F118L, F124L, I125R, V127T, S131P, |
| | | D132N, A145P, R153E, E160D, H162K, |
| | | K163M, K166R, H172R, L174T, V175A, |
| | | E176D, S179T, I180T, M182K, L189M, |
| | | Y192A, Y194F, S197R, N205D, I210V, |
| | | T217A, H222S, L223T, F224G |
| Q9U6Y8 | 6 | N6S, R17H, T21S, V22M, H41T, N42Q, |
| | | V44A, A57S, K70R, V71A, Y72F, V73T, |
| | | K83Y, L85Q, S111T, Q114E, D115G, |
| | | C117T, F118L, F124L, I125R, V127T, |
| | | S131P, A145P, R153E, E160D, H162K, |
| | | K163M, K166R, H172R, L174R, V175A, |
| | | E176D, S179T, I180T, M182K, L189M, |
| | | Y192A, Y194F, S197R, N205D, I210V, |
| | | T217S, G219A, H222S, L223T, F224G |
| Q9U6Y8 | 7 | N6S, R17H, T21S, V22M, H41T, N42Q, |
| | | V44A, A57S, K70R, V71A, Y72F, V73T, |
| | | K83Y, L85Q, S111T, Q114E, C117T, |
| | | F118L, F124L, I125R, V127T, S131P, |
| | | A145P, R153E, E160D, H162K, K163M, |
| | | K166R, H172R, L174R, V175A, E176D, |
| | | S179T, I180T, M182K, L189M, Y192A, |
| | | Y194F, S197R, N205D, I210V, T217S, |
| | | G219A, H222S, L223T, F224G |
| P42212 | 1 | S28K, S30R, F64L, S72A, N105C, N146I, |
| | | H148G, M153T, K158N, V163A, N164E, |
| | | I167M, N170P, S175G, N198Y, S202H, |
| | | Q204R, A206V |
| P42212 | 2 | S28K, S30R, F64L, S72A, N105C, N146I, |
| | | H148G, M153T, K158N, V163A, N164E, |
| | | I167M, N170P, S175G, N198Y, Q204H, |
| | | A206V |
| P42212 | 3 | S28K, S30R, F64L, S72A, N105C, N146I, |
| | | H148G, M153T, K158N, V163A, N164E, |
| | | I167M, N170P, S175G, N198Y, Q204H, |
| | | A206V, V224L |
| P42212 | 4 | S30R, F64L, S72A, N105C, N146I, H148G, |
| | | M153T, K158N, V163A, N170P, S175G, |
| | | S202H, A206V, V224L |
| P42212 | 5 | S30R, F64L, S72A, N146I, H148G, M153T, |
| | | K158N, V163A, S175G, S202H, V224L |
| P42212 | 6 | S30R, F64L, S72A, N105C, N146I, H148G, |
| | | M153T, K158N, V163A, N170P, S175G, |
| | | N198Y, S202H, A206V, V224L |
| P42212 | 7 | S28K, S30R, F64L, S72A, N105C, N146I, |
| | | H148G, M153T, K158N, V163A, N170P, |
| | | S175G, N198Y, S202H, A206V, V224L |
| P08659 | 1 | A4G, K8I, A12P, D19P, Q25L, V36H, T39R, |
| | | I47T, E48G, V49Q, Y56L, E58Q, M59W, |
| | | S60A, V61N, L63M, M67L, T74K, N75G, |
| | | R77V, M90V, A101T, A103H, I108N, |
| | | N110T, R112D, L114I, N116H, S117Q, |
| | | M118L, T124K, V128T, K130P, G132A, |
| | | Q134P, V168M, T169K, H171Y, G175N, |
| | | V182K, K190Q, T191Q, T214N, A222C, |
| | | D234G, G246A, T251C, T252V, I257R, |
| | | C258Q, L264V, Y266R, S276A, Q283T, |
| | | T290S, S293V, G316A, H332N, A361S, |
| | | V384P, M396N, S399K, N409K, L411T, |
| | | S420T, W426F, E430G, S440E, S456A, |
| | | I457V, Q460S, N463D, F465A, L472Y, |
| | | E488K, H489P, V499I, T507S, T508P, |
| | | A509H, V522I, G525T, L526P, L530I, |
| | | A532R |
| P08659 | 2 | A4G, K8I, D19P, T39R, I47T, E48G, Y56L, |
| | | M59W, V61C, L63V, M67L, R77V, M90V, |
| | | A101T, A103H, I108N, N110T, N116H, |
| | | S117Q, M118L, T124K, K130P, V168M, |
| | | H171Y, T191Q, T214N, T251H, I257R, |
| | | L264V, Y266K, S276A, L277V, Q283T, |
| | | T290P, S293V, G316A, V384P, S420T, |
| | | W426F, E430G, S440E, F465A, L472Y, |
| | | E488K, H489P, T507S, T508P, A509Y, |
| | | V522I, G525N, A532K |
| P08659 | 3 | A4G, K8I, D19P, T39R, I47T, E48G, Y56L, |
| | | V61C, L63V, M67L, R77V, M90V, |
| | | A101T, A103H, I108N, N110T, N116H, |
| | | S117Q, M118L, T124K, K130P, V168M, |
| | | H171Y, T191Q, T214N, T251H, I257R, |
| | | L264V, Y266K, S276A, L277V, Q283T, |
| | | T290P, S293V, G316A, V384P, S420T, |
| | | W426F, E430G, S440E, F465A, L472Y, |
| | | E488K, H489P, T507S, T508P, A509Y, |
| | | V522I, G525N, A532K |
| P08659 | 4 | A4G, K8I, D19P, I47T, E48G, Y56L, V61C, |
| | | L63V, M67L, R77V, M90V, A101T, A103H, |
| | | I108N, N116H, S117Q, M118L, K130P, |
| | | V168M, H171Y, T191Q, T214N, T251H, |
| | | I257R, L264V, Y266K, S276A, K281R, |
| | | Q283T, T290P, S293V, V384P, S420T, |
| | | W426F, E430G, S440E, F465A, L472Y, |
| | | E488K, H489P, T508P, A509Y, V522I, |
| | | G525N, A532K |
| P08659 | 5 | A4G, K8I, D19P, I47T, E48G, Y56L, |
| | | M59W, V61C, L63V, M67L, R77V, M90V, |
| | | A101T, A103H, I108N, S117Q, M118L, |
| | | K130P, V168M, H171Y, T251H, I257R, |
| | | L264V, Y266K, S276A, L277V, K281R, |
| | | Q283T, T290P, S293V, V384P, S420T, |
| | | W426F, E430G, S440E, F465A, L472Y, |
| | | E488K, H489P, T508P, A509H, V522I, |
| | | A532K |
| P08659 | 6 | A4G, K8I, A12P, F14R, D19P, Q25L, V36H, |
| | | T39R, I47T, E48G, V49Q, Y56L, E58Q, |
| | | M59W, S60A, V61N, L63M, M67L, T74K, |
| | | N75G, R77V, Q87E, M90V, A101T, A103H, |
| | | I108N, N110T, R112D, L114I, N116H, |
| | | S117Q, M118L, T124K, V128T, K130P, |
| | | G132A, Q134P, Q147K, T156Q, Q159N, |
| | | V168M, T169K, H171Y, G175N, V182K, |
| | | K190Q, T191Q, T214N, A222C, D234G, |
| | | G246A, T251C, T252V, I257R, C258Q, |
| | | L264V, Y266R, S276A, L277I, Q283T, |
| | | T290S, S293V, N308S, H310T, G316A, |
| | | H332G, I349V, A361S, F368G, V384P, |
| | | M396N, S399K, N409K, L411T, S420T, |
| | | W426F, E430G, S440E, S456A, I457V, |
| | | Q460S, N463K, F465A, L472Y, E488K, |
| | | H489P, V499I, T507S, T508P, A509Y, |
| | | V522I, G525T, L526P, L530I, A532R |
| P08659 | 7 | A4G, K8I, A12P, D19P, T39R, I47T, E48G, |
| | | V49Q, Y56L, S60A, V61C, L63V, M67L, |
| | | T74K, R77V, M90V, A101T, A103H, I108T, |
| | | N110T, N116H, S117Q, M118L, T124K, |
| | | V128T, K130P, G132A, Q134P, V168M, |
| | | H171Y, G175N, T191Q, T214N, A215I, |
| | | D234G, G246A, Y255H, I257R, C258Q, |
| | | L264V, Y266K, S276A, L277V, Q283T, |
| | | T290P, S293V, G316A, H332N, A361S, |
| | | V384P, S399K, N409K, S420T, W426F, |
| | | E430G, S440E, S456A, I457V, Q460S, |
| | | N463D, F465A, L472Y, E488K, H489P, |
| | | V499I, T507S, T508P, A509Y, V522I, |
| | | G525S, L526P, L530I, A532K |
| P08659 | 8 | A4G, K8I, A12P, F14R, D19P, Q25L, A29L, |
| | | V36H, T39R, I47T, E48G, V49Q, Y56L, |
| | | E58Q, M59W, S60A, V61N, L63M, M67L, |
| | | T74K, N75G, R77V, Q87E, M90V, A101V, |
| | | A103H, I108N, N110T, R112D, L114I, |
| | | N116H, S117Q, M118L, T124K, V128T, |
| | | K130P, G132A, Q134P, Q147K, T156Q, |
| | | Q159N, V168M, T169K, H171Y, G175N, |
| | | V182K, K190Q, T191Q, T214N, A215I, |
| | | A222C, D234G, G246A, T251C, T252V, |
| | | I257Y, C258Q, L264V, Y266R, S276A, |
| | | L277I, Q283T, T290S, F292M, S293V, |
| | | N308S, H310T, G316A, H332G, I349V, |
| | | A361S, F368G, V384P, M396N, S399K, |
| | | N409K, L411T, S420T, W426F, E430G, |
| | | S440E, S456A, I457V, Q460S, N463K, |
| | | F465A, L472Y, E488K, H489P, V499I, |
| | | T507S, T508P, A509Y, V522I, G525T, |
| | | L526P, L530I, A532R |
| P08659 | 9 | A4G, K8I, D19P, I47T, E48G, Y56L, |
| | | M59W, V61C, L63V, M67L, R77V, M90V, |
| | | A101T, A103T, I108N, N116H, S117Q, |
| | | M118L, K130S, V168M, H171Y, T191Q, |
| | | T214N, T251H, I257R, L264V, Y266K, |
| | | S276A, L277V, K281R, Q283T, S284V, |
| | | T290P, S293V, V384P, S420T, W426F, |
| | | E430G, S440E, F465A, L472Y, E488K, |
| | | H489P, T508P, A509Y, V522I, G525N, |
| | | A532K |
| P08659 | 10 | A4G, K8I, A12P, D19P, Q25L, V36H, T39R, |
| | | I47T, E48G, V49Q, Y56L, E58Q, M59W, |
| | | S60A, V61N, L63M, M67L, T74K, N75G, |
| | | R77V, M90V, A101T, A103H, I108N, |
| | | N110T, R112D, L114I, N116H, S117Q, |
| | | M118L, T124K, V128T, K130P, G132A, |
| | | Q134P, V168M, T169K, H171Y, G175N, |
| | | V182K, K190Q, T191Q, T214N, A222C, |
| | | D234G, G246A, T251C, T252V, I257R, |
| | | C258Q, L264I, Y266R, S276A, L277V, |
| | | Q283T, T290S, S293V, G316A, H332N, |
| | | A361S, V384P, M396N, S399K, N409K, |
| | | L411T, S420T, W426F, E430G, S440E, |
| | | S456A, I457V, Q460S, N463D, F465A, |
| | | L472Y, E488K, H489P, V499I, T507S, |
| | | T508P, A509H, V522I, G525T, L526P, |
| | | L530I, A532R |
| P08659 | 11 | A4G, K8I, D19P, I47T, E48G, Y56L, V61C, |
| | | L63V, M67L, R77V, M90V, A101T, A103H, |
| | | I108N, S117Q, M118L, K130P, V168M, |
| | | H171Y, T251H, I257R, L264V, Y266K, |
| | | S276A, L277V, K281R, Q283T, S284V, |
| | | T290P, S293V, V384P, S420T, W426F, |
| | | E430G, S440E, F465A, L472Y, E488K, |
| | | H489P, T508P, A509Y, V522I, A532K |
| P08659 | 12 | A4G, K8I, A12P, D19P, T39R, I47T, E48G, |
| | | Y56L, M59W, V61C, L63V, M67L, R77V, |
| | | M90V, A101V, A103T, I108T, N110T, |
| | | N116H, S117Q, M118L, T124K, K130S, |
| | | Q134P, V168M, H171Y, G175N, T191Q, |
| | | T214N, R223S, T251H, I257Y, L264V, |
| | | Y266K, S276A, Q283T, T290P, S293V, |
| | | G316A, A361S, V384P, S399K, N409K, |
| | | S420T, W426F, E430G, S440E, F465A, |
| | | L472Y, E488K, H489P, V499I, T507S, |
| | | T508P, A509H, V522I, G525N, A532K |
| P08659 | 13 | A4G, K8I, A12P, D19P, T39R, I47T, E48G, |
| | | V49Q, Y56L, M59W, S60A, V61C, L63V, |
| | | M67L, T74K, R77V, M90V, A101V, |
| | | A103H, I108T, N110T, N116H, S117Q, |
| | | M118L, T124K, V128T, K130P, G132A, |
| | | V168M, H171Y, G175N, T191Q, T214N, |
| | | D234G, G246A, T251H, I257R, C258Q, |
| | | L264V, Y266K, S276A, L277V, Q283T, |
| | | T290P, S293V, G316A, H332N, A361S, |
| | | V384P, S399K, N409K, S420T, W426F, |
| | | E430G, S440E, S456A, I457V, Q460S, |
| | | N463D, F465A, L472Y, E488K, H489P, |
| | | V499I, T507S, T508P, A509Y, V522I, |
| | | G525N, L526P, L530I, A532K |
| P08659 | 14 | A4G, K8I, A12P, D19P, T39R, I47T, E48G, |
| | | Y56L, V61C, L63V, M67L, R77V, M90V, |
| | | A101V, A103T, I108T, N110T, N116H, |
| | | S117Q, M118L, T124K, K130P, Q134P, |
| | | V168M, H171Y, G175N, T191Q, T214N, |
| | | I257R, L264V, Y266K, S276A, K281R, |
| | | Q283T, T290P, S293V, G316A, A361S, |
| | | V384P, S399K, N409K, S420T, W426F, |
| | | E430G, S440E, F465A, L472Y, E488K, |
| | | H489P, V499I, T507S, T508P, A509H, |
| | | V522I, G525N, A532K |
| D0VWW2 | 1 | N8D, N9P, R22H, F46T, A49M, F88L, |
| | | D120G, E122C, S136P, A150P, S152T, |
| | | A161T, K167T, T179R, S180C, A197N, |
| | | I199F, G201D, K203R, D205Y, N210D, |
| | | G224A |
| D0VWW2 | 2 | N8D, N9P, R22H, S26C, F46T, A49M, |
| | | K50R, F88L, D120G, E122C, S136P, |
| | | D137N, A150P, S152T, A161T, K167T, |
| | | R169A, T179R, S180C, A197N, I199F, |
| | | G201D, K203R, D205Y, N210D, Q218E, |
| | | G224A |
| D0VWW2 | 3 | N8D, R22H, F46T, F88L, E122C, A150P, |
| | | S152T, A161T, T179R, S180C, I199F, |
| | | G201D, K203R, D205Y, G224A |
| D0VWW2 | 4 | N8D, N9P, R22H, F46T, A49M, F88L, |
| | | D120G, E122C, S136P, A150P, S152T, |
| | | A161T, K167T, T179R, S180C, A197N, |
| | | I199F, G201D, K203R, D205E, N210D, |
| | | G224A |
| D0VWW2 | 5 | N8D, R22H, F46T, A49M, F88L, D120G, |
| | | E122C, A150P, S152T, A161T, T179R, |
| | | S180C, I199F, G201D, K203R, D205Y, |
| | | N210D, G224A |
| D0VWW2 | 6 | N8D, R22H, F46T, F88L, A150P, S152C, |
| | | A161T, T179R, S180C, I199F, G201N, |
| | | K203R, D205Y, G224A |
| D0VWW2 | 7 | N8D, R22H, F46T, F88L, A150P, S152C, |
| | | A161T, T179R, S180C, I199F, G201N, |
| | | D205V, G224A |
| A0A1D5B367 | 1 | A12P, D16N, A20S, V25T, D28E, S119F, |
| | | A122E, V127L, G143E, T144I, M145L, |
| | | G151P, T154E, A163C |
| A0A1D5B367 | 2 | A12P, D16N, A20S, V25T, D28E, S119F, |
| | | A122E, V127M, G143E, T144I, M145L, |
| | | G151P, T154E, A163C |
| A0A1D5B367 | 3 | M1L, A12P, E15P, D16N, A19P, A20S, |
| | | V25T, D28E, K35T, A42I, I114T, N115D, |
| | | A118D, S119F, A121V, A122E, V127M, |
| | | G143E, T144I, M145L, G151P, T154K, |
| | | L156I, A162N, A163C |
| A0A1D5B367 | 4 | M1L, A12P, E15P, D16N, A19P, A20S, |
| | | V25T, D28E, K35T, A42I, I114T, N115D, |
| | | A118D, S119F, A121V, A122E, V127L, |
| | | G143E, T144I, M145L, G151P, T154E, |
| | | L156I, A162N, A163C |
| A0A1D5B367 | 5 | K4R, A12P, D16N, A20S, V25T, D28E, |
| | | K35T, A42I, N115D, A118D, S119F, |
| | | A122E, V127M, G143E, T144I, M145L, |
| | | G151P, T154K, A163C |
| A0A1D5B367 | 6 | A12P, D28E, K35P, S119F, A122E, V127M, |
| | | G143E, M145L, G151P, T154E, A163C |
| A0A1D5B367 | 7 | K4R, A12P, D16N, A20S, V25T, D28E, |
| | | K35Q, A42I, N115D, A118D, S119F, |
| | | A121V, A122E, V127L, G143E, T144I, |
| | | M145L, G151P, T154K, A163C |
| A0A1D5B367 | 8 | A12P, D16N, A20S, V25T, D28E, K35T, |
| | | N115D, S119F, A122E, V127M, G143E, |
| | | T144I, M145L, G151P, T154K, A163C |
| A0A1D5B367 | 9 | A12P, A20S, D28E, S119F, A122E, V127M, |
| | | G143E, T144I, M145L, G151P, T154E, |
| | | A163C |
| A0A1D5B367 | 10 | A12P, D28E, S119F, A122E, V127M, |
| | | G143E, M145L, G151P, T154E |
| A0A1D5B367 | 11 | A12P, A20S, D28E, K35P, S119F, A122E, |
| | | V127M, G143E, T144I, M145L, G151P, |
| | | T154E, A163C |
| Q9U6Y4 | 1 | K9T, T30E, I34R, K39E, I49V, E50K, G94E, |
| | | S96T, I106T, C107A, N108R, I125E, |
| | | N127H, M129T, D134N, K139Q, M141R, |
| | | T143V, N144G, A147P, M153Y, K157N, |
| | | K162R, S166T, L170K, V184T, S189K, |
| | | S192K, E196G, Q201E, L205E, T220Y, |
| | | I224V, F226H, A229P |
| Q9U6Y4 | 2 | K9T, T30E, I34R, K39E, I49V, E50K, G94E, |
| | | S96T, I106T, C107A, N108R, I125K, |
| | | N127H, M129T, D134N, K139Q, M141R, |
| | | T143V, N144G, A147P, M153Y, K157N, |
| | | S166T, L170K, V184T, S189K, S192K, |
| | | E196G, Q201E, L205E, T220Y, I224V, |
| | | F226H, A229P |
| Q9U6Y4 | 3 | G94E, K139Q, T143V, A147P, M153Y, |
| | | K162R, A229P |
| Q9U6Y4 | 4 | G94E, K139Q, T143I, A147P, M153Y, |
| | | A229P |
| Q9U6Y4 | 5 | K9T, T30E, I34R, K39E, G94E, S96T, |
| | | I106T, C107A, N108S, I125K, N127H, |
| | | M129T, K139Q, M141K, T143V, N144G, |
| | | A147P, M153Y, K157N, K162R, S166T, |
| | | L170K, V184T, E196G, T220Q, I224V, |
| | | F226H, A229P |
| Q9U6Y4 | 6 | K9T, T30E, I34R, K39E, G94E, S96T, |
| | | I106T, C107A, N108S, I125K, N127H, |
| | | M129T, K139Q, M141K, T143L, N144G, |
| | | A147P, M153Y, K157N, S166T, L170K, |
| | | V184T, E196G, T220H, I224V, F226H, |
| | | A229P |
| Q9U6Y4 | 7 | T30E, I34E, K39E, G94E, N108S, N127H, |
| | | M129T, K139Q, M141R, T143V, A147P, |
| | | M153Y, K162R, E196G, T220E, F226H, |
| | | A229P |
| Q9U6Y4 | 8 | I34E, G94E, N127H, K139Q, T143V, |
| | | A147P, M153Y, K162R, T220E, A229P |
| Q9U6Y4 | 9 | K9T, T30E, I34E, K39E, G94E, S96T, |
| | | I106T, N108S, I125K, N127H, M129T, |
| | | K139Q, M141R, T143V, A147P, M153Y, |
| | | K162R, V184T, E196G, T220E, F226H, |
| | | A229P |
| Q9U6Y4 | 10 | K9T, T30E, I34E, K39E, G94E, S96T, |
| | | I106T, N108T, I125K, N127H, M129T, |
| | | K139Q, M141K, T143V, A147P, M153Y, |
| | | V184T, E196G, T220Q, F226H, A229P |
| A0A1S4NYF2 | 1 | V52H, D53E, E69M, T139R, K152H, |
| | | S166K, W172P, K177M, T188D, G196K, |
| | | S203C |
| A0A1S4NYF2 | 2 | V52H, D53E, E69Q, S131I, T139R, S143D, |
| | | K152H, T164Q, S166K, A169G, W172P, |
| | | K177M, T188D, K190T, T194K, G196E, |
| | | N197D, S203C, T208H, N218D, Y226F |
| A0A1S4NYF2 | 3 | S166K, K177M, G196E, S203C |
| A0A1S4NYF2 | 4 | V52H, D53E, N65Y, E69Q, A110Q, S131I, |
| | | T139R, S143D, K152H, A158P, T164Q, |
| | | S166K, A169G, W172P, S175E, K177M, |
| | | T178L, T188D, K190T, T194K, G196E, |
| | | N197D, S203C, T204Q, T208H, N218D, |
| | | Y226F |
| A0A1S4NYF2 | 5 | E69M, T139R, S166K, K177M, T188D, |
| | | G196E, S203C |
| A0A1S4NYF2 | 6 | V52H, D53E, E69M, T139R, K152H, |
| | | S166K, W172P, K177M, G196K, S203C |
| A0A1S4NYF2 | 7 | V52H, D53E, N65Y, E69Q, A110Q, S131C, |
| | | T139R, S143D, K152H, A158P, T164Q, |
| | | S166K, A169G, W172P, K177M, T188E, |
| | | K190T, T194K, G196E, N197D, S203C, |
| | | T204Q, T208H, N218D, Y226F |
| A0A1S4NYF2 | 8 | V52H, D53E, E69Q, S131I, T139R, S143D, |
| | | K152H, T164Q, S166K, A169G, W172P, |
| | | S175E, K177M, T188D, K190T, T194K, |
| | | G196E, N197D, S203C, T208H, N218D, |
| | | Y226F |
| A0A1S4NYF2 | 9 | V52H, D53E, E69Q, T139R, K152H, S166K, |
| | | W172P, K177M, K190T, G196E, N197D, |
| | | S203C, Y226F |
| A0A1S4NYF2 | 10 | V52H, D53E, E69Q, S131I, T139R, S143D, |
| | | K152H, S166K, A169G, W172P, K177M, |
| | | T188E, K190T, G196E, N197D, S203C, |
| | | Y226F |
| A0A1S4NYF2 | 11 | E69M, T139R, S166K, K177M, G196E, |
| | | S203C |
| B1PNC0 | 1 | E35Q, S97I, Y102F, I118H, W138P, T141E, |
| | | T160K, G162K, N174T, K213W |
| B1PNC0 | 2 | D19E, E35Q, K79C, S97I, N101H, Y102F, |
| | | I118H, W138P, T141E, S153G, T160K, |
| | | G162K, T170E, N174T, K213W |
| B1PNC0 | 3 | E35Q, S97I, Y102F, I118H, W138P, T154E, |
| | | T160K, G162K, T170E, N174T, K213 |
| B1PNC0 | 4 | V18H, D19E, E35Q, S49N, A76K, K79C, |
| | | S97I, N101H, Y102F, I118H, S132K, |
| | | W138P, T141E, S153G, T160K, G162K, |
| | | T170E, N174T, K213W |
| B1PNC0 | 5 | D19E, E35Q, K79C, S97I, N101H, Y102F, |
| | | I118H, W138P, T141E, S153G, T154E, |
| | | T160K, G162K, T170E, N174T, K213W |
| B1PNC0 | 6 | A5T, V18H, D19E, E35Q, S49N, A76K, |
| | | K79C, S971, N101H, Y102F, S109N, I118H, |
| | | S132K, W138P, T141E, S153G, T160K, |
| | | G162K, T170E, N174T, N184D, K213W |
| B1PNC0 | 7 | E35Q, S97I, Y102F, I118H, W138P, T160K, |
| | | G162K, N174T, K213W |
| B1PNC0 | 8 | M1A, A5T, V18H, D19E, E35Q, S49N, |
| | | A76K, K79C, S97I, S100A, N101H, Y102F, |
| | | T105R, S109N, K112I, I118H, S132K, |
| | | W138P, T141E, S153G, T160K, G162K, |
| | | T170E, N184D, K213W |
| J9PBR7 | 1 | G13N, T19E, S20G, A33M, K34R, A51M, |
| | | I85V, T86M, T100I, N104D, L107F, K112E, |
| | | N114R, A134P, A142R, S144G, R147E, |
| | | H149R, Q151N, V157K, Y161H, H163I, |
| | | S165H, F184V, F186Y, R189H, K190R, |
| | | E196H, K199D, M208V |
| J9PBR7 | 2 | G13N, T19E, S20G, K25N, A33M, K34R, |
| | | A51M, I85V, T86M, Y88F, L94C, T100I, |
| | | N104D, L107F, N114R, S120P, A134P, |
| | | A142R, S144G, R147E, H149R, Q151N, |
| | | V157K, Y161H, H163I, S165H, F184V, |
| | | F186Y, R189H, K190R, E196H, K199D, |
| | | T201N, M208V |
| J9PBR7 | 3 | G13N, T19E, S20G, A33M, K34R, A51M, |
| | | I85V, T86M, L94C, T100I, N104D, L107F, |
| | | N114R, A134P, A142R, S144G, R147E, |
| | | H149R, Q151N, V157K, Y161H, H163I, |
| | | S165H, F184V, F186Y, R189H, K190R, |
| | | E196H, K199D, M208V |
| J9PBR7 | 4 | G13N, S20G, A33M, K34R, I85V, T86M, |
| | | N104D, L107F, N114R, A134P, A142C, |
| | | S144G, R147E, H149R, Q151D, Y161H, |
| | | H163I, S165H, F184V, F186Y, K190R, |
| | | E196H, K199D, M208V |
| J9PBR7 | 5 | G13N, S20G, A33M, K34R, I85V, T86M, |
| | | N104D, L107F, N114R, A134P, A142C, |
| | | S144G, R147E, H149R, Q151D, V157K, |
| | | Y161H, H163I, S165H, F184V, F186Y, |
| | | K190R, E196H, K199D, M208V |
| J9PBR7 | 6 | G13N, S20G, A33M, K34R, I85V, T86M, |
| | | T100I, N104D, L107F, K112E, N114R, |
| | | A134P, A142R, S144G, R147E, H149R, |
| | | Q151N, V157K, Y161H, H163I, S165H, |
| | | F184V, F186Y, R189Y, K190R, E196H, |
| | | K199D, M208V |
| J9PBR7 | 7 | G13N, S20G, A33M, K34R, I85V, T86M, |
| | | N104D, L107F, N114R, A134P, A142C, |
| | | S144G, R147E, H149R, Q151D, V157K, |
| | | Y161H, H163I, S165H, F184V, F186Y, |
| | | R189Y, K190R, E196H, K199D, M208V |
| J9PBR7 | 8 | G13N, S20G, A33M, K34R, I85V, T86M, |
| | | T100I, N104D, L107F, K112E, N114R, |
| | | A134P, A142C, S144G, R147E, H149R, |
| | | Q151D, V157K, Y161H, H163I, S165H, |
| | | F184V, F186Y, R189Y, K190R, E196H, |
| | | K199D, M208V |
| J9PBR7 | 9 | G13N, T19E, S20G, K25N, A33M, K34R, |
| | | A51M, I85V, T86M, Y88F, L94C, T100I, |
| | | N104D, L107F, K112E, N114R, S120P, |
| | | A134P, A142C, S144G, R147E, H149R, |
| | | Q151N, V157K, Y161H, H163I, S165H, |
| | | F184V, F186Y, R189H, K190R, E196H, |
| | | K199D, T201N, M208V |
| A0A182DW87 | 1 | L8V, E11P, H14K, L19M, H32G, N45M, |
| | | A63M, M65L, K71E, A132P, A154R, |
| | | K164M, V169K, N177H, D227H |
| A0A182DW87 | 2 | E11P, H14K, L19M, H32G, K37N, N45M, |
| | | R46K, A63M, K71E, A132P, A154R, |
| | | K164M, V169K, N177H, T189K, N211D, |
| | | V220W, D227H |
| A0A182DW87 | 3 | E11P, H14K, L19M, H32G, N45M, A63M, |
| | | M65L, K71E, A154R, K164M, V169K, |
| | | N177H |
| A0A182DW87 | 4 | L8V, E11P, H14K, L19M, H32G, K37N, |
| | | N45M, R46K, A63M, K71E, T112I, A132P, |
| | | A154K, K164M, V169K, N177H, T189K, |
| | | N211D, D227H |
| A0A182DW87 | 5 | L8V, E11P, H14K, L19M, H32G, N45M, |
| | | A63M, K71E, T112I, A154R, K164M, |
| | | V169K, N177H |
| A0A182DW87 | 6 | E11P, H14K, L19M, H32G, K37N, N45M, |
| | | R46K, V50T, A58S, A63M, K71E, L106C, |
| | | T112V, L114I, A132P, L151M, A154R, |
| | | K164M, V169K, N177H, T189K, N211D, |
| | | V220H, D227H |
| A0A182DW87 | 7 | E11P, H14K, L19M, H32G, K37N, N45M, |
| | | R46K, V50T, A58S, A63M, M65L, K71E, |
| | | L106C, T112I, A132P, L151M, A154N, |
| | | K164M, V169K, N177H, T189K, N211D, |
| | | V220Q, D227H |
| A0A182DW87 | 8 | E11P, H14K, L19M, N45M, K71E, A154R, |
| | | K164M, V169K, N177H, K179R |
| A0A182DW87 | 9 | E11P, H14K, S15T, L19M, H32G, K37N, |
| | | N45M, R46K, A63M, K71E, T112I, A132P, |
| | | A154K, K164M, V169K, N177H, T189K, |
| | | N211D, D227H |
| A0A182DW87 | 10 | H14K, L19M, N45M, K71E, A154R, |
| | | K164M, V169K |
| A0A182DW87 | 11 | E11P, H14K, L19M, H32G, N45M, A63M, |
| | | K71E, A132P, A154R, K164M, V169K, |
| | | N177H, D227H |
| A0A182DW87 | 12 | E11P, H14K, L19M, N45M, K71E, A154R, |
| | | K164M, V169K, N177H |
| A0A182DW87 | 13 | E11P, H14K, L19M, H32G, K37N, N45M, |
| | | R46K, A63M, K71E, T112I, A132P, A154R, |
| | | K164M, V169K, N177H, T189K, N211D, |
| | | D227H |
| F2XBU9 | 1 | S108M, F130Y, A214P, L243Q, S304A, |
| | | E315G, A323Y, N342D, T408E, E436D |
| F2XBU9 | 2 | S108M, F130Y, A214P, L243Q, S304A, |
| | | E315G, A323Y, T408F, E436D |
| F2XBU9 | 3 | Y76M, V103M, S108M, F130Y, A214P, |
| | | G219A, L243Q, S304A, T309E, A313K, |
| | | E315G, E316T, A323Y, N342D, N405E, |
| | | T408Q, E436D |
| F2XBU9 | 4 | Y76M, V103M, S108M, F130Y, A214P, |
| | | L243Q, S304A, T309E, A313K, E315G, |
| | | A323Y, N342D, T408Q, E436D |
| F2XBU9 | 5 | Y76M, V103M, S108M, F130Y, A214P, |
| | | G219A, L243Q, S304A, T309E, A313K, |
| | | E315G, A323Y, N342D, N405E, T408Q, |
| | | E436D |
| F2XBU9 | 6 | A74E, Y76M, V103M, S108M, F130Y, |
| | | Q196E, Q210L, A214P, G219A, L243Q, |
| | | S304A, K305Q, T309E, A313R, E315G, |
| | | E316T, A323Y, K335E, N342D, N405E, |
| | | T406A, T408E, E436D |
| F2XBU9 | 7 | Y76M, V103M, S108M, F130Y, A214P, |
| | | G219A, L243Q, S304A, T309E, A313R, |
| | | E315G, E316T, A323Y, N342D, T408Q, |
| | | E436D |
| F2XBU9 | 8 | A74E, Y76M, V103M, S108M, F130Y, |
| | | Q196E, Q210L, A214P, G219A, L243Q, |
| | | S304A, K305Q, T309E, A313K, E315G, |
| | | E316T, A323Y, K335E, N342D, N405E, |
| | | T406A, T408Q, E436D |
| F2XBU9 | 9 | Y76M, V103M, S108M, F130Y, A214P, |
| | | G219A, L243Q, S304A, T309E, A313K, |
| | | E315G, A323Y, N342D, T408Q, E436D |
| Q05489 | 1 | A63G, N64G, S74E, S78R, A106E, S132A, |
| | | F161Y, T168Y, T176P, N183W, D196N, |
| | | R204K, Q210G, T211A, Y214F, R216Q, |
| | | A234P, S241N, Q242T, A297G, Q300P, |
| | | F312Y, S319N, V334I, N338F, T348N, |
| | | L355N, V358L |
| Q05489 | 2 | A63G, N64G, S74E, S78Q, S132A, F161Y, |
| | | T168Y, T176P, D196N, R204K, Q210G, |
| | | T211A, Y214F, A234P, S241N, Q242T, |
| | | A297G, Q300P, F312Y, S319N, V334I, |
| | | T348N, L355N, V358L |
| Q05489 | 3 | N64G, S132A, D196N, A234P, S241N, |
| | | Q242T, A297G, Q300P, S319N, T348N, |
| | | L355N, V358L |
| Q05489 | 4 | A63G, N64G, S74E, S78Q, S132A, F161Y, |
| | | T168Y, T176P, D196N, R204K, Q210G, |
| | | T211A, Y214F, A234P, S241N, Q242T, |
| | | A297G, Q300P, F312Y, S319N, V334I, |
| | | T348Q, L355N, V358L |
| Q05489 | 5 | S241N, A297G, Q300P, S319N, V358L |
| Q05489 | 6 | N64G, S132A, D196N, A234P, S241N, |
| | | Q242T, A297G, Q300P, F312Y, S319N, |
| | | T348N, L355N, V358L |
| Q05489 | 7 | S241N, A297G, Q300P, S319N, T348N, |
| | | V358L |
| Q93D82 | 1 | T3V, F12G, S17A, E33P, Q37D, K38P, |
| | | L41R, K52H, E56A, T67I, S68K, S78A, |
| | | D80A, A83P, Q87R, K91Q, V94P, G99A, |
| | | R107G, A110R, N113R, S117P, K140P, |
| | | E141G, C153I, T154A, N157K, S160A, |
| | | V205Q, K215E |
| Q93D82 | 2 | F12G, E33P, Q37D, L41R, T67I, S68K, |
| | | D80A, A83P, Q87R, V94P, G99A, N113R, |
| | | S117P, K140P, C153I, T154A, N157K |
| Q93D82 | 3 | F12G, E33P, Q37D, L41R, T67I, S68K, |
| | | A83P, Q87R, V94P, G99A, N113R, S117P, |
| | | C153I, T154A, N157K |
| Q93D82 | 4 | T3V, K10R, F12G, S17A, S22A, E33P, |
| | | Q37D, K38P, L41R, K52H, E56A, I63V, |
| | | T67I, S68K, S78A, D80A, A83P, Q87R, |
| | | K91E, A93D, V94P, G99K, Q105L, R107G, |
| | | A110R, N113Q, S117P, F136L, W139L, |
| | | E141G, S147A, C153N, T154A, N157K, |
| | | S160A, V205Q, K215E, M245V |
| Q93D82 | 5 | F12G, S17A, E33P, Q37D, K38P, L41R, |
| | | K52H, E56A, T67I, S68K, D80A, A83P, |
| | | Q87R, V94P, G99A, R107G, A110R, |
| | | N113R, S117P, C153N, T154A, N157K, |
| | | V205Q, K215E |
| Q93D82 | 6 | T3V, K10R, F12G, S17A, S22A, E33P, |
| | | Q37D, K38P, L41R, K52H, I63V, A65R, |
| | | T67I, S68E, S78A, D80A, A83P, Q87R, |
| | | K91E, A93D, V94P, G99A, Q105L, R107G, |
| | | A110R, N113R, S117P, F136L, W139L, |
| | | E141G, S147A, C153I, T154A, N157K, |
| | | S160A, V205Q, K215E |
| Q93D82 | 7 | F12G, S17A, E33P, Q37D, K38P, L41R, |
| | | K52H, E56A, T67I, S68K, D80A, A83P, |
| | | Q87R, V94P, G99A, R107G, A110R, |
| | | N113Q, S117P, K140P, C153I, T154A, |
| | | N157K, V205Q, K215E |
| Q93D82 | 8 | T3V, K10R, F12G, S17A, S22A, E33P, |
| | | Q37D, K38P, L41R, K52H, E56A, T67I, |
| | | S68E, S78A, D80A, A83P, Q87R, K91E, |
| | | A93S, V94P, G99A, R107G, A110R, |
| | | N113R, S117P, W139L, E141G, S147A, |
| | | C153I, T154A, N157K, S160A, V205Q, |
| | | T206V, K215E |
| Q93D82 | 9 | F12G, E33P, L41R, T67I, S68K, A83P, |
| | | Q87R, V94P, G99A, N113R, S117P, K140P, |
| | | C153I, T154A, N157K |
| Q93D82 | 10 | T3V, K10R, F12G, S17A, S22A, E33P, |
| | | Q37D, K38P, L41R, K52H, E56A, T67I, |
| | | S68E, S78A, D80A, A83P, Q87R, K91E, |
| | | A93S, V94P, G99A, R107G, A110R, |
| | | N113R, S117P, W139L, K140P, E141G, |
| | | S147A, C153I, T154A, N157K, S160A, |
| | | V205Q, K215E |
| Q93D82 | 11 | T3V, F12G, S17A, E33P, Q37D, K38P, |
| | | L41R, K52H, E56A, T67I, S68K, S78A, |
| | | D80A, A83P, Q87R, K91Q, V94P, G99A, |
| | | R107G, A110R, N113Q, S117P, E141G, |
| | | C153N, T154A, N157K, S160A, V205Q, |
| | | K215E |
| Q93D82 | 12 | F12G, E33P, Q37D, K38P, L41R, K52H, |
| | | E56A, T67I, S68K, D80A, A83P, Q87R, |
| | | V94P, G99A, R107G, A110R, N113R, |
| | | S117P, K140P, C153I, T154A, N157K |
| Q93D82 | 13 | F12G, E33P, L41R, T67I, S68K, D80A, |
| | | A83P, Q87R, V94P, G99A, N113R, S117P, |
| | | K140P, C153N, T154A, N157K |
| Q7NWG4 | 1 | G39A, F71L, S101E, A109P, D112N, |
| | | R113H, S123A, V124N, L146I, V202L, |
| | | V203H, A217P, V236I, A239D, A286I, |
| | | K304D, A312E, H333L, A337K, E368P, |
| | | V379L, Q380A, D396N, F397E, T402W, |
| | | A455E |
| Q7NWG4 | 2 | D70E, D112N, R113H, S123A, V124N, |
| | | A217P, A337R, E368P, Q380A, D396N, |
| | | F397E |
| Q7NWG4 | 3 | G39A, D70E, F71L, S101E, A109P, D112N, |
| | | R113H, S123A, V124N, L146I, A217P, |
| | | V236I, A286I, H333L, A337K, E368P, |
| | | V379L, Q380A, D396N, F397E, A455E |
| Q7NWG4 | 4 | G39A, D112N, R113H, S123A, V124N, |
| | | A217P, A337R, E368P, V379L, Q380A, |
| | | D396N, F397E |
| Q7NWG4 | 5 | D70E, D112N, R113H, S123A, V124N, |
| | | A217P, A286V, A337R, E368P, V379L, |
| | | Q380A, D396N, F397E |
| Q7NWG4 | 6 | G39A, D70E, F71L, S101K, A109P, D112N, |
| | | R113H, S123A, V124N, L146I, V202L, |
| | | V203H, A217P, V236I, A239D, A286I, |
| | | K304D, A312E, H333L, A337R, E368P, |
| | | V379L, Q380A, D396N, F397E, T402W, |
| | | A455E |
| Q7NWG4 | 7 | G39A, D70E, F71L, K90Q, V97I, S101E, |
| | | A109P, D112N, R113H, S123A, V124N, |
| | | T145V, L146I, G152A, V202L, V203H, |
| | | A217P, V236I, A239D, H276Y, A286I, |
| | | K304D, A312E, H333L, A337K, Q346E, |
| | | E368P, V379I, Q380A, N387D, D396N, |
| | | F397E, T402W, R410N, T452E, A455K |
| Q7NWG4 | 8 | M36L, G39A, D70E, F71L, K90Q, V97I, |
| | | S101K, A109P, D112N, R113H, S123A, |
| | | V124N, T145V, L146I, G152A, V202L, |
| | | V203K, A217P, V236I, A239D, H276Y, |
| | | A286I, K304D, A312E, H333L, A337K, |
| | | Q346E, K358Q, E368P, V379M, Q380A, |
| | | N387D, K390T, D396N, F397E, T402W, |
| | | R410N, S424A, T452E, A455K |
| Q7NWG4 | 9 | M36L, G39A, D70E, F71L, R76Y, K90Q, |
| | | V97I, S101K, A109P, D112N, R113H, |
| | | S123A, V124N, T145V, L146I, G152A, |
| | | V202L, V203K, A217P, V236I, A239D, |
| | | H276Y, A286I, K304D, A312E, H333L, |
| | | A337R, Q346E, K358Q, E368P, Q380A, |
| | | N387D, K390T, D396N, F397E, T402W, |
| | | R410N, S424A, T452E, A455E |
| Q7NWG4 | 10 | G39A, D70E, S101E, A109P, D112N, |
| | | R113H, S123A, V124N, L146I, A217P, |
| | | A286I, H333L, A337K, E368P, V379M, |
| | | Q380A, D396N, F397E |
| Q7NWG4 | 11 | G39A, D70E, F71L, S101E, A109P, D112N, |
| | | R113H, S123A, V124N, L146I, A217P, |
| | | V236I, A286I, D315E, H333L, A337R, |
| | | E368P, Q380A, D396N, F397E, A455E |
| Q7NWG4 | 12 | G39A, D70E, F71L, K90Q, V97I, S101E, |
| | | A109P, D112N, R113H, S123A, V124N, |
| | | T145V, L146I, G152A, S162A, V202L, |
| | | V203H, A217P, V236I, A239D, H276Y, |
| | | A286I, K304D, A312E, H333L, A337R, |
| | | Q346E, E368P, V379I, Q380A, N387D, |
| | | D396N, F397E, T402W, R410N, T452E, |
| | | A455K |
| Q7NWG4 | 13 | G39A, D70E, D112N, R113H, S123A, |
| | | V124N, A217P, A286V, A337R, E368P, |
| | | V379L, Q380A, D396N, F397E |
| Q7NWG4 | 14 | G39A, S101E, A109P, D112N, R113H, |
| | | S123A, V124N, L146I, A217P, A286I, |
| | | H333L, A337R, E368P, V379L, Q380A, |
| | | D396N, F397E |
| A5I055 | 1 | E91Q, I101T, A140R, K183G, K246H, |
| | | S265A, T277M, K289N, N308D, E339Y, |
| | | A375P, S395T, G437T, S479P |
| A5I055 | 2 | I101T, K183G, T277M, K289N, N308D, |
| | | A375P, S395T, S479P |
| A5I055 | 3 | D69N, E91Q, I101T, K137D, A140R, |
| | | S141N, H143Y, V168K, K183G, K196P, |
| | | N200D, K246H, S265A, T277M, K289N, |
| | | N308D, E339Y, T346K, A375P, S395T, |
| | | Q398K, G437T, M469I, N477Q, S479P |
| A5I055 | 4 | I101T, K183G, T277M, K289N, A375P, |
| | | S395T, S479P |
| A5I055 | 5 | D69N, E91Q, I101T, K137D, A140R, |
| | | S141N, H143Y, V168K, K183G, K196P, |
| | | N200D, K246H, S265A, T277M, K289N, |
| | | N308D, E339Y, T346K, A375P, S395T, |
| | | Q398K, G437T, N477Q, S479P |
| A5I055 | 6 | I101T, K183G, K246H, S253V, T277M, |
| | | K289N, N308D, A375P, S395T, G437T, |
| | | S479P |
| A5I055 | 7 | I101T, K183G, K246H, T277M, K289N, |
| | | N308D, A375P, S395T, G437T, S479P |
| A5I055 | 8 | K183G, T277M, A375P, S395T, S479P |
| P12311 | 1 | K10G, K11E, Q14K, V15I, E19P, K20V, |
| | | K22T, S24G, V66I, I67V, Q102W, R108H, |
| | | Q110K, A112T, A127D, K133H, V159E, |
| | | V179M, L196I, G197D, Q206E, K216L, |
| | | H217K, D218E, A220P, Q222K, W223F, |
| | | I224M, T234A, A242P, E245Q, S246Q, |
| | | K249R, A256T, I267M, I269V, V279I, |
| | | V308I, Q311R, N315K, D318E, K335D, |
| | | V336L, D337E |
| P12311 | 2 | K10G, K11E, Q14K, V15I, E19P, K20V, |
| | | K22T, S24G, V66I, I67V, Q102W, R108H, |
| | | Q110K, A112T, A127D, K133H, V179M, |
| | | L196I, G197D, Q206E, K216L, H217K, |
| | | D218E, A220P, Q222K, W223F, I224M, |
| | | T234A, A242P, E245Q, S246Q, K249R, |
| | | A256T, C257V, I267M, V279I, V308I, |
| | | Q311R, N315K, D318E, K335D, V336L, |
| | | D337E |
| P12311 | 3 | K10G, K11Q, Q14V, V15I, E19P, K20I, |
| | | K22T, S24G, V66I, I67V, E68V, E69A, |
| | | Y89W, Q102W, R108H, A112T, Y120F, |
| | | A127D, K133H, T153V, V159E, V179M, |
| | | L196I, G197D, E199D, Q206K, K216L, |
| | | H217K, D218E, A220P, Q222K, W223F, |
| | | I224M, E226K, T234A, A242P, S246Q, |
| | | K249R, 1251L, A256T, C257L, E265G, |
| | | I267M, I269V, V279I, T306P, V308I, |
| | | Q311R, N315D, D318E, L324R, N329D, |
| | | K335D, V336L, D337E |
| P12311 | 4 | K10G, K11Q, Q14K, V15I, E19P, K20I, |
| | | K22T, S24G, V66I, I67V, Q102W, R108H, |
| | | Q110K, A112T, Y120F, A127D, K133H, |
| | | F147L, T153V, V159E, V179M, L196I, |
| | | G197D, Q206E, K216L, H217K, D218E, |
| | | A220P, Q222K, W223F, I224M, T234A, |
| | | A242P, E245Q, S246Q, K249R, A256T, |
| | | C257L, I267M, I269V, V279I, T306V, |
| | | V308I, Q311R, N315D, D318E, N329D, |
| | | K335D, V336L, D337E |
| P12311 | 5 | K10G, K11Q, Q14V, V15I, E19P, K20I, |
| | | K22T, S24G, V28I, V66I, I67V, E68V, |
| | | E69A, Y89W, Q102W, R108H, A112T, |
| | | Y120F, A127D, K133H, T153V, V159E, |
| | | V179M, L196I, G197D, E199D, Q206K, |
| | | K216L, H217K, D218E, A220P, Q222K, |
| | | W223F, I224M, E226K, T234A, A242P, |
| | | S246Q, K249R, 1251L, A256T, C257L, |
| | | E265G, I267M, I269V, V279I, T306P, |
| | | V308I, Q311R, N315D, D318E, L324R, |
| | | N329D, K335D, V336L, D337E |
| P12311 | 6 | K10G, K11Q, Q14K, V15I, E19P, K20I, |
| | | K22T, S24G, V66I, I67V, Q102W, R108H, |
| | | Q110K, A112T, A127D, K133H, V159M, |
| | | V179M, L196I, G197D, Q206E, K216L, |
| | | H217K, D218E, A220P, Q222K, W223F, |
| | | I224M, T234A, A242P, E245Q, S246Q, |
| | | K249R, A256T, C257V, I267F, I269L, |
| | | V279I, T306P, V308I, Q311R, D318E, |
| | | N329K, K335D, V336L, D337E |
| P12311 | 7 | K10G, K11E, Q14K, V15I, E19P, K20V, |
| | | K22T, S24G, V66I, I67V, Q102W, R108H, |
| | | Q110K, A112T, A127D, K133H, V159E, |
| | | V179M, L196I, G197D, Q206E, K216L, |
| | | H217K, D218E, A220P, Q222K, W223F, |
| | | I224M, T234A, A242P, E245Q, S246Q, |
| | | K249R, A256T, I267M, V279I, V308I, |
| | | N315K, D318E, K335D, V336L, D337E |
| P12311 | 8 | K10G, K11E, Q14K, V15I, E19P, K22T, |
| | | S24G, V66I, I67V, Q102W, R108H, Q110K, |
| | | A127D, K133H, V159E, V179M, L196I, |
| | | G197D, Q206E, K216L, H217K, D218E, |
| | | A220P, Q222K, W223F, I224M, T234A, |
| | | A242P, E245Q, K249N, A256T, I267M, |
| | | I269V, V279I, V308I, N315K, D318E, |
| | | K335D, V336L, D337E |
| P12311 | 9 | K10G, K11E, Q14K, V15I, E19P, K20V, |
| | | K22T, S24G, V66I, I67V, Q102W, R108H, |
| | | Q110K, A112T, A127D, K133H, V159E, |
| | | V179M, L196I, G197D, Q206E, K216L, |
| | | H217K, D218E, A220P, Q222K, W223F, |
| | | I224M, T234A, A242P, E245Q, S246Q, |
| | | K249R, A256T, I267M, I269V, V279I, |
| | | V308I, N315K, D318E, K335D, V336L, |
| | | D337E |
| P12311 | 10 | K10G, K11Q, Q14V, V15I, E19P, K20I, |
| | | K22T, S24G, V66I, I67V, E69A, Y89W, |
| | | Q102W, R108H, A112T, Y120F, A127D, |
| | | K133H, T153V, V159E, V179M, L196I, |
| | | G197D, Q206E, K216L, H217K, D218E, |
| | | A220P, Q222K, W223F, I224M, T234A, |
| | | A242P, E245Q, S246Q, K249N, 1251L, |
| | | A256T, C257L, I267M, I269L, V279I, |
| | | T306P, V308I, Q311R, N315D, D318E, |
| | | N329D, K335D, V336L, D337E |
| P12311 | 11 | K10G, K11Q, Q14V, V15I, E19P, K20I, |
| | | K22T, S24G, V66I, I67V, E69A, Y89W, |
| | | Q102W, R108H, A112T, Y120F, A127D, |
| | | K133H, T153V, V159E, V179M, L196I, |
| | | G197D, Q206E, K216L, H217K, D218E, |
| | | A220P, Q222K, W223F, I224M, T234A, |
| | | A242P, E245Q, S246Q, K249R, 1251L, |
| | | A256T, C257L, I267M, I269V, V279I, |
| | | T306P, V308I, Q311R, N315D, D318E, |
| | | N329D, K335D, V336L, D337E |
| P12311 | 12 | K10G, K11Q, Q14K, V15I, E19P, K20I, |
| | | K22T, S24G, V66I, I67V, Q102W, R108H, |
| | | Q110K, A112T, A127D, K133H, F147L, |
| | | V159M, V179M, L196I, G197D, K205R, |
| | | Q206E, K216L, H217K, D218E, A220P, |
| | | Q222K, W223F, I224M, T234A, A242P, |
| | | E245Q, S246Q, K249R, A256T, C257V, |
| | | I267M, I269V, V279I, T306V, V308I, |
| | | Q311R, D318E, N329K, K335D, V336L, |
| | | D337E |
| P12311 | 13 | K10G, K11Q, Q14K, V15I, E19P, K20I, |
| | | K22T, S24G, V66I, I67V, Y89W, Q102W, |
| | | R108H, Q110K, A112T, Y120F, A127D, |
| | | K133H, T153V, V159E, V179M, L196I, |
| | | G197D, Q206E, K216L, H217K, D218E, |
| | | A220P, Q222K, W223F, I224M, T234A, |
| | | A242P, E245Q, S246Q, K249R, A256T, |
| | | C257L, I267F, I269L, V279I, T306V, V308I, |
| | | Q311R, N315D, D318E, N329D, K335D, |
| | | V336L, D337E |
| P12311 | 14 | K10G, K11E, Q14K, V15I, E19P, K22T, |
| | | S24G, V66I, I67V, Q102W, R108H, Q110K, |
| | | A127D, K133H, V159E, V179M, L196I, |
| | | G197D, Q206E, K216L, H217K, D218E, |
| | | A220P, Q222K, W223F, I224M, T234A, |
| | | A242P, E245Q, K249R, A256T, I267F, |
| | | I269V, V279I, V308I, N315K, D318E, |
| | | K335D, V336L, D337E |
| C4S833 | 1 | S42A, Q46P, D52N, D56H, K57P, A65P, |
| | | T77R, G81Q, F82Y, Y83F, N89Q, Q106W, |
| | | I109S, L115A, A136P, V141P, E149K, |
| | | Q154T, S157P, T158K, H161R, R162A, |
| | | E165M, Q167R, A170G, S173D, E174A, |
| | | K181D, P182D, Q185L, G187Q, E188Q, |
| | | T193C, A194N, S200A, S207T, N221S, |
| | | Q222K, Q223E, S230E, S236A, G240A, |
| | | N247Y, S248A, H257G, A262E, V266R, |
| | | Q275K, K289Q, V298L, K306D, G324A, |
| | | G334A, Q342T, Q345G, G355A, N363D, |
| | | D365K, M380L, S386A, K392T, S393N, |
| | | H394N, I409T, T411D, Q416P, A425K |
| C4S833 | 2 | Q46F, D56H, K57P, A65P, T77K, G81Q, |
| | | F82Y, Y83F, N89Q, T102E, Q106W, |
| | | L115A, V141M, Q154T, S157P, T158E, |
| | | H161R, R162A, E165M, A170G, E174A, |
| | | Q185L, G187Q, T193C, A194N, S200K, |
| | | S230E, S236A, G240A, N247Y, S248A, |
| | | A262E, V266R, V298L, K306D, G324A, |
| | | G334A, Q345G, N363D, S386A, I409T, |
| | | T411D |
| C4S833 | 3 | Q46F, K57P, A65P, T77K, G81R, N89Q, |
| | | Q106W, L115A, Q154R, S157P, T158E, |
| | | H161K, E165M, A170G, E174A, Q185L, |
| | | G187Q, T193C, S236A, G240A, N247Y, |
| | | S248A, L269M, V298L, K306D, G324A, |
| | | G334A, Q345G, T411D |
| C4S833 | 4 | Q46F, D56Q, K57P, A65P, T77K, G81R, |
| | | F82Y, Y83F, N89Q, T102E, Q106W, |
| | | I109T, L115A, V141P, Q154T, S157P, |
| | | T158E, H161R, R162A, E165M, A170G, |
| | | E174A, Q185L, G187Q, E188Q, T193C, |
| | | A194N, S200K, Q222K, Q223E, S230E, |
| | | S236A, G240A, N247Y, S248A, H257G, |
| | | A262E, V266R, S267E, L269M, V298L, |
| | | K306D, G324A, G334A, Q342T, Q345G, |
| | | G355A, N363D, S386A, S393N, H394N, |
| | | I409T, T411D, A425K |
| C4S833 | 5 | S42A, Q46F, D52N, D56H, K57P, A65P, |
| | | T77R, G81Q, F82Y, Y83F, N89Q, T102E, |
| | | Q106W, I109T, L115A, A136P, V141P, |
| | | E149K, Q154T, S157P, T158K, H161K, |
| | | R162A, E165M, Q167R, A170G, S173D, |
| | | E174A, K181D, P182S, Q185L, G187E, |
| | | E188Q, I189V, T193K, A194N, S200A, |
| | | S207T, N221S, Q222K, Q223E, S230E, |
| | | S236A, G240A, N247Y, S248A, H257G, |
| | | A262E, V266R, Q275K, K289Q, V298L, |
| | | K306D, G324A, G334A, Q342T, Q345G, |
| | | G355A, N363D, D365K, S386A, K392T, |
| | | S393N, H394N, I409T, T411D, Q416P, |
| | | A425K |
| C4S833 | 6 | Q46F, K57P, A65P, T77K, G81R, F82Y, |
| | | Y83F, N89Q, T102E, Q106W, L115A, |
| | | Q154T, S157P, T158E, H161K, E165M, |
| | | A170G, E174A, Q185L, G187Q, T193C, |
| | | S200K, S236A, G240A, N247Y, S248A, |
| | | V266R, L269M, V298L, K306D, G324A, |
| | | G334A, Q345G, T411D |
| C4S833 | 7 | Q46F, A65P, T77K, G81Q, N89Q, T102E, |
| | | Q106W, S157P, A170G, Q185L, G187Q, |
| | | T193C, S236A, G240A, N247Y, S248A, |
| | | L269M, V298L, K306D, G324A, G334A, |
| | | Q345G, T411D |
| C4S833 | 8 | Q46F, K57P, A65P, T77K, G81Q, F82Y, |
| | | Y83F, N89Q, T102E, Q106W, L115A, |
| | | Q154T, S157P, T158E, H161K, E165M, |
| | | A170G, E174A, Q185L, G187Q, T193C, |
| | | S200K, S236A, G240A, N247Y, S248A, |
| | | V266R, Q275Y, V298L, K306D, G324A, |
| | | G334A, Q345G, T411D |
| C4S833 | 9 | Q46F, D56H, K57P, A65P, T77K, G81R, |
| | | F82Y, Y83F, N89Q, T102E, Q106W, |
| | | L115A, V141M, Q154T, S157P, T158E, |
| | | H161R, R162A, E165M, A170G, E174A, |
| | | Q185L, G187Q, I189V, T193C, A194N, |
| | | S200K, S230E, S236A, G240A, N247Y, |
| | | S248A, A262E, V266R, L269M, V298L, |
| | | K306D, G324A, G334A, Q345G, N363D, |
| | | S386A, I409T, T411D |
| C4S833 | 10 | N89Q, T102E, Q106W, S157P, A170G, |
| | | Q185L, G187Q, S236A, G240A, N247Y, |
| | | S248A, V298L, G324A, G334A, Q345G, |
| | | T411D |
| C4S833 | 11 | N89Q, Q106W, S157P, A170G, Q185L, |
| | | G187Q, S236A, G240A, N247Y, S248A, |
| | | L269M, Q275Y, V298L, G324A, G334A, |
| | | Q345G, T411D |
| C4S833 | 12 | Q46F, D56H, K57P, A65P, T77K, G81Q, |
| | | F82Y, Y83F, N89Q, T102E, Q106W, I109T, |
| | | L115A, V141P, Q154T, S157P, T158E, |
| | | H161R, R162A, E165M, A170G, E174A, |
| | | Q185L, G187Q, E188Q, T193C, A194N, |
| | | S200K, Q222K, Q223E, S230E, S236A, |
| | | G240A, N247Y, S248A, H257G, A262E, |
| | | V266R, S267E, L269M, Q275Y, V298L, |
| | | K306D, G324A, G334A, Q342T, Q345G, |
| | | G355A, N363D, S386A, S393N, H394N, |
| | | I409T, T411D, A425K |
| C4S833 | 13 | Q46F, K57P, A65P, T77K, G81Q, N89Q, |
| | | T102E, Q106W, L115A, Q154R, S157P, |
| | | T158E, H161K, E165M, A170G, E174A, |
| | | Q185L, G187Q, T193C, S236A, G240A, |
| | | N247Y, S248A, V298L, K306D, G324A, |
| | | G334A, Q345G, T411D |
| Q4WZ70 | 1 | A8S, K12Q, H20N, I26V, T30M, G37D, |
| | | Q38N, G50A, D66F, T68S, M72G, P84E, |
| | | R86I, E91K, S94D, R95A, II26V, C151Y, |
| | | A153E, N164E, T184I, K186D, Q192T, |
| | | T210V, V217I, E240D, A250E, M252L, |
| | | D256K, L268Q, E270N, E304R, K305W, |
| | | Q309W, Y368W, L371R, H372N |
| Q4WZ70 | 2 | A8S, H20N, I26V, T30M, G37D, Q38N, |
| | | G50A, D66F, T68S, M72G, R86I, E91K, |
| | | S94D, C151Y, A153E, N164E, T184I, |
| | | K186D, Q192T, V217I, A250E, Q309W, |
| | | Y368W, L371R, H372Q |
| Q4WZ70 | 3 | A8S, I26V, T30M, G37D, Q38N, G50A, |
| | | D66F, T68S, M72G, R86I, S94D, C151Y, |
| | | A153E, N164E, T184I, K186D, Q192T, |
| | | V217I, Q309W, Y368W, L371R |
| Q4WZ70 | 4 | A8S, K12Q, H20N, I26V, T30M, G37D, |
| | | Q38N, G50A, D66F, T68S, M72G, P84E, |
| | | R86I, E91K, S94D, R95A, C151Y, A153E, |
| | | N164E, T184I, K186D, Q192T, T210V, |
| | | V217I, A250E, Q309W, Y368W, L371R, |
| | | H372Q |
| Q4WZ70 | 5 | A8S, K12Q, H20N, I26V, T30M, G37D, |
| | | Q38N, G50A, D66F, T68S, M72G, P84E, |
| | | R86I, E91K, S94D, R95A, C151Y, A153E, |
| | | N164E, T184I, K186D, Q192T, T210V, |
| | | V217I, A250E, M252L, Q309W, Y368W, |
| | | L371R, H372N |
| Q4WZ70 | 6 | A8S, K12Q, H20N, I26V, T30M, G37D, |
| | | Q38N, G50A, T68S, M72G, P84E, R86I, |
| | | E91K, S94D, R95A, II26V, C151I, A153E, |
| | | N164E, T184I, K186D, Q192T, T210V, |
| | | V217I, E240D, A250E, M252L, D256K, |
| | | L268Q, E270N, E304R, K305W, Q309W, |
| | | Y368W, L371R, H372N |
| Q4WZ70 | 7 | I26V, G50A, D66F, T68S, M72G, R86I, |
| | | C151Y, K186D, Q192T, Y368W |
| Q4WZ70 | 8 | I26V, T30M, G50A, D66F, T68S, M72G, |
| | | R86I, S94D, C151Y, A153E, K186D, |
| | | Q192T, Y368W |
| Q4WZ70 | 9 | A8S, I26V, T30M, G37D, G50A, D66F, |
| | | T68S, M72G, R86I, S94D, C151Y, A153E, |
| | | N164E, T184I, K186D, Q192T, Y368W, |
| | | L371R |
| O28608 | 1 | E2K, Q8R, N22E, L33E, S89D, T104I, |
| | | S106A, S125A, F128V, K235R, D277P, |
| | | S310E, S315T, K316F |
| O28608 | 2 | Q8R, S106A, K235R, D277P, S310E, |
| | | S315T, K316F |
| O28608 | 3 | E2K, Q8R, N22E, S89D, S106A, S125A, |
| | | K235R, D277P, S310E, S315T, K316F |
| O28608 | 4 | E2K, Q8R, N22E, S89D, S106A, S125A, |
| | | F128V, D277P, S310E, S315T, K316F |
| O28608 | 5 | E2K, Q8R, S13Q, N22E, L33E, G72Q, |
| | | S89D, T104I, S106A, S125A, F128V, |
| | | T135E, K235R, D277P, V300A, S310E, |
| | | S315T, K316F, I317V |
| O28608 | 6 | E2K, Q8R, N22E, L33E, S89D, T104I, |
| | | S106A, S125A, F128V, D277P, V300A, |
| | | S310E, S315T, K316F |
| O28608 | 7 | E2K, Q8R, N22E, L33E, S89D, T104I, |
| | | S106A, S125A, F128V, T135E, K235R, |
| | | D277P, V300A, S310E, S315T, K316F |
| O28608 | 8 | Q8R, S106A, K235R, D277P, S315T, K316F |
| O28608 | 9 | E2K, Q8R, S13Q, N22E, L33E, G72Q, |
| | | S89D, T104I, S106A, S125A, F128V, |
| | | K235R, D277P, V300A, S310E, S315T, |
| | | K316F, I317V |
| O28608 | 10 | E2K, Q8R, N22E, S89D, S106A, S125A, |
| | | F128V, K235R, D277P, S310E, S315T, |
| | | K316F |
| O28608 | 11 | E2K, Q8R, N22E, L33E, S89D, T104I, |
| | | S106A, S125A, F128V, D277P, S310E, |
| | | S315T, K316F |
| Q9S1A6 | 1 | T28A, E41Q, N44D, N62A, L72A, A83K, |
| | | E84A, S90P, G91D, G92D, A94P, N95K, |
| | | G99P, S103M, E107P, A112W, L113I, |
| | | H114Y, S170A, I171F, Q185E, N188D, |
| | | L197Y, A215G, T225A, Q234A, Q235D |
| Q9S1A6 | 2 | T28A, E41Q, N44D, N62A, L72A, A83K, |
| | | E84A, S90P, G91D, G92D, A94P, N95K, |
| | | G99P, S103M, E107P, A112W, L113I, |
| | | H114Y, S170A, T175F, Q185D, N188D, |
| | | L197Y, A215G, T225A, Q234A, Q235D |
| Q9S1A6 | 3 | E41Q, N44D, N62A, L72A, A83K, G91D, |
| | | G92D, A94P, G99P, E107P, H114W, I171M, |
| | | T175F, Q185D, L197Y, Q234A |
| Q9S1A6 | 4 | N44D, N62A, L72A, A83K, G91D, G92D, |
| | | G99E, E107P, H114Y, I171L, Q185E, |
| | | L197Y |
| Q9S1A6 | 5 | E41Q, N44D, N62A, L72A, A83K, S90P, |
| | | G91D, G92D, A94P, N95K, G99P, E107P, |
| | | A112W, L113I, H114Y, S170A, I171M, |
| | | T175F, Q185D, L197Y, A215G, T225A, |
| | | Q234A |
| Q9S1A6 | 6 | E41Q, N44D, N62A, L72A, A83K, G91D, |
| | | G92D, A94P, G99P, E107P, H114Y, I171M, |
| | | T175F, Q185E, L197Y, Q234A |
| Q9S1A6 | 7 | N44D, N62L, L72A, A83Q, G91D, G92D, |
| | | G99E, E107P, H114Y, T175F, Q185D, |
| | | L197Y |
| Q9S1A6 | 8 | N44D, N62Y, L72A, G91D, G92D, E107P, |
| | | H114Y, T175F, L197Y |
| Q9S1A6 | 9 | T28A, E41Q, N44D, N62A, L72A, L81Q, |
| | | A83K, E84A, S90P, G91D, G92D, N95K, |
| | | G99P, S103M, E107P, K108E, A112W, |
| | | L113I, H114Y, I121F, S170A, V178I, |
| | | Q185E, N188D, L197Y, A215G, T225A, |
| | | Q234A, Q235D, Q237A |
| Q9S1A6 | 10 | N44D, N62A, L72A, A83Q, G91D, G92D, |
| | | G99E, E107P, H114Y, I171L, T175V, |
| | | Q185E, L197Y |
| Q9S1A6 | 11 | T28A, E41Q, N44D, N62A, L72A, L81Q, |
| | | A83K, E84A, S90P, G91D, G92D, A94P, |
| | | N95K, G99P, S103M, E107P, K108E, |
| | | A112W, L113I, H114Y, S170A, I171A, |
| | | V178I, Q185E, N188D, L197Y, A215G, |
| | | T225A, Q234A, Q235D, Q237A |
| P28581 | 1 | E40D, K47A, E54T, Q59D, L61K, N62R, |
| | | M72A, A89S, A90K, G91D, T95K, G99E, |
| | | E107K, M137Q, E148T, N151T, K153D, |
| | | Q155E, S170A, T175V, V182I, A185D, |
| | | N186R, Q187A, L197Y, D213E, A215G, |
| | | T225A, D229N, Q233R, Q235D, S247L, |
| | | Q248R, K249Q |
| P28581 | 2 | M72A, A89S, G91E, T95K, G99E, M137Q, |
| | | E148T, N151T, K153D, S170A, T175V, |
| | | V182I, A185E, Q187R, L197Y, A215G, |
| | | T225A, Q235D, K246L |
| P28581 | 3 | M72A, A89S, G91D, T95K, G99E, M137Q, |
| | | E148T, N151T, K153D, S170A, T175V, |
| | | V182I, A185E, Q187R, L197Y, A215G, |
| | | T225A, Q235D, K246L |
| P28581 | 4 | A89S, T175V, Q187R, L197Y, A215G |
| P28581 | 5 | E40D, K47A, E54T, Q59D, L61K, N62R, |
| | | M72A, A89S, A90K, G91D, T95K, G99E, |
| | | E107K, M137Q, E148T, N151T, K153D, |
| | | Q155E, S170A, T175V, V182I, A185E, |
| | | N186R, Q187A, L197Y, D213E, A215G, |
| | | T225A, D229N, Q233R, Q235D, S247L, |
| | | Q248R, K249Q |
| P28581 | 6 | K47A, Q59D, M72A, A89S, G91D, T95K, |
| | | G99E, E107K, M137Q, E148T, N151T, |
| | | K153D, Q155E, S170A, T175V, V182I, |
| | | A185E, N186R, Q187A, L197Y, A215G, |
| | | T225A, D229N, Q235D, K246L, S247R |
| P28581 | 7 | M72A, A89S, G91E, T95K, G99E, M137Q, |
| | | E148T, N151T, K153D, S170A, T175V, |
| | | V182I, A185E, N186R, Q187R, L197Y, |
| | | A215G, T225A, Q235D, K246L |
| P28581 | 8 | A89S, G91E, G99E, K153D, T175V, Q187R, |
| | | L197Y, A215G, Q235D, K246L |
| P28581 | 9 | K47A, Q59D, M72A, A89S, G91E, T95K, |
| | | G99E, E107K, M137Q, E148T, N151T, |
| | | K153D, Q155E, S170A, T175V, V182I, |
| | | A185E, N186R, Q187A, L197Y, A215G, |
| | | T225A, D229N, Q235D, K246L, S247R |
| P28581 | 10 | A89S, G91E, G99E, K153D, T175V, Q187R, |
| | | L197Y, A215G |
| O87188 | 1 | Q44A, T45V, S46D, T53P, Y64N, E66M, |
| | | M74A, V85R, V90L, E101C, V119L, |
| | | Y147F, M150S, N153T, T162K, V180I, |
| | | S182A, I186P, E194K, M199Y, S221A, |
| | | Q237D |
| O87188 | 2 | N33H, D41E, G42S, Q44A, T45V, S46D, |
| | | T53P, L63K, Y64N, E66M, A67N, M74A, |
| | | D82K, V85R, V90L, N97Q, E101C, I105M, |
| | | K109P, T111K, V119L, L120R, G127N, |
| | | A145M, Y147F, M150S, N153T, E155K, |
| | | Q157E, Q158E, S161R, T162K, I173L, |
| | | T177V, V180I, S182A, I186P, Q189A, |
| | | N190D, E194K, Y197R, Q198E, M199Y, |
| | | S221A, D231N, Q237D, Q249R |
| O87188 | 3 | G42S, Q44A, T45V, S46D, T53P, L63K, |
| | | Y64N, E66M, M74A, D82K, V85R, V90L, |
| | | E101C, K109P, V119L, L120R, G127N, |
| | | A145M, Y147F, M150S, N153T, E155K, |
| | | Q157E, Q158E, S161R, T162K, T171A, |
| | | I173L, T177V, S182A, I186P, N190D, |
| | | E194K, Y197R, Q198E, M199Y, S221A, |
| | | D231N, Q237D, Q249R |
| O87188 | 4 | Q44A, S46D, T53P, Y64N, E66M, V85R, |
| | | V90L, V119L, Y147F, M150S, N153T, |
| | | T177V, I186P, M199Y, S221A |
| O87188 | 5 | G42E, Q44A, T45V, S46D, T53P, Y64N, |
| | | E66M, M74A, D82K, V85R, V90L, E101C, |
| | | V119L, L120Q, A145M, Y147F, M150S, |
| | | N153T, E155K, Q157E, S161R, T162K, |
| | | T177V, V180I, S182A, I186P, M199Y, |
| | | S221A, Q237D |
| O87188 | 6 | Q44A, S46D, T53P, Y64N, E66M, V85R, |
| | | V90L, V119L, N153T, I186P, M199Y, |
| | | S221A |
| O87188 | 7 | G42S, Q44A, T45V, S46D, T53P, L63K, |
| | | Y64N, E66M, M74A, D82K, V85R, V90L, |
| | | E101C, K109P, V119L, L120R, G127N, |
| | | A145M, Y147F, M150S, N153T, E155K, |
| | | Q157E, Q158E, S161R, T162K, I173L, |
| | | T177V, V180I, S182A, I186P, N190D, |
| | | E194K, Y197R, Q198E, M199Y, S221A, |
| | | D231N, Q237D, Q249R |
| O87188 | 8 | Q44A, T45V, S46D, T53P, Y64N, E66M, |
| | | M74A, V85R, V90L, E101C, V119L, |
| | | A145M, Y147F, M150S, N153T, T162K, |
| | | V180I, S182A, I186P, E194K, M199Y, |
| | | S221A, Q237D |
| O87188 | 9 | N33H, D41E, G42S, Q44A, T45V, S46D, |
| | | T53P, L63E, Y64N, E66R, A67N, M74A, |
| | | D82K, V85R, V90L, N97Q, E101C, I105M, |
| | | K109P, T111K, V119L, L120R, G127N, |
| | | A145M, Y147F, M150S, N153T, E155K, |
| | | Q157E, Q158E, S161R, T162K, T171A, |
| | | I173L, T177V, V180I, S182A, I186P, |
| | | Q189A, N190D, E194K, Y197R, Q198E, |
| | | M199Y, S221A, D231N, Q237D, Q249R |
| O87188 | 10 | G42E, Q44A, T45V, S46D, T53P, Y64N, |
| | | E66M, M74A, D82K, V85R, V90L, E101C, |
| | | V119L, L120Q, A145M, Y147F, M150S, |
| | | N153T, E155K, Q157E, S161R, T162K, |
| | | V180I, S182A, I186P, E194K, M199Y, |
| | | S221A, Q237D |
| O87188 | 11 | Q44A, T45V, S46D, T53P, Y64N, E66M, |
| | | V85R, V90L, V119L, Y147F, M150S, |
| | | N153T, T177V, S182A, I186P, M199Y, |
| | | S221A, Q237D |
| O87188 | 12 | Q44S, T45I, S46D, T53P, Y64N, E66M, |
| | | V85R, V90L, V119L, Y147F, M150S, |
| | | N153T, T177V, I186P, M199Y, S221A |
| Q50EL0 | 1 | L28R, C50Q, T57F, A74R, S125A, H128R, |
| | | K134P, H146N, H160K, A185T, T188V, |
| | | V211I, V216Q, S237P, A285P, S292E, |
| | | V319Q, Q335P, E341Q, T378E, A387Q, |
| | | R406N, T407K |
| Q50EL0 | 2 | L28R, C50Q, T57F, S125A, H128R, K134P, |
| | | H146N, S152R, H160K, A185V, T188V, |
| | | V211I, V216Q, S237P, A285P, S292E, |
| | | S304P, V319Q, Q335P, A387Q, T407K |
| Q50EL0 | 3 | A3T, L28R, C50Q, T57F, C68P, A74R, |
| | | N96H, S97N, S125A, H128R, K134P, |
| | | H146N, S152R, H160K, A185T, T188V, |
| | | T202S, V211I, V216Q, S237P, A285P, |
| | | S292E, V319Q, Q335P, V339Y, E341Q, |
| | | T378E, A387Q, R406N, T407Q |
| Q50EL0 | 4 | K2Q, A3T, L28R, T43D, T47S, T48L, C50E, |
| | | T57F, C61H, C68P, A74R, N96H, S97N, |
| | | K114N, S125A, H128R, L130K, K134P, |
| | | H146N, S152R, S155A, H160K, A185V, |
| | | T188V, T202S, V211I, V216Q, N226D, |
| | | S237P, M266N, A285P, S292E, V319Q, |
| | | I320V, Q335P, V339Y, E341Q, T378E, |
| | | A387Q, R406N, T407Q |
| Q50EL0 | 5 | A3T, L28R, C50E, T57F, C68P, A74R, |
| | | S97N, S125A, H128R, K134P, H146N, |
| | | S152R, H160K, A185V, T188V, T202S, |
| | | V211I, V216Q, S237P, A285P, S292E, |
| | | S304P, V319Q, Q335P, V339Y, E341Q, |
| | | T378E, A387Q, R406N, T407K |
| Q50EL0 | 6 | K2Q, A3T, L28R, T43D, T47S, T48L, |
| | | C50Q, T57F, C61H, C68P, A74R, S97N, |
| | | K114N, S125A, H128Q, L130K, K134P, |
| | | H146N, S152R, S155A, H160K, A185T, |
| | | T188V, T202S, V211I, V216Q, N226D, |
| | | S237P, M266N, A285P, S292E, S304P, |
| | | V319Q, I320V, Q335P, V339Y, E341Q, |
| | | T378E, A387Q, R406N, T407K |
| Q50EL0 | 7 | L28R, C50Q, T57F, A74R, S125A, H128R, |
| | | K134P, H146N, H160K, A185V, T188V, |
| | | V211I, V216Q, S237P, A285P, S292E, |
| | | S304P, V319Q, Q335P, T378E, A387Q, |
| | | R406N, T407K |
| Q50EL0 | 8 | K2Q, A3T, A9N, R17K, A18Y, L28R, |
| | | T43M, T47S, T48L, C50Q, T57F, C61H, |
| | | C68P, A74R, N96H, S97N, K114N, S125A, |
| | | H128Q, L130K, K134P, H146N, S152R, |
| | | S155A, F157Y, H160K, G167D, A185T, |
| | | T188A, T202S, V207I, G209E, V211I, |
| | | R213K, V216Q, N226D, S237P, A254D, |
| | | Q265R, M266Q, A285P, S292E, S304P, |
| | | V319Q, I320V, Q335P, N336G, V339Y, |
| | | E341Q, S370T, T378E, T379A, A387Q, |
| | | D390S, D405N, R406N, T407Q, V453K |
| Q50EL0 | 9 | C50E, T57F, S125A, H128R, K134P, |
| | | A185T, S237P, A285P, S292E, S304P, |
| | | Q335P, T407K |
| Q50EL0 | 10 | L28R, C50E, T57F, S125A, H128R, K134P, |
| | | H146N, H160K, A185V, T188V, V211I, |
| | | V216Q, S237P, A285P, S292E, S304P, |
| | | V319Q, Q335P, A387Q, T407K |
| Q50EL0 | 11 | L28R, C50E, T57F, S125A, H128R, K134P, |
| | | A185T, S237P, A285P, S292E, S304P, |
| | | Q335P, A387Q, T407K |
| Q50EL0 | 12 | K2Q, A3T, A9N, R17K, A18Y, L28R, |
| | | T43D, T47S, T48L, C50E, T57F, C61H, |
| | | C68P, A74R, N96H, S97N, K114N, S125A, |
| | | H128R, L130K, K134P, H146N, S152R, |
| | | S155A, F157Y, H160K, G167D, A185V, |
| | | T188A, T202S, V207I, G209E, V211I, |
| | | R213K, V216Q, N226D, S237P, A254D, |
| | | Q265R, M266Q, A285P, S292E, S304P, |
| | | V319Q, I320V, Q335P, N336G, V339Y, |
| | | E341Q, S370T, T378E, A387Q, D390S, |
| | | D405N, R406N, T407Q, V453K |
| Q50EL0 | 13 | C50E, T57F, S125A, H128R, K134P, |
| | | A185T, S237P, A285P, S292E, Q335P, |
| | | T407K |
| P42327 | 1 | K10G, A12P, E19P, R20I, L23I, E24G, |
| | | E25P, I51V, A71G, K72P, K75T, I77V, |
| | | E94H, Q102W, L110Q, G112T, Y120F, |
| | | A132V, V143A, T153V, I179M, L181V, |
| | | S197D, S201L, D206K, A212T, G215A, |
| | | H225Q, D226K, V228I, S236V, K241P, |
| | | Q249R, K252R, L257I, V259L, N264P, |
| | | A265G, L267M, V279I, S280T, K282R, |
| | | K290Q, A311R, E312R, K335D |
| P42327 | 2 | A12P, E19P, R20I, L23I, E24G, E25R, I51V, |
| | | A71G, K72P, K75T, E94R, Q102W, G112T, |
| | | A132V, V143A, L181V, S197D, S201L, |
| | | A212T, G215A, D226K, S236V, K241P, |
| | | Q249R, K252R, V259L, N264P, A265G, |
| | | S280T, K282R, K290Q, A311R, E312K, |
| | | K335D |
| P42327 | 3 | N7R, K10G, A12P, E19P, R20I, L23I, E24G, |
| | | E25P, I51V, A71G, K72P, K75T, I77V, |
| | | E94H, Q102W, L110Q, G112T, Y120F, |
| | | A132V, V143A, T153V, I179M, L181V, |
| | | S197D, S201L, D206K, A212T, 1213V, |
| | | G215A, H225Q, D226K, V228I, S236V, |
| | | K241P, Q249R, K252R, L257V, V259L, |
| | | N264P, A265G, L267M, V279I, S280T, |
| | | V281I, K282R, K290Q, A311R, E312R, |
| | | N329D, K335D |
| P42327 | 4 | A12P, E19P, R20I, L23I, E24G, E25R, |
| | | A71G, K72P, E94H, Q102W, G112T, |
| | | A132V, V143A, V159E, L181V, S201L, |
| | | A212T, G215A, S236V, K241P, Q249R, |
| | | K252R, V259L, N264P, A265G, S280T, |
| | | K282R, K290Q, E312R, K335D |
| P42327 | 5 | A12P, E19P, R20I, L23I, E24G, E25R, I51V, |
| | | A71G, K72P, K75T, E94H, Q102W, G112T, |
| | | A132V, V143A, V159E, L181V, S197D, |
| | | S201L, A212T, G215A, D226K, S236V, |
| | | K241P, Q249R, K252R, L257V, V259L, |
| | | N264P, A265G, S280T, K282R, K290Q, |
| | | A311R, E312R, K335D |
| P42327 | 6 | A12P, E19P, R20I, L23I, E24G, E25P, I51V, |
| | | A71G, K72P, K75T, E94H, Q102W, L110Q, |
| | | G112T, A132V, V143A, L181V, S197D, |
| | | S201L, D206K, A212T, G215A, D226K, |
| | | V228I, S236V, K241P, Q249R, K252R, |
| | | V259L, N264P, A265G, S280T, K282R, |
| | | K290Q, A311R, E312R, K335D |
| P42327 | 7 | A12P, E19P, R20I, L23I, E24G, E25R, I51V, |
| | | A71G, K72P, E94H, Q102W, G112T, |
| | | A132V, V143A, L181V, S201L, A212T, |
| | | G215A, S236V, K241P, Q249R, K252R, |
| | | V259L, N264P, A265G, S280T, K282R, |
| | | K290Q, E312R, K335D |
| P42327 | 8 | K10G, A12P, E19P, R20I, L23I, E24G, |
| | | E25P, I51V, A71G, K72P, K75T, I77V, |
| | | E94H, Q102W, L110Q, G112T, Y120F, |
| | | A132V, V143A, T153V, I179M, L181V, |
| | | S197D, S201L, D206K, A212T, G215A, |
| | | H225Q, D226K, V228I, S236V, K241P, |
| | | Q249R, K252R, V259L, N264P, A265G, |
| | | L267M, V279I, S280T, K282R, K290Q, |
| | | M292L, A311R, E312R, K335D |
| P42327 | 9 | K10G, A12P, E19P, R20I, L23I, E24G, |
| | | E25P, V28I, I51V, A71G, K72P, K75T, |
| | | I77V, E94H, Q102R, L110Q, G112T, |
| | | Y120F, A132V, V143A, T153V, V159E, |
| | | I179M, L181V, S197D, S201L, D206K, |
| | | A212T, 1213V, G215A, H225Q, D226K, |
| | | V228I, S236V, K241P, Q249R, K252R, |
| | | L257I, V259L, N264P, A265G, L267M, |
| | | V279I, S280T, V281I, K282R, K290Q, |
| | | A311R, E312R, N329D, K335D |
| P42327 | 10 | A12P, E19P, R20I, L23I, E24G, E25R, I51V, |
| | | A71G, K72P, E94H, Q102W, G112T, |
| | | A132V, V143A, V159E, L181V, S201L, |
| | | A212T, G215A, S236V, K241P, Q249R, |
| | | K252R, V259L, N264P, A265G, S280T, |
| | | K282R, K290Q, E312R, K335D |
| P42327 | 11 | N7T, K10G, A12P, E19P, R20I, K22T, L23I, |
| | | E24G, E25P, V28I, I51V, E69A, A71G, |
| | | K72P, K75T, I77V, Y89W, E94H, Q102R, |
| | | L110Q, G112T, Y120F, P127H, A132V, |
| | | V143A, T153V, V159E, I179M, L181V, |
| | | S197D, S201L, D206K, A212T, 1213V, |
| | | G215A, K222Q, H225Q, D226K, V228I, |
| | | S236V, K241P, Q249R, K252R, V259L, |
| | | N264P, A265G, L267M, V279I, S280T, |
| | | V281I, K282R, K290Q, A311R, E312R, |
| | | E318D, N329D, K335D |
| P42327 | 12 | N7T, K10G, A12P, E19P, R20I, K22T, L23I, |
| | | E24G, E25P, V28I, I51V, E69A, A71G, |
| | | K72P, K75T, I77V, Y89W, E94H, L99R, |
| | | Q102W, L110Q, G112T, Y120F, P127H, |
| | | V141L, V143A, T153V, V159E, A162T, |
| | | I179M, L181V, S197D, S201L, D206K, |
| | | A212T, 1213V, G215A, K222Q, H225Q, |
| | | D226K, V228I, S236V, K241P, Q249R, |
| | | K252R, V259L, N264P, A265G, L267M, |
| | | V279I, S280T, K282R, K290Q, M292L, |
| | | A311R, E312R, E318D, N329D, K335D |
| P42327 | 13 | A12P, E19P, R20I, L23I, E24G, E25P, V28I, |
| | | I51V, A71G, K72P, K75T, E94R, Q102W, |
| | | L110Q, G112T, A132V, V143A, L181V, |
| | | S197D, S201L, D206K, A212T, G215A, |
| | | H225R, D226K, V228I, S236V, K241P, |
| | | Q249R, K252R, V259L, N264P, A265G, |
| | | S280T, K282R, K290Q, A311R, E312K, |
| | | K335D |
| Q0RV69 | 1 | V36A, K44E, A46L, D70P, S73T, I77V, |
| | | V83A, T91R, S97T, Q99M, L105A, N108G, |
| | | V110A, T111A, E113C, L114Q, N116D, |
| | | A132C, A133M, T136A, Q139E, F140Y, |
| | | S141A, T154D, V160A, S161A, T164G, |
| | | E179G, A182P, A187V, R200Q, A201G, |
| | | N206G, G208R, V210I, A211V, F222W, |
| | | S226F, F230H, T233A, H239A, D241L, |
| | | Y247W, T254V, V255I, V261G, F270M, |
| | | E271R |
| Q0RV69 | 2 | K44D, D70P, S73T, V83A, L105A, V110A, |
| | | T111A, E113C, L114Q, N116D, A133M, |
| | | T136A, F140Y, S141T, T154D, V160A, |
| | | T164S, A182P, R200Q, N206G, A211V, |
| | | S226F, F230H, T233A, H239A, D241L, |
| | | Y247W, V261G, E271R |
| Q0RV69 | 3 | K44E, D70P, S73T, I77V, V83A, L105A, |
| | | V110A, T111A, E113C, L114M, N116D, |
| | | A132C, A133M, T136A, F140Y, S141T, |
| | | T154D, V160A, T164S, A182P, R200Q, |
| | | N206G, G208R, A211V, F222M, S226F, |
| | | F230H, T233A, H239A, D241L, Y247W, |
| | | V261G, F270M, E271R |
| Q0RV69 | 4 | V36A, K44D, A46L, D70P, S73T, I77V, |
| | | V83A, S97T, Q99M, L105A, V110A, T111A |
| | | E113C, L114Q, N116D, A132C, A133M, |
| | | T136A, Q139E, F140Y, S141A, T154D, |
| | | V160A, S161A, T164G, A182P, A187I, |
| | | R200Q, A201G, N206G, G208R, A211V, |
| | | F222Q, S226F, F230H, T233A, H239A, |
| | | D241L, Y247W, T254V, V255I, V261G, |
| | | F270M, E271R |
| Q0RV69 | 5 | V36A, K44D, A46L, D70P, S73T, I77V, |
| | | V83A, T91R, S97T, Q99M, S100Q, L105A, |
| | | N108G, V110A, T111A, E113C, L114M, |
| | | N116D, I128V, A132C, A133M, T136A, |
| | | Q139E, F140Y, S141A, T154D, W159K, |
| | | V160A, S161A, T164G, S173A, A177T, |
| | | G178A, E179G, K181Q, A182P, A187V, |
| | | V199I, R200Q, A201G, V203R, N206G, |
| | | G208R, V210I, A211V, 1213V, S218E, |
| | | F222W, Q225K, S226F, D229T, F230H, |
| | | Y232F, T233A, A235M, H239A, D241L, |
| | | Y247W, V249Q, T254V, V255I, V261G, |
| | | F270M, E271R |
| Q0RV69 | 6 | V36A, K44D, A46L, D70P, S73T, I77V, |
| | | V83A, T91R, S97T, Q99M, S100Q, L105A, |
| | | N108G, V110A, T111A, E113C, L114M, |
| | | N116D, I128V, A132C, A133M, T136A, |
| | | Q139E, F140Y, S141A, T154D, W159K, |
| | | V160A, S161A, T164G, S173A, A177T, |
| | | G178A, E179G, K181Q, A182P, A187V, |
| | | V199I, R200Q, A201G, V203R, N206G, |
| | | G208R, V210I, A211V, 1213V, S218D, |
| | | L220F, F222Q, Q225K, S226F, D229T, |
| | | F230H, Y232F, T233A, A235M, H239A, |
| | | D241L, Y247W, V249Q, T254V, V255I, |
| | | V261G, F270M, E271R |
| Q0RV69 | 7 | V36A, K44E, D70P, S73T, 177V, V83A, |
| | | S97T, Q99M, L105A, V110A, T111A, |
| | | E113A, L114M, N116D, A132C, A133M, |
| | | T136A, Q139E, F140Y, S141A, T154D, |
| | | V160A, T164S, A182P, R200Q, A201G, |
| | | N206G, G208R, A211V, F222W, S226F, |
| | | F230H, T233A, H239A, D241L, Y247W, |
| | | T254V, V255I, V261G, F270M, E271R |
| Q0RV69 | 8 | D70P, S73T, V83A, L105A, V110A, T111A, |
| | | E113C, L114Q, N116D, A133M, T136A, |
| | | F140Y, S141T, T154D, V160A, T164S, |
| | | A182P, R200Q, N206G, A211V, S226F, |
| | | F230H, T233A, H239A, D241L, V261G, |
| | | E271R |
| Q0RV69 | 9 | V36A, K44E, D70P, S73T, I77V, V83A, |
| | | S97T, Q99M, L105A, V110A, T111A, |
| | | E113C, L114Q, N116D, A132C, A133M, |
| | | T136A, Q139E, F140Y, S141A, T154D, |
| | | V160A, T164S, A182P, A187I, R200Q, |
| | | A201G, N206G, G208R, A211V, F222M, |
| | | S226F, F230H, T233A, H239A, D241L, |
| | | Y247W, T254V, V255I, V261G, F270M, |
| | | E271R |
| Q0RV69 | 10 | V36A, K44D, A46L, D70P, S73T, I77V, |
| | | V83A, T91R, S97T, Q99M, L105A, N108G, |
| | | V110A, T111A, E113C, L114Q, N116D, |
| | | A132C, A133M, T136A, Q139E, F140Y, |
| | | S141A, T154D, V160A, S161A, T164G, |
| | | E179G, A182P, R200Q, A201G, N206G, |
| | | G208R, V210I, A211V, F222M, S226F, |
| | | F230H, T233A, H239A, D241L, Y247W, |
| | | T254V, V255I, V261G, F270M, E271R |
| Q0RV69 | 11 | K44D, D70P, S73T, V83C, L105A, V110A, |
| | | T111A, E113C, L114Q, N116D, A133M, |
| | | T136A, F140Y, S141T, T154D, V160A, |
| | | T164S, A182P, R200Q, N206G, A211V, |
| | | S226F, F230H, T233A, H239A, D241L, |
| | | Y247W, V261G, E271R |
| Q0RV69 | 12 | K44E, D70P, S73T, I77V, V83A, L105A, |
| | | V110A, T111A, E113A, L114Q, N116D, |
| | | A132C, A133M, T136A, F140Y, S141T, |
| | | T154D, V160A, T164S, A182P, R200Q, |
| | | N206G, G208R, A211V, F222M, S226F, |
| | | F230H, T233A, H239A, D241L, Y247W, |
| | | V261G, F270M, E271R |
| Q0RV69 | 13 | D70P, S73T, V83A, L105A, V110A, T111A, |
| | | E113C, L114Q, N116D, A133M, T136A, |
| | | F140Y, S141T, T154D, V160A, T164G, |
| | | A182P, R200Q, N206G, A211V, S226F, |
| | | F230H, T233A, H239A, D241L, V261G, |
| | | E271R |
| Q0RV69 | 14 | V36A, K44E, A46L, D70P, S73T, I77V, |
| | | V83A, S97T, Q99M, L105A, V110A, |
| | | T111A, E113C, L114Q, N116D, A132C, |
| | | A133M, T136A, Q139E, F140Y, S141A, |
| | | T154D, V160A, S161A, T164S, A182P, |
| | | R200Q, A201G, N206G, G208R, A211V, |
| | | F222M, S226F, F230H, T233A, H239A, |
| | | D241L, Y247W, T254V, V255I, V261G, |
| | | F270M, E271R |
| Q7BEK9 | 1 | N41Q, I55P, G56D, L72A, N95K, S110A, |
| | | N119R, R160K, T175I, Q235D |
| Q7BEK9 | 2 | N41Q, I55P, G56D, A59D, L72A, N95K, |
| | | S110A, N119R, N151T, D156K, R160K, |
| | | T175M, K192Q, Q235D, N246L |
| Q7BEK9 | 3 | D40S, N41Q, A42S, I55P, G56D, A59D, |
| | | L61K, L72A, L81Q, N95K, K108E, S110A, |
| | | T118K, N119R, N151T, D156K, R160K, |
| | | S170A, T175I, K192Q, Q235D, N246L |
| Q7BEK9 | 4 | D40S, N41Q, A42S, I55P, G56D, A59D, |
| | | L61K, L72K, L81Q, N95K, K108E, S110A, |
| | | T118R, N119R, N151T, D156K, R160K, |
| | | S170A, T175I, K192Q, Q235D, N246L |
| Q7BEK9 | 5 | N41Q, L72A, S110A, T175I, Q235D |
| Q7BEK9 | 6 | L72A, S110A, T175I |
| Q7BEK9 | 7 | L72K, S110A, T175I |
| Q7BEK9 | 8 | N41Q, I55P, G56D, A59D, L72A, N95K, |
| | | S110A, N119R, N151T, D156K, R160T, |
| | | T175M, K192Q, Q235D, N246L |
| Q7BEK9 | 9 | N41Q, I55P, G56D, L72A, N95K, S110A, |
| | | N119R, N151T, D156Q, R160K, T175I, |
| | | Q235D, N246L |
| Q5P8T0 | 1 | A51G, V54T, D76T, A79K, A83R, E96P, |
| | | A97P, I99M, C115A, L117Y, S128A, |
| | | N154E, S162T, T185R, H198R, S205D, |
| | | S212R, Q213N, Q215D, A217V, S218K, |
| | | C227R, V266E, Q277A, N291E, T300H, |
| | | F303K, S309V, L312F, T314Q, L321H, |
| | | Q322L, S332C |
| Q5P8T0 | 2 | S8A, L45Y, A51G, V54T, N57G, I71V, |
| | | D76T, A79K, A83R, H91D, E96P, A97P, |
| | | I99M, C115A, L117Y, R120A, S128A, |
| | | T142C, I152V, Y153S, N154H, A159P, |
| | | S162T, T185R, H198R, S205D, C211Q, |
| | | S212R, Q213N, Q215D, A217V, S218K, |
| | | C227R, V266E, K274M, R275K, Q277A, |
| | | N291E, T300H, F303K, S309V, T311S, |
| | | L312F, T314E, L321H, Q322L, K323E, |
| | | S332C |
| Q5P8T0 | 3 | K2R, S8A, L45Y, A51G, V54T, N57G, |
| | | I71V, D76T, A79K, A83R, H91D, E96P, |
| | | A97P, I99A, C115A, L117Y, R120A, |
| | | S128A, T142C, I152V, Y153A, N154E, |
| | | A159P, S162T, T185R, A188V, H198R, |
| | | S205D, C211Q, S212R, Q213D, Q215D, |
| | | A217R, S218K, C227R, V266E, K274M, |
| | | R275K, Q277A, Q289M, N291E, T300H, |
| | | F303K, S309V, T311S, L312F, T314E, |
| | | L321H, Q322L, K323E, A324S, S332C |
| Q5P8T0 | 4 | A51G, V54T, D76T, A79K, A83R, E96P, |
| | | A97P, I99M, C115A, L117Y, R120A, |
| | | S128A, T142C, Y153S, N154D, A159P, |
| | | S162T, T185R, H198R, S205D, S212R, |
| | | Q213N, Q215D, A217V, S218K, C227R, |
| | | V266E, Q277A, N291E, T300H, F303K, |
| | | S309V, L312F, T314Q, L321H, Q322L, |
| | | S332C |
| Q5P8T0 | 5 | K2R, S8A, R14N, E16T, L45Y, G46L, S49T, |
| | | A51G, V54T, A55E, N57G, I71V, D76T, |
| | | A79K, A83R, H91D, L93I, E96P, A97P, |
| | | I99A, A100E, V110I, C115A, L117Y, |
| | | R120A, T123D, S128A, Q136E, T142C, |
| | | I152V, Y153G, N154E, A159P, S162T, |
| | | T185R, A188V, H198R, R200K, S205D, |
| | | C211Q, S212T, Q213N, Q215D, A217R, |
| | | S218K, C227R, V266E, K274M, R275K, |
| | | Q277A, Q289M, N291E, T300H, F303R, |
| | | S309V, T311S, L312F, T314Q, L321H, |
| | | Q322L, K323Q, A324S, S332C |
| Q5P8T0 | 6 | A51G, V54T, D76T, A79K, A83R, E96P, |
| | | A97P, I99M, C115A, L117Y, R120F, |
| | | S128A, N154E, S162T, T185R, H198R, |
| | | S205D, Q213N, Q215D, A217V, S218K, |
| | | C227R, V266E, Q277A, N291E, T300H, |
| | | F303K, S309V, L312F, T314Q, L321H, |
| | | Q322L, S332C |
| Q5P8T0 | 7 | S8A, L45Y, A51G, V54T, N57G, I71V, |
| | | D76T, A79K, A83R, H91D, E96P, A97P, |
| | | I99M, C115A, L117Y, R120A, S128A, |
| | | T142C, I152V, Y153S, N154E, A159P, |
| | | S162T, T185R, H198R, S205D, C211Q, |
| | | S212R, Q213N, Q215D, A217R, S218K, |
| | | C227R, V266E, K274M, R275K, Q277A, |
| | | Q289M, N291E, T300H, F303K, S309V, |
| | | T311S, L312F, T314E, L321H, K323E, |
| | | S332C |
| Q5P8T0 | 8 | A51G, V54T, D76T, A83R, E96P, A97P, |
| | | L117Y, S128A, N154E, S162T, T185R, |
| | | H198R, S205D, Q213N, Q215D, A217V, |
| | | S218K, C227R, V266E, Q277A, N291E, |
| | | T300H, F303K, S309V, L312F, T314Q, |
| | | L321H, S332C |
| Q5P8T0 | 9 | A51G, V54T, D76T, A79K, A83R, E96P, |
| | | A97P, I99M, C115A, L117Y, R120A, |
| | | S128A, T142C, Y153S, N154D, A159P, |
| | | S162T, T185R, H198R, S205D, S212R, |
| | | Q213N, Q215D, A217V, S218K, C227R, |
| | | V266E, Q277A, N291E, T300H, F303K, |
| | | S309V, T311P, L312F, T314Q, L321H, |
| | | Q322L, S332C |
| Q5P8T0 | 10 | K2R, S8A, R14N, E16T, L45Y, G46L, S49T, |
| | | A51G, V54T, A55E, N57G, I71V, D76T, |
| | | A79K, A83R, H91D, L93I, E96P, A97P, |
| | | I99A, A100E, V110I, C115A, L117Y, |
| | | R120A, T123D, S128A, Q136E, T142C, |
| | | I152V, Y153A, N154E, A159P, S162T, |
| | | T185R, A188V, H198R, S205D, C211Q, |
| | | S212Q, Q213N, Q215D, A217R, S218K, |
| | | C227R, V266E, K274M, R275K, Q277A, |
| | | Q289M, N291E, T300H, F303R, S309V, |
| | | T311S, L312F, T314Q, L321H, Q322L, |
| | | K323E, A324S, S332C |
| Q5P8T0 | 11 | A51G, V54T, D76T, A83R, E96P, A97P, |
| | | C115R, L117Y, S128A, N154E, S162T, |
| | | T185R, H198R, S205D, Q213N, Q215D, |
| | | A217V, S218K, C227R, V266E, Q277A, |
| | | N291E, T300H, F303K, S309V, L312F, |
| | | T314E, L321H, S332C |
| Q5P8T0 | 12 | A51G, V54T, D76T, A83R, E96P, A97P, |
| | | L117Y, N154E, S162T, T185R, H198R, |
| | | S205D, Q213D, Q215D, S218K, C227R, |
| | | V266E, Q277A, T300H, F303K, S309V, |
| | | L312F, T314Q, L321H, S332C |
| Q5P8T0 | 13 | A51G, V54T, D76T, A83R, E96P, A97P, |
| | | C115R, L117Y, N154E, S162T, T185R, |
| | | H198R, S205D, Q213D, Q215D, S218K, |
| | | C227R, V266E, Q277A, T300H, F303K, |
| | | S309V, L312F, T314Q, L321H, S332C |
| W6R2Y2 | 1 | F35Y, E54H, S78P, E87D, Q102L, L128V, |
| | | S158P, G168A, G172E, M173W |
| W6R2Y2 | 2 | A34G, F35Y, K49Q, S52K, E54H, E57D, |
| | | S78P, E83R, E87D, Q102L, S118A, L128V, |
| | | V138Q, T142Q, T149K, S158P, G168A, |
| | | G172E, M173L, E187P, I188Q |
| W6R2Y2 | 3 | A34G, F35Y, S52K, E54H, E57D, S78P, |
| | | E87D, Q102L, S118A, L128V, T142Q, |
| | | S158P, G168A, G172E, M173L, E187P, |
| | | I188Q |
| W6R2Y2 | 4 | A34G, F35Y, S52K, E54H, E57D, S78P, |
| | | E87D, Q102L, S118A, L128V, V138Q, |
| | | T142Q, T149K, S158P, G168A, G172E, |
| | | M173L, E187P, I188Q, L189I |
| W6R2Y2 | 5 | A34G, F35Y, K49Q, S52K, E54H, E57D, |
| | | S78P, E83R, E87D, Q102L, V115I, S118A, |
| | | L128V, V138Q, T142Q, T149E, E153K, |
| | | S158P, G168A, V170N, G172E, M173L, |
| | | A182S, E183A, E187A, I188Q, T196Q |
| W6R2Y2 | 6 | A34G, F35Y, K49Q, S52K, E54H, E57D, |
| | | S78P, E83R, E87D, Q102L, V115I, S118A, |
| | | L128V, V138R, T142E, T149A, E153K, |
| | | S158P, G168A, V170N, G172E, M173L, |
| | | A182S, E183A, E187A, I188Q, T196Q |
| W6R2Y2 | 7 | A34G, F35Y, S52K, E54H, E57D, S78P, |
| | | E87D, Q102L, S118A, L128V, V138Q, |
| | | T142Q, T149K, S158P, G168A, G172E, |
| | | M173L, E187P, I188Q |
| W6R2Y2 | 8 | F35Y, E54H, E57D, S78P, E87D, Q102L, |
| | | L128V, T142Q, S158P, G168A, G172E, |
| | | M173L, E187P, L189I |
| W6R2Y2 | 9 | F35Y, E54H, S78P, E87D, Q102L, L128V, |
| | | S158P, G168A, G172E, M173W, L189I |
| W6R2Y2 | 10 | A34G, F35Y, K49Q, S52K, E54H, E57D, |
| | | S78P, E83R, E87D, Q102L, S118A, L128V, |
| | | V138R, T142E, T149K, S158P, G168A, |
| | | G172E, M173L, E187P, I188Q |
| W6R2Y2 | 11 | F35Y, E54H, S78P, E87D, Q102L, L128V, |
| | | S158P, G168A |
| P42588 | 1 | A12E, H16R, H28E, A33Q, K43R, V102K, |
| | | S105K, N109E, A112D, Q119N, L121F, |
| | | D123N, K131H, A135E, T137A, K140D, |
| | | S144V, S153A, K162R, S194E, T195E, |
| | | F196Y, N214D, M218L, T220K, N223E, |
| | | L248I, T255R, F264Y, M268L, K282R, |
| | | Q292V, A299G, L344I, N348H, Q353E, |
| | | A357E, Q361E, M365Y, V381I, Q382R, |
| | | M389L, N398E, S406A, T418S, T424V, |
| | | I435D, C438I, E439D, L440K, M452I |
| P42588 | 2 | N109E, L121F, K140D, S144V, S153A, |
| | | T195E, M218L, N223E, L248I, T255R, |
| | | K282R, Q292V, A299G, N348H, M365Y, |
| | | Q382R, M389L, S406K, T424V, I435D, |
| | | C438L |
| P42588 | 3 | N109E, L121F, A135E, K140D, S144V, |
| | | S153A, K162R, T195E, M218L, T220K, |
| | | N223E, L248I, T255R, K282R, Q292V, |
| | | A299G, N348H, M365Y, Q382R, M389L, |
| | | S406A, T424V, I435D, C438I |
| P42588 | 4 | A12E, H28E, A33Q, S105K, N109E, L121F, |
| | | K131H, A135E, T137A, K140D, S144V, |
| | | S153A, K162R, S194E, T195E, N214D, |
| | | M218L, T220K, N223E, L248I, T255R, |
| | | F264H, K282R, Q292V, A299G, N348H, |
| | | Q353E, Q361E, M365Y, V381I, Q382R, |
| | | M389L, S406A, T424V, I435D, C438I, |
| | | L440Q |
| P42588 | 5 | N109E, L121F, K140D, S144V, S153A, |
| | | T195E, M218L, N223E, L248I, T255R, |
| | | K282R, Q292V, A299G, N348H, M365Y, |
| | | Q382R, M389L, S406K, T424V, I435D, |
| | | C438I |
| P42588 | 6 | A12E, S105K, N109E, L121F, K131H, |
| | | A135E, T137A, K140D, S144V, S153A, |
| | | K162R, S194E, T195E, N214D, M218L, |
| | | T220K, N223E, L248I, T255R, F264H, |
| | | K282R, Q292V, A299G, N348H, M365Y, |
| | | Q382R, M389L, S406A, T424V, I435D, |
| | | C438I, L440R |
| P42588 | 7 | N109E, L121F, A135E, K140D, S144V, |
| | | S153A, K162R, T195E, T220K, N223E, |
| | | L248I, T255R, K282R, Q292V, A299G, |
| | | N348H, M365Y, Q382R, M389L, S406A, |
| | | T424V, I435D, C438I |
| P42588 | 8 | A12E, H28E, A33Q, S105K, N109E, L121F, |
| | | K131H, A135E, T137A, K140D, S144V, |
| | | S153A, K162R, S194E, T195E, N214D, |
| | | M218L, T220K, N223E, L248I, T255R, |
| | | F264Y, K282R, Q292V, A299G, N348H, |
| | | Q353E, Q361E, M365Y, V381I, Q382R, |
| | | M389L, S406A, T424V, I435D, C438I, |
| | | L440Q |
| P42588 | 9 | A12E, S105K, N109E, L121F, K131H, |
| | | A135E, T137A, K140D, S144V, S153A, |
| | | K162R, S194E, T195E, N214D, M218L, |
| | | T220K, N223E, L248I, T255R, K282R, |
| | | Q292V, A299G, N348H, M365Y, Q382R, |
| | | M389L, S406A, T424V, I435D, C438I |
| P42588 | 10 | L121F, S144V, S153A, T195E, N223E, |
| | | L248I, T255R, K282R, Q292V, A299G, |
| | | N348H, M365Y, M389L, S406K, T424V, |
| | | I435D, C438I |
| P42588 | 11 | A12E, A15Q, H16R, R24D, H28E, A33Q, |
| | | R36K, E44K, F50Y, A59K, V102K, S105K, |
| | | N109E, A112D, Q114L, Q119N, L121F, |
| | | D123N, K131H, A135E, T137A, K140D, |
| | | S144V, S153A, K162R, A175T, A192S, |
| | | S194E, T195E, F196Y, N214D, M218L, |
| | | T220K, N223E, L248I, T255R, F264H, |
| | | M268L, K282R, N290G, Q292V, A299G, |
| | | L322F, L344I, T346A, N348K, Q353E, |
| | | A357E, Q361E, M365Y, V381I, Q382R, |
| | | M389L, N398E, G401A, S406A, R412G, |
| | | T418S, A422S, T424V, I435D, C438I, |
| | | E439D, L440K, M452I, S455Q |
| P42588 | 12 | A12E, A15Q, H16R, R24D, H28E, A33Q, |
| | | R36K, K43R, F50Y, A59K, F84Y, V102K, |
| | | S105K, N109E, A112D, Q114L, Q119N, |
| | | L121F, D123N, K131H, A135E, K140D, |
| | | S144V, S153A, K162R, A175T, A192S, |
| | | S194E, T195E, F196Y, N214D, M218L, |
| | | T220K, N223E, L248I, T255R, F264Y, |
| | | M268L, K282R, N290G, Q292V, A299G, |
| | | L322F, L344I, T346A, N348K, Q353E, |
| | | A357E, Q361E, M365Y, V381I, Q382R, |
| | | M389L, N398D, G401A, S406A, R412G, |
| | | T418S, A422S, T424V, I435D, C438I, |
| | | E439D, L440K, M452I, S455Q |
| P42588 | 13 | A12E, H16R, H28E, A33Q, K43R, V102K, |
| | | S105K, N109E, A112D, Q119N, L121F, |
| | | D123N, K131H, A135E, T137A, K140D, |
| | | S144V, S153A, K162R, S194E, T195E, |
| | | F196Y, N214D, M218L, T220K, N223E, |
| | | L248I, T255R, F264Y, M268L, K282R, |
| | | Q292V, A299G, L344I, N348H, Q353E, |
| | | A357E, Q361E, M365R, V381I, Q382R, |
| | | M389L, N398E, S406A, T418S, T424V, |
| | | I435D, C438I, E439D, L440K, M452I |
| P42588 | 14 | L121F, S144V, S153A, T195E, N223E, |
| | | L248I, T255R, K282R, Q292V, A299G, |
| | | N348H, M365Y, M389L, S406K, T424V, |
| | | I435D |

Any patent, patent application publication, or scientific publication, cited herein, is incorporated by reference herein in its entirety. The examples are presented to more fully illustrate embodiments of the invention. They should in no way be construed, however, as limiting the broad scope of the invention. While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A pyruvate transaminase variant of a wild-type pyruvate transaminase from Vibrio fluvialis of SEQ ID NO: 39 comprising one or more mutations selected from A74E, Y76M, V103M, S108M, F130Y, Q196E, Q210L, A214P, G219A, L243Q, S304A, K305Q, T309E, A313K or A313R, E315G, E316T, A323Y, K335E, N342D, N405E; T406A, T408E, T408F or T408Q and E436D.

2. A pyruvate transaminase variant of a wild-type pyruvate transaminase from Vibrio fluvialis of SEQ ID NO: 39 comprising mutations:
(a) S108M, F130Y, A214P, L243Q, S304A, E315G, A323Y, N342D, T408E and E436D;
(b) S108M, F130Y, A214P, L243Q, S304A, E315G, A323Y, T408F and E436D;
(c) Y76M, V103M, S108M, F130Y, A214P, G219A, L243Q, S304A, T309E, A313K, E315G, E316T, A323Y, N342D, N405E, T408Q and E436D;
(d) Y76M, V103M, S108M, F130Y, A214P, L243Q, S304A, T309E, A313K, E315G, A323Y, N342D, T408Q and E436D;
(e) Y76M, V103M, S108M, F130Y, A214P, G219A, L243Q, S304A, T309E, A313K, E315G, A323Y, N342D, N405E, T408Q and E436D;
(f) A74E, Y76M, V103M, S108M, F130Y, Q196E, Q210L, A214P, G219A, L243Q, S304A, K305Q, T309E, A313R, E315G, E316T, A323Y, K335E, N342D, N405E, T406A, T408E and E436D;
(g) Y76M, V103M, S108M, F130Y, A214P, G219A, L243Q, S304A, T309E, A313R, E315G, E316T, A323Y, N342D, T408Q and E436D;
(h) A74E, Y76M, V103M, S108M, F130Y, Q196E, Q210L, A214P, G219A, L243Q, S304A, K305Q, T309E, A313K, E315G, E316T, A323Y, K335E, N342D, N405E, T406A, T408Q and E436D; or
(i) Y76M, V103M, S108M, F130Y, A214P, G219A, L243Q, S304A, T309E, A313K, E315G, A323Y, N342D, T408Q and E436D.

3. A triacylglycerol lipase variant of a wild-type triacylglycerol lipase from Burkholderia glumae of SEQ ID NO: 40 comprising one or more mutations selected from A63G, N64G, S74E, S78R, A106E, S132A, F161Y, T168Y, T176P, N183W, D196N, R204K, Q210G, T211A, Y214F, R216Q, A234P, S241N, Q242T, A297G, Q300P, F312Y, S319N, V334I, N338F, T348N or T348Q, L355N and V358L.

4. A triacylglycerol lipase variant of a wild-type triacylglycerol lipase from Burkholderia glumae of SEQ ID NO: 40 comprising mutations:
(a) A63G, N64G, S74E, S78R, A106E, S132A, F161Y, T168Y, T176P, N183W, D196N, R204K, Q210G, T211A, Y214F, R216Q, A234P, S241N, Q242T, A297G, Q300P, F312Y, S319N, V334I, N338F, T348N, L355N and V358L;
(b) A63G, N64G, S74E, S78Q, S132A, F161Y, T168Y, T176P, D196N, R204K, Q210G, T211A, Y214F, A234P, S241N, Q242T, A297G, Q300P, F312Y, S319N, V334I, T348N, L355N and V358L;
(c) N64G, S132A, D196N, A234P, S241N, Q242T, A297G, Q300P, S319N, T348N, L355N and V358L;
(d) A63G, N64G, S74E, S78Q, S132A, F161Y, T168Y, T176P, D196N, R204K, Q210G, T211A, Y214F, A234P, S241N, Q242T, A297G, Q300P, F312Y, S319N, V334I, T348Q, L355N and V358L;
(e) S241N, A297G, Q300P, S319N and V358L;
(f) N64G, S132A, D196N, A234P, S241N, Q242T, A297G, Q300P, F312Y, S319N, T348N, L355N and V358L; or
(g) S241N, A297G, Q300P, S319N, T348N and V358L.

5. A halohydrin dehalogenase variant of a wild-type halohydrin dehalogenase from Rhizobium radiobacter of SEQ ID NO: 41 comprising one or more mutations selected from T3V, K10R, F12G, S17A, S22A, E33P, Q37D, K38P, L41R, K52H, E56A, I63V, A65R, T67I; S68K or S68E; S78A, D80A, A83P, Q87R; K91Q or K91E; A93D or A93S; V94P; G99A or G99K; Q105L, R107G, A110R, N113R or N113Q, S117P, F136L, W139L, K140P, E141G, S147A; C153I or C153N; T154A, N157K, S160A, V205Q, T206V, K215E and M245V.

6. A halohydrin dehalogenase variant of a wild-type halohydrin dehalogenase from Rhizobium radiobacter of SEQ ID NO: 41 comprising mutations:
(a) T3V, F12G, S17A, E33P, Q37D, K38P, L41R, K52H, E56A, T67I, S68K, S78A, D80A, A83P, Q87R, K91Q, V94P, G99A, R107G, A110R, N113R, S117P, K140P, E141G, C153I, T154A, N157K, S160A, V205Q and K215E;
(b) F12G, E33P, Q37D, L41R, T67I, S68K, D80A, A83P, Q87R, V94P, G99A, N113R, S117P, K140P, C153I, T154A and N157K;
(c) F12G, E33P, Q37D, L41R, T67I, S68K, A83P, Q87R, V94P, G99A, N113R, S117P, C153I, T154A and N157K;
(d) T3V, K10R, F12G, S17A, S22A, E33P, Q37D, K38P, L41R, K52H, E56A, I63V, T67I, S68K, S78A, D80A, A83P, Q87R, K91E, A93D, V94P, G99K, Q105L, R107G, A110R, N113Q, S117P, F136L, W139L, E141G, S147A, C153N, T154A, N157K, S160A, V205Q, K215E and M245V;
(e) F12G, S17A, E33P, Q37D, K38P, L41R, K52H, E56A, T67I, S68K, D80A, A83P, Q87R, V94P, G99A, R107G, A110R, N113R, S117P, C153N, T154A, N157K, V205Q and K215E;
(f) T3V, K10R, F12G, S17A, S22A, E33P, Q37D, K38P, L41R, K52H, I63V, A65R, T67I, S68E, S78A, D80A, A83P, Q87R, K91E, A93D, V94P, G99A, Q105L, R107G, A110R, N113R, S117P, F136L, W139L, E141G, S147A, C153I, T154A, N157K, S160A, V205Q and K215E;
(g) F12G, S17A, E33P, Q37D, K38P, L41R, K52H, E56A, T67I, S68K, D80A, A83P, Q87R, V94P, G99A, R107G, A110R, N113Q, S117P, K140P, C153I, T154A, N157K, V205Q and K215E;
(h) T3V, K10R, F12G, S17A, S22A, E33P, Q37D, K38P, L41R, K52H, E56A, T67I, S68E, S78A, D80A, A83P, Q87R, K91E, A93S, V94P, G99A, R107G, A110R, N113R, S117P, W139L, E141G, S147A, C153I, T154A, N157K, S160A, V205Q, T206V and K215E;
(i) F12G, E33P, L41R, T67I, S68K, A83P, Q87R, V94P, G99A, N113R, S117P, K140P, C153I, T154A and N157K;
(j) T3V, K10R, F12G, S17A, S22A, E33P, Q37D, K38P, L41R, K52H, E56A, T67I, S68E, S78A, D80A, A83P, Q87R, K91E, A93S, V94P, G99A, R107G, A110R, N113R, S117P, W139L, K140P, E141G, S147A, C153I, T154A, N157K, S160A, V205Q and K215E;
(k) T3V, F12G, S17A, E33P, Q37D, K38P, L41R, K52H, E56A, T67I, S68K, S78A, D80A, A83P, Q87R, K91Q, V94P, G99A, R107G, A110R, N113Q, S117P, E141G, C153N, T154A, N157K, S160A, V205Q and K215E;
(l) F12G, E33P, Q37D, K38P, L41R, K52H, E56A, T67I, S68K, D80A, A83P, Q87R, V94P, G99A, R107G, A110R, N113R, S117P, K140P, C153I, T154A and N157K; or
(m) F12G, E33P, L41R, T67I, S68K, D80A, A83P, Q87R, V94P, G99A, N113R, S117P, K140P, C153N, T154A and N157K.

7. A aminotransferase variant of a wild-type aminotransferase from Chromobacterium violaceum of SEQ ID NO: 42 comprising one or more mutations selected from M36L, G39A, D70E, F71L, R76Y, K90Q, V97I, S101E or S101K, A109P, D112N, R113H, S123A, V124N, T145V, L146I, G152A, S162A, V202L; V203H or V203K; A217P, D315E, A337R, V236I, A239D, H276Y; A286I or A286V; K304D, A312E, H333L; A337K or A337R; Q346E, K358Q, E368P, V379L, V379I or V379M; Q380A, N387D, K390T, D396N, F397E, T402W, R410N, S424A, T452E and A455E or A455K.

8. A aminotransferase variant of a wild-type aminotransferase from Chromobacterium violaceum of SEQ ID NO: 42 comprising mutations:
(a) G39A, F71L, S101E, A109P, D112N, R113H, S123A, V124N, L146I, V202L, V203H, A217P, V236I, A239D, A286I, K304D, A312E, H333L, A337K, E368P, V379L, Q380A, D396N, F397E, T402W and A455E;
(b) D70E, D112N, R113H, S123A, V124N, A217P, A337R, E368P, Q380A, D396N and F397E;
(c) G39A, D70E, F71L, S101E, A109P, D112N, R113H, S123A, V124N, L146I, A217P, V236I, A286I, H333L, A337K, E368P, V379L, Q380A, D396N, F397E and A455E;
(d) G39A, D112N, R113H, S123A, V124N, A217P, A337R, E368P, V379L, Q380A, D396N and F397E;
(e) D70E, D112N, R113H, S123A, V124N, A217P, A286V, A337R, E368P, V379L, Q380A, D396N and F397E;
(f) G39A, D70E, F71L, S101K, A109P, D112N, R113H, S123A, V124N, L146I, V202L, V203H, A217P, V236I, A239D, A286I, K304D, A312E, H333L, A337R, E368P, V379L, Q380A, D396N, F397E, T402W and A455E;
(g) G39A, D70E, F71L, K90Q, V97I, S101E, A109P, D112N, R113H, S123A, V124N, T145V, L146I, G152A, V202L, V203H, A217P, V236I, A239D, H276Y, A286I, K304D, A312E, H333L, A337K, Q346E, E368P, V379I, Q380A, N387D, D396N, F397E, T402W, R410N, T452E and A455K;
(h) M36L, G39A, D70E, F71L, K90Q, V97I, S101K, A109P, D112N, R113H, S123A, V124N, T145V, L146I, G152A, V202L, V203K, A217P, V236I, A239D, H276Y, A286I, K304D, A312E, H333L, A337K, Q346E, K358Q, E368P, V379M, Q380A, N387D, K390T, D396N, F397E, T402W, R410N, S424A, T452E and A455K;
(i) M36L, G39A, D70E, F71L, R76Y, K90Q, V97I, S101K, A109P, D112N, R113H, S123A, V124N, T145V, L146I, G152A, V202L, V203K, A217P, V236I, A239D, H276Y, A286I, K304D, A312E, H333L, A337R, Q346E, K358Q, E368P, Q380A, N387D, K390T, D396N, F397E, T402W, R410N, S424A, T452E and A455E;
(j) G39A, D70E, S101E, A109P, D112N, R113H, S123A, V124N, L146I, A217P, A286I, H333L, A337K, E368P, V379M, Q380A, D396N and F397E;
(k) G39A, D70E, F71L, S101E, A109P, D112N, R113H, S123A, V124N, L146I, A217P, V236I, A286I, D315E, H333L, A337R, E368P, Q380A, D396N, F397E and A455E;
(l) G39A, D70E, F71L, K90Q, V97I, S101E, A109P, D112N, R113H, S123A, V124N, T145V, L146I, G152A, S162A, V202L, V203H, A217P, V236I, A239D, H276Y, A286I, K304D, A312E, H333L, A337R, Q346E, E368P, V379I, Q380A, N387D, D396N, F397E, T402W, R410N, T452E and A455K;
(m) G39A, D70E, D112N, R113H, S123A, V124N, A217P, A286V, A337R, E368P, V379L, Q380A, D396N and F397E; or
(n) G39A, S101E, A109P, D112N, R113H, S123A, V124N, L146I, A217P, A286I, H333L, A337R, E368P, V379L, Q380A, D396N and F397E.

9. A putative secreted lipase variant of a wild-type putative secreted lipase from Clostridium botulinum of SEQ ID NO: 43 comprising one or more mutations selected from D69N, E91Q, I101T, K137D, A140R, S141N, H143Y, V168K, K183G, K196P, N200D, K246H, S253V, S265A, T277M, K289N, N308D, E339Y, T346K, A375P, S395T, Q398K, G437T, M469I, N477Q and S479P.

10. A putative secreted lipase variant of a wild-type putative secreted lipase from Clostridium botulinum of SEQ ID NO: 43 comprising mutations:
(a) E91Q, I101T, A140R, K183G, K246H, S265A, T277M, K289N, N308D, E339Y, A375P, S395T, G437T and S479P;
(b) I101T, K183G, T277M, K289N, N308D, A375P, S395T and S479P;
(c) D69N, E91Q, I101T, K137D, A140R, S141N, H143Y, V168K, K183G, K196P, N200D, K246H, S265A, T277M, K289N, N308D, E339Y, T346K, A375P, S395T, Q398K, G437T, M469I, N477Q and S479P;
(d) I101T, K183G, T277M, K289N, A375P, S395T and S479P;
(e) D69N, E91Q, I101T, K137D, A140R, S141N, H143Y, V168K, K183G, K196P, N200D, K246H, S265A, T277M, K289N, N308D, E339Y, T346K, A375P, S395T, Q398K, G437T, N477Q and S479P;
(f) I101T, K183G, K246H, S253V, T277M, K289N, N308D, A375P, S395T, G437T and S479P;
(g) I101T, K183G, K246H, T277M, K289N, N308D, A375P, S395T, G437T and S479P; or
(h) K183G, T277M, A375P, S395T and S479P.

11. An alcohol dehydrogenase variant of a wild-type alcohol dehydrogenase from Geobacillus stearothermophilus of SEQ ID NO: 44 comprising one or more mutations selected from K10G; K11E or K11Q; Q14K or Q14V; V15I, E19P, K20V or K20I, K22T, S24G, V66I, I67V, E68V, E69A, Y89W, Q102W, R108H, Q110K, A112T, Y120F, A127D, K133H, F147L, T153V, V159E or V159M; V179M, L196I, G197D; E199D, K205R, Q206E or Q206K; K216L, H217K, D218E, A220P, Q222K, W223F, I224M, E226K, T234A, A242P, E245Q, S246Q, K249R or K249N; I251L, A256T, C257V or C257L; E265G, I267M or I267F, I269V or I269L; V279I, V28I, T306P or T306V; V308I, Q311R; N315K or N315D; D318E, L324R, N329D or N329K, K335D, V336L, D337E, K335D, V336L and D337E.

12. An alcohol dehydrogenase variant of a wild-type alcohol dehydrogenase from Geobacillus stearothermophilus of SEQ ID NO: 44 comprising mutations:
(a) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, I269V, V279I, V308I, Q311R, N315K, D318E, K335D, V336L and D337E;
(b) K10G, K11E, Q14K, V15I, E19P, K20V, K22T, S24G, V66I, I67V, Q102W, R108H, Q110K, A112T, A127D, K133H, V179M, L196I, G197D, Q206E, K216L, H217K, D218E, A220P, Q222K, W223F, I224M, T234A, A242P, E245Q, S246Q, K249R, A256T, C257V, I267M, V279I, V308I, Q311R, N315K, D318E, K335D, V336L and D337E;
(c) K10G, K11Q, Q14V, V15I, E19P, K20I, K22T, S24G, V66I, I67V, E68V, E69A, Y89W, Q102W, R108H, A112T, Y120F, A127D, K133H, T153V, V159E, V179M, L196I, G197D, E199D, Q206K, K216L, H217K, D218E, A220P, Q222K, W223F, I224M, E226K, T234A, A242P, S246Q, K249R, I251L, A256T, C257L, E265G, I267M, I269V, V279I, T306P, V308I, Q311R, N315D, D318E, L324R, N329D, K335D, V336L and D337E;
(d) K10G, K11Q, Q14K, V15I, E19P, K20I, K22T, 524G, V66I, I67V, Q102W, R108H, Q110K, A112T, Y120F, A127D, K133H, F147L, T153V, V159E, V179M, L196I, G197D, Q206E, K216L, H217K, D218E, A220P, Q222K, W223F, I224M, T234A, A242P, E245Q, S246Q, K249R, A256T, C257L, I267M, I269V, V279I, T306V, V308I, Q311R, N315D, D318E, N329D, K335D, V336L and D337E;
(e) K10G, K11Q, Q14V, V15I, E19P, K20I, K22T, S24G, V28I, V66I, I67V, E68V, E69A, Y89W, Q102W, R108H, A112T, Y120F, A127D, K133H, T153V, V159E, V179M, L196I, G197D, E199D, Q206K, K216L, H217K, D218E, A220P, Q222K, W223F, I224M, E226K, T234A, A242P, S246Q, K249R, I251L, A256T, C257L, E265G, I267M, I269V, V279I, T306P, V308I, Q311R, N315D, D318E, L324R, N329D, K335D, V336L and D337E;
(f) K10G, K11Q, Q14K, V15I, E19P, K20I, K22T, S24G, V66I, I67V, Q102W, R108H, Q110K, A112T, A127D, K133H, V159M, V179M, L196I, G197D, Q206E, K216L, H217K, D218E, A220P, Q222K, W223F, I224M, T234A, A242P, E245Q, S246Q, K249R, A256T, C257V, I267F, I269L, V279I, T306P, V308I, Q311R, D318E, N329K, K335D, V336L and D337E;
(g) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, V279I, V308I, N315K, D318E, K335D, V336L and D337E;
(h) K10G, K11E, Q14K, V15I, E19P, K22T, S24G, V66I, I67V, Q102W, R108H, Q110K, A127D, K133H, V159E, V179M, L196I, G197D, Q206E, K216L, H217K, D218E, A220P, Q222K, W223F, I224M, T234A, A242P, E245Q, K249N, A256T, I267M, I269V, V279I, V308I, N315K, D318E, K335D, V336L and D337E;
(i) K10G, K11E, Q14K, V15I, E19P, K20V, K22T, S24G, V66I, I67V, Q102W, R108H, Q110K, A112T, A127D, K133H, V159E, V179M, L196I, G197D, Q206E, K216L, H217K, D218E, A220P, Q222K, W223F, I224M, T234A, A242P, E245Q, S246Q, K249R, A256T, I267M, I269V, V279I, V308I, N315K, D318E, K335D, V336L and D337E;
(j) K10G, K11Q, Q14V, V15I, E19P, K20I, K22T, S24G, V66I, I67V, E69A, Y89W, Q102W, R108H, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249N, I251L, A256T, C257L, I267M, I269L, V279I, T306P, V308I, Q311R, N315D, D318E, N329D, K335D, V336L and D337E;
(k) K10G, K11Q, Q14V, V15I, E19P, K20I, K22T, S24G, V66I, I67V, E69A, Y89W, Q102W, R108H, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249R, I251L, A256T, C257L, I267M, I269V, V279I, T306P, V308I, Q311R, N315D, D318E, N329D, K335D, V336L and D337E;
(l) K10G, K11Q, Q14K, V15I, E19P, K20I, K22T, S24G, V66I, I67V, Q102W, R108H, Q110K, A112T, A127D, K133H, F147L, V159M, V179M, L196I, G197D, K205R, Q206E, K216L, H217K, D218E, A220P, Q222K, W223F, I224M, T234A, A242P, E245Q, S246Q, K249R, A256T, C257V, I267M, I269V, V279I, T306V, V308I, Q311R, D318E, N329K, K335D, V336L and D337E;
(m) K10G, K11Q, Q14K, V15I, E19P, K20I, K22T, S24G, V66I, I67V, Y89W, Q102W, R108H, Q110K, A112T, Y120F, A127D, K133H, T153V, V159E, V179M, L196I, G197D, Q206E, K216L, H217K, D218E, A220P, Q222K, W223F, I224M, T234A, A242P, E245Q, S246Q, K249R, A256T, C257L, I267F, I269L, V279I, T306V, V308I, Q311R, N315D, D318E, N329D, K335D, V336L and D337E; or
(n) K10G, K11E, Q14K, V15I, E19P, K22T, S24G, V66I, I67V, Q102W, R108H, Q110K, A127D, K133H, V159E, V179M, L196I, G197D, Q206E, K216L, H217K, D218E, A220P, Q222K, W223F, I224M, T234A, A242P, E245Q, K249R, A256T, I267F, I269V, V279I, V308I, N315K, D318E, K335D, V336L and D337E.

13. A histidine acid phosphatase variant of a wild-type histidine acid phosphatase from Yersinia mollaretii of SEQ ID NO: 45 comprising one or more mutations selected from S42A, Q46P or Q46F, D52N, D56H or D56Q, K57P, A65P, T77R or T77K, G81Q or G81R, F82Y, Y83F, N89Q, T102E, Q106W, I109S or I109T, L115A, A136P, V141P or V141M, E149K, Q154T or Q154R, S157P, T158K or T158E, H161R or H161K, R162A, E165M, Q167R, A170G, S173D, E174A, K181D, P182D or P182S, Q185L, G187Q or G187E, E188Q, I189V, T193C or T193K, A194N, 5200A or 5200K, 5207T, N221S, Q222K, Q223E, S230E, S236A, G240A, N247Y, S248A, H257G, A262E, V266R, S267E, L269M, Q275K or Q275Y, K289Q, V298L, K306D, G324A, G334A, Q342T, Q345G, G355A, N363D, D365K, M380L, S386A, K392T, S393N, H394N, 1409T, T411D, Q416P and A425K.

14. A histidine acid phosphatase variant of a wild-type histidine acid phosphatase from Yersinia mollaretii of SEQ ID NO: 45 comprising mutations:
(a) S42A, Q46P, D52N, D56H, K57P, A65P, T77R, G81Q, F82Y, Y83F, N89Q, Q106W, I109 S, L115A, A136P, V141P, E149K, Q154T, S157P, T158K, H161R, R162A, E165M, Q167R, A170G, S173D, E174A, K181D, P182D, Q185L, G187Q, E188Q, T193C, A194N, S200A, 5207T, N221S, Q222K, Q223E, S230E, S236A, G240A, N247Y, S248A, H257G, A262E, V266R, Q275K, K289Q, V298L, K306D, G324A, G334A, Q342T, Q345G, G355A, N363D, D365K, M380L, S386A, K392T, S393N, H394N, I409T, T411D, Q416P and A425K;
(b) Q46F, D56H, K57P, A65P, T77K, G81Q, F82Y, Y83F, N89Q, T102E, Q106W, L115A, V141M, Q154T, S157P, T158E, H161R, R162A, E165M, A170G, E174A, Q185L, G187Q, T193C, A194N, S200K, S230E, S236A, G240A, N247Y, S248A, A262E, V266R, V298L, K306D, G324A, G334A, Q345G, N363D, S386A, I409T and T411D;
(c) Q46F, K57P, A65P, T77K, G81R, N89Q, Q106W, L115A, Q154R, S157P, T158E, H161K, E165M, A170G, E174A, Q185L, G187Q, T193C, S236A, G240A, N247Y, S248A, L269M, V298L, K306D, G324A, G334A, Q345G and T411D;
(d) Q46F, D56Q, K57P, A65P, T77K, G81R, F82Y, Y83F, N89Q, T102E, Q106W, I109T, L115A, V141P, Q154T, S157P, T158E, H161R, R162A, E165M, A170G, E174A, Q185L, G187Q, E188Q, T193C, A194N, S200K, Q222K, Q223E, S230E, S236A, G240A, N247Y, S248A, H257G, A262E, V266R, S267E, L269M, V298L, K306D, G324A, G334A, Q342T, Q345G, G355A, N363D, S386A, S393N, H394N, I409T, T411D and A425K;
(e) S42A, Q46F, D52N, D56H, K57P, A65P, T77R, G81Q, F82Y, Y83F, N89Q, T102E, Q106W, I109T, L115A, A136P, V141P, E149K, Q154T, S157P, T158K, H161K, R162A, E165M, Q167R, A170G, S173D, E174A, K181D, P182S, Q185L, G187E, E188Q, I189V, T193K, A194N, S200A, S207T, N221S, Q222K, Q223E, S230E, S236A, G240A, N247Y, S248A, H257G, A262E, V266R, Q275K, K289Q, V298L, K306D, G324A, G334A, Q342T, Q345G, G355A, N363D, D365K, S386A, K392T, S393N, H394N, I409T, T411D, Q416P and A425K;
(f) Q46F, K57P, A65P, T77K, G81R, F82Y, Y83F, N89Q, T102E, Q106W, L115A, Q154T, S157P, T158E, H161K, E165M, A170G, E174A, Q185L, G187Q, T193C, S200K, S236A, G240A, N247Y, S248A, V266R, L269M, V298L, K306D, G324A, G334A, Q345G and T411D;
(g) Q46F, A65P, T77K, G81Q, N89Q, T102E, Q106W, S157P, A170G, Q185L, G187Q, T193C, S236A, G240A, N247Y, S248A, L269M, V298L, K306D, G324A, G334A, Q345G and T411D;
(h) Q46F, K57P, A65P, T77K, G81Q, F82Y, Y83F, N89Q, T102E, Q106W, L115A, Q154T, S157P, T158E, H161K, E165M, A170G, E174A, Q185L, G187Q, T193C, S200K, S236A, G240A, N247Y, S248A, V266R, Q275Y, V298L, K306D, G324A, G334A, Q345G and T411D;
(i) Q46F, D56H, K57P, A65P, T77K, G81R, F82Y, Y83F, N89Q, T102E, Q106W, L115A, V141M, Q154T, S157P, T158E, H161R, R162A, E165M, A170G, E174A, Q185L, G187Q, I189V, T193C, A194N, S200K, S230E, S236A, G240A, N247Y, S248A, A262E, V266R, L269M, V298L, K306D, G324A, G334A, Q345G, N363D, S386A, I409T and T411D;
(j) N89Q, T102E, Q106W, S157P, A170G, Q185L, G187Q, S236A, G240A, N247Y, S248A, V298L, G324A, G334A, Q345G and T411D;
(k) N89Q, Q106W, S157P, A170G, Q185L, G187Q, S236A, G240A, N247Y, S248A, L269M, Q275Y, V298L, G324A, G334A, Q345G and T411D;
(l) Q46F, D56H, K57P, A65P, T77K, G81Q, F82Y, Y83F, N89Q, T102E, Q106W, I109T, L115A, V141P, Q154T, S157P, T158E, H161R, R162A, E165M, A170G, E174A, Q185L, G187Q, E188Q, T193C, A194N, S200K, Q222K, Q223E, S230E, S236A, G240A, N247Y, S248A, H257G, A262E, V266R, S267E, L269M, Q275Y, V298L, K306D, G324A, G334A, Q342T, Q345G, G355A, N363D, S386A, S393N, H394N, I409T, T411D and A425K; or
(m) Q46F, K57P, A65P, T77K, G81Q, N89Q, T102E, Q106W, L115A, Q154R, S157P, T158E, H161K, E165M, A170G, E174A, Q185L, G187Q, T193C, S236A, G240A, N247Y, S248A, V298L, K306D, G324A, G334A, Q345G and T411D.

15. A chanoclavine-I aldehyde reductase easA variant of a wild-type chanoclavine-I aldehyde reductase easA from Neosartorya fumigata of SEQ ID NO: 46 comprising one or more mutations selected from A8S, K12Q, H20N, I26V, T30M, G37D, Q38N, G50A, D66F, T68S, M72G, P84E, R86I, E91K, S94D, R95A, I126V, C151Y or C151I, A153E, N164E, T184I, K186D, Q192T, T210V, V217I, E240D, A250E, M252L, D256K, L268Q, E270N, E304R, K305W, Q309W, Y368W, L371R and H372N or H372Q.

16. A chanoclavine-I aldehyde reductase easA variant of a wild-type chanoclavine-I aldehyde reductase easA from Neosartorya fumigata of SEQ ID NO: 46 comprising mutations:
(a) A8S, K12Q, H20N, I26V, T30M, G37D, Q38N, G50A, D66F, T68S, M72G, P84E, R86I, E91K, S94D, R95A, I126V, C151Y, A153E, N164E, T184I, K186D, Q192T, T210V, V217I, E240D, A250E, M252L, D256K, L268Q, E270N, E304R, K305W, Q309W, Y368W, L371R and H372N;
(b) A8S, H20N, I26V, T30M, G37D, Q38N, G50A, D66F, T68S, M72G, R86I, E91K, S94D, C151Y, A153E, N164E, T184I, K186D, Q192T, V217I, A250E, Q309W, Y368W, L371R and H372Q;
(c) A8S, I26V, T30M, G37D, Q38N, G50A, D66F, T68S, M72G, R86I, S94D, C151Y, A153E, N164E, T184I, K186D, Q192T, V217I, Q309W, Y368W and L371R;
(d) A8S, K12Q, H20N, I26V, T30M, G37D, Q38N, G50A, D66F, T68S, M72G, P84E, R86I, E91K, S94D, R95A, C151Y, A153E, N164E, T184I, K186D, Q192T, T210V, V217I, A250E, Q309W, Y368W, L371R and H372Q;
(e) A8S, K12Q, H20N, I26V, T30M, G37D, Q38N, G50A, D66F, T68S, M72G, P84E, R86I, E91K, S94D, R95A, C151Y, A153E, N164E, T184I, K186D, Q192T, T210V, V217I, A250E, M252L, Q309W, Y368W, L371R and H372N;
(f) A8S, K12Q, H20N, I26V, T30M, G37D, Q38N, G50A, T68S, M72G, P84E, R86I, E91K, S94D, R95A, I126V, C151I, A153E, N164E, T184I, K186D, Q192T, T210V, V217I, E240D, A250E, M252L, D256K, L268Q, E270N, E304R, K305W, Q309W, Y368W, L371R and H372N;
(g) I26V, G50A, D66F, T68S, M72G, R86I, C151Y, K186D, Q192T and Y368W;
(h) I26V, T30M, G50A, D66F, T68S, M72G, R86I, S94D, C151Y, A153E, K186D, Q192T and Y368W; or
(i) A8S, I26V, T30M, G37D, G50A, D66F, T68S, M72G, R86I, S94D, C151Y, A153E, N164E, T184I, K186D, Q192T, Y368W and L371R.

17. An alanine dehydrogenase variant of a wild-type alanine dehydrogenase from Archaeoglobus fulgidus of SEQ ID NO: 47 comprising one or more mutations selected from E2K, Q8R, S13Q, N22E, L33E, G72Q, S89D, T104I, S106A, S125A, F128V, T135E, K235R, D277P, V300A, S310E, S315T, K316F and I317V.

18. An alanine dehydrogenase variant of a wild-type alanine dehydrogenase from Archaeoglobus fulgidus of SEQ ID NO: 47 comprising mutations:
(a) E2K, Q8R, N22E, L33E, S89D, T104I, S106A, S125A, F128V, K235R, D277P, S310E, S315T and K316F;
(b) Q8R, S106A, K235R, D277P, S310E, S315T and K316F;
(c) E2K, Q8R, N22E, S89D, S106A, S125A, K235R, D277P, S310E, S315T and K316F;
(d) E2K, Q8R, N22E, S89D, S106A, S125A, F128V, D277P, S310E, S315T and K316F;
(e) E2K, Q8R, S13Q, N22E, L33E, G72Q, S89D, T104I, S106A, S125A, F128V, T135E, K235R, D277P, V300A, S310E, S315T, K316F and I317V;
(f) E2K, Q8R, N22E, L33E, S89D, T104I, S106A, S125A, F128V, D277P, V300A, S310E, S315T and K316F;
(g) E2K, Q8R, N22E, L33E, S89D, T104I, S106A, S125A, F128V, T135E, K235R, D277P, V300A, S310E, S315T and K316F;
(h) Q8R, S106A, K235R, D277P, S315T and K316F;
(i) E2K, Q8R, S13Q, N22E, L33E, G72Q, S89D, T104I, S106A, S125A, F128V, K235R, D277P, V300A, S310E, S315T, K316F and I317V;
(j) E2K, Q8R, N22E, S89D, S106A, S125A, F128V, K235R, D277P, S310E, S315T and K316F; or
(k) E2K, Q8R, N22E, L33E, S89D, T104I, S106A, S125A, F128V, D277P, S310E, S315T and K316F.

19. An acid phosphatase variant of a wild-type acid phosphatase from Shimwellia blattae of SEQ ID NO: 48 comprising one or more mutations selected from T28A, E41Q, N44D; N62A, N62L or N62Y; L72A; L81Q, A83K or A83Q; E84A, S90P, G91D, G92D, A94P, N95K; G99P or G99E; S103M, E107P, K108E, A112W, L113I, H114Y or H114W; I121F, S170A; I171F, I171M, I171L or I171A; T175F or T175V; V178I, Q185E or Q185D, N188D, L197Y, A215G, T225A, Q234A, Q235D and Q237A.

20. An acid phosphatase variant of a wild-type acid phosphatase from Shimwellia blattae of SEQ ID NO: 48 comprising mutations:
(a) T28A, E41Q, N44D, N62A, L72A, A83K, E84A, S90P, G91D, G92D, A94P, N95K, G99P, S103M, E107P, A112W, L113I, H114Y, S170A, I171F, Q185E, N188D, L197Y, A215G, T225A, Q234A and Q235D;
(b) T28A, E41Q, N44D, N62A, L72A, A83K, E84A, S90P, G91D, G92D, A94P, N95K, G99P, S103M, E107P, A112W, L113I, H114Y, S170A, T175F, Q185D, N188D, L197Y, A215G, T225A, Q234A and Q235D;
(c) E41Q, N44D, N62A, L72A, A83K, G91D, G92D, A94P, G99P, E107P, H114W, I171M, T175F, Q185D, L197Y and Q234A;
(d) N44D, N62A, L72A, A83K, G91D, G92D, G99E, E107P, H114Y, I171L, Q185E and L197Y;
(e) E41Q, N44D, N62A, L72A, A83K, 590P, G91D, G92D, A94P, N95K, G99P, E107P, A112W, L113I, H114Y, S170A, I171M, T175F, Q185D, L197Y, A215G, T225A and Q234A;
(f) E41Q, N44D, N62A, L72A, A83K, G91D, G92D, A94P, G99P, E107P, H114Y, I171M, T175F, Q185E, L197Y and Q234A;
(g) N44D, N62L, L72A, A83Q, G91D, G92D, G99E, E107P, H114Y, T175F, Q185D and L197Y;
(h) N44D, N62Y, L72A, G91D, G92D, E107P, H114Y, T175F and L197Y;
(i) T28A, E41Q, N44D, N62A, L72A, L81Q, A83K, E84A, S90P, G91D, G92D, N95K, G99P, S103M, E107P, K108E, A112W, L113I, H114Y, I121F, S170A, V178I, Q185E, N188D, L197Y, A215G, T225A, Q234A, Q235D and Q237A;
(j) N44D, N62A, L72A, A83Q, G91D, G92D, G99E, E107P, H114Y, I171L, T175V, Q185E and L197Y; or
(k) T28A, E41Q, N44D, N62A, L72A, L81Q, A83K, E84A, S90P, G91D, G92D, A94P, N95K, G99P, S103M, E107P, K108E, A112W, L113I, H114Y, S170A, I171A, V178I, Q185E, N188D, L197Y, A215G, T225A, Q234A, Q235D and Q237A.

21. A major phosphate-irrepressible acid phosphatase variant of a wild-type major phosphate-irrepressible acid phosphatase from Morganella morganii of SEQ ID NO: 49 comprising one or more mutations selected from E40D, K47A, E54T, Q59D, L61K, N62R, M72A, A89S, A90K, G91D or G91E; T95K, G99E, E107K, M137Q, E148T, N151T, K153D, Q155E, S170A, T175V, V182I, A185D or A185E; N186R, Q187A or Q187R; L197Y, D213E, A215G, T225A, D229N, Q233R, Q235D, K246L, S247L or S247R; Q248R and K249Q.

22. A major phosphate-irrepressible acid phosphatase variant of a wild-type major phosphate-irrepressible acid phosphatase from Morganella morganii of SEQ ID NO: 49 comprising mutations:
(a) E40D, K47A, E54T, Q59D, L61K, N62R, M72A, A89S, A90K, G91D, T95K, G99E, E107K, M137Q, E148T, N151T, K153D, Q155E, S170A, T175V, V182I, A185D, N186R, Q187A, L197Y, D213E, A215G, T225A, D229N, Q233R, Q235D, S247L, Q248R and K249Q;
(b) M72A, A89S, G91E, T95K, G99E, M137Q, E148T, N151T, K153D, S170A, T175V, V182I, A185E, Q187R, L197Y, A215G, T225A, Q235D and K246L;
(c) M72A, A89S, G91D, T95K, G99E, M137Q, E148T, N151T, K153D, S170A, T175V, V182I, A185E, Q187R, L197Y, A215G, T225A, Q235D and K246L;
(d) A89S, T175V, Q187R, L197Y and A215G;
(e) E40D, K47A, E54T, Q59D, L61K, N62R, M72A, A89S, A90K, G91D, T95K, G99E, E107K, M137Q, E148T, N151T, K153D, Q155E, S170A, T175V, V182I, A185E, N186R, Q187A, L197Y, D213E, A215G, T225A, D229N, Q233R, Q235D, S247L, Q248R and K249Q;
(f) K47A, Q59D, M72A, A89S, G91D, T95K, G99E, E107K, M137Q, E148T, N151T, K153D, Q155E, S170A, T175V, V182I, A185E, N186R, Q187A, L197Y, A215G, T225A, D229N, Q235D, K246L and S247R;
(g) M72A, A89S, G91E, T95K, G99E, M137Q, E148T, N151T, K153D, S170A, T175V, V182I, A185E, N186R, Q187R, L197Y, A215G, T225A, Q235D and K246L;
(h) A89S, G91E, G99E, K153D, T175V, Q187R, L197Y, A215G, Q235D and K246L;
(i) K47A, Q59D, M72A, A89S, G91E, T95K, G99E, E107K, M137Q, E148T, N151T, K153D, Q155E, S170A, T175V, V182I, A185E, N186R, Q187A, L197Y, A215G, T225A, D229N, Q235D, K246L and S247R; or
(j) A89S, G91E, G99E, K153D, T175V, Q187R, L197Y and A215G.

23. An acid phosphatase variant of a wild-type acid phosphatase from Prevotella intermedia of SEQ ID NO: 50 comprising one or more mutations selected from N33H, D41E, G42S or G42E; Q44A or Q44S; T45V or T45I, S46D, T53P, L63K or L63E; Y64N, E66M or E66R; A67N, M74A, D82K, V85R, V90L, N97Q, E101C, I105M, K109P, T111K, V119L, L120R or L120Q; G127N, A145M, Y147F, M150S, N153T, E155K, Q157E, Q158E, S161R, T162K, T171A, I173L, T177V, V180I, S182A, I186P, Q189A, N190D, E194K, Y197R, Q198E, M199Y, S221A, D231N, Q237D and Q249R.

24. An acid phosphatase variant of a wild-type acid phosphatase from Prevotella intermedia of SEQ ID NO: 50 comprising mutations:
(a) Q44A, T45V, S46D, T53P, Y64N, E66M, M74A, V85R, V90L, E101C, V119L, Y147F, M150S, N153T, T162K, V180I, S182A, I186P, E194K, M199Y, S221A and Q237D;
(b) N33H, D41E, G42S, Q44A, T45V, S46D, T53P, L63K, Y64N, E66M, A67N, M74A, D82K, V85R, V90L, N97Q, E101C, I105M, K109P, T111K, V119L, L120R, G127N, A145M, Y147F, M150S, N153T, E155K, Q157E, Q158E, S161R, T162K, I173L, T177V, V180I, S182A, I186P, Q189A, N190D, E194K, Y197R, Q198E, M199Y, S221A, D231N, Q237D and Q249R;
(c) G42S, Q44A, T45V, S46D, T53P, L63K, Y64N, E66M, M74A, D82K, V85R, V90L, E101C, K109P, V119L, L120R, G127N, A145M, Y147F, M150S, N153T, E155K, Q157E, Q158E, S161R, T162K, T171A, I173L, T177V, S182A, I186P, N190D, E194K, Y197R, Q198E, M199Y, S221A, D231N, Q237D and Q249R;
(d) Q44A, S46D, T53P, Y64N, E66M, V85R, V90L, V119L, Y147F, M150S, N153T, T177V, I186P, M199Y and S221A;
(e) G42E, Q44A, T45V, S46D, T53P, Y64N, E66M, M74A, D82K, V85R, V90L, E101C, V119L, L120Q, A145M, Y147F, M150S, N153T, E155K, Q157E, S161R, T162K, T177V, V180I, S182A, I186P, M199Y, S221A and Q237D;
(f) Q44A, S46D, T53P, Y64N, E66M, V85R, V90L, V119L, N153T, I186P, M199Y and S221A;
(g) G42S, Q44A, T45V, S46D, T53P, L63K, Y64N, E66M, M74A, D82K, V85R, V90L, E101C, K109P, V119L, L120R, G127N, A145M, Y147F, M150S, N153T, E155K, Q157E, Q158E, S161R, T162K, I173L, T177V, V180I, S182A, I186P, N190D, E194K, Y197R, Q198E, M199Y, S221A, D231N, Q237D and Q249R;
(h) Q44A, T45V, S46D, T53P, Y64N, E66M, M74A, V85R, V90L, E101C, V119L, A145M, Y147F, M150S, N153T, T162K, V180I, S182A, I186P, E194K, M199Y, S221A and Q237D;
(i) N33H, D41E, G42S, Q44A, T45V, S46D, T53P, L63E, Y64N, E66R, A67N, M74A, D82K, V85R, V90L, N97Q, E101C, I105M, K109P, T111K, V119L, L120R, G127N, A145M, Y147F, M150S, N153T, E155K, Q157E, Q158E, S161R, T162K, T171A, I173L, T177V, V180I, S182A, I186P, Q189A, N190D, E194K, Y197R, Q198E, M199Y, S221A, D231N, Q237D and Q249R;
(j) G42E, Q44A, T45V, S46D, T53P, Y64N, E66M, M74A, D82K, V85R, V90L, E101C, V119L, L120Q, A145M, Y147F, M150S, N153T, E155K, Q157E, S161R, T162K, V180I, S182A, I186P, E194K, M199Y, S221A and Q237D;
(k) Q44A, T45V, S46D, T53P, Y64N, E66M, V85R, V90L, V119L, Y147F, M150S, N153T, T177V, S182A, I186P, M199Y, S221A and Q237D; or
(l) Q44S, T45I, S46D, T53P, Y64N, E66M, V85R, V90L, V119L, Y147F, M150S, N153T, T177V, I186P, M199Y and S221A.

25. A tryptophan dimethylallyltransferase variant of a wild-type tryptophan dimethylallyltransferase from Neosartorya fumigata of SEQ ID NO: 51 comprising one or more mutations selected from K2Q, A3T, A9N, R17K, A18Y, L28R, T43D or T43M, T47S, T48L, C50Q or C50E; T57F, C61H, C68P, A74R, N96H, S97N, K114N, S125A, H128R or H128Q, L130K, K134P, H146N, S152R, S155A, F157Y, H160K, G167D, A185T or A185V; T188V or T188A; T202S, V207I, G209E, V211I, R213K, V216Q, N226D, S237P, A254D, Q265R, M266N or M266Q, A285P, S292E, 5304P, V319Q, I320V, Q335P, N336G, V339Y, E341Q, S370T, T378E, T379A, A387Q, D390S, D405N, R406N, T407K or T407Q; and V453K.

26. A tryptophan dimethylallyltransferase variant of a wild-type tryptophan dimethylallyltransferase from Neosartorya fumigata of SEQ ID NO: 51 comprising mutations:
(a) L28R, C50Q, T57F, A74R, S125A, H128R, K134P, H146N, H160K, A185T, T188V, V211I, V216Q, S237P, A285P, S292E, V319Q, Q335P, E341Q, T378E, A387Q, R406N and T407K;
(b) L28R, C50Q, T57F, S125A, H128R, K134P, H146N, S152R, H160K, A185V, T188V, V211I, V216Q, S237P, A285P, S292E, 5304P, V319Q, Q335P, A387Q and T407K;
(c) A3T, L28R, C50Q, T57F, C68P, A74R, N96H, S97N, S125A, H128R, K134P, H146N, S152R, H160K, A185T, T188V, T202S, V211I, V216Q, S237P, A285P, S292E, V319Q, Q335P, V339Y, E341Q, T378E, A387Q, R406N and T407Q;
(d) K2Q, A3T, L28R, T43D, T47S, T48L, C50E, T57F, C61H, C68P, A74R, N96H, S97N, K114N, S125A, H128R, L130K, K134P, H146N, S152R, S155A, H160K, A185V, T188V, T202S, V211I, V216Q, N226D, S237P, M266N, A285P, S292E, V319Q, I320V, Q335P, V339Y, E341Q, T378E, A387Q, R406N and T407Q;
(e) A3T, L28R, C50E, T57F, C68P, A74R, S97N, S125A, H128R, K134P, H146N, S152R, H160K, A185V, T188V, T202S, V211I, V216Q, S237P, A285P, S292E, 5304P, V319Q, Q335P, V339Y, E341Q, T378E, A387Q, R406N and T407K;
(f) K2Q, A3T, L28R, T43D, T47S, T48L, C50Q, T57F, C61H, C68P, A74R, S97N, K114N, S125A, H128Q, L130K, K134P, H146N, S152R, S155A, H160K, A185T, T188V, T202S, V211I, V216Q, N226D, S237P, M266N, A285P, S292E, 5304P, V319Q, I320V, Q335P, V339Y, E341Q, T378E, A387Q, R406N and T407K;
(g) L28R, C50Q, T57F, A74R, S125A, H128R, K134P, H146N, H160K, A185V, T188V, V211I, V216Q, S237P, A285P, S292E, 5304P, V319Q, Q335P, T378E, A387Q, R406N and T407K;
(h) K2Q, A3T, A9N, R17K, A18Y, L28R, T43M, T47S, T48L, C50Q, T57F, C61H, C68P, A74R, N96H, S97N, K114N, S125A, H128Q, L130K, K134P, H146N, S152R, S155A, F157Y, H160K, G167D, A185T, T188A, T202S, V207I, G209E, V211I, R213K, V216Q, N226D, S237P, A254D, Q265R, M266Q, A285P, S292E, 5304P, V319Q, I320V, Q335P, N336G, V339Y, E341Q, S370T, T378E, T379A, A387Q, D390S, D405N, R406N, T407Q and V453K;
(i) C50E, T57F, S125A, H128R, K134P, A185T, S237P, A285P, S292E, 5304P, Q335P and T407K;
(j) L28R, C50E, T57F, S125A, H128R, K134P, H146N, H160K, A185V, T188V, V211I, V216Q, S237P, A285P, S292E, 5304P, V319Q, Q335P, A387Q and T407K;
(k) L28R, C50E, T57F, S125A, H128R, K134P, A185T, S237P, A285P, S292E, S304P, Q335P, A387Q and T407K;
(l) K2Q, A3T, A9N, R17K, A18Y, L28R, T43D, T47S, T48L, C50E, T57F, C61H, C68P, A74R, N96H, S97N, K114N, S125A, H128R, L130K, K134P, H146N, S152R, S155A, F157Y, H160K, G167D, A185V, T188A, T202S, V207I, G209E, V211I, R213K, V216Q, N226D, S237P, A254D, Q265R, M266Q, A285P, S292E, S304P, V319Q, I320V, Q335P, N336G, V339Y, E341Q, S370T, T378E, A387Q, D390S, D405N, R406N, T407Q and V453K; or
(m) C50E, T57F, S125A, H128R, K134P, A185T, S237P, A285P, S292E, Q335P and T407K.

27. An alcohol dehydrogenase variant of a wild-type alcohol dehydrogenase from Geobacillus stearothermophilus of SEQ ID NO: 52 comprising one or more mutations selected from N7R or N7T; K10G, A12P, E19P, R20I, K22T, L23I, E24G, E25P or E25R, V28I, I51V, E69A, A71G, K72P, K75T, I77V, Y89W, E94H or E94R; L99R, Q102W or Q102R; L110Q, G112T, Y120F, P127H, A132V, V141L, V143A, T153V, V159E, A162T, I179M, L181V, S197D, S201L, D206K, A212T, I213V, G215A, K222Q, H225Q, D226K, V228I, S236V, K241P, Q249R, K252R, L257I or L257V; V259L, N264P, A265G, L267M, V279I, S280T, V281I, K282R, K290Q, M292L, A311R, E312R or E312K; E318D, N329D and K335D.

28. An alcohol dehydrogenase variant of a wild-type alcohol dehydrogenase from Geobacillus stearothermophilus of SEQ ID NO: 52 comprising mutations:
(a) K10G, A12P, E19P, R20I, L23I, E24G, E25P, I51V, A71G, K72P, K75T, I77V, E94H, Q102W, L110Q, G112T, Y120F, A132V, V143A, T153V, I179M, L181V, S197D, S201L, D206K, A212T, G215A, H225Q, D226K, V228I, S236V, K241P, Q249R, K252R, L257I, V259L, N264P, A265G, L267M, V279I, S280T, K282R, K290Q, A311R, E312R and K335D;
(b) A12P, E19P, R20I, L23I, E24G, E25R, I15V, A71G, K72P, K75T, E94R, Q102W, G112T, A132V, V143A, L181V, S197D, S201L, A212T, G215A, D226K, S236V, K241P, Q249R, K252R, V259L, N264P, A265G, S280T, K282R, K290Q, A311R, E312K and K335D;
(c) N7R, K10G, A12P, E19P, R20I, L23I, E24G, E25P, I15V, A71G, K72P, K75T, I77V, E94H, Q102W, L110Q, G112T, Y120F, A132V, V143A, T153V, I179M, L181V, S197D, S201L, D206K, A212T, I213V, G215A, H225Q, D226K, V228I, S236V, K241P, Q249R, K252R, L257V, V259L, N264P, A265G, L267M, V279I, S280T, V281I, K282R, K290Q, A311R, E312R, N329D and K335D;
(d) A12P, E19P, R20I, L23I, E24G, E25R, A71G, K72P, E94H, Q102W, G112T, A132V, V143A, V159E, L181V, S201L, A212T, G215A, S236V, K241P, Q249R, K252R, V259L, N264P, A265G, S280T, K282R, K290Q, E312R and K335D;
(e) A12P, E19P, R20I, L23I, E24G, E25R, I51V, A71G, K72P, K75T, E94H, Q102W, G112T, A132V, V143A, V159E, L181V, S197D, S201L, A212T, G215A, D226K, S236V, K241P, Q249R, K252R, L257V, V259L, N264P, A265G, S280T, K282R, K290Q, A311R, E312R and K335D;
(f) A12P, E19P, R20I, L23I, E24G, E25P, I51V, A71G, K72P, K75T, E94H, Q102W, L110Q, G112T, A132V, V143A, L181V, S197D, S201L, D206K, A212T, G215A, D226K, V228I, S236V, K241P, Q249R, K252R, V259L, N264P, A265G, S280T, K282R, K290Q, A311R, E312R and K335D;
(g) A12P, E19P, R20I, L23I, E24G, E25R, I51V, A71G, K72P, E94H, Q102W, G112T, A132V, V143A, L181V, S201L, A212T, G215A, S236V, K241P, Q249R, K252R, V259L, N264P, A265G, S280T, K282R, K290Q, E312R and K335D;
(h) K10G, A12P, E19P, R20I, L23I, E24G, E25P, I51V, A71G, K72P, K75T, I77V, E94H, Q102W, L110Q, G112T, Y120F, A132V, V143A, T153V, I179M, L181V, S197D, S201L, D206K, A212T, G215A, H225Q, D226K, V228I, S236V, K241P, Q249R, K252R, V259L, N264P, A265G, L267M, V279I, S280T, K282R, K290Q, M292L, A311R, E312R and K335D;
(i) K10G, A12P, E19P, R20I, L23I, E24G, E25P, V28I, I51V, A71G, K72P, K75T, I77V, E94H, Q102R, L110Q, G112T, Y120F, A132V, V143A, T153V, V159E, I179M, L181V, S197D, S201L, D206K, A212T, I213V, G215A, H225Q, D226K, V228I, S236V, K241P, Q249R, K252R, L257I, V259L, N264P, A265G, L267M, V279I, S280T, V281I, K282R, K290Q, A311R, E312R, N329D and K335D;
(j) A12P, E19P, R20I, L23I, E24G, E25R, I51V, A71G, K72P, E94H, Q102W, G112T, A132V, V143A, V159E, L181V, S201L, A212T, G215A, S236V, K241P, Q249R, K252R, V259L, N264P, A265G, S280T, K282R, K290Q, E312R and K335D;
(k) N7T, K10G, A12P, E19P, R20I, K22T, L23I, E24G, E25P, V28I, I51V, E69A, A71G, K72P, K75T, I77V, Y89W, E94H, Q102R, L110Q, G112T, Y120F, P127H, A132V, V143A, T153V, V159E, I179M, L181V, S197D, S201L, D206K, A212T, I213V, G215A, K222Q, H225Q, D226K, V228I, S236V, K241P, Q249R, K252R, V259L, N264P, A265G, L267M, V279I, S280T, V281I, K282R, K290Q, A311R, E312R, E318D, N329D and K335D;
(l) N7T, K10G, A12P, E19P, R20I, K22T, L23I, E24G, E25P, V28I, I51V, E69A, A71G, K72P, K75T, I77V, Y89W, E94H, L99R, Q102W, L110Q, G112T, Y120F, P127H, V141L, V143A, T153V, V159E, A162T, I179M, L181V, S197D, S201L, D206K, A212T, I213V, G215A, K222Q, H225Q, D226K, V228I, S236V, K241P, Q249R, K252R, V259L, N264P, A265G, L267M, V279I, S280T, K282R, K290Q, M292L, A311R, E312R, E318D, N329D and K335D; or
(m) A12P, E19P, R20I, L23I, E24G, E25P, V28I, I51V, A71G, K72P, K75T, E94R, Q102W, L110Q, G112T, A132V, V143A, L181V, S197D, S201L, D206K, A212T, G215A, H225R, D226K, V228I, S236V, K241P, Q249R, K252R, V259L, N264P, A265G, S280T, K282R, K290Q, A311R, E312K and K335D.

29. An alcohol dehydrogenase variant of a wild-type alcohol dehydrogenase from Rhodococcus jostii of SEQ ID NO: 53 comprising one or more mutations selected from V36A, K44E or K44D, A46L, D70P, S73T, I77V, V83A or V83C, T91R, S97T, Q99M, S100Q, L105A, N108G, V110A, T111A, E113C or E113A; L114Q or L114M; N116D, I128V, A132C, A133M, T136A, Q139E, F140Y, S141A or S141T; T154D, W159K, V160A, S161A, T164G or T164S; S173A, A177T, G178A, E179G, K181Q, A182P, A187V or A1871; V199I, R200Q, A201G, V203R, N206G, G208R, V210I, I213V, S218E or S218D, L220F, F222W, F222M or F222Q; Q225K, S226F, D229T, F230H, Y232F, T233A, A235M, H239A, D241L, Y247W, V249Q, T254V, V255I, V261G, F270M and E271R.

30. An alcohol dehydrogenase variant of a wild-type alcohol dehydrogenase from Rhodococcus jostii of SEQ ID NO: 53 comprising mutations:
(a) V36A, K44E, A46L, D70P, S73T, I77V, V83A, T91R, S97T, Q99M, L105A, N108G, V110A, T111A, E113C, L114Q, N116D, A132C, A133M, T136A, Q139E, F140Y, S141A, T154D, V160A, S161A, T164G, E179G, A182P, A187V, R200Q, A201G, N206G, G208R, V210I, A211V, F222W, S226F, F230H, T233A, H239A, D241L, Y247W, T254V, V255I, V261G, F270M and E271R;
(b) K44D, D70P, S73T, V83A, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, Y247W, V261G and E271R;
(c) K44E, D70P, S73T, I77V, V83A, L105A, V110A, T111A, E113C, L114M, N116D, A132C, A133M, T136A, F140Y, S141T, T154D, V160A, T164S, A182P, R200Q, N206G, G208R, A211V, F222M, S226F, F230H, T233A, H239A, D241L, Y247W, V261G, F270M and E271R;
(d) V36A, K44D, A46L, D70P, S73T, I77V, V83A, S97T, Q99M, L105A, V110A, T111A, E113C, L114Q, N116D, A132C, A133M, T136A, Q139E, F140Y, S141A, T154D, V160A, S161A, T164G, A182P, A187I, R200Q, A201G, N206G, G208R, A211V, F222Q, S226F, F230H, T233A, H239A, D241L, Y247W, T254V, V255I, V261G, F270M and E271R;
(e) V36A, K44D, A46L, D70P, S73T, I77V, V83A, T91R, S97T, Q99M, S100Q, L105A, N108G, V110A, T111A, E113C, L114M, N116D, I128V, A132C, A133M, T136A, Q139E, F140Y, S141A, T154D, W159K, V160A, S161A, T164G, S173A, A177T, G178A, E179G, K181Q, A182P, A187V, V199I, R200Q, A201G, V203R, N206G, G208R, V210I, A211V, I213V, S218E, F222W, Q225K, S226F, D229T, F230H, Y232F, T233A, A235M, H239A, D241L, Y247W, V249Q, T254V, V255I, V261G, F270M and E271R;
(f) V36A, K44D, A46L, D70P, S73T, I77V, V83A, T91R, S97T, Q99M, S100Q, L105A, N108G, V110A, T111A, E113C, L114M, N116D, I128V, A132C, A133M, T136A, Q139E, F140Y, S141A, T154D, W159K, V160A, S161A, T164G, S173A, A177T, G178A, E179G, K181Q, A182P, A187V, V199I, R200Q, A201G, V203R, N206G, G208R, V210I, A211V, I213V, S218D, L220F, F222Q, Q225K, S226F, D229T, F230H, Y232F, T233A, A235M, H239A, D241L, Y247W, V249Q, T254V, V255I, V261G, F270M and E271R;
(g) V36A, K44E, D70P, S73T, I77V, V83A, S97T, Q99M, L105A, V110A, T111A, E113A, L114M, N116D, A132C, A133M, T136A, Q139E, F140Y, S141A, T154D, V160A, T164S, A182P, R200Q, A201G, N206G, G208R, A211V, F222W, S226F, F230H, T233A, H239A, D241L, Y247W, T254V, V255I, V261G, F270M and E271R;
(h) D70P, S73T, V83A, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, V261G and E271R;
(i) V36A, K44E, D70P, S73T, I77V, V83A, S97T, Q99M, L105A, V110A, T111A, E113C, L114Q, N116D, A132C, A133M, T136A, Q139E, F140Y, S141A, T154D, V160A, T164S, A182P, A187I, R200Q, A201G, N206G, G208R, A211V, F222M, S226F, F230H, T233A, H239A, D241L, Y247W, T254V, V255I, V261G, F270M and E271R;
(j) V36A, K44D, A46L, D70P, S73T, I77V, V83A, T91R, S97T, Q99M, L105A, N108G, V110A, T111A, E113C, L114Q, N116D, A132C, A133M, T136A, Q139E, F140Y, S141A, T154D, V160A, S161A, T164G, E179G, A182P, R200Q, A201G, N206G, G208R, V210I, A211V, F222M, S226F, F230H, T233A, H239A, D241L, Y247W, T254V, V255I, V261G, F270M and E271R;
(k) K44D, D70P, S73T, V83C, L105A, V110A, T111A, E113C, L114Q, N116D, A133M, T136A, F140Y, S141T, T154D, V160A, T164S, A182P, R200Q, N206G, A211V, S226F, F230H, T233A, H239A, D241L, Y247W, V261G and E271R;
(l) K44E, D70P, S73T, I77V, V83A, L105A, V110A, T111A, E113A, L114Q, N116D, A132C, A133M, T136A, F140Y, S141T, T154D, V160A, T164S, A182P, R200Q, N206G, G208R, A211V, F222M, S226F, F230H, T233A, H239A, D241L, Y247W, V261G, F270M and E271R;
(m) D70P, S73T, V83A, L105A, V110A, T111A, E113C, L114Q, N116D, A133M, T136A, F140Y, S141T, T154D, V160A, T164G, A182P, R200Q, N206G, A211V, S226F, F230H, T233A, H239A, D241L, V261G and E271R; or
(n) V36A, K44E, A46L, D70P, S73T, I77V, V83A, S97T, Q99M, L105A, V110A, T111A, E113C, L114Q, N116D, A132C, A133M, T136A, Q139E, F140Y, S141A, T154D, V160A, S161A, T164S, A182P, R200Q, A201G, N206G, G208R, A211V, F222M, S226F, F230H, T233A, H239A, D241L, Y247W, T254V, V255I, V261G, F270M and E271R.

31. An acid phosphatase variant of a wild-type acid phosphatase from Shigella flexneri of SEQ ID NO: 54 comprising one or more mutations selected from D40S, N41Q, A425, I55P, G56D, A59D, L61K, L72A or L72K; L81Q, N95K, K108E, S110A, T118R or T118K, N119R, N151T, D156K or D156Q; R160K or R160T; 5170A, T1751 or T175M; K192Q, Q235D and N246L.

32. An acid phosphatase variant of a wild-type acid phosphatase from Shigella flexneri of SEQ ID NO: 54 comprising mutations:
(a) N41Q, I55P, G56D, L72A, N95K, S110A, N119R, R160K, T1751 and Q235D;
(b) N41Q, I55P, G56D, A59D, L72A, N95K, S110A, N119R, N151T, D156K, R160K, T175M, K192Q, Q235D and N246L;
(c) D40S, N41Q, A425, I55P, G56D, A59D, L61K, L72A, L81Q, N95K, K108E, S110A, T118K, N119R, N151T, D156K, R160K, S170A, T175I, K192Q, Q235D and N246L;
(d) D40S, N41Q, A425, I55P, G56D, A59D, L61K, L72K, L81Q, N95K, K108E, S110A, T118R, N119R, N151T, D156K, R160K, S170A, T175I, K192Q, Q235D and N246L;
(e) N41Q, L72A, S110A, T1751 and Q235D;
(f) L72A, S110A and T1751;
(g) L72K, S110A and T1751;
(h) N41Q, I55P, G56D, A59D, L72A, N95K, S110A, N119R, N151T, D156K, R160T, T175M, K192Q, Q235D and N246L; or
(i) N41Q, I55P, G56D, L72A, N95K, S110A, N119R, N151T, D156Q, R160K, T175I, Q235D and N246L.

33. An alcohol dehydrogenase variant of a wild-type alcohol dehydrogenase from Aromatoleum aromaticum of SEQ ID NO: 55 comprising one or more mutations selected from K2R, S8A, R14N, E16T, L45Y, G46L, S49T, A51G, V54T, A55E, N57G, I71V, D76T, A79K, A83R, H91D, L93I, E96P, A97P, I99M or I99A; A100E, V110I, C115A or C115R, L117Y, R120A or R120F, T123D, S128A, Q136E, T142C, I152V, Y1535, Y153A or Y153G; N154E, N154H or N154D; A159P, S162T, T185R, A188V, H198R, R200K, 5205D, C211Q, S212R or S212T or S212Q, Q213N or Q213D; Q215D, A217V or A217R, S218K, C227R, V266E, K274M, R275K, Q277A, Q289M, N291E, T300H, F303K or F303R, 5309V, T3115 or T311P, L312F, T314Q or T314E, L321H, Q322L, K323E or K323Q, A324S and S332C.

34. An alcohol dehydrogenase variant of a wild-type alcohol dehydrogenase from Aromatoleum aromaticum of SEQ ID NO: 55 comprising mutations:
(a) A51G, V54T, D76T, A79K, A83R, E96P, A97P, I99M, C115A, L117Y, S128A, N154E, S162T, T185R, H198R, 5205D, S212R, Q213N, Q215D, A217V, S218K, C227R, V266E, Q277A, N291E, T300H, F303K, 5309V, L312F, T314Q, L321H, Q322L and S332C;
(b) S8A, L45Y, A51G, V54T, N57G, I71V, D76T, A79K, A83R, H91D, E96P, A97P, I99M, C115A, L117Y, R120A, S128A, T142C, I152V, Y1535, N154H, A159P, S162T, T185R, H198R, 5205D, C211Q, S212R, Q213N, Q215D, A217V, S218K, C227R, V266E, K274M, R275K, Q277A, N291E, T300H, F303K, 5309V, T3115, L312F, T314E, L321H, Q322L, K323E and S332C;
(c) K2R, S8A, L45Y, A51G, V54T, N57G, I71V, D76T, A79K, A83R, H91D, E96P, A97P, I99A, C115A, L117Y, R120A, S128A, T142C, I152V, Y153A, N154E, A159P, S162T, T185R, A188V, H198R, 5205D, C211Q, S212R, Q213D, Q215D, A217R, S218K, C227R, V266E, K274M, R275K, Q277A, Q289M, N291E, T300H, F303K, 5309V, T3115, L312F, T314E, L321H, Q322L, K323E, A324S and S332C;
(d) A51G, V54T, D76T, A79K, A83R, E96P, A97P, I99M, C115A, L117Y, R120A, S128A, T142C, Y153S, N154D, A159P, S162T, T185R, H198R, S205D, S212R, Q213N, Q215D, A217V, S218K, C227R, V266E, Q277A, N291E, T300H, F303K, S309V, L312F, T314Q, L321H, Q322L and S332C;
(e) K2R, S8A, R14N, E16T, L45Y, G46L, S49T, A51G, V54T, A55E, N57G, I71V, D76T, A79K, A83R, H91D, L93I, E96P, A97P, I99A, A100E, V110I, C115A, L117Y, R120A, T123D, S128A, Q136E, T142C, I152V, Y153G, N154E, A159P, S162T, T185R, A188V, H198R, R200K, S205D, C211Q, S212T, Q213N, Q215D, A217R, S218K, C227R, V266E, K274M, R275K, Q277A, Q289M, N291E, T300H, F303R, S309V, T311S, L312F, T314Q, L321H, Q322L, K323Q, A324S and S332C;
(f) A51G, V54T, D76T, A79K, A83R, E96P, A97P, I99M, C115A, L117Y, R120F, S128A, N154E, S162T, T185R, H198R, S205D, Q213N, Q215D, A217V, S218K, C227R, V266E, Q277A, N291E, T300H, F303K, S309V, L312F, T314Q, L321H, Q322L and S332C;
(g) S8A, L45Y, A51G, V54T, N57G, I71V, D76T, A79K, A83R, H91D, E96P, A97P, I99M, C115A, L117Y, R120A, S128A, T142C, I152V, Y153S, N154E, A159P, S162T, T185R, H198R, S205D, C211Q, S212R, Q213N, Q215D, A217R, S218K, C227R, V266E, K274M, R275K, Q277A, Q289M, N291E, T300H, F303K, S309V, T311S, L312F, T314E, L321H, K323E and S332C;
(h) A51G, V54T, D76T, A83R, E96P, A97P, L117Y, S128A, N154E, S162T, T185R, H198R, S205D, Q213N, Q215D, A217V, S218K, C227R, V266E, Q277A, N291E, T300H, F303K, S309V, L312F, T314Q, L321H and S332C;
(i) A51G, V54T, D76T, A79K, A83R, E96P, A97P, I99M, C115A, L117Y, R120A, S128A, T142C, Y153S, N154D, A159P, S162T, T185R, H198R, S205D, S212R, Q213N, Q215D, A217V, S218K, C227R, V266E, Q277A, N291E, T300H, F303K, S309V, T311P, L312F, T314Q, L321H, Q322L and S332C;
(j) K2R, S8A, R14N, E16T, L45Y, G46L, S49T, A51G, V54T, A55E, N57G, I71V, D76T, A79K, A83R, H91D, L93I, E96P, A97P, I99A, A100E, V110I, C115A, L117Y, R120A, T123D, S128A, Q136E, T142C, I152V, Y153A, N154E, A159P, S162T, T185R, A188V, H198R, S205D, C211Q, S212Q, Q213N, Q215D, A217R, S218K, C227R, V266E, K274M, R275K, Q277A, Q289M, N291E, T300H, F303R, S309V, T311S, L312F, T314Q, L321H, Q322L, K323E, A324S and S332C;
(k) A51G, V54T, D76T, A83R, E96P, A97P, C115R, L117Y, S128A, N154E, S162T, T185R, H198R, S205D, Q213N, Q215D, A217V, S218K, C227R, V266E, Q277A, N291E, T300H, F303K, S309V, L312F, T314E, L321H and S332C;
(l) A51G, V54T, D76T, A83R, E96P, A97P, L117Y, N154E, S162T, T185R, H198R, S205D, Q213D, Q215D, S218K, C227R, V266E, Q277A, T300H, F303K, S309V, L312F, T314Q, L321H and S332C; or
(m) A51G, V54T, D76T, A83R, E96P, A97P, C115R, L117Y, N154E, S162T, T185R, H198R, S205D, Q213D, Q215D, S218K, C227R, V266E, Q277A, T300H, F303K, S309V, L312F, T314Q, L321H and S332C.

35. An arylesterase variant of a wild-type arylesterase from Pseudomonas pseudoalcaligenes of SEQ ID NO: 56 comprising one or more mutations selected from A34G, F35Y, K49Q, S52K, E54H, E57D, S78P, E83R, E87D, Q102L, V115I, S118A, L128V, V138Q or V138R; T142Q or T142E, T149K or T149E; E153K, S158P, G168A, V170N, G172E, M173W or M173L; A182S, E183A, E187P or E187A, I188Q, L1891 and T196Q.

36. An arylesterase variant of a wild-type arylesterase from Pseudomonas pseudoalcaligenes of SEQ ID NO: 56 comprising mutations:
(a) F35Y, E54H, S78P, E87D, Q102L, L128V, S158P, G168A, G172E and M173W;
(b) A34G, F35Y, K49Q, S52K, E54H, E57D, S78P, E83R, E87D, Q102L, S118A, L128V, V138Q, T142Q, T149K, S158P, G168A, G172E, M173L, E187P and I188Q;
(c) A34G, F35Y, S52K, E54H, E57D, S78P, E87D, Q102L, S118A, L128V, T142Q, S158P, G168A, G172E, M173L, E187P and I188Q;
(d) A34G, F35Y, S52K, E54H, E57D, S78P, E87D, Q102L, S118A, L128V, V138Q, T142Q, T149K, S158P, G168A, G172E, M173L, E187P, I188Q and L1891;
(e) A34G, F35Y, K49Q, S52K, E54H, E57D, S78P, E83R, E87D, Q102L, V115I, S118A, L128V, V138Q, T142Q, T149E, E153K, S158P, G168A, V170N, G172E, M173L, A182S, E183A, E187A, I188Q and T196Q;
(f) A34G, F35Y, K49Q, S52K, E54H, E57D, S78P, E83R, E87D, Q102L, V115I, S118A, L128V, V138R, T142E, T149A, E153K, S158P, G168A, V170N, G172E, M173L, A182S, E183A, E187A, I188Q and T196Q;
(g) A34G, F35Y, S52K, E54H, E57D, S78P, E87D, Q102L, S118A, L128V, V138Q, T142Q, T149K, S158P, G168A, G172E, M173L, E187P and I188Q;
(h) F35Y, E54H, E57D, S78P, E87D, Q102L, L128V, T142Q, S158P, G168A, G172E, M173L, E187P and L1891;
(i) F35Y, E54H, S78P, E87D, Q102L, L128V, S158P, G168A, G172E, M173W and L1891;
(j) A34G, F35Y, K49Q, S52K, E54H, E57D, S78P, E83R, E87D, Q102L, S118A, L128V, V138R, T142E, T149K, S158P, G168A, G172E, M173L, E187P and I188Q; or
(k) F35Y, E54H, S78P, E87D, Q102L, L128V, S158P and G168A.

37. A putrescine aminotransferase variant of a wild-type putrescine aminotransferase from Escherichia coli of SEQ ID NO: 57 comprising one or more mutations selected from A12E, A15Q, H16R, R24D, H28E, A33Q, R36K, K43R, E44K, F50Y, A59K, F84Y, V102K, S105K, N109E, A112D, Q114L, Q119N, L121F, D123N, K131H, A135E, T137A, K140D, S144V, S153A, K162R, A175T, A1925, S194E, T195E, F196Y, N214D, M218L, T220K, N223E, L248I, T255R, F264Y or F264H; M268L, K282R, N290G, Q292V, A299G, L322F, L344I, T346A, N348H, Q353E, A357E, Q361E, M365Y or M365R, V381I, Q382R, M389L, N398E or N398D; G401A, 5406A, R412G, T4185, A4225, T424V, I435D, C438I, E439D, L440K or L440Q; M452I and S455Q.

38. A putrescine aminotransferase variant of a wild-type putrescine aminotransferase from Escherichia coli of SEQ ID NO: 57 comprising mutations:
(a) A12E, H16R, H28E, A33Q, K43R, V102K, S105K, N109E, A112D, Q119N, L121F, D123N, K131H, A135E, T137A, K140D, S144V, S153A, K162R, S194E, T195E, F196Y, N214D, M218L, T220K, N223E, L248I, T255R, F264Y, M268L, K282R, Q292V, A299G, L344I, N348H, Q353E, A357E, Q361E, M365Y, V381I, Q382R, M389L, N398E, S406A, T4185, T424V, I435D, C438I, E439D, L440K and M452I;
(b) N109E, L121F, K140D, S144V, S153A, T195E, M218L, N223E, L248I, T255R, K282R, Q292V, A299G, N348H, M365Y, Q382R, M389L, S406K, T424V, I435D and C438L;
(c) N109E, L121F, A135E, K140D, S144V, S153A, K162R, T195E, M218L, T220K, N223E, L248I, T255R, K282R, Q292V, A299G, N348H, M365Y, Q382R, M389L, S406A, T424V, I435D and C438I;
(d) A12E, H28E, A33Q, S105K, N109E, L121F, K131H, A135E, T137A, K140D, S144V, S153A, K162R, S194E, T195E, N214D, M218L, T220K, N223E, L248I, T255R, F264H, K282R, Q292V, A299G, N348H, Q353E, Q361E, M365Y, V381I, Q382R, M389L, S406A, T424V, I435D, C438I and L440Q;
(e) N109E, L121F, K140D, S144V, S153A, T195E, M218L, N223E, L248I, T255R, K282R, Q292V, A299G, N348H, M365Y, Q382R, M389L, S406K, T424V, I435D and C438I;
(f) A12E, S105K, N109E, L121F, K131H, A135E, T137A, K140D, S144V, S153A, K162R, S194E, T195E, N214D, M218L, T220K, N223E, L248I, T255R, F264H, K282R, Q292V, A299G, N348H, M365Y, Q382R, M389L, S406A, T424V, I435D, C438I and L440R;
(g) N109E, L121F, A135E, K140D, S144V, S153A, K162R, T195E, T220K, N223E, L248I, T255R, K282R, Q292V, A299G, N348H, M365Y, Q382R, M389L, S406A, T424V, I435D and C438I;
(h) A12E, H28E, A33Q, S105K, N109E, L121F, K131H, A135E, T137A, K140D, S144V, S153A, K162R, S194E, T195E, N214D, M218L, T220K, N223E, L248I, T255R, F264Y, K282R, Q292V, A299G, N348H, Q353E, Q361E, M365Y, V381I, Q382R, M389L, S406A, T424V, I435D, C438I and L440Q;
(i) A12E, S105K, N109E, L121F, K131H, A135E, T137A, K140D, S144V, S153A, K162R, S194E, T195E, N214D, M218L, T220K, N223E, L248I, T255R, K282R, Q292V, A299G, N348H, M365Y, Q382R, M389L, S406A, T424V, I435D and C438I;
(j) L121F, S144V, S153A, T195E, N223E, L248I, T255R, K282R, Q292V, A299G, N348H, M365Y, M389L, S406K, T424V, I435D and C438I;
(k) A12E, A15Q, H16R, R24D, H28E, A33Q, R36K, E44K, F50Y, A59K, V102K, S105K, N109E, A112D, Q114L, Q119N, L121F, D123N, K131H, A135E, T137A, K140D, S144V, S153A, K162R, A175T, A1925, S194E, T195E, F196Y, N214D, M218L, T220K, N223E, L248I, T255R, F264H, M268L, K282R, N290G, Q292V, A299G, L322F, L344I, T346A, N348K, Q353E, A357E, Q361E, M365Y, V381I, Q382R, M389L, N398E, G401A, S406A, R412G, T4185, A4225, T424V, I435D, C438I, E439D, L440K, M452I and S455Q;
(l) A12E, A15Q, H16R, R24D, H28E, A33Q, R36K, K43R, F50Y, A59K, F84Y, V102K, S105K, N109E, A112D, Q114L, Q119N, L121F, D123N, K131H, A135E, K140D, S144V, S153A, K162R, A175T, A1925, S194E, T195E, F196Y, N214D, M218L, T220K, N223E, L248I, T255R, F264Y, M268L, K282R, N290G, Q292V, A299G, L322F, L344I, T346A, N348K, Q353E, A357E, Q361E, M365Y, V381I, Q382R, M389L, N398D, G401A, S406A, R412G, T4185, A4225, T424V, I435D, C438I, E439D, L440K, M452I and S455Q;
(m) A12E, H16R, H28E, A33Q, K43R, V102K, S105K, N109E, A112D, Q119N, L121F, D123N, K131H, A135E, T137A, K140D, S144V, S153A, K162R, S194E, T195E, F196Y, N214D, M218L, T220K, N223E, L248I, T255R, F264Y, M268L, K282R, Q292V, A299G, L344I, N348H, Q353E, A357E, Q361E, M365R, V381I, Q382R, M389L, N398E, S406A, T418S, T424V, I435D, C438I, E439D, L440K and M452I; or
(n) L121F, S144V, S153A, T195E, N223E, L248I, T255R, K282R, Q292V, A299G, N348H, M365Y, M389L, S406K, T424V and I435D.

39. A green to red photoconvertible GFP-like protein EosFP variant of a wild-type green to red photoconvertible GFP-like protein EosFP from Lobophyllia hemprichii of SEQ ID NO: 58 comprising one or more mutations selected from N11K, D26E, D28E, K32N, F34Y, M40I, L93M, T94I, I100V or I102T; T113C, N116H, K117E, R119K or R119E, A127P, L137I, K145M, K145S or K145Y; T154V, I157C or I157V, A160F, N166G, A167G, Y169L, F173C, F173V or F173I; E181K, L186M, F191L, V192I and C195R.

40. A green to red photoconvertible GFP-like protein EosFP variant of a wild-type green to red photoconvertible GFP-like protein EosFP from Lobophyllia hemprichii of SEQ ID NO: 58 comprising mutations:
(a) N11K, K32N, F34Y, L93M, T94I, I100V, I102T, T113C, N116H, R119K, A127P, K145M, T154V, I157C, A160F, N166G, A167G, F173C, E181K, F191L, V192I and C195R;
(b) N11K, K32N, L93M, T94I, I100V, I102T, T113C, R119E, A127P, K145S, A160F, N166G, A167G, F173V, E181K, F191L, V192I and C195R;
(c) N11K, K32N, F34Y, L93M, I100V, I102T, T113C, N116H, K117E, R119E, A127P, K145M, T154V, I157C, A160F, N166G, A167G, F173C, E181K, F191L, V192I and C195R;
(d) N11K, K32N, L93M, I100V, R119E, A127P, K145M, T154V, I157C, A160F, N166G, A167G, F173C, E181K, F191L, V192I and C195R;
(e) L93M, R119E, A127P, K145M, N166G, A167G, F173C, F191L and C195R;
(f) N11K, D26E, D28E, K32N, F34Y, M40I, L93M, I100C, I102T, T113C, N116H, K117E, R119E, A127P, L137I, K145M, T154V, I157C, A160F, N166G, A167G, F173I, E181K, L186M, F191L, V192I and C195R;
(g) N11K, K32N, L93M, I100V, R119E, A127P, K145M, T154V, A160F, N166G, A167G, F173V, E181K, F191L, V192I and C195R;
(h) N11K, K32N, L93M, I100V, T113C, R119E, A127P, K145M, T154V, I157C, A160F, N166G, A167G, Y169L, F173V, E181K, F191L, V192I and C195R;
(i) N11K, K32N, L93M, I100V, T113C, R119E, A127P, K145M, T154V, A160F, N166G, A167G, F173C, E181K, F191L, V1921 and C195R;
(j) N11K, D26E, D28E, K32N, F34Y, M40I, L93M, T94I, I100V, I102T, T113C, N116H, R119E, A127P, L137I, K145Y, T154V, I157V, A160F, N166G, A167G, F173V, E181K, L186M, F191L, V1921 and C195R;
(k) N11K, K32N, L93M, I100V, R119E, A127P, K145S, N166G, A167G, F173C, F191L, V1921 and C195R; or
(l) N11K, K32N, L93M, I100V, I102T, T113C, R119E, A127P, K145M, I157C, A160F, N166G, A167G, F173C, E181K, F191L, V1921 and C195R.

41. A red fluorescent protein drFP583 variant of a wild-type red fluorescent protein drFP583 from Discosoma sp. of SEQ ID NO: 59 comprising one or more mutations selected from M1A, N6S, R17H, T21S, V22M, H41T, N42Q, V44A, A57S, K70R, V71A, Y72F, V73T, K83Y, L85Q, N98V, S111T, Q114E, D115G, C117T, F118L, K123W, F124L, I125R, V127T, S131P, D132N, A145P, R153E, E160D, H162K, K163M, K166R, H172R, L174R or L174T; V175A, E176D or E176Q; S179T, I180T, M182K, L189M, Y192A or Y192S; Y194F, S197R, D200E, N205D, 1210V, T217S or T217A; G219A, H222S, L223T and F224G.

42. A red fluorescent protein drFP583 variant of a wild-type red fluorescent protein drFP583 from Discosoma sp. of SEQ ID NO: 59 comprising mutations:
(a) M1A, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, I210V, T217S, H222S, L223T and F224G;
(b) N6S, R17H, T21S, V22M, H41T, N42Q, V44A, A57S, K70R, V71A, Y72F, V73T, K83Y, L85Q, N98V, S111T, Q114E, D115G, F118L, K123W, F124L, I125R, V127T, S131P, D132N, A145P, R153E, E160D, H162K, K163M, K166R, L174T, V175A, E176Q, S179T, 1180T, M182K, L189M, Y192S, Y194F, S197R, D200E, N205D, I210V, T217A, G219A, H222S, L223T and F224G;
(c) M1A, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T217S, H222S, L223T and F224G;
(d) N6S, R17H, T21S, V22M, H41T, N42Q, V44A, A57S, K70R, V71A, Y72F, V73T, K83Y, L85Q, S111T, Q114E, D115G, C117T, F118L, F124L, I125R, V127T, S131P, A145P, R153E, E160D, H162K, K163M, K166R, H172R, L174T, V175A, E176D, S179T, I180T, M182K, L189M, Y192A, Y194F, S197R, N205D, I210V, T217S, G219A, H222S, L223T and F224G;
(e) N6S, R17H, T21S, V22M, H41T, N42Q, V44A, A57S, K70R, V71A, Y72F, V73T, K83Y, L85Q, S111T, Q114E, D115G, F118L, F124L, I125R, V127T, S131P, D132N, A145P, R153E, E160D, H162K, K163M, K166R, H172R, L174T, V175A, E176D, S179T, I180T, M182K, L189M, Y192A, Y194F, S197R, N205D, I210V, T217A, H222S, L223T and F224G;
(f) N6S, R17H, T21S, V22M, H41T, N42Q, V44A, A57S, K70R, V71A, Y72F, V73T, K83Y, L85Q, S111T, Q114E, D115G, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T217S, G219A, H222S, L223T and F224G; or
(g) N6S, R17H, T21S, V22M, H41T, N42Q, V44A, A57S, K70R, V71A, Y72F, V73T, K83Y, L85Q, S111T, Q114E, C117T, F118L, F124L, I125R, V127T, S131P, A145P, R153E, E160D, H162K, K163M, K166R, H172R, L174R, V175A, E176D, S179T, I180T, M182K, L189M, Y192A, Y194F, S197R, N205D, I210V, T217S, G219A, H222S, L223T and F224G.

43. A green fluorescent protein variant of a wild-type green fluorescent protein from Aequorea victoria of SEQ ID NO: 60 comprising one or more mutations selected from S28K, 530R, F64L, S72A, N105C, N146I, H148G, M153T, K158N, V163A, N164E, I167M, N170P, S175G, N198Y, 5202H, Q204R or Q204H; A206V and V224L.

44. A green fluorescent protein variant of a wild-type green fluorescent protein from Aequorea victoria of SEQ ID NO: 60 comprising mutations:
(a) S28K, 530R, F64L, S72A, N105C, N146I, H148G, M153T, K158N, V163A, N164E, I167M, N170P, S175G, N198Y, 5202H, Q204R and A206V;
(b) S28K, 530R, F64L, S72A, N105C, N146I, H148G, M153T, K158N, V163A, N164E, I167M, N170P, S175G, N198Y, Q204H and A206V;
(c) S28K, 530R, F64L, S72A, N105C, N146I, H148G, M153T, K158N, V163A, N164E, I167M, N170P, S175G, N198Y, Q204H, A206V and V224L;
(d) 530R, F64L, S72A, N105C, N146I, H148G, M153T, K158N, V163A, N170P, S175G, 5202H, A206V and V224L;
(e) 530R, F64L, S72A, N146I, H148G, M153T, K158N, V163A, S175G, 5202H and V224L;
(f) 530R, F64L, S72A, N105C, N146I, H148G, M153T, K158N, V163A, N170P, S175G, N198Y, S202H, A206V and V224L; or
(g) S28K, 530R, F64L, S72A, N105C, N146I, H148G, M153T, K158N, V163A, N170P, S175G, N198Y, S202H, A206V and V224L.

45. A luciferin 4-monooxygenase variant of a wild-type luciferin 4-monooxygenase from Photinus pyralis of SEQ ID NO: 61 comprising one or more mutations selected from A4G, K8I, A12P, F14R, D19P, Q25L, A29L, V36H, T39R, I47T, E48G, V49Q, Y56L, E58Q, M59W, 560A, V61N or V61C; L63M or L63V; M67L, T74K, N75G, R77V, Q87E, M90V, A101T or A101V; A103H or A103T; I108N or I108T, N110T, R112D, L114I, N116H, S117Q, M118L, T124K, V128T, K130P or K1305; G132A, Q134P, Q147K, T156Q, Q159N, V168M, T169K, H171Y, G175N, V182K, K190Q, T191Q, T214N, A215I, A222C, R223S, D234G, G246A, T251C or T251H; T252V, Y255H, I257R or I257Y; C258Q, L264V, Y266R or Y266K; S276A, L277V or L277I; K281R, Q283T, S284V, T290S or T290P, F292M, S293V, N3085, H310T, G316A, H332N or H332G; I349V, A361S, F368G, V384P, M396N, S399K, N409K, L411T, 5420T, W426F, E430G, S440E, S456A, I457V, Q460S, N463D or N463K; F465A, L472Y, E488K, H489P, V499I, T5075, T508P, A509H or A509Y; V522I, G525T, G525N or G5255; L526P, L530I and A532R or A532K.

46. A luciferin 4-monooxygenase variant of a wild-type luciferin 4-monooxygenase from Photinus pyralis of SEQ ID NO: 61 comprising mutations:
(a) A4G, K8I, A12P, D19P, Q25L, V36H, T39R, I47T, E48G, V49Q, Y56L, E58Q, M59W, 560A, V61N, L63M, M67L, T74K, N75G, R77V, M90V, A101T, A103H, I108N, N110T, R112D, L114I, N116H, S117Q, M118L, T124K, V128T, K130P, G132A, Q134P, V168M, T169K, H171Y, G175N, V182K, K190Q, T191Q, T214N, A222C, D234G, G246A, T251C, T252V, I257R, C258Q, L264V, Y266R, S276A, Q283T, T290S, S293V, G316A, H332N, A361S, V384P, M396N, S399K, N409K, L411T, 5420T, W426F, E430G, S440E, S456A, I457V, Q460S, N463D, F465A, L472Y, E488K, H489P, V499I, T5075, T508P, A509H, V522I, G525T, L526P, L530I and A532R;
(b) A4G, K8I, D19P, T39R, I47T, E48G, Y56L, M59W, V61C, L63V, M67L, R77V, M90V, A101T, A103H, I108N, N110T, N116H, S117Q, M118L, T124K, K130P, V168M, H171Y, T191Q, T214N, T251H, I257R, L264V, Y266K, S276A, L277V, Q283T, T290P, S293V, G316A, V384P, 5420T, W426F, E430G, S440E, F465A, L472Y, E488K, H489P, T5075, T508P, A509Y, V522I, G525N and A532K;
(c) A4G, K8I, D19P, T39R, I47T, E48G, Y56L, V61C, L63V, M67L, R77V, M90V, A101T, A103H, I108N, N110T, N116H, S117Q, M118L, T124K, K130P, V168M, H171Y, T191Q, T214N, T251H, I257R, L264V, Y266K, S276A, L277V, Q283T, T290P, S293V, G316A, V384P, S420T, W426F, E430G, S440E, F465A, L472Y, E488K, H489P, T507S, T508P, A509Y, V522I, G525N and A532K;
(d) A4G, K8I, D19P, I47T, E48G, Y56L, V61C, L63V, M67L, R77V, M90V, A101T, A103H, I108N, N116H, S117Q, M118L, K130P, V168M, H171Y, T191Q, T214N, T251H, I257R, L264V, Y266K, S276A, K281R, Q283T, T290P, S293V, V384P, S420T, W426F, E430G, S440E, F465A, L472Y, E488K, H489P, T508P, A509Y, V522I, G525N and A532K;
(e) A4G, K8 I, D19P, I47T, E48G, Y56L, M59W, V61C, L63V, M67L, R77V, M90V, A101T, A103H, I108N, S117Q, M118L, K130P, V168M, H171Y, T251H, I257R, L264V, Y266K, S276A, L277V, K281R, Q283T, T290P, S293V, V384P, S420T, W426F, E430G, S440E, F465A, L472Y, E488K, H489P, T508P, A509H, V522I and A532K;
(f) A4G, K8I, A12P, F14R, D19P, Q25L, V36H, T39R, I47T, E48G, V49Q, Y56L, E58Q, M59W, S60A, V61N, L63M, M67L, T74K, N75G, R77V, Q87E, M90V, A101T, A103H, I108N, N110T, R112D, L114I, N116H, S117Q, M118L, T124K, V128T, K130P, G132A, Q134P, Q147K, T156Q, Q159N, V168M, T169K, H171Y, G175N, V182K, K190Q, T191Q, T214N, A222C, D234G, G246A, T251C, T252V, I257R, C258Q, L264V, Y266R, S276A, L277I, Q283T, T290S, S293V, N308S, H310T, G316A, H332G, I349V, A361S, F368G, V384P, M396N, S399K, N409K, L411T, S420T, W426F, E430G, S440E, S456A, I457V, Q460S, N463K, F465A, L472Y, E488K, H489P, V499I, T507S, T508P, A509Y, V522I, G525T, L526P, L530I and A532R;
(g) A4G, K8I, A12P, D19P, T39R, I47T, E48G, V49Q, Y56L, S60A, V61C, L63V, M67L, T74K, R77V, M90V, A101T, A103H, I108T, N110T, N116H, S117Q, M118L, T124K, V128T, K130P, G132A, Q134P, V168M, H171Y, G175N, T191Q, T214N, A215I, D234G, G246A, Y255H, I257R, C258Q, L264V, Y266K, S276A, L277V, Q283T, T290P, S293V, G316A, H332N, A361S, V384P, S399K, N409K, S420T, W426F, E430G, S440E, S456A, I457V, Q460S, N463D, F465A, L472Y, E488K, H489P, V499I, T507S, T508P, A509Y, V522I, G525S, L526P, L530I and A532K;
(h) A4G, K8I, A12P, F14R, D19P, Q25L, A29L, V36H, T39R, I47T, E48G, V49Q, Y56L, E58Q, M59W, S60A, V61N, L63M, M67L, T74K, N75G, R77V, Q87E, M90V, A101V, A103H, I108N, N110T, R112D, L114I, N116H, S117Q, M118L, T124K, V128T, K130P, G132A, Q134P, Q147K, T156Q, Q159N, V168M, T169K, H171Y, G175N, V182K, K190Q, T191Q, T214N, A215I, A222C, D234G, G246A, T251C, T252V, I257Y, C258Q, L264V, Y266R, S276A, L277I, Q283T, T290S, F292M, S293V, N308S, H310T, G316A, H332G, I349V, A361S, F368G, V384P, M396N, S399K, N409K, L411T, S420T, W426F, E430G, S440E, S456A, I457V, Q460S, N463K, F465A, L472Y, E488K, H489P, V499I, T507S, T508P, A509Y, V522I, G525T, L526P, L530I and A532R;
(i) A4G, K8I, D19P, I47T, E48G, Y56L, M59W, V61C, L63V, M67L, R77V, M90V, A101T, A103T, I108N, N116H, S117Q, M118L, K130S, V168M, H171Y, T191Q, T214N, T251H, I257R, L264V, Y266K, S276A, L277V, K281R, Q283T, S284V, T290P, S293V, V384P, S420T, W426F, E430G, S440E, F465A, L472Y, E488K, H489P, T508P, A509Y, V522I, G525N and A532K;
(j) A4G, K8I, A12P, D19P, Q25L, V36H, T39R, I47T, E48G, V49Q, Y56L, E58Q, M59W, S60A, V61N, L63M, M67L, T74K, N75G, R77V, M90V, A101T, A103H, I108N, N110T, R112D, L114I, N116H, S117Q, M118L, T124K, V128T, K130P, G132A, Q134P, V168M, T169K, H171Y, G175N, V182K, K190Q, T191Q, T214N, A222C, D234G, G246A, T251C, T252V, I257R, C258Q, L264I, Y266R, S276A, L277V, Q283T, T290S, S293V, G316A, H332N, A361S, V384P, M396N, S399K, N409K, L411T, S420T, W426F, E430G, S440E, S456A, I457V, Q460S, N463D, F465A, L472Y, E488K, H489P, V499I, T507S, T508P, A509H, V522I, G525T, L526P, L530I and A532R;
(k) A4G, K8I, D19P, I47T, E48G, Y56L, V61C, L63V, M67L, R77V, M90V, A101T, A103H, I108N, S117Q, M118L, K130P, V168M, H171Y, T251H, I257R, L264V, Y266K, S276A, L277V, K281R, Q283T, S284V, T290P, S293V, V384P, S420T, W426F, E430G, S440E, F465A, L472Y, E488K, H489P, T508P, A509Y, V522I and A532K;
(l) A4G, K8I, A12P, D19P, T39R, I47T, E48G, Y56L, M59W, V61C, L63V, M67L, R77V, M90V, A101V, A103T, I108T, N110T, N116H, S117Q, M118L, T124K, K130S, Q134P, V168M, H171Y, G175N, T191Q, T214N, R223S, T251H, I257Y, L264V, Y266K, S276A, Q283T, T290P, S293V, G316A, A361S, V384P, S399K, N409K, S420T, W426F, E430G, S440E, F465A, L472Y, E488K, H489P, V499I, T507S, T508P, A509H, V522I, G525N and A532K;
(m) A4G, K8I, A12P, D19P, T39R, I47T, E48G, V49Q, Y56L, M59W, S60A, V61C, L63V, M67L, T74K, R77V, M90V, A101V, A103H, I108T, N110T, N116H, S117Q, M118L, T124K, V128T, K130P, G132A, V168M, H171Y, G175N, T191Q, T214N, D234G, G246A, T251H, I257R, C258Q, L264V, Y266K, S276A, L277V, Q283T, T290P, S293V, G316A, H332N, A361S, V384P, S399K, N409K, S420T, W426F, E430G, S440E, S456A, I457V, Q460S, N463D, F465A, L472Y, E488K, H489P, V499I, T507S, T508P, A509Y, V522I, G525N, L526P, L530I and A532K; or
(n) A4G, K8I, A12P, D19P, T39R, I47T, E48G, Y56L, V61C, L63V, M67L, R77V, M90V, A101V, A103T, I108T, N110T, N116H, S117Q, M118L, T124K, K130P, Q134P, V168M, H171Y, G175N, T191Q, T214N, I257R, L264V, Y266K, S276A, K281R, Q283T, T290P, S293V, G316A, A361S, V384P, S399K, N409K, S420T, W426F, E430G, S440E, F465A, L472Y, E488K, H489P, V499I, T507S, T508P, A509H, V522I, G525N and A532K.

47. A mOrange variant of a wild-type mOrange from Discosoma sp. of SEQ ID NO: 62 comprising one or more mutations selected from N8D, N9P, R22H, S26C, F46T, A49M, K50R, F88L, D120G, E122C, S136P, D137N, A150P, S152T or S152C, A161T, K167T, T179R, S180C, A197N, I199F, G201D or G201N, K203R; D205Y, D205E or D205V; N210D, Q218E and G224A.

48. A mOrange variant of a wild-type mOrange from Discosoma sp. of SEQ ID NO: 62 comprising mutations:
(a) N8D, N9P, R22H, F46T, A49M, F88L, D120G, E122C, S136P, A150P, S152T, A161T, K167T, T179R, S180C, A197N, I199F, G201D, K203R, D205Y, N210D and G224A;
(b) N8D, N9P, R22H, S26C, F46T, A49M, K50R, F88L, D120G, E122C, S136P, D137N, A150P, S152T, A161T, K167T, R169A, T179R, S180C, A197N, I199F, G201D, K203R, D205Y, N210D, Q218E and G224A;
(c) N8D, R22H, F46T, F88L, E122C, A150P, S152T, A161T, T179R, S180C, I199F, G201D, K203R, D205Y and G224A;
(d) N8D, N9P, R22H, F46T, A49M, F88L, D120G, E122C, S136P, A150P, S152T, A161T, K167T, T179R, S180C, A197N, I199F, G201D, K203R, D205E, N210D and G224A;
(e) N8D, R22H, F46T, A49M, F88L, D120G, E122C, A150P, S152T, A161T, T179R, S180C, I199F, G201D, K203R, D205Y, N210D and G224A;
(f) N8D, R22H, F46T, F88L, A150P, S152C, A161T, T179R, S180C, I199F, G201N, K203R, D205Y and G224A; or
(g) N8D, R22H, F46T, F88L, A150P, S152C, A161T, T179R, S180C, I199F, G201N, D205V and G224A.

49. A sandercyanin fluorescent protein variant of a wild-type sandercyanin fluorescent protein from Sander vitreus of SEQ ID NO: 63 comprising one or more mutations selected from M1L, K4R, A12P, E15P, D16N, A19P, A205, A42I, V25T, D28E; K35T, K35P or K35Q; A42I, I114T, N115D, A118D, S119F, A121V, A122E, V127L or V127M; G143E, T144I, M145L, G151P, T154E or T154K; L156I, A162N and A163C.

50. A sandercyanin fluorescent protein variant of a wild-type sandercyanin fluorescent protein from Sander vitreus of SEQ ID NO: 63 comprising mutations:
(a) A12P, D16N, A205, V25T, D28E, S119F, A122E, V127L, G143E, T144I, M145L, G151P, T154E and A163C;
(b) A12P, D16N, A205, V25T, D28E, S119F, A122E, V127M, G143E, T144I, M145L, G151P, T154E and A163C;
(c) M1L, A12P, E15P, D16N, A19P, A205, V25T, D28E, K35T, A42I, I114T, N115D, A118D, S119F, A121V, A122E, V127M, G143E, T144I, M145L, G151P, T154K, L156I, A162N and A163C;
(d) M1L, A12P, E15P, D16N, A19P, A205, V25T, D28E, K35T, A42I, I114T, N115D, A118D, S119F, A121V, A122E, V127L, G143E, T144I, M145L, G151P, T154E, L156I, A162N and A163C;
(e) K4R, A12P, D16N, A205, V25T, D28E, K35T, A42I, N115D, A118D, S119F, A122E, V127M, G143E, T144I, M145L, G151P, T154K and A163C;
(f) A12P, D28E, K35P, S119F, A122E, V127M, G143E, M145L, G151P, T154E and A163C;
(g) K4R, A12P, D16N, A205, V25T, D28E, K35Q, A42I, N115D, A118D, S119F, A121V, A122E, V127L, G143E, T144I, M145L, G151P, T154K and A163C;
(h) A12P, D16N, A205, V25T, D28E, K35T, N115D, S119F, A122E, V127M, G143E, T1441, M145L, G151P, T154K and A163C;
(i) A12P, A205, D28E, S119F, A122E, V127M, G143E, T144I, M145L, G151P, T154E and A163C;
(j) A12P, D28E, S119F, A122E, V127M, G143E, M145L, G151P and T154E; or
(k) A12P, A205, D28E, K35P, S119F, A122E, V127M, G143E, T144I, M145L, G151P, T154E and A163C.

51. A GFP-like fluorescent chromoprotein FP538 variant of a wild-type GFP-like fluorescent chromoprotein FP538 from Zoanthus sp. of SEQ ID NO: 64 comprising one or more mutations selected from K9T, T30E, I34R or I34E; K39E, I49V, E50K, G94E, S96T, I106T, C107A, N108R, N108S or N108T; I125E or I125K; N127H, M129T, D134N, K139Q, M141R or M141K; T143V, T1431 or T143L; N144G, A147P, M153Y, K157N, K162R, S166T, L170K, V184T, S189K, S192K, E196G, Q201E, L205E, T220Y, T220Q, T220E or T220H, I224V, F226H and A229P.

52. A GFP-like fluorescent chromoprotein FP538 variant of a wild-type GFP-like fluorescent chromoprotein FP538 from Zoanthus sp. of SEQ ID NO: 64 comprising mutations:
(a) K9T, T30E, I34R, K39E, I49V, E50K, G94E, S96T, I106T, C107A, N108R, I125E, N127H, M129T, D134N, K139Q, M141R, T143V, N144G, A147P, M153Y, K157N, K162R, S166T, L170K, V184T, S189K, S192K, E196G, Q201E, L205E, T220Y, I224V, F226H and A229P;
(b) K9T, T30E, I34R, K39E, I49V, E50K, G94E, S96T, I106T, C107A, N108R, I125K, N127H, M129T, D134N, K139Q, M141R, T143V, N144G, A147P, M153Y, K157N, S166T, L170K, V184T, S189K, S192K, E196G, Q201E, L205E, T220Y, I224V, F226H and A229P;
(c) G94E, K139Q, T143V, A147P, M153Y, K162R and A229P;
(d) G94E, K139Q, T143I, A147P, M153Y and A229P;
(e) K9T, T30E, I34R, K39E, G94E, S96T, I106T, C107A, N108S, I125K, N127H, M129T, K139Q, M141K, T143V, N144G, A147P, M153Y, K157N, K162R, S166T, L170K, V184T, E196G, T220Q, I224V, F226H and A229P;
(f) K9T, T30E, I34R, K39E, G94E, S96T, I106T, C107A, N108S, I125K, N127H, M129T, K139Q, M141K, T143L, N144G, A147P, M153Y, K157N, S166T, L170K, V184T, E196G, T220H, I224V, F226H and A229P;
(g) T30E, I34E, K39E, G94E, N108S, N127H, M129T, K139Q, M141R, T143V, A147P, M153Y, K162R, E196G, T220E, F226H and A229P;
(h) I34E, G94E, N127H, K139Q, T143V, A147P, M153Y, K162R, T220E and A229P;
(i) K9T, T30E, I34E, K39E, G94E, S96T, I106T, N108S, I125K, N127H, M129T, K139Q, M141R, T143V, A147P, M153Y, K162R, V184T, E196G, T220E, F226H and A229P; or
(j) K9T, T30E, I34E, K39E, G94E, S96T, I106T, N108T, I125K, N127H, M129T, K139Q, M141K, T143V, A147P, M153Y, V184T, E196G, T220Q, F226H and A229P.

53. An mNeonGreen variant of a wild-type mNeonGreen from Branchiostoma lanceolatum of SEQ ID NO: 65 comprising one or more mutations selected from V52H, D53E, N65Y, E69M or E69Q, A110Q, S1311 or S131C, T139R, S143D, K152H, A158P, T164Q, S166K, A169G, W172P, S175E, K177M, T178L, T188D or T188E, K190T, T194K, G196K or G196E, N197D, S203C, T204Q, T208H, N218D and Y226F.

54. An mNeonGreen variant of a wild-type mNeonGreen from Branchiostoma lanceolatum of SEQ ID NO: 65 comprising mutations:
(a) V52H, D53E, E69M, T139R, K152H, S166K, W172P, K177M, T188D, G196K and 5203C;
(b) V52H, D53E, E69Q, S131 I, T139R, S143D, K152H, T164Q, S166K, A169G, W172P, K177M, T188D, K190T, T194K, G196E, N197D, 5203C, T208H, N218D and Y226F;
(c) S166K, K177M, G196E and 5203C;
(d) V52H, D53E, N65Y, E69Q, A110Q, S131I, T139R, S143D, K152H, A158P, T164Q, S166K, A169G, W172P, S175E, K177M, T178L, T188D, K190T, T194K, G196E, N197D, 5203C, T204Q, T208H, N218D and Y226F;
(e) E69M, T139R, S166K, K177M, T188D, G196E and 5203C;
(f) V52H, D53E, E69M, T139R, K152H, S166K, W172P, K177M, G196K and 5203C;
(g) V52H, D53E, N65Y, E69Q, A110Q, S131C, T139R, S143D, K152H, A158P, T164Q, S166K, A169G, W172P, K177M, T188E, K190T, T194K, G196E, N197D, 5203C, T204Q, T208H, N218D and Y226F;
(h) V52H, D53E, E69Q, S131 I, T139R, S143D, K152H, T164Q, S166K, A169G, W172P, S175E, K177M, T188D, K190T, T194K, G196E, N197D, 5203C, T208H, N218D and Y226F;
(i) V52H, D53E, E69Q, T139R, K152H, S166K, W172P, K177M, K190T, G196E, N197D, 5203C and Y226F;
(j) V52H, D53E, E69Q, S131 I, T139R, S143D, K152H, S166K, A169G, W172P, K177M, T188E, K190T, G196E, N197D, 5203C and Y226F; or
(k) E69M, T139R, S166K, K177M, G196E and 5203C.

55. A green fluorescent protein blFP-Y3 variant of a wild-type green fluorescent protein blFP-Y3 from Branchiostoma lanceolatum of SEQ ID NO: 66 comprising one or more mutations selected from M1A, AST, V18H, D19E, E35Q, S49N, A76K, K79C, S97I, S100A, N101H, Y102F, T105R, 5109N, K112I, I118H, S132K, W138P, T141E, S153G, T154E, T160K, G162K, T170E, N174T, N184D and K213W.

56. A green fluorescent protein blFP-Y3 variant of a wild-type green fluorescent protein blFP-Y3 from Branchiostoma lanceolatum of SEQ ID NO: 66 comprising mutations:
(a) E35Q, S97I, Y102F, I118H, W138P, T141E, T160K, G162K, N174T and K213W;
(b) D19E, E35Q, K79C, S97I, N101H, Y102F, I118H, W138P, T141E, S153G, T160K, G162K, T170E, N174T and K213W;
(c) E35Q, S97I, Y102F, I118H, W138P, T154E, T160K, G162K, T170E, N174T and K213;
(d) V18H, D19E, E35Q, S49N, A76K, K79C, S97I, N101H, Y102F, I118H, S132K, W138P, T141E, S153G, T160K, G162K, T170E, N174T and K213W;
(e) D19E, E35Q, K79C, S97I, N101H, Y102F, I118H, W138P, T141E, S153G, T154E, T160K, G162K, T170E, N174T and K213W;
(f) A5T, V18H, D19E, E35Q, S49N, A76K, K79C, S97I, N101H, Y102F, S109N, I118H, S132K, W138P, T141E, S153G, T160K, G162K, T170E, N174T, N184D and K213W;
(g) E35Q, S97I, Y102F, I118H, W138P, T160K, G162K, N174T and K213W; or
(h) M1A, A5T, V18H, D19E, E35Q, S49N, A76K, K79C, S97I, S100A, N101H, Y102F, T105R, S109N, K112I, I118H, S132K, W138P, T141E, S153G, T160K, G162K, T170E, N184D and K213W.

57. A far-red fluorescent protein eqFP650 variant of a wild-type far-red fluorescent protein eqFP650 from Entacmaea quadricolor of SEQ ID NO: 67 comprising one or more mutations selected from G13N, T19E, 520G, K25N, A33M, K34R, A51M, I85V, T86M, Y88F, L94C, T100I, N104D, L107F, K112E, N114R, S120P, A134P, A142R or A142C; S144G, R147E, H149R, Q151N or Q151D; V157K, Y161H, H163I, S165H, F184V, F186Y, R189H or R189Y; K190R, E196H, K199D, T201N and M208V.

58. A far-red fluorescent protein eqFP650 variant of a wild-type far-red fluorescent protein eqFP650 from Entacmaea quadricolor of SEQ ID NO: 67 comprising mutations:
(a) G13N, T19E, 520G, A33M, K34R, A51M, I85V, T86M, T100I, N104D, L107F, K112E, N114R, A134P, A142R, S144G, R147E, H149R, Q151N, V157K, Y161H, H163I, S165H, F184V, F186Y, R189H, K190R, E196H, K199D and M208V;
(b) G13N, T19E, 520G, K25N, A33M, K34R, A51M, I85V, T86M, Y88F, L94C, T100I, N104D, L107F, N114R, S120P, A134P, A142R, S144G, R147E, H149R, Q151N, V157K, Y161H, H163I, S165H, F184V, F186Y, R189H, K190R, E196H, K199D, T201N and M208V;
(c) G13N, T19E, S20G, A33M, K34R, A51M, I85V, T86M, L94C, T100I, N104D, L107F, N114R, A134P, A142R, S144G, R147E, H149R, Q151N, V157K, Y161H, H163I, S165H, F184V, F186Y, R189H, K190R, E196H, K199D and M208V;
(d) G13N, S20G, A33M, K34R, I85V, T86M, N104D, L107F, N114R, A134P, A142C, S144G, R147E, H149R, Q151D, Y161H, H163I, S165H, F184V, F186Y, K190R, E196H, K199D and M208V;
(e) G13N, S20G, A33M, K34R, I85V, T86M, N104D, L107F, N114R, A134P, A142C, S144G, R147E, H149R, Q151D, V157K, Y161H, H163I, S165H, F184V, F186Y, K190R, E196H, K199D and M208V;
(f) G13N, S20G, A33M, K34R, I85V, T86M, T100I, N104D, L107F, K112E, N114R, A134P, A142R, S144G, R147E, H149R, Q151N, V157K, Y161H, H163I, S165H, F184V, F186Y, R189Y, K190R, E196H, K199D and M208V;
(g) G13N, S20G, A33M, K34R, I85V, T86M, N104D, L107F, N114R, A134P, A142C, S144G, R147E, H149R, Q151D, V157K, Y161H, H163I, S165H, F184V, F186Y, R189Y, K190R, E196H, K199D and M208V;
(h) G13N, S20G, A33M, K34R, I85V, T86M, T100I, N104D, L107F, K112E, N114R, A134P, A142C, S144G, R147E, H149R, Q151D, V157K, Y161H, H163I, S165H, F184V, F186Y, R189Y, K190R, E196H, K199D and M208V; or
(i) G13N, T19E, S20G, K25N, A33M, K34R, A51M, I85V, T86M, Y88F, L94C, T100I, N104D, L107F, K112E, N114R, S120P, A134P, A142C, S144G, R147E, H149R, Q151N, V157K, Y161H, H163I, S165H, F184V, F186Y, R189H, K190R, E196H, K199D, T201N and M208V.

59. A fluorescent protein cyOFP variant of a wild-type fluorescent protein cyOFP from Escherichia coli of SEQ ID NO: 68 comprising one or more mutations selected from L8V, EllP, H14K, S15T, L19M, H32G, K37N, N45M, R46K, V50T, A58S, A63M, M65L, K71E, L106C, T1121 or T112V; L114I, A132P, L151M; A154R, A154K or A154N; K164M, V169K, N177H, K179R, T189K, N211D, V220W, V220H or V220Q and D227H.

60. A fluorescent protein cyOFP variant of a wild-type fluorescent protein cyOFP from Escherichia coli of SEQ ID NO: 68 comprising mutations:
(a) L8V, E11P, H14K, L19M, H32G, N45M, A63M, M65L, K71E, A132P, A154R, K164M, V169K, N177H and D227H;
(b) E11P, H14K, L19M, H32G, K37N, N45M, R46K, A63M, K71E, A132P, A154R, K164M, V169K, N177H, T189K, N211D, V220W and D227H;
(c) E11P, H14K, L19M, H32G, N45M, A63M, M65L, K71E, A154R, K164M, V169K and N177H;
(d) L8V, E11P, H14K, L19M, H32G, K37N, N45M, R46K, A63M, K71E, T112I, A132P, A154K, K164M, V169K, N177H, T189K, N211D and D227H;
(e) L8V, E11P, H14K, L19M, H32G, N45M, A63M, K71E, T112I, A154R, K164M, V169K and N177H;
(f) E11P, H14K, L19M, H32G, K37N, N45M, R46K, V50T, A58S, A63M, K71E, L106C, T112V, L114I, A132P, L151M, A154R, K164M, V169K, N177H, T189K, N211D, V220H and D227H;
(g) E11P, H14K, L19M, H32G, K37N, N45M, R46K, V50T, A58S, A63M, M65L, K71E, L106C, T112I, A132P, L151M, A154N, K164M, V169K, N177H, T189K, N211D, V220Q and D227H;
(h) E11P, H14K, L19M, N45M, K71E, A154R, K164M, V169K, N177H and K179R;
(i) E11P, H14K, S15T, L19M, H32G, K37N, N45M, R46K, A63M, K71E, T112I, A132P, A154K, K164M, V169K, N177H, T189K, N211D and D227H;
(j) H14K, L19M, N45M, K71E, A154R, K164M and V169K;
(k) E11P, H14K, L19M, H32G, N45M, A63M, K71E, A132P, A154R, K164M, V169K, N177H and D227H;
(l) E11P, H14K, L19M, N45M, K71E, A154R, K164M, V169K and N177H; or
(m) E11P, H14K, L19M, H32G, K37N, N45M, R46K, A63M, K71E, T112I, A132P, A154R, K164M, V169K, N177H, T189K, N211D and D227H.


SEQUENCE LISTING (68 sequences)

SEQ ID NO: 1-38 - skipped (38 entries)

SEQ ID NO: 39 - protein, 453 aa, Vibrio fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 40 - protein, 358 aa, Burkholderia glumae
MVRSMRSRVAARAVAWALAVMPLAGAAGLTMAASPAAVAADTYAATRYPVILVHGLAGTDKFANVVDYWYGIQSDLQSHGAKVYVANLSGFQSDDGPNGRGEQLLAYVKQVLAATGATKVNLIGHSQGGLTSRYVAAVAPQLVASVTTIGTPHRGSEFADFVQDVLKTDPTGLSSTVIAAFVNVFGTLVSSSHNTDQDALAALRTLTTAQTATYNRNFPSAGLGAPGSCQTGAATETVGGSQHLLYSWGGTAIQPTSTVLGVTGATDTSTGTLDVANVTDPSTLALLATGAVMINRASGQNDGLVSRCSSLFGQVISTSYHWNHLDEINQLLGVRGANAEDPVAVIRTHVNRLKLQGV

SEQ ID NO: 41 - protein, 254 aa, Rhizobium radiobacter
MSTAIVTNVKHFGGMGSALRLSEAGHTVACHDESFKQKDELEAFAETYPQLKPMSEQEPAELIEAVTSAYGQVDVLVSNDIFAPEFQPIDKYAVEDYRGAVEALQIRPFALVNAVASQMKKRKSGHIIFITSATPFGPWKELSTYTSARAGACTLANALSKELGEYNIPVFAIGPNYLHSEDSPYFYPTEPWKTNPEHVAHVKKVTALQRLGTQKELGELVAFLASGSCDYLTGQVFWLAGGFPMIERWPGMPE

SEQ ID NO: 42 - protein, 459 aa, Chromobacterium violaceum
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 43 - protein, 480 aa, Clostridium botulinum
MKKNLNKIATIILLVFSMTLTNFSMIVRAAEPKAQGTQKVESSTTKKEVKDAEETIKIPTLEDIDNLIDSAEEVKSEEDINKMPPLKFPVEFPEVNTRSIIGGNNYPIVLVHGFMGFGRDELLGYKYWGGVVDLQEKLNASGHETYTATVGPVSSNWDRACELYAYIVGGTVDYGEAHAKKFKHNRYGRTYPGIYKNISNENKIHLIGHSMGGQTIRTLTQLLSEGSEEEINCGQENISPLFEGGKHWIHSVSTISTPNDGTTLSDLMPAKDLISYTFGVLGTITGKNKLFSSIYDLKLDQWGLKKQNGESQRDYIERVLDSNIWNSTKDIATYDLSTEGAQELNTWVKAQPDVYYFSWTTQATKESILTGHSVAQIGPMNPIFYPTANLMGRYSRNQKDLPIIDKKWFPNDGVVNCISQDGPKLGSNDVIEQYNGGVKIGQWNAMPRIINTDHMDIVGTFGNVKDWYMDYASFLSNLSR

SEQ ID NO: 44 - protein, 337 aa, Geobacillus stearothermophilus
MKAAVVEQFKKPLQVKEVEKPKISYGEVLVRIKACGVCHTDLHAAHGDWPVKPKLPLIPGHEGVGVIEEVGPGVTHLKVGDRVGIPWLYSACGHCDYCLSGQETLCERQQNAGYSVDGGYAEYCRAAADYVVKIPDNLSFEEAAPIFCAGVTTYKALKVTGAKPGEWVAIYGIGGLGHVAVQYAKAMGLNVVAVDLGDEKLELAKQLGADLVVNPKHDDAAQWIKEKVGGVHATVVTAVSKAAFESAYKSIRRGGACVLVGLPPEEIPIPIFDTVLNGVKIIGSIVGTRKDLQEALQFAAEGKVKTIVEVQPLENINDVFDRMLKGQINGRVVLKVD

SEQ ID NO: 45 - protein, 434 aa, Yersinia mollaretii
MRLTALGLMLGSFAISAAPVAAPVTGYTLERVVILSRHGVRSPTKQTELMNDVTPDKWPQWPVPAGYLTPRGAQLVTLMGGFYGDYFRNQGLLPAGCPADGTLYAQADIDQRTRLTGQAFLDGIAPGCGLKVHYQADLKKVDPLFHPVEAGVCQLDSTQTHRAIEAQLGAPLSELSQRYAKPFAQMGEILNFTASPYCKSLQQQGKSCDFATFAANEVKVNQQGTKVSLSGPLALSSTLGEIFLLQNSQGMPDVAWHRLSGAENWVSLLSLHNAQFDLMAKTPYIARHKGTPLLQQIVTALVLQRKGQGQTLPLSEQTKLLFLGGHDTNIANIGGMLGANWQLPQQPDNTPPGGGLVFELWQNPDNHQQYVAVKMFYQTMDQLRNSEKLDLKSHPAGIVPIEIEGCENIGTDKLCQLDTFQKRVAQVIEPACHI

SEQ ID NO: 46 - protein, 376 aa, Neosartorya fumigate
MREEPSSAQLFKPLKVGRCHLQHRMIMAPTTRFRADGQGVPLPFVQEYYGQRASVPGTLLITEATDITPKAMGYKHVPGIWSEPQREAWREIVSRVHSKKCFIFCQLWATGRAADPDVLADMKDLISSSAVPVEEKGPLPRALTEDEIQQCIADFAQAARNAINAGFDGVEIHGANGYLIDQFTQKSCNHRQDRWGGSIENRARFAVEVTRAVIEAVGADRVGVKLSPYSQYLGMGTMDELVPQFEYLIAQMRRLDVAYLHLANSRWLDEEKPHPDPNHEVFVRVWGQSSPILLAGGYDAASAEKVTEQMAAATYTNVAIAFGRYFISTPDLPFRVMAGIQLQKYDRASFYSTLSREGYLDYPFSAEYMALHNFPV

SEQ ID NO: 47 - protein, 322 aa, Archaeoglobus fulgidus
METLILTQEEVESLISMDEAMNAVEEAFRLYALGKAQMPPKVYLEFEKGDLRAMPAHLMGYAGLKWVNSHPGNPDKGLPTVMALMILNSPETGFPLAVMDATYTTSLRTGAAGGIAAKYLARKNSSVFGFIGCGTQAYFQLEALRRVFDIGEVKAYDVREKAAKKFVSYCEDRGISASVQPAEEASRCDVLVTTTPSRKPVVKAEWVEEGTHINAIGADGPGKQELDVEILKKAKIVVDDLEQAKHGGEINVAVSKGVIGVEDVHATIGEVIAGLKDGRESDEEITIFDSTGLAIQDVAVAKVVYENALSKNVGSKIKFFRI

SEQ ID NO: 48 - protein, 249 aa, Shimwellia blattae
MKKRVLAVCFAALFSSQALALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPAFQQQLQKAKAEFAQHQKK

SEQ ID NO: 49 - protein, 249 aa, Morganella morganii
MKKNIIAGCLFSLFSLSALAAIPAGNDATTKPDLYYLKNEQAIDSLKLLPPPPEVGSIQFLNDQAMYEKGRMLRNTERGKQAQADADLAAGGVATAFSGAFGYPITEKDSPELYKLLTNMIEDAGDLATRSAKEHYMRIRPFAFYGTETCNTKDQKKLSTNGSYPSGHTSIGWATALVLAEVNPANQDAILERGYQLGQSRVICGYHWQSDVDAARIVGSAAVATLHSDPAFQAQLAKAKQEFAQKSQK

SEQ ID NO: 50 - protein, 263 aa, Prevotella intermedia
MTKKTLLVGLLAMFSVTTFAQNAAKKIKDARTNPDLYYLQDGQQTSSLELLPTPPQPGSIQFLYDEAQYQWGKMQRNTPRGDQAVADARVGGDGVPNAFSEAFGIKISKETTPEIYKLVLNMREDAGDLATRSAKDHYMRVRPFAFYNEMTCNPEQQQELSTNGSYPSGHTAIGWATALVLSEINIDRQNEILERGYQMGQSRVICGYHWQSDVDAARVVSAAVVARLHADPAFQAQLEKAKKEFAKLQKEGKVAKSTFKAAN

SEQ ID NO: 51 - protein, 459 aa, Neosartorya fumigate
MKAANASSAEAYRVLSRAFRFDNEDQKLWWHSTAPMFAKMLETANYTTPCQYQYLITYKECVIPSLGCYPTNSAPRWLSILTRYGTPFELSLNCSNSIVRYTFEPINQHTGTDKDPFNTHAIWESLQHLLPLEKSIDLEWFRHFKHDLTLNSEESAFLAHNDRLVGGTIRTQNKLALDLKDGRFALKTYIYPALKAVVTGKTIHELVFGSVRRLAVREPRILPPLNMLEEYIRSRGSKSTASPRLVSCDLTSPAKSRIKIYLLEQMVSLEAMEDLWTLGGRRRDASTLEGLSLVRELWDLIQLSPGLKSYPAPYLPLGVIPDERLPLMANFTLHQNDPVPEPQVYFTTFGMNDMAVADALTTFFERRGWSEMARTYETTLKSYYPHADHDKLNYLHAYISFSYRDRTPYLSVYLQSFETGDWAVANLSESKVKCQDAACQPTSLPPDLSKTGVYYSGLH

SEQ ID NO: 52 - protein, 339 aa, Geobacillus stearothermophilus
MKAAVVNEFKKALEIKEVERPKLEEGEVLVKIEACGVCHTDLHAAHGDWPIKPKLPLIPGHEGVGIVVEVAKGVKSIKVGDRVGIPWLYSACGECEYCLTGQETLCPHQLNGGYSVDGGYAEYCKAPADYVAKIPDNLDPVEVAPILCAGVTTYKALKVSGARPGEWVAIYGIGGLGHIALQYAKAMGLNVVAVDISDEKSKLAKDLGADIAINGLKEDPVKAIHDQVGGVHAAISVAVNKKAFEQAYQSVKRGGTLVVVGLPNADLPIPIFDTVLNGVSVKGSIVGTRKDMQEALDFAARGKVRPIVETAELEEINEVFERMEKGKINGRIVLKLKED

SEQ ID NO: 53 - protein, 369 aa, Rhodococcus jostii
MKTKAAALTGVHRDWEVLEFDLDDPGPGEVLVQMKVAGLCHSDKHAKFGGMTYPLVGGHEGAGVVEAVGDGVSRVSIGDHVAVSWIPSCGTCRWCASGQSNLCDLGANMVTGELANGGFRFHRDGTDIGAMAATGTFSQFSVVDQRSVVKVDPTIPFEWVSLVTCGVATGWGSVVNAGEVKAGDTVAVYGCGGIGSNAVRAAVGANAGLVAAIDPVESKLEFAKQSGADFVYTSAEEAHADLWERTYGVGADVTVVTVGVVTSEVVRAAFELTRKGGTVVLTGVSDDVMENTIELPGGILTLFQKRIVGSLYGHCNPLVDIPRLLRMAQAGKLVLDDLVTRRYALEDINQGFDDMLAGHNIRGLIVHDH

SEQ ID NO: 54 - protein, 249 aa, Shigella flexneri
MKRQLFTLSIVGVFSLNTFASFPPGNDVTTKPDLYYLTNDNAIDSLALLPPPPQIGSIAFLNDQAMYEKGRLLRNTERGKLAAEDANLSSGGVANVFSAAFGSPITAKDSPELHKLLTNMIEDAGDLATRSAKEYYMRIRPFAFYGVSTCNTKEQDTLSRNGSYPSGHTSIGWATALVLSEINPARQDTILKRGYELGDSRVICGYHWQSDVDAARIVGSAIVATLHSNPVFQAQLQKAKDEFANNQKK

SEQ ID NO: 55 - protein, 335 aa, Aromatoleum aromaticum
MKAITLRSPGGLDRLELVELPDPGQPGPGEIRVRIHASSLNFHDLGVVSGAMPVADNRIPMSDGAGVVEAIGEGVDEFAVGDAVVSTFFPHWLDGEAPIADFSTTPGDGVDGYACELVVRPATAFTHSPRGYSHAQAATLTTAGLTAWRALIYNGGLKAGDSVLVLGTGGVSIFALQFAKAMGATVIATSSSDEKLEHARALGASHTINYCSQAQWASRVLDFTGGCGVDHVIEVGGPGTLAQSIQACRIGGHIALIGVLTGGAGVVPTAALMKRQQRLQGLIVGNRRQQNELVRAIEATGIFPVIDRSFTLETIADAFRLQKAGGHFGKISLEF

SEQ ID NO: 56 - protein, 201 aa, Pseudomonas pseudoalcaligenes
MRAWWLSGALALMFWAQGAVAGTLLVVGDSISAAFGLDSRQGWVALLEKRLSEEGFEHSVVNASISGDTSAGGAARLSALLAEHKPELVIIELGGNDGLRGQPPAQLQQNLASMVEQSQQAGAKVLLLGMKLPPNYGVRYTTAFAQVFTDLAEQKQVSLVPFFLEGVGGVPGMMQADGIHPAEAAQEILLDNVWPTLKPML

SEQ ID NO: 57 - protein, 459 aa, Escherichia coli
MNRLPSSASALACSAHALNLIEKRTLDHEEMKALNREVIEYFKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRTALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 58 - protein, 226 aa, Lobophyllia hemprichii
MSAIKPDMKINLRMEGNVNGHHFVIDGDGTGKPFEGKQSMDLEVKEGGPLPFAFDILTTAFHYGNRVFAEYPDHIQDYFKQSFPKGYSWERSLTFEDGGICIARNDITMEGDTFYNKVRFHGVNFPANGPVMQKKTLKWEPSTEKMYVRDGVLTGDITMALLLEGNAHYRCDFRTTYKAKEKGVKLPGYHFVDHCIEILSHDKDYNKVKLYEHAVAHSGLPDNARR

SEQ ID NO: 59 - protein, 225 aa, Discosoma sp.
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 60 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 61 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 62 - protein, 236 aa, Discosoma sp.
MVSKGEENNMAIIKEFMRFKVRMEGSVNGHEFEIEGEGEGRPYEGFQTAKLKVTKGGPLPFAWDILSPQFGYGSKAYVKHPADIPDYFKLSFPEGFKWERVMNFEDGGVVTVTQDSSLQDGEFIYKVKLRGTNFPSDGPVMQKKTMGWEASSERMYPEDGALKGEIKMRLKLKDGGHYTSEVKTTYKAKKPVQLPGAYIVGIKLDITSHNEDYTIVEQYERAEGRHSTGGMDELYK

SEQ ID NO: 63 - protein, 183 aa, Sander vitreus
MFIKPGRCPKPAVQEDFDAARYLGVWYDIQRLPNKFQKGECATATYSLSPGVGFSVFNRERLANGTIKSVIGSAIAEDPCEPAKLQFFHENAAPVPYWVLSTDYDNYALVYSCINLGASHAAYASIVSRQPTLPEETIKKLQGTMSSFGVGVDTLLTTNQDAAYCSAMNQKLAAALEHHHHHH

SEQ ID NO: 64 - protein, 231 aa, Zoanthus sp.
MAHSKHGLKEEMTMKYHMEGCVNGHKFVITGEGIGYPFKGKQTINLCVIEGGPLPFSEDILSAGFKYGDRIFTEYPQDIVDYFKNSCPAGYTWGRSFLFEDGAVCICNVDITVSVKENCIYHKSIFNGMNFPADGPVMKKMTTNWEASCEKIMPVPKQGILKGDVSMYLLLKDGGRYRCQFDTVYKAKSVPSKMPEWHFIQHKLLREDRSDAKNQKWQLTEHAIAFPSALA

SEQ ID NO: 65 - protein, 260 aa, Branchiostoma lanceolatum
MASMTGGQQMGRDLYDDDDKDPLEMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 66 - protein, 219 aa, Branchiostoma lanceolatum
MSLPATHELHIFGSFNGVDFDMVGRGTGNPNDGYEELNLKSTKGALQFSPWILVPQIGYGFHQYLPFPDGMSPFQAAMKDGSGYQVHRTMQFEDGASLTSNYRYTYEGSHIKGEFQVIGTGFPADGPVMTNSLTAADWCVTKMLYPNDKTIISTFDWTYTTGSGKRYQSTVRTNYTFAKPMAANILKNQPMFVFRKTELKHSKTELNFKEWQKAFTDVM

SEQ ID NO: 67 - protein, 223 aa, Entacmaea quadricolor
MHMKLYMEGTVNGHHFKCTSEGEGKPYEGTQTAKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRKLERIKEADKETYVEQHEMAVARYCDLPSKLGHS

SEQ ID NO: 68 - protein, 244 aa, Escherichia coli
MVSKGEELIKENMHSKLYLEGSVNGHQFKCTHEGEGKPYEGTQTNRIKVVEGGPLPFAFDILATMFMYGSKTFIKYPKGLPDYFKQSFPEGFTWERVMVFEDGGVLTATQDTSLQDGCLIYNVKLRGVNFPANGPVMQKKTLGWEPSTETLYPADGGLEGRCDKALKLVGGGHLHCNFKTTYRSKKPATNLKMPGVHYVDRRLERIKEADNETYVEQHEVAVARYCDLPSKLGHKLNGMDELYK